(19) United States
(12) Patent Application Publication
(10) Pub. No.: US 2021/0191264 A1
(43) Pub. Date: Jun. 24, 2021

(54) NEGATIVE PHOTOSENSITIVE RESIN COMPOSITION, CURED FILM, AND ORGANIC EL DISPLAY AND MANUFACTURING METHOD THEREFOR

(71) Applicant: TORAY INDUSTRIES, INC., Tokyo (JP)

(72) Inventors: Yugo TANIGAKI, Shiga (JP); Kazuto MIYOSHI, Shiga (JP)

(73) Assignee: TORAY INDUSTRIES, INC., Tokyo (JP)

(21) Appl. No.: 16/759,140

(22) PCT Filed: Oct. 26, 2018
(86) PCT No.: PCT/JP2018/039958

§ 371 Date: Apr. 24, 2020

(30) Foreign Application Priority Data
Oct. 31, 2017 (JP) 2017-211395

(51) Int. Cl. G03F 7/031; C08G 73/10; G03F 7/038; G03F 7/037; C09B 67/22; H01L 27/32; H01L 51/56; H01L 51/52

(52) U.S. Cl. CPC G03F 7/031; C08G 73/1039; G03F 7/0382; G03F 7/037; G03F 7/0387; G02F 1/133512; H01L 27/3246; H01L 51/56; H01L 51/5284; H01L 27/3258; C09B 67/0041

(57) ABSTRACT

An object of the invention is to provide a cured film which is high in sensitivity, capable of forming a pattern in a low-taper shape after development, capable of the change in pattern opening width between before and after thermal curing, an excellent in light-blocking property, and a negative photosensitive resin composition that forms the film. The negative photosensitive resin composition contains an (A) alkali-soluble resin, a (C1) photo initiator, and a (Da) black colorant, where the (A) alkali-soluble resin contains a (A1) first resin including one or more selected from the group consisting of a (A1-1) polyimide, a (A1-2) polyimide precursor, a (A1-3) polybenzoxazole, and a (A1-4) polybenzoxazole precursor, and has a structural unit having a fluorine atom at a specific ratio, and the (C1) photo initiator contains an (C1-1) oxime ester-based photo initiator that has a specific structure.

### TECHNICAL FIELD

The present invention relates to a negative photosensitive resin composition, a cured film, and an organic EL display and a method for manufacturing an organic EL display.

### BACKGROUND ART

In recent years, many products that use organic electroluminescence (hereinafter, “EL”) displays have been developed in display devices including thin displays, such as smartphones, tablet PCs, and televisions.

In general, an organic EL display has a transparent electrode such as an indium tin oxide (hereinafter referred to as an “ITO”) on the light-extraction side of a light-emitting element, and a metal electrode such as an alloy of magnesium and silver on the side of the light-emitting element, from which no light is extracted. In addition, in order to define the pixels of the light-emitting element, an insulation layer referred to as a pixel defining layer is formed between the transparent electrode and the metal electrode. After the pixel defining layer is formed, a light-emitting material is deposited by evaporation through an evaporation mask in a region corresponding to the pixel region, where the pixel defining layer has an opening to expose the underlying transparent electrode or metal electrode, thereby forming a light-emitting layer. The transparent electrode and metal electrode are commonly formed by sputtering, but the pixel defining layer requires a low-taper pattern shape in order to prevent disconnection of the formed transparent electrode or metal electrode.

Furthermore, the organic EL display has thin-film-transistors (hereinafter, “TFTs”) for controlling the light-emitting element, which include a driving TFT, a switching TFT, and the like formed. In general, these TFTs are formed as laminated structures located further below the transparent electrode or the metal electrode, which serves as a base for the pixel defining layer mentioned above. The level differences due to the TFTs and a TFT array with a metal wiring or the like formed for connecting the TFTs to each other deteriorate uniformity in the subsequent formation of transparent electrodes, metal electrodes, pixel defining layers, and light-emitting layers, thereby causing the display characteristics and reliability of the organic EL display to be deteriorated. For that reason, after forming the TFT array, it is common to form a TFT planarization layer and/or a TFT protective layer for reducing or smoothing the level difference due to the TFT array.

Organic EL displays have a self-light-emitting element that emits light with the use of energy generated by recombination of electrons injected from a cathode and holes injected from an anode. Thus, the presence of a substance which inhibits the movement of electrons or holes, a substance that forms an energy level which inhibits recombination of electrons and holes, or the like, makes influences such as the decreased light emission efficiency of the light-emitting element or the deactivation of the light-emitting material, thus leading to the decreased lifetime of the light-emitting element. Since the pixel defining layer is formed at a position adjacent to the light-emitting element, degassing and ionic component outflow from the pixel defining layer can contribute to the decreased lifetime of the organic EL display. For that reason, high heat resistance is required for the pixel defining layer. As photosensitive resin compositions with high heat resistance, negative photosensitive resin compositions are known which include a high heat-resistance polyimide and an oxime ester-based photo initiator (for example, see Patent Document 1).

In addition, since the organic EL display has the self-light-emitting element, incident external light such as sunlight outdoors decreases the visibility and contrast due to reflection of the external light. Thus, a technique for reducing external light reflection is required.

As a technique for blocking external light and then reducing external light reflection, a photosensitive resin composition containing an alkali-soluble polyimide and a colorant is known (for example, see Patent Document 2). More specifically, there is a method of reducing external light reflection by forming a pixel defining layer with high heat resistance and light-blocking property with the use of a photosensitive resin composition containing a polyimide and a colorant such as a pigment.

### PRIOR ART DOCUMENTS

### Patent Documents

Patent Document 1: Japanese Patent Laid-open Publication No. 2016-191905

Patent Document 2: International Publication No. 2016/158672

### SUMMARY OF THE INVENTION

### Problems to be Solved by the Invention

From the viewpoint of improving the reliability of organic EL displays, in addition to the requirement of high heat resistance for the pixel defining layer adjacent to the light-emitting element, high heat resistance is also required for the TFT planarization layer and the TFT protective layer, because the layers are also formed at positions close to the light-emitting layer with the pixel defining layer interposed therebetween. In the case of containing a colorant such as a pigment in order to impart a light-blocking property to the photosensitive resin composition, however, ultraviolet rays and the like during pattern exposure are also blocked as the content of the colorant is increased, thus decreasing sensitivity for the exposure. Accordingly, conventionally known photosensitive resin compositions containing a colorant all have insufficient characteristics for use as a material for forming pixel defining layers, TFT planarization layers, or TFT protective layers of organic EL displays. Specifically, any of the sensitivity, light-blocking property, or patternability for low-taper shapes has been insufficient.

For example, in the case of improving the light-blocking property of the photosensitive resin composition, the deep part of the film is insufficiently cured during pattern exposure, and the deep part of the film is side-etched during development. For that reason, an inverse tapered shape is obtained after the development, which becomes an obstructive factor against the pattern formation in a low-taper shape. On the other hand, sufficient curing down to the deep part of the film, it is necessary to increase the exposure energy for pattern exposure, thereby promoting UV curing. The increased exposure energy makes, however, the film excessively crosslinked during the UV curing, thereby decreasing the reflow property for thermal curing, and thus forming a pattern in a high-taper shape. Accordingly, for example, there has been a problem that the photosensitive resin composition containing an alkali-soluble polyimide and a colorant such as a pigment, described in Patent Document 2, has difficulty in combining characteristics such as sensitivity, light-blocking property, and pattern formation in a low-taper shape.

Furthermore, in the case of forming a pattern in a high-taper shape after development and forming a pattern in a low-taper shape by reflow during thermal curing, pattern skirt reflow also caused during the thermal curing. For that reason, the pattern opening width after the thermal curing is smaller as compared with the pattern opening width after development, thus causing an error in the pixel design or the like for a display device such as an organic EL display. In addition, the variation in pattern opening width due to reflow during the thermal curing causes a decrease in panel manufacturing yield. Thus, there has been a problem that it is difficult to achieve a balance between the pattern formation in a low-taper shape and the suppression of the change in pattern opening width between before and after thermal. In order to achieve the balance between the both characteristics, it is necessary to form a pattern in a low-taper shape after development, suppress reflow during thermal curing, and suppress the change in pattern opening width.

The present invention has been achieved in view of the foregoing, and an object of the invention is to provide a negative photosensitive resin composition capable of achieving a cured film which is high in sensitivity, capable of forming a pattern in a low-taper shape after development, capable of suppressing the change in pattern opening width between before and after thermal curing, and excellent in light-blocking property.

In addition, a further object of the present invention is to provide a cured film which is high in sensitivity, capable of forming a pattern in a low-taper shape after development, capable of suppressing the change in pattern opening width between before and after thermal curing, and excellent in light-blocking property, and an organic EL display.

### Solutions to the Problems

In order to solve the above-described problems and achieve the above-mentioned objects, a negative photosensitive resin composition according to an aspect of the present invention is a negative photosensitive resin composition containing an (A) alkali-soluble resin, a (C1) photo initiator, and a (Da) black colorant, where the (A) alkali-soluble resin contains a (A1) first resin including one or more selected from the group consisting of a (A1-1) polyimide, a (A1-2) polyimide precursor, a (A1-3) polybenzoxazole, and a (A1-4) polybenzoxazole precursor, one or more selected from the group consisting of the (A1-1) polyimide, the (A1-2) polyimide precursor, the (A1-3) polybenzoxazole, and the (A1-4) polybenzoxazole precursor have a structural unit having a fluorine atom at 10 to 100 mol % of all of structural units, the (C1) photo initiator contains an (C1-1) oxime ester-based photo initiator, and the (C1-1) oxime ester-based photo initiator has one or more structures selected from the group consisting of (I), (II), and (III).

(I) One or more structures selected from the group consisting of a naphthalenecarbonyl structure, a trimethylbenzoyl structure, a thiophenecarbonyl structure, and a furancarbonyl structure

(II) a nitro group, a carbazole structure, and a group represented by the general formula (11)

(III) a nitro group and one or more structures selected from the group consisting of a fluorene structure, a dibenzofuran structure, a dibenzothiophene structure, a naphthalene structure, a diphenylmethane structure, a diphenylamine structure, a diphenyl ether structure, and a diphenyl sulfide structure

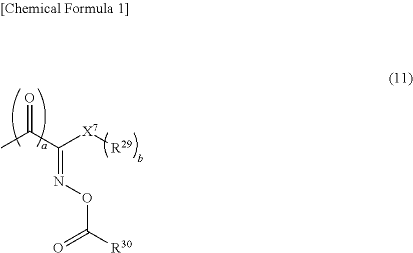

(In the general formula (11), X⁷ represents a direct bond, an alkylene group having 1 to 10 carbon atoms, a cycloalkylene group having 4 to 10 carbon atoms, or an arylene group having 6 to 15 carbon atoms. In a case where X⁷ represents a direct bond, an alkylene group having 1 to 10 carbon atoms, or a cycloalkylene group having 4 to 10 carbon atoms, R²⁹ represents hydrogen, an alkyl group having 1 to 10 carbon atoms, or a cycloalkyl group having 4 to 10 carbon atoms, an alkoxy group having 1 to 10 carbon atoms, a haloalkyl group having 1 to 10 carbon atoms, a haloalkoxy group having 1 to 10 carbon atoms, an acyl group having 2 to 10 carbon atoms, or a nitro group. In a case where X⁷ represents an arylene group having 6 to 15 carbon atoms, R²⁹ represents hydrogen, an alkyl group having 1 to 10 carbon atoms, a cycloalkyl group having 4 to 10 carbon atoms, an aryl group having 6 to 15 carbon atoms, a haloalkyl group having 1 to 10 carbon atoms, a haloalkoxy group having 1 to 10 carbon atoms, a heterocyclic group having 4 to 10 carbon atoms, a heterocyclic oxy group having 4 to 10 carbon atoms, an acyl group having 2 to 10 carbon atoms, or a nitro group. R³⁰ represents hydrogen, an alkyl group having 1 to 10 carbon atoms, a cycloalkyl group having 4 to 10 carbon atoms, or an aryl group having 6 to 15 carbon atoms. a represents 0 or 1, and b represents an integer of 0 to 10.)

A cured film according to one embodiment of the present invention is obtained by curing the negative photosensitive resin composition according to the invention mentioned above.

In an organic EL display according to an aspect of the present invention, the optical density of the cured film according to the above-mentioned invention per film thickness of 1 μm is 0.3 to 5.0, and the cured film is included as one or more selected from the group consisting of a pixel defining layer, an electrode insulation layer, a wiring insulation layer, an interlayer insulation layer, a TFT planarization layer, an electrode planarization layer, a wiring planarization layer, a TFT protective layer, an electrode protective layer, a wiring protective layer, and a gate insulation layer.

A method for manufacturing an organic EL display according to an aspect of the present invention is a method for manufacturing an organic EL display for the manufacture of the organic EL display according to the above-mentioned invention, the method includes: a step of forming, on a substrate, a coating film of the negative photosensitive resin composition according to the above-mentioned invention; a step of irradiating the coating film of the negative photosensitive resin composition with an active actinic ray through a photomask; a step of developing with an alkaline solution to form a pattern of the negative photosensitive resin composition; and a step of heating the pattern to obtain a cured pattern of the negative photosensitive resin composition.

### Effects of the Invention

The negative photosensitive resin composition according to the present invention makes it possible to achieve a cured film which is high in sensitivity, capable of forming a pattern in a low-taper shape after development, capable of suppressing the change in pattern opening width between before and after thermal curing, and excellent in light-blocking property.

Furthermore, the cured film, and the organic EL display and the manufacturing method therefor according to the present invention make it possible to obtain a cured film which is high in sensitivity, capable of forming a pattern in a low-taper shape after development, capable of suppressing the change in pattern opening width between before and after thermal curing, and excellent in light-blocking property, and makes it possible to obtain an organic EL display including the cured film.

### BRIEF DESCRIPTION OF THE DRAWINGS

FIG. 1 is a process diagram illustrating, in schematic cross-sectional views, a manufacturing process of Step 1 to Step 7 in an organic EL display that uses a cured film of a negative photosensitive resin composition according to the present invention.

FIG. 2 is a process diagram illustrating, in schematic cross-sectional views, a manufacturing process of Step 1 to Step 13 in a liquid crystal display that uses a cured film of a negative photosensitive resin composition according to the present invention.

FIG. 3 is a cross-sectional view illustrating a cross section example of a cured pattern with a step shape.

FIG. 4 is a schematic view illustrating, in plan views, a manufacturing process of Step 1 to Step 4 for a substrate of an organic EL display for use in the evaluation of light-emitting characteristics.

FIG. 5 is a schematic view illustrating a schematic cross section of an organic EL display without any polarizing layer.

FIG. 6 is a schematic view illustrating the arrangement and dimensions of a light-transmitting portion, a light-blocking portion, and a partial light-transmitting portion in a halftone photomask for use in halftone characteristic evaluation.

### EMBODIMENTS OF THE INVENTION

A negative photosensitive resin composition according to the present invention is a negative photosensitive resin composition containing an (A) alkali-soluble resin, a (C1) photo initiator, and a (Da) black colorant, where the (A) alkali-soluble resin contains a (A1) first resin including one or more selected from the group consisting of a (A1-1) polyimide, a (A1-2) polyimide precursor, a (A1-3) polybenzoxazole, and a (A1-4) polybenzoxazole precursor, one or more selected from the group consisting of the (A1-1) polyimide, the (A1-2) polyimide precursor, the (A1-3) polybenzoxazole, and the (A1-4) polybenzoxazole precursor have a structural unit having a fluorine atom at 10 to 100 mol % of all of structural units, the (C1) photo initiator contains an (C1-1) oxime ester-based photo initiator, and the (C1-1) oxime ester-based photo initiator has one or more structures selected from the group consisting of (I), (II), and (III).

(I) one or more structures selected from the group consisting of a naphthalenecarbonyl structure, a trimethylbenzoyl structure, a thiophenecarbonyl structure, and a furancarbonyl structure;

(II) a nitro group, a carbazole structure, and a group represented by the general formula (11);

(III) a nitro group and one or more structures selected from the group consisting of a fluorene structure, a dibenzofuran structure, a dibenzothiophene structure, a naphthalene structure, a diphenylmethane structure, a diphenylamine structure, a diphenyl ether structure, and a diphenyl sulfide structure.

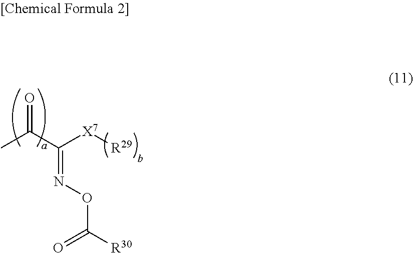

(In the general formula (11), X⁷ represents a direct bond, an alkylene group having 1 to 10 carbon atoms, a cycloalkylene group having 4 to 10 carbon atoms, or an arylene group having 6 to 15 carbon atoms. In a case where X⁷ represents a direct bond, an alkylene group having 1 to 10 carbon atoms, or a cycloalkylene group having 4 to 10 carbon atoms, R²⁹ represents hydrogen, an alkyl group having 1 to 10 carbon atoms, or a cycloalkyl group having 4 to 10 carbon atoms, an alkoxy group having 1 to 10 carbon atoms, a haloalkyl group having 1 to 10 carbon atoms, a haloalkoxy group having 1 to 10 carbon atoms, an acyl group having 2 to 10 carbon atoms, or a nitro group. In a case where X⁷ represents an arylene group having 6 to 15 carbon atoms, R² represents hydrogen, an alkyl group having 1 to 10 carbon atoms, a cycloalkyl group having 4 to 10 carbon atoms, an aryl group having 6 to 15 carbon atoms, a haloalkyl group having 1 to 10 carbon atoms, a haloalkoxy group having 1 to 10 carbon atoms, a heterocyclic group having 4 to 10 carbon atoms, a heterocyclic oxy group having 4 to 10 carbon atoms, an acyl group having 2 to 10 carbon atoms, or a nitro group. R³⁰ represents hydrogen, an alkyl group having 1 to 10 carbon atoms, a cycloalkyl group having 4 to 10 carbon atoms, or an aryl group having 6 to 15 carbon atoms. a represents 0 or 1, and b represents an integer of 0 to 10.)

### <(A1) First Resin>

The negative photosensitive resin composition according to the present invention contains at least the (A1) first resin as the (A) alkali-soluble resin. The composition contains, as the (A1) first resin, one or more selected from a (A1-1) polyimide, a (A1-2) polyimide precursor, a (A1-3) polybenzoxazole, and a (A1-4) polybenzoxazole precursor. According to the present invention, the (A1-1) polyimide, the (A1-2) polyimide precursor, the (A1-3) polybenzoxazole, and the (A1-4) polybenzoxazole precursor may be any single resin or copolymer thereof.

As the (A) alkali-soluble resin, from the viewpoint of improving the halftone characteristics, improving the heat resistance of the cured film, and improving the reliability of the light emitting element, it is preferable to contain, as the (A1) first resin, at least one selected from the group consisting of the (A1-1) polyimide, the (A1-2) polyimide precursor, the (A1-3) polybenzoxazole, and the (A1-4) polybenzoxazole precursor, more preferable to contain the (A1-1) polyimide and/or the (A1-3) polybenzoxazole, and still more preferable to contain the (A1-1) polyimide.

### <(A1-1) Polyimide and (A1-2) Polyimide Precursor>

Examples of the (A1-2) polyimide precursor include products obtained by reacting a tetracarboxylic acid, a corresponding tetracarboxylic dianhydride or tetracarboxylic diester dichloride, or the like, with a diamine, a corresponding diisocyanate compound or trimethylsilylated diamine, or the like, which have a tetracarboxylic acid residue and/or a derivative residue thereof, and a diamine residue and/or a derivative residue thereof. Examples of the (A1-2) polyimide precursor include a polyamide acid, a polyamide acid ester, polyamide acid amide, and a polyisoimide.

Examples of the (A1-1) polyimide include products obtained by dehydration and cyclization of the above-described polyamide acid, polyamide acid ester, polyamide acid amide, or polyisoimide through heating or through a reaction with the use of an acid, a base, or the like, which have a tetracarboxylic acid residue and/or a derivative residue thereof, and a diamine residue and/or a derivative residue thereof.

The (A1-2) polyimide precursor, which is a thermosetting resin, is thermally cured at high temperature for dehydration and cyclization to form a highly heat-resistance imide bond, thereby providing the (A1-1) polyimide. Accordingly, the negative photosensitive resin composition contains therein the (A1-1) polyimide having the highly heat-resistance imide bond, thereby making it possible to remarkably improve the heat resistance of the cured film obtained. For that reason, the cured film is suitable in such a case of using the cured film for applications which require high heat resistance. In addition, the (A1-2) polyimide precursor, which is a resin with heat resistance improved after dehydration and cyclization, is suitable in such a case of using the precursor for applications which have a desire to achieve a balance between characteristics of the precursor structure before dehydration and cyclization and the heat resistance of the cured film.

Furthermore, the (A1-1) polyimide and the (A1-2) polyimide precursor have an imide bond and/or an amide bond as a bond with polarity. For that reason, in the case of containing, in particular, a (D1) pigment as a (D) colorant described later, the bond interacts strongly with the (D1) pigment, thus allowing the dispersion stability of the (D1) pigment to be improved.

The (A1-1) polyimide for use in the present invention preferably contains a structural unit represented by the following general formula (1), from the viewpoint of improving the heat resistance of the cured film.

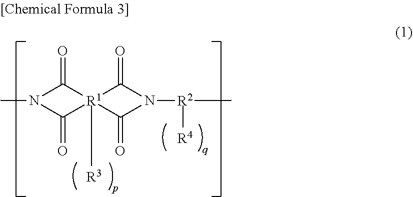

In the general formula (1), R¹ represents a tetravalent to decavalent organic group, and R² represents a divalent to decavalent organic group. R³ and R⁴ each independently represent a phenolic hydroxyl group, a sulfonic acid group, a mercapto group, or a substituent represented by the general formula (5) or the general formula (6). p represents an integer of 0 to 6, and q represents an integer of 0 to 8.

R¹ of the general formula (1) represents a tetracarboxylic acid residue and/or a derivative residue thereof, and R² represents a diamine residue and/or a derivative residue thereof. Examples of the tetracarboxylic acid derivative include a tetracarboxylic dianhydride, a tetracarboxylic acid dichloride, or a tetracarboxylic acid active diester. Examples of the diamine derivative include a diisocyanate compound or a trimethylsilylated diamine.

In the general formula (1), R^{j} preferably represents a tetravalent to decavalent organic group having one or more selected from an aliphatic structure having 2 to 20 carbon atoms, an alicyclic structure having 4 to 20 carbon atoms, and an aromatic structure having 6 to 30 carbon atoms. Furthermore, R² preferably represents a divalent to decavalent organic group having one or more selected from an aliphatic structure having 2 to 20 carbon atoms, an alicyclic structure having 4 to 20 carbon atoms, and an aromatic structure having 6 to 30 carbon atoms. q is preferably 1 to 8. The above-described aliphatic structure, alicyclic structure, and aromatic structure may have a hetero atom, and may be either unsubstituted or substituted.

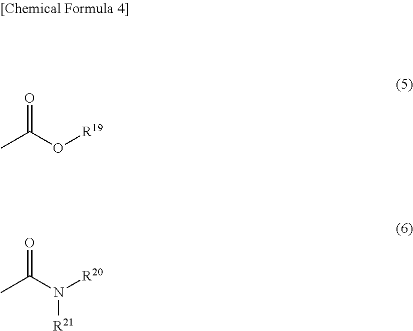

In the general formulas (5) and (6), R¹⁹ to R²¹ each independently represent hydrogen, an alkyl group having 1 to 10 carbon atoms, an acyl group having 2 to 6 carbon atoms, or an aryl group having 6 to 15 carbon atoms. In the general formulas (5) and (6), R¹⁹ to R²¹ each independently preferably represent hydrogen, an alkyl group having 1 to 6 carbon atoms, an acyl group having 2 to 4 carbon atoms, or an aryl group having 6 to 10 carbon atoms. The above-described alkyl group, acyl group, and aryl group may be either unsubstituted or substituted.

The (A1-1) polyimide preferably contains the structural unit represented by general formula (1) as a main component, and the content ratio of the structural unit represented by general formula (1) to all of structural units in the (A1-1) polyimide is preferably 50 to 100 mol %, more preferably 60 to 100 mol %, still more preferably 70 to 100 mol %. When the content ratio is 50 to 100 mol %, the heat resistance of the cured film can be improved.

The (A1-2) polyimide precursor for use in the present invention preferably contains a structural unit represented by the following general formula (3) from the viewpoint of improving the heat resistance of the cured film and improving the resolution after development.

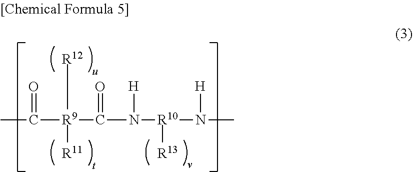

In the general formula (3), R⁹ represents a tetravalent to decavalent organic group, and R¹⁰ represents a divalent to decavalent organic group. R¹¹ represents a substituent represented by the above-described general formula (5) or general formula (6), R¹² represents a phenolic hydroxyl group, a sulfonic acid group, or a mercapto group, and R¹ represents a phenolic hydroxyl group, a sulfonic acid group, a mercapto group, or a substituent represented by the above-described general formula (5) or general formula (6). t represents an integer of 2 to 8, u represents an integer of 0 to 6, and v represents an integer of 0 to 8, and 2≤t+u≤8.

R⁹ of the general formula (3) represents a tetracarboxylic acid residue and/or a derivative residue thereof, and R¹⁰ represents a diamine residue and/or a derivative residue thereof. Examples of the tetracarboxylic acid derivative include a tetracarboxylic dianhydride, a tetracarboxylic acid dichloride, or a tetracarboxylic acid active diester. Examples of the diamine derivative include a diisocyanate compound or a trimethylsilylated diamine.

In the general formula (3), R⁹ preferably represents a tetravalent to decavalent organic group having one or more selected from an aliphatic structure having 2 to 20 carbon atoms, an alicyclic structure having 4 to 20 carbon atoms, and an aromatic structure having 6 to 30 carbon atoms. Furthermore, R¹⁰ preferably represents a divalent to decavalent organic group having one or more selected from an aliphatic structure having 2 to 20 carbon atoms, an alicyclic structure having 4 to 20 carbon atoms, and an aromatic structure having 6 to 30 carbon atoms. v is preferably 1 to 8. The above-described aliphatic structure, alicyclic structure, and aromatic structure may have a hetero atom, and may be either unsubstituted or substituted.

The (A1-2) polyimide precursor preferably contains the structural unit represented by the general formula (3) as a main component, and the content ratio of the structural unit represented by the general formula (3) to all of structural units in the (A1-2) polyimide precursor is preferably 50 to 100 mol %, more preferably 60 to 100 mol %, still more preferably 70 to 100 mol %. When the content ratio is 50 to 100 mol %, the resolution can be improved.

As the (A1-2) polyimide precursor, in a case where R¹¹ in the structural unit represented by general formula (3) represents a substituent represented by general formula (5), the structural unit where R¹⁹ represents hydrogen is referred to as an amide acid structural unit. The amide acid structural unit in the (A1-2) polyimide precursor has a carboxy group as a tetracarboxylic acid residue and/or a derivative residue thereof. It is to be noted that the (A1-2) polyimide precursor where R¹¹ in the structural unit represented by general formula (3) is composed of only a substituent represented by general formula (5), and R¹¹ represents hydrogen is referred to as a (A1-2a) polyamide acid.

As the (A1-2) polyimide precursor, in a case where R¹¹ in the structural unit represented by general formula (3) represents a substituent represented by general formula (5), the structural unit where R¹⁹ represents an alkyl group having 1 to 10 carbon atoms, an acyl group having 2 to 6 carbon atoms, or an aryl group having 6 to 15 carbon atoms is referred to as an amide acid ester unit. The amide acid ester structural unit in the (A1-2) polyimide precursor has a carboxylic acid ester group as a tetracarboxylic acid residue and/or an esterified derivative residue thereof. It is to be noted that the (A1-2) polyimide precursor where R¹¹ in the structural unit represented by general formula (3) is composed of only a substituent represented by general formula (5), and R¹⁹ represents an alkyl group having 1 to 10 carbon atoms, an acyl group having 2 to 6 carbon atoms, or an aryl group having 6 to 15 carbon atoms is referred to as a (A1-2b) polyamide acid ester.

As the (A1-2) polyimide precursor, in a case where R¹¹ in the structural unit represented by general formula (3) represents a substituent represented by general formula (6), the structural unit is referred to as an amide acid amide structural unit. The amide acid amide structural unit in the (A1-2) polyimide precursor has a carboxylic acid amide group as a tetracarboxylic acid residue and/or an amidated derivative residue thereof. It is to be noted that the (A1-2) polyimide precursor where R¹¹ in the structural unit represented by general formula (3) is composed of only a substituent represented by general formula (6) is referred to as a (A1-2c) polyamide acid amide.

From the viewpoint of improving the resolution after development and forming a pattern in a low taper shape after development, the (A1-2) polyimide precursor preferably contains the amide acid structural unit, and the amide acid ester structural unit and/or the amide acid amide structural unit. It is to be noted that the (A1-2) polyimide precursor containing the amide acid structural unit and the amide acid ester structural unit is referred to as a (A1-2-1) polyamide acid partial ester. On the other hand, the (A1-2) polyimide precursor containing the amide acid structural unit and the amide acid amide structural unit is referred to as a (A1-2-2) polyamide acid partial amide. Furthermore, the (A1-2) polyimide precursor containing the amide acid structural unit, the amide acid ester structural unit, and the amide acid amide structural unit is referred to as a (A1-2-3) polyamide acid partial ester amide. These polyimide precursors containing the amide acid structural unit and the amide acid ester structural unit and/or the amide acid amide structural unit can be synthesized by esterifying a part of the carboxy group and/or amidating a part of the carboxy group from the (A1-2a) polyamide acid having a tetracarboxylic acid residue and/or a carboxy group as a derivative residue thereof.

The content ratio of the polyamide acid unit to all the structural units in the (A1-2) polyimide precursor is preferably 10 mol % or higher, more preferably 20 mol % or higher, still more preferably 30 mol % or higher. When the content ratio is 10 mol % or higher, the resolution after development can be improved. On the other hand, the content ratio of the polyamide acid unit is preferably 60 mol % or lower, more preferably 50 mol % or lower, still more preferably 40 mol % or lower. When the content ratio is 60 mol % or lower, a pattern in a low taper shape can be formed after development.

The total content ratio of the polyamide acid ester unit and the polyamide acid amide unit to all of structural units in the (A1-2) polyimide precursor is preferably 40 mol % or higher, more preferably 50 mol % or higher, still more preferably 60 mol % or higher. When the total content ratio is 40 mol % or higher, a pattern in a low taper shape can be formed after development. On the other hand, the total content ratio of the polyamide acid ester unit and the polyamide acid amide unit is preferably 90 mol % or lower, more preferably 80 mol % or lower, still more preferably 70 mol %. When the total content ratio is 90 mol % or lower, the resolution after development can be improved.

### <(A1-3) Polybenzoxazole and (A1-4) Polybenzoxazole Precursor>

Examples of the (A1-4) polybenzoxazole precursor include products obtained by reacting a dicarboxylic acid, a corresponding dicarboxylic acid dichloride dicarboxylic acid active diester, or the like with a bisaminophenol compound as a diamine, and which have a dicarboxylic acid residue and/or a derivative residue thereof, and a bisaminophenol compound residue and/or a derivative residue thereof. Examples of the (A1-4) polybenzoxazole precursor include a polyhydroxyamide.

Examples of the (A1-3) polybenzoxazole include products obtained by dehydration and cyclization of a dicarboxylic acid and a bisaminophenol compound as a diamine through a reaction with the use of a polyphosphoric acid, and products obtained by dehydration and cyclization of the polyhydroxyamide described above through heating or reaction with the use of a phosphoric anhydride, a base or a carbodiimide compound, or the like, which have a dicarboxylic acid residue and/or a derivative residue thereof, a bisaminophenol compound residues and/or a derivative residue thereof.

The (A1-4) polybenzoxazole precursor, which is a thermosetting resin, is thermally cured at high temperature for dehydration and cyclization to form a highly heat-resistance and rigid benzoxazole ring, thereby providing the (A1-3) polybenzoxazole. Accordingly, the negative photosensitive resin composition contains therein the (A1-3) polybenzoxazole having the highly heat-resistance and rigid benzoxazole ring, thereby making it possible to remarkably improve the heat resistance of the cured film obtained. For that reason, the cured film is suitable in such a case of using the cured film for applications which require high heat resistance. In addition, the (A1-4) polybenzoxazole precursor, which is a resin with heat resistance improved after dehydration and cyclization, is suitable in such a case of using the precursor for applications which have a desire to achieve a balance between characteristics of the precursor structure before dehydration and cyclization and the heat resistance of the cured film.

Furthermore, the (A1-3) polybenzoxazole and the (A1-4) polybenzoxazole precursor have an imide bond and/or an oxazole bond as a bond with polarity. For that reason, in the case of containing, in particular, a (D1) pigment as a (D) colorant described later, the bond interacts strongly with the (D1) pigment, thus allowing the dispersion stability of the (D1) pigment to be improved.

The (A1-3) polybenzoxazole for use in the present invention preferably contains a structural unit represented by general formula (2), from the viewpoint of improving the heat resistance of the cured film.

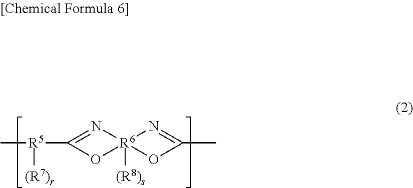

In the general formula (2), R⁵ represents a divalent to decavalent organic group, and R⁶ represents a tetravalent to decavalent organic group that has an aromatic structure. R⁷ and R⁸ each independently represent a phenolic hydroxyl group, a sulfonic acid group, a mercapto group, or a substituent represented by the general formula (5) or general formula (6) described above. r represents an integer of 0 to 8, and s represents an integer of 0 to 6.

R⁵ of the general formula (2) represents a dicarboxylic acid residue and/or a derivative residue thereof, and R⁶ represents a bisaminophenol compound residue and/or a derivative residue thereof. Examples of the dicarboxylic acid derivative include a dicarboxylic anhydride, a dicarboxylic acid chloride, a dicarboxylic acid active ester, a tricarboxylic anhydride, a tricarboxylic acid chloride, a tricarboxylic acid active ester, and a diformyl compound.

In the general formula (2), R⁵ preferably represents a divalent to decavalent organic group having one or more selected from an aliphatic structure having 2 to 20 carbon atoms, an alicyclic structure having 4 to 20 carbon atoms, and an aromatic structure having 6 to 30 carbon atoms. Furthermore, R⁶ is preferably a tetravalent to decavalent organic group that has an aromatic structure having 6 to 30 carbon atoms. s preferably represents 1 to 8. The above-described aliphatic structure, alicyclic structure, and aromatic structure may have a hetero atom, and may be either unsubstituted or substituted.

The (A1-3) polybenzoxazole preferably contains the structural unit represented by general formula (2) as a main component, and the content ratio of the structural unit represented by general formula (2) to all of structural units in the (A1-3) polybenzoxazole is preferably 50 to 100 mol %, more preferably 60 to 100 mol %, still more preferably 70 to 100 mol %. When the content ratio is 50 to 100 mol %, the heat resistance of the cured film can be improved.

The (A1-4) polybenzoxazole precursor for use in the present invention preferably contains a structural unit represented by general formula (4), from the viewpoint of improving the heat resistance of the cured film and improving the resolution after development.

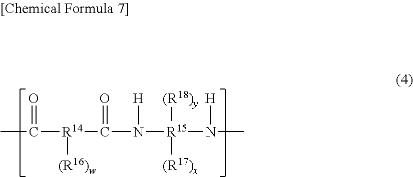

In the general formula (4), R¹⁴ represents a divalent to decavalent organic group, and R¹⁵ represents a tetravalent to decavalent organic group that has an aromatic structure. R¹⁶ represents a phenolic hydroxyl group, a sulfonic acid group, a mercapto group, or a substituent represented by general formula (5) or general formula (6) described above, R¹⁷ represents a phenolic hydroxyl group, and R¹⁸ represents a sulfonic acid, a mercapto group, or a substituent represented by general formula (5) or general formula (6) described above. w represents an integer of 0 to 8, x represents an integer of 2 to 8, y represents an integer of 0 to 6, and 2≤x+y≤8.

R¹⁴ of the general formula (4) represents a dicarboxylic acid residue and/or a derivative residue thereof, and R¹⁵ represents a bisaminophenol compound residue and/or a derivative residue thereof. Examples of the dicarboxylic acid derivative include a dicarboxylic anhydride, a dicarboxylic acid chloride, a dicarboxylic acid active ester, a tricarboxylic anhydride, a tricarboxylic acid chloride, a tricarboxylic acid active ester, and a diformyl compound.

In the general formula (4), R¹⁴ preferably represents a divalent to decavalent organic group having one or more selected from an aliphatic structure having 2 to 20 carbon atoms, an alicyclic structure having 4 to 20 carbon atoms, and an aromatic structure having 6 to 30 carbon atoms. Furthermore, R¹⁵ is preferably a tetravalent to decavalent organic group that has an aromatic structure having 6 to 30 carbon atoms. The above-described aliphatic structure, alicyclic structure, and aromatic structure may have a hetero atom, and may be either unsubstituted or substituted.

The (A1-4) polybenzoxazole precursor preferably contains the structural unit represented by general formula (4) as a main component, and the content ratio of the structural unit represented by general formula (4) to all of structural units in the (A1-4) polybenzoxazole precursor is preferably 50 to 100 mol %, more preferably 60 to 100 mol %, still more preferably 70 to 100 mol %. When the content ratio is 50 to 100 mol %, the resolution can be improved.

### <Tetracarboxylic Acid and Dicarboxylic Acid and Derivatives Thereof>

Examples of the tetracarboxylic acid include an aromatic tetracarboxylic acid, an alicyclic tetracarboxylic acid, and an aliphatic tetracarboxylic acid. These tetracarboxylic acids may have a hetero atom in addition to the oxygen atoms of the carboxy groups.

As the dicarboxylic acid and derivative thereof in the (A1-3) polybenzoxazole and the (A1-4) polybenzoxazole precursor, a tricarboxylic acid and/or a derivative thereof may be used. Examples of the dicarboxylic acid and tricarboxylic acid include an aromatic dicarboxylic acid, an aromatic tricarboxylic acid, an alicyclic dicarboxylic acid, an alicyclic tricarboxylic acid, an aliphatic dicarboxylic acid, and an aliphatic tricarboxylic acid. These dicarboxylic acid and tricarboxylic acid may have a hetero atom other than oxygen atoms, in addition to the oxygen atoms of the carboxy groups.

Examples of the tetracarboxylic acid, the dicarboxylic acid, and the tricarboxylic acid, and derivatives thereof include the compounds described in International Publication No. 2017/057281.

### <Diamine and Derivatives Thereof>

Examples of the diamine and derivatives thereof include aromatic diamines, bisaminophenol compounds, alicyclic diamines, alicyclic dihydroxydiamines, aliphatic diamines, and aliphatic dihydroxydiamines. These diamines and derivatives thereof may have a hetero atom in addition to the nitrogen atoms and oxygen atoms of the amino group and derivatives thereof.

Examples of the diamine and derivatives thereof include the compounds described in International Publication No. 2017/057281.

### <Structural Unit Having Fluorine Atom>

One or more selected from a (A1-1) polyimide, a (A1-2) polyimide precursor, a (A1-3) polybenzoxazole, and a (A1-4) polybenzoxazole precursor contains a structural unit having a fluorine atom at 10 to 100 mol % of all of the structural units. One or more selected from a (A1-1) polyimide, a (A1-2) polyimide precursor, a (A1-3) polybenzoxazole, and a (A1-4) polybenzoxazole precursor contains a structural unit having a fluorine atom, thereby improving the transparency, allowing the sensitivity for exposure to be improved, and allowing a pattern in a low-taper shape to be formed after development. In addition, halftone characteristics can be improved. This is presumed to be because improved transparency of the film has allowed radical curing in the deep part of the film. In addition, it is believed to be because in a case where the (C1-1) specific oxime ester-based photo initiator described later has a group substituted with a halogen, the compatibility between the resin and the initiator can be increased, thereby causing UV curing during exposure to proceed efficiently even in the deep part of the film. In addition, it is believed to be because fluorine atoms allow water repellency to be imparted to the film surface, thereby making it possible to suppress penetration of the developer into the film surface during alkali development, and suppress side etching by the developer. The exposure herein refers to irradiation with active actinic rays (radiation), and examples thereof include irradiation with visible light, ultraviolet rays, electron beams, X-rays or the like. From the viewpoint of a light source commonly used, for example, an ultra-high pressure mercury lamp light source capable of irradiation with visible light or ultraviolet rays is preferred, and more preferred is irradiation with j-lines (wavelength: 313 nm), i-lines (wavelength: 365 nm), h-lines (wavelength: 405 nm), or g-lines (wavelength: 436 nm). Hereinafter, the exposure refers to irradiation with active actinic rays (radiation).

In addition, in general, in the case of using the (A1-1) polyimide, the (A1-2) polyimide precursor, the (A1-3) polybenzoxazole, and/or the (A1-4) polybenzoxazole precursor, it is necessary to use, as an after-mentioned solvent that is used for dissolution of the foregoing resins, a highly polar solvent such as N-methyl-2-pyrrolidone, dimethyl sulfoxide, N,N-dimethylformamide, or γ-butyrolactone. In a case where, in particular, the (D1) pigment is contained as the (D) colorant described later, however, these highly polar solvents interact strongly with the (D1) pigment, and the effect of improving the dispersion stability with the (A1) first resin, the (A2) second resin described layer, or the (E) dispersant described later may be thus insufficient.

One or more selected from a (A1-1) polyimide, a (A1-2) polyimide precursor, a (A1-3) polybenzoxazole, and a (A1-4) polybenzoxazole precursor contain a structural unit having a fluorine atom, thereby allowing the solubility in the solvent to be improved. Thus, it is possible to reduce the content of the highly polar solvent described above or dissolve the foregoing resins without using the highly polar solvent, thereby allowing the dispersion stability of the (D1) pigment to be improved.

Examples of the structural unit having a fluorine atom, which is contained in the (A1-1) polyimide and/or the (A1-2) polyimide precursor, include a structural unit derived from a tetracarboxylic acid having a fluorine atom and/or a structural unit derived from a derivative of the tetracarboxylic acid, or a structural unit derived from a diamine having a fluorine atom and/or a structural unit derived from a derivative of the diamine.

Examples of the structural unit having a fluorine atom, which is contained in the (A1-3) polybenzoxazole and/or the (A1-4) polybenzoxazole precursor, include a structural unit derived from a dicarboxylic acid having a fluorine atom and/or a structural unit derived from a derivative of the dicarboxylic acid, or a structural unit derived from a bisaminophenol compound having a fluorine atom and/or a structural unit derived from a derivative of the bisaminophenol compound.

The content ratio of the structural unit having a fluorine atom to all of structural units is preferably 30 to 100 mol % in one or more resins selected from the (A1-1) polyimide, the (A1-2) polyimide precursor, the (A1-3) polybenzoxazole, and the (A1-4) polybenzoxazole precursor. The content ratio of the structural unit having a fluorine atom is more preferably 50 mol % or higher, still more preferably 70 mol % or higher. When the content ratio is 30 to 100 mol %, the sensitivity for exposure can be improved.

The content ratio of structural units derived from one or more selected from a tetracarboxylic acid having a fluorine atom, a tetracarboxylic acid derivative having a fluorine atom, a dicarboxylic acid having a fluorine atom, and a dicarboxylic acid derivative having a fluorine atom to the total of structural units derived from all of carboxylic acids and structural units derived from derivatives of the acids is preferably 30 to 100 mol % in one or more resins selected from the (A1-1) polyimide, the (A1-2) polyimide precursor, the (A1-3) polybenzoxazole, and the (A1-4) polybenzoxazole precursor. The content ratio of the structural unit having a fluorine atom is more preferably 50 mol % or higher, still more preferably 70 mol % or higher. When the content ratio is 30 to 100 mol %, the sensitivity for exposure can be improved.

The content ratio of structural units derived from one or more selected from a diamine having a fluorine atom, a diamine derivative having a fluorine atom, a bisaminophenol compound having a fluorine atom, and a bisaminophenol compound derivative having a fluorine atom to the total of structural units derived from all of amines and structural units derived from derivatives of the amines is preferably 30 to 100 mol % in one or more resins selected from the (A1-1) polyimide, the (A1-2) polyimide precursor, the (A1-3) polybenzoxazole, and the (A1-4) polybenzoxazole precursor. The content ratio of the structural unit having a fluorine atom is more preferably 50 mol % or higher, still more preferably 70 mol % or higher. When the content ratio is 30 to 100 mol %, the sensitivity for exposure can be improved.

<Structural Units Derived from Aromatic Carboxylic Acid and Derivative Thereof>

The (A1-1) polyimide and/or the (A1-2) polyimide precursor preferably contains a structural unit derived from an aromatic carboxylic acid and/or a structural unit derived from a derivative of the acid. The (A1-1) polyimide and/or the (A1-2) polyimide precursor contains a structural unit derived from an aromatic carboxylic acid and/or a structural unit derived from a derivative of the acid, thereby allowing the heat resistance of the aromatic group to improve the heat resistance of the cured film. As the aromatic carboxylic acid and the derivative thereof, an aromatic tetracarboxylic acid and/or a derivative thereof are preferred.

The content ratio of the structural unit derived from an aromatic carboxylic acid and/or the structural unit derived from a derivative of the acid to the total of structural units derived from all of carboxylic acids and structural units derived from derivatives of the acids is preferably 50 to 100 mol %, more preferably 60 to 100 mol %, still more preferably 70 to 100 mol % in (A1-1) polyimide and/or (A1-2) polyimide precursor. When the content ratio is 50 to 100 mol %, the heat resistance of the cured film can be improved.

The (A1-3) polybenzoxazole and/or the (A1-4) polybenzoxazole precursor preferably contains a structural unit derived from an aromatic carboxylic acid and/or a structural unit derived from a derivative of the acid. The (A1-3) polybenzoxazole and/or the (A1-4) polybenzoxazole precursor contains a structural unit derived from an aromatic carboxylic acid and/or a structural unit derived from a derivative of the acid, thereby allowing the heat resistance of the aromatic group to improve the heat resistance of the cured film. As the aromatic carboxylic acid and the derivative thereof, an aromatic dicarboxylic acid or an aromatic tricarboxylic acids and/or derivatives thereof are preferred, and an aromatic dicarboxylic acid and/or a derivative thereof are more preferred.

The content ratio of the structural unit derived from an aromatic carboxylic acid and/or the structural unit derived from a derivative of the acid to the total of structural units derived from all of carboxylic acids and structural units derived from derivatives of the acids is preferably 50 to 100 mol %, more preferably 60 to 100 mol %, still more preferably 70 to 100 mol % in the (A1-3) polybenzoxazole and/or the (A1-4) polybenzoxazole precursor. When the content ratio is 50 to 100 mol %, the heat resistance of the cured film can be improved.

<Structural Units Derived from Aromatic Amine and Derivative>

One or more selected from the (A1-1) polyimide, the (A1-2) polyimide precursor, the (A1-3) polybenzoxazole, and the (A1-4) polybenzoxazole precursor preferably contain a structural unit derived from an aromatic amine and/or a structural unit derived from a derivative of the amine. One or more selected from the (A1-1) polyimide, the (A1-2) polyimide precursor, the (A1-3) polybenzoxazole, and the (A1-4) polybenzoxazole precursor contain a structural unit derived from an aromatic amine and/or a structural unit derived from a derivative of the amine, thereby allowing the heat resistance of the aromatic group to improve the heat resistance of the cured film. As the aromatic amine and the derivative thereof, an aromatic diamine, a bisaminophenol compound, an aromatic triamine, or a trisaminophenol compound, and/or a derivative thereof are preferred, and an aromatic diamine or a bisaminophenol compound, and/or a derivatives thereof are more preferred.

The content ratio of the structural unit derived from an aromatic amine and/or the structural unit derived from a derivative of the amine to the total of structural units derived from all of amines and structural units derived from derivatives of the amines is preferably 50 to 100 moly, more preferably 60 to 100 mol %, still more preferably 70 to 100 mol % in one or more resins selected from the (A1-1) polyimide, the (A1-2) polyimide precursor, the (A1-3) polybenzoxazole, and the (A1-4) polybenzoxazole precursor. When the content ratio is 50 to 100 mol %, the heat resistance of the cured film can be improved.

<Structural Units Derived from Diamine Having Silyl Group or Siloxane Bond and Derivative Thereof>

One or more selected from the (A1-1) polyimide, the (A1-2) polyimide precursor, the (A1-3) polybenzoxazole, and the (A1-4) polybenzoxazole precursor preferably contain a structural unit derived from a diamine having a silyl group or a siloxane bond and/or a structural unit derived from a derivative of the diamine. One or more selected from the (A1-1) polyimide, the (A1-2) polyimide precursor, the (A1-3) polybenzoxazole, and the (A1-4) polybenzoxazole precursor contain a structural unit derived from a diamine having a silyl group or a siloxane bond and/or a structural unit derived from a derivative of the diamine, thereby increasing the interaction between the cured film of the negative photosensitive resin composition and the underlying substrate interface, and then allowing the adhesion property to the underlying substrate and the chemical resistance of the cured film to be improved.

<Structural Units Derived from Amine Having Oxyalkylene Structure and Derivative Thereof>

One or more selected from the (A1-1) polyimide, the (A1-2) polyimide precursor, the (A1-3) polybenzoxazole, and the (A1-4) polybenzoxazole precursor preferably contain a structural unit derived from an amine that has an oxyalkylene structure and/or a structural unit derived from a derivative of the amine. One or more selected from the (A1-1) polyimide, the (A1-2) polyimide precursor, the (A1-3) polybenzoxazole, and the (A1-4) polybenzoxazole precursor contain a structural unit derived from an amine that has an oxyalkylene structure and/or a structural unit derived from a derivative of the amine, thereby allowing a cured film in a pattern in a low-taper shape to be obtained, and allowing the mechanical characteristic of the cured film and the patternability thereof with an alkaline developer to be improved.

### <End-Capping Agent>

For one or more selected from the (A1-1) polyimide, the (A1-2) polyimide precursor, the (A1-3) polybenzoxazole, and the (A1-4) polybenzoxazole precursor, the terminals of the resins may be sealed with an end-capping agent such as a monoamine, a dicarboxylic anhydride, a monocarboxylic acid, a monocarboxylic acid chloride, or monocarboxylic acid active ester. The terminals of the resins are sealed with the end-capping agent, thereby making it possible to improve the storage stability of a coating liquid with the resin composition containing one or more selected from the (A1-1) polyimide, the (A1-2) polyimide precursor, the (A1-3) polybenzoxazole, and the (A1-4) polybenzoxazole precursor.

The content ratio of the structural units derived from various types of carboxylic acids or amines and derivatives thereof to the (A1-1) polyimide, the (A1-2) polyimide precursor, the (A1-3) polybenzoxazole, and/or the (A1-4) polybenzoxazole precursor can be determined by combining ¹H-NMR, ¹³C-NMR, ¹⁵N-NMR, IR, TOF-MS, elemental analysis, ash measurement, and the like.

### <Introduction of Ethylenically Unsaturated Double Bond Group>

One or more selected from the (A1-1) polyimide, the (A1-2) polyimide precursor, the (A1-3) polybenzoxazole, and the (A1-4) polybenzoxazole precursor preferably have an ethylenically unsaturated double group. The resins are also preferred, which have an ethylenically unsaturated double bond group introduced into the side chains of the resins by a reaction for introducing an ethylenically unsaturated double bond group. With an ethylenically unsaturated double bond group, a pattern in a low-taper shape can be formed after development.

One or more selected from the (A1-1) polyimide, the (A1-2) polyimide precursor, the (A1-3) polybenzoxazole, and the (A1-4) polybenzoxazole precursor are also preferably obtained by reacting some phenolic hydroxyl groups and/or carboxy groups thereof with a compound having an ethylenically unsaturated double bond group. The reaction described above allows an ethylenically unsaturated double bond group to be introduced into the side chain of the resin.

The compound having an ethylenically unsaturated double bond group is preferably an electrophilic compound having an ethylenically unsaturated double bond group from the viewpoint of reactivity. Examples of the electrophilic compound include isocyanate compounds, isothiocyanate compounds, epoxy compounds, aldehyde compounds, thioaldehyde compounds, ketone compounds, thioketone compounds, acetate compounds, carboxylic acid chlorides, carboxylic anhydrides, and carboxylic acid active ester compounds, carboxylic acid compounds, halogenated alkyl compounds, alkyl azide compounds, triflate alkyl compounds, alkyl mesylate compounds, alkyl tosylate compounds, or alkyl cyanide compounds, and from the viewpoints of reactivity and compound utility, isocyanate compounds, epoxy compounds, aldehyde compounds, ketone compounds, or carboxylic anhydrides is preferred, and isocyanate compounds or epoxy compounds is more preferred.

### <Physical Properties of (A1-1) Polyimide, (A1-2) Polyimide Precursor, (A1-3) Polybenzoxazole, and (A1-4) Polybenzoxazole Precursor>

The weight average molecular weight (hereinafter, “Mw”) of one or more selected from the (A1-1) polyimide, the (A1-2) polyimide precursor, the (A1-3) polybenzoxazole, and the (A1-4) polybenzoxazole precursor is preferably 1,000 or more, more preferably 3,000 or more, still more preferably 5,000 or more in terms of polystyrene measured by gel permeation chromatography (hereinafter, “GPC”). When the Mw is 1,000 or more, the resolution after development can be improved. On the other hand, the Mw is preferably 500,000 or less, more preferably 300,000 or less, still more preferably 100,000 or less. When the Mw is 500,000 or less, the leveling property in the case of coating and the patternability with an alkaline developer can be improved.

Furthermore, the number average molecular weight (hereinafter, “Mn”) is preferably 1,000 or more, more preferably 3,000 or more, still more preferably 5,000 or more in terms of polystyrene measured by GPC. When the Mn is 1,000 or more, the resolution after development can be improved. On the other hand, the Mn is preferably 500,000 or less, more preferably 300,000 or less, still more preferably 100,000 or less. When the Mn is 500,000 or less, the leveling property in the case of coating and the patternability with an alkaline developer can be improved.

The Mw and Mn of the (A1-1) polyimide, (A1-2) polyimide precursor, (A1-3) polybenzoxazole, and (A1-4) polybenzoxazole precursor can be easily measured as a value in terms of polystyrene by GPC, a light scattering method, an X-ray small angle scattering method, or the like. The repetition number n of structural units in the (A1-1) polyimide, the (A1-2) polyimide precursor, the (A1-3) polybenzoxazole, and the (A1-4) polybenzoxazole precursor can be determined from n=Mw/M where M represents the molecular weight of the structural unit, and Mw represents the weight average molecular weight of the resins.

The (A1-1) polyimide and the (A1-2) polyimide precursor can be synthesized by known methods. The methods include a method of reacting a tetracarboxylic dianhydride and a diamine (partially substituted with a monoamine as an end-capping agent) at 80° C. to 200° C. in a polar solvent such as N-methyl-2-pyrrolidone, or a method of reacting a tetracarboxylic dianhydride (partially substituted with a dicarboxylic anhydride, a monocarboxylic acid, a monocarboxylic acid chloride, or a monocarboxylic acid active ester as an end-capping agent) and a diamine at 80° C. to 200° C.

The (A1-3) polybenzoxazole and the (A1-4) polybenzoxazole precursor can be synthesized by known methods. The methods include a method of reacting a dicarboxylic acid active diester and a bisaminophenol compound (partially substituted with a monoamine as an end-capping agent) at 80° C. to 250° C. in a polar solvent such as N-methyl-2-pyrrolidone, or a method of reacting a dicarboxylic acid active diester (partially substituted with a dicarboxylic anhydride, a monocarboxylic acid, a monocarboxylic acid chloride, or a monocarboxylic acid active ester as an end-capping agent) and a bisaminophenol compound at 80° C. to 250° C.

### <(A2) Second Resin>

The negative photosensitive resin composition according to the present invention preferably contains the (A2) second resin as the (A) alkali-soluble resin. It is preferable to contain, as the (A2) second resin, one or more selected from a (A2-1) polysiloxane, a (A2-2) polycyclic side chain-containing resin, an (A2-3) acid-modified epoxy resin, and an (A2-4) acrylic resin, from the viewpoints of improving the sensitivity for exposure and reducing the taper by controlling the pattern shape after development. According to the present invention, the (A2-1) polysiloxane, the (A2-2) polycyclic side chain-containing resin, the (A2-3) acid-modified epoxy resin, and the (A2-4) acrylic resin may be any of single resins or copolymers thereof.

As the (A) alkali-soluble resin, from the viewpoints of improving halftone characteristics, improving the sensitivity during exposure, and reducing the taper by pattern shape control after development, it is preferable to contain, as the (A2) second resin, one or more selected from the group consisting of the (A2-1) polysiloxane, the (A2-2) polycyclic side chain-containing resin, the (A2-3) acid-modified epoxy resin, and the (A2-4) acrylic resin, it is more preferable to contain one or more selected from the group consisting of the (A2-1) polysiloxane, the (A2-2) polycyclic side chain-containing resin, and the (A2-3) acid-modified epoxy resin, it is still more preferable to contain the (A2-1) polysiloxane and/or the (A2-2) polycyclic side chain-containing resin, and it is particularly preferable to contain the (A2-1) polysiloxane. Containing the (A2-1) polysiloxane makes it possible to form a pattern in a low-taper shape after thermal curing, and allows the change in pattern opening width between before and after thermal curing to be suppressed.

### <(A2-1) Polysiloxane>

Examples of the (A2-1) polysiloxane for use in the present invention include a polysiloxane obtained by hydrolyzing, and then dehydrating condensing one or more selected from a trifunctional organosilane, a tetrafunctional organosilane, a bifunctional organosilane, and a monofunctional organosilane.

The (A2-1) polysiloxane, which is a thermosetting resin, is thermally cured at high temperature for dehydration and condensation to form a high heat-resistance siloxane bond (Si—O). Accordingly, the negative photosensitive resin composition contains therein the (A2-1) polysiloxane having the highly heat-resistance siloxane bond, thereby making it possible improve the heat resistance of the cured film obtained. In addition, the (A2-1) polysiloxane, which is a resin with heat resistance improved after dehydration and condensation, is thus suitable in such a case of using the precursor for applications which have a desire to achieve a balance between characteristics before dehydration and condensation and the heat resistance of the cured film.

Furthermore, the (A2-1) polysiloxane has a silanol group as a reactive group. Thus, in the case of containing, in particular, the (D1) pigment is as the (D) colorant described later, the silanol group is capable of interacting with and/or binding to the surface of the (D1) pigment, and capable of interacting with and/or binding to the surface modifying group of the (D1) pigment. Accordingly, the dispersion stability of the (D1) pigment can be improved.

### <Trifunctional Organosilane Unit, Tetrafunctional Organosilane Unit, Bifunctional Organosilane Unit, and Monofunctional Organosilane Unit>

The (A2-1) polysiloxane for use in the present invention preferably contains a trifunctional organosilane unit and/or a tetrafunctional organosilane unit, from the viewpoint of improving the heat resistance of the cured film and improving the resolution after development. The trifunctional organosilane is preferably an organosilane unit represented by general formula (7). The tetrafunctional organosilane unit is preferably an organosilane unit represented by general formula (8). In addition, the polysiloxane may contain a bifunctional organosilane unit from the viewpoint of reducing the taper of the pattern shape and improving the mechanical characteristic of the cured film. The bifunctional organosilane is preferably an organosilane unit represented by general formula (9). In addition, the polysiloxane may contain a monofunctional organosilane unit from the viewpoint of improving the storage stability of the coating liquid with the resin composition. The monofunctional organosilane unit is preferably an organosilane unit represented by general formula (10).

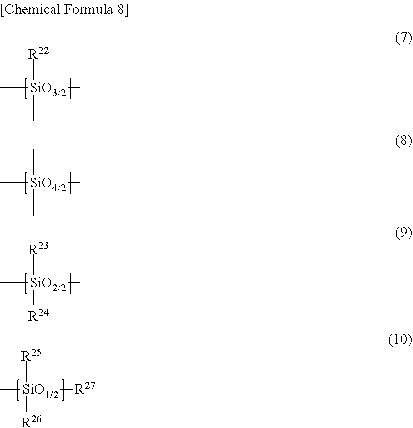

In the general formulas (7) to (10), R²² to R²⁷ each independently represent hydrogen, an alkyl group, a cycloalkyl group, an alkenyl group, or an aryl group. In the general formulas (7) to (10), R²² to R²⁷ each independently preferably represents hydrogen, an alkyl group having 1 to 10 carbon atoms, a cycloalkyl group having 4 to 10 carbon atoms, an alkenyl group having 2 to 10 carbon atoms, or an aryl group having 6 to 15 carbon atoms. The alkyl group, cycloalkyl group, alkenyl group, and aryl group described above may have a hetero atom, and may be either unsubstituted or substituted.

Examples of the organosilane having an organosilane unit represented by the general formula (7), the general formula (8), the general formula (9), or the general formula (10) include the compound described in International Publication No. 2017/057281.

The content ratio of the organosilane unit represented by general formula (7) to the (A2-1) polysiloxane is preferably 50 to 100 mol %, more preferably 60 to 100 mol %, still more preferably 70 to 100 mol % in terms of Si atom mol ratio. When the content ratio is 50 to 100 mol %, the heat resistance of the cured film can be improved. The organosilane unit represented by the general formula (7) is preferably an organosilane unit having an epoxy group. The (A2-1) polysiloxane contains an organosilane unit having an epoxy group, thereby making it possible to improve the patternability during alkali development and improve the sensitivity for exposure.

The content ratio of the organosilane unit represented by general formula (8) to the (A2-1) polysiloxane is preferably 0 to 40 mol %, more preferably 0 to 30 mol %, still more preferably 0 to 20 mol % in terms of Si atom mol ratio. The content ratio of 0 to 40 mol % makes it possible to improve the patternability during alkali development, improve the sensitivity for exposure, and improve the heat resistance of the cured film. In addition, a pattern in a low-taper shape can be formed after development, and the change in pattern opening width between before and after thermal curing can be suppressed.

The content ratio of the organosilane unit represented by general formula (9) to the (A2-1) polysiloxane is preferably 0 to 60 mol %, more preferably 0 to 50 mol %, still more preferably 0 to 40 mol % in terms of Si atom mol ratio. When the content ratio is 0 to 60 mol %, the heat resistance of the cured film and the resolution after development can be improved.

The content ratio of the organosilane unit represented by general formula (10) to the (A2-1) polysiloxane is preferably 0 to 20 mol %, more preferably 0 to 10 mol %, still more preferably 0 to 5 mol % in terms of Si atom mol ratio. When the content ratio is 0 to 20 mol %, the heat resistance of the cured film can be improved.

The polysiloxane (A2-1) for use in the present invention is preferably the polysiloxane (A2-1) obtained by hydrolyzing, and then dehydrating and condensing one or more selected from an organosilane represented by general formula (7a), an organosilane represented by general formula (8a), and an organosilane represented by general formula (9a), and an organosilane represented by general formula (10a).

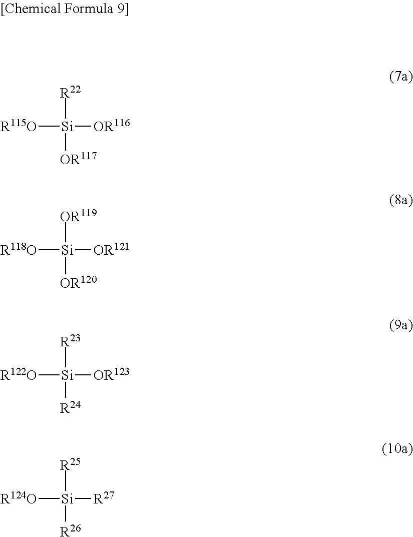

In the general formulas (7a) to (10a), R²² to R²⁷ each independently represent hydrogen, an alkyl group, a cycloalkyl group, an alkenyl group, or an aryl group, and R¹¹⁵ to R¹²⁴ each independently represent hydrogen, an alkyl group, an acyl group, or an aryl group. In the general formulas (7a) to (10a), R²² to R²⁷ each independently preferably represent hydrogen, an alkyl group having 1 to 10 carbon atoms, a cycloalkyl group having 4 to 10 carbon atoms, an alkenyl group having 2 to 10 carbon atoms, or an aryl group having 6 to 15 carbon atoms. R¹¹⁵ to R¹²⁴ each independently preferably represent hydrogen, an alkyl group having 1 to 6 carbon atoms, an acyl group having 2 to 6 carbon atoms, or an aryl group having 6 to 15 carbon atoms. The alkyl group, cycloalkyl group, alkenyl group, aryl group, and acyl group described above may have a hetero atom, and may be either unsubstituted or substituted.

In the (A2-1) polysiloxane, the organosilane unit represented by general formula (7), the organosilane unit represented by general formula (8), the organosilane unit represented by general formula (9), and the organosilane unit represented by general formula (10) may have a regular arrangement or an irregular arrangement. Examples of the regular arrangement include alternating copolymerization, periodic copolymerization, block copolymerization, or graft copolymerization. Examples of the irregular arrangement include random copolymerization.

In addition, in the (A2-1) polysiloxane, the organosilane unit represented by general formula (7), the organosilane unit represented by general formula (8), the organosilane unit represented by general formula (9), and the organosilane unit represented by general formula (10) may have a two-dimensional arrangement or a three-dimensional arrangement. Examples of the two-dimensional arrangement include a linear shape. Examples of the three-dimensional arrangement include a ladder shape, a basket shape, and a mesh shape.

### <Organosilane Unit Having Aromatic Group>

The (A2-1) polysiloxane for use in the present invention preferably contains an organosilane unit having an aromatic group. Such a (A2-1) polysiloxane is preferably obtained with the use of an organosilane having an aromatic group as the organosilane having an organosilane unit represented by general formula (7), the general formula (9), or the general formula (10). The (A2-1) polysiloxane contains the organosilane unit having an aromatic group, thereby allowing the heat resistance of the aromatic group to improve the heat resistance of the cured film.

In addition, in the case of containing, in particular, the (D1) pigment as the (D) colorant described later, the (A2-1) polysiloxane contains the organosilane unit having an aromatic group, thereby allowing the steric hindrance of the aromatic group to improve the dispersion stability of the (D1) pigment. Furthermore, in a case where the (D1) pigment is an (D1-1) organic pigment, the aromatic group in the (A2-1) polysiloxane interacts with an aromatic group of the (D1-1) organic pigment, thus allowing the dispersion stability of the (D1-1) organic pigment to be improved.

The content ratio of the organosilane unit having an aromatic group to the polysiloxane (A2-1) is preferably 5 mol % or higher, more preferably 10 mol % or higher, still more preferably 15 mol % or higher in terms of Si atom mol ratio. When the content ratio is 5 mol % or higher, the heat resistance of the cured film can be improved. On the other hand, the content ratio is preferably 80 mol % or lower, more preferably 75 mol % or lower, still more preferably 70 mol % or lower. When the content ratio is 80 mol % or lower, the patternability with an alkaline developer can be improved. In particular, the Si atom mol ratio derived from the organosilane unit represented by general formula (7), the general formula (9), or the general formula (10) and having an aromatic group is 5 mol % or higher and 80 mol % or lower.

The content ratio of various types of organosilane units in the (A2-1) polysiloxane can be determined by combining ¹H-NMR, ¹³C-NMR, ²⁹Si-NMR, IR, TOF-MS, elemental analysis, ash measurement, and the like.

### <Physical Properties of (A2-1) Polysiloxane>

The Mw of the (A2-1) polysiloxane for use in the present invention is preferably 500 or more, more preferably 700 or more, still more preferably 1,000 or more in terms of polystyrene measured by GPC. When the Mw is 500 or more, the resolution after development can be improved. On the other hand, the Mw is preferably 100,000 or less, more preferably 50,000 or less, still more preferably 20,000 or less. When the Mw is 100,000 or less, the leveling property in the case of coating and the patternability with an alkaline developer can be improved.

The (A2-1) polysiloxane can be synthesized by known methods. The methods include a method in which an organosilane is hydrolyzed in a reaction solvent and subjected to dehydration and condensation. Examples of the method for hydrolyzing and dehydrating, and condensing the organosilane include a method of further adding a reaction solvent and water, and if necessary, a catalyst, to the mixture containing the organosilane, and heating and stirring the mixture for about 0.5 to 100 hours at 50 to 150° C., preferably 90 to 130° C. Further, during the heating and stirring, if necessary, hydrolysis by-products (alcohols such as methanol) and condensation by-products (water) may be distilled away by distillation.

### <(A2-2) Polycyclic Side Chain-Containing Resin>

Examples of the (A2-2) polycyclic side chain-containing resin for use in the present invention include the following (I) to (IV) polycyclic side chain-containing resins:

(I) The polycyclic side chain-containing resin obtained by reacting an epoxy compound with the compound obtained by reacting a polyfunctional phenol compound and a polyfunctional carboxylic anhydride.

(II) The polycyclic side chain-containing resin obtained by reacting a polyfunctional carboxylic anhydride with the compound obtained by reacting a polyfunctional phenol compound and an epoxy compound.

(III) The polycyclic side chain-containing resin obtained by reacting an epoxy compound with the compound obtained by reacting a polyfunctional epoxy compound with a polyfunctional carboxylic acid compound.

(IV) The polycyclic side chain-containing resin obtained by reacting a polyfunctional carboxylic anhydride with the compound obtained by reacting a polyfunctional epoxy compound with a carboxylic acid compound.

Examples of the phenol compound, epoxy compound, carboxylic anhydride, and carboxylic acid compound include the compounds described in International Publication No. 2017/057281.

The (A2-2) polycyclic side chain-containing resin, which is a thermosetting resin, has a structure with a main chain and a bulky side chain connected by one atom, and has, as the bulky side chain, a ring structure such as a high heat-resistance and rigid fluorene ring. Accordingly, the negative photosensitive resin composition contains therein the (A2-2) polycyclic side chain-containing resin that has a ring structure such as a high heat-resistance and rigid fluorene ring, thereby making it possible to improve the heat resistance of the cured film obtained. For that reason, the cured film is suitable in such a case of using the cured film for applications which require heat resistance.

The (A2-2) polycyclic side chain-containing resin for use in the present invention preferably has an ethylenically unsaturated double bond group. The negative photosensitive resin composition contains therein the (A2-2) polycyclic side chain-containing resin having an ethylenically unsaturated double bond group, thereby making it possible to improve the sensitivity for exposure. In addition, the three-dimensional crosslinked structure to be formed has, as its main component, an alicyclic structure or an aliphatic structure, thus keeping the softening point of the resin from being increased, making it possible to obtain a pattern in a low-taper shape, and making it possible to improve the mechanical characteristic of the cured film obtained. For that reason, the cured film is suitable in such a case of using the cured film for applications which require a mechanical characteristic.

The (A2-2) polycyclic side chain-containing resin for use in the present invention, from the viewpoint of improving the heat resistance of the cured film, preferably contains one or more selected from a structural unit represented by general formula (47), a structural unit represented by general formula (48), a structural unit represented by general formula (49), and a structural unit represented by general formula (50). In addition, the (A2-2) polycyclic side chain-containing resin for use in the present invention preferably contains an ethylenically unsaturated double bond group for any one or more of the main chain, the side chain, and the terminal, from the viewpoint of improving the sensitivity for exposure and improving the mechanical characteristic of the cured film.

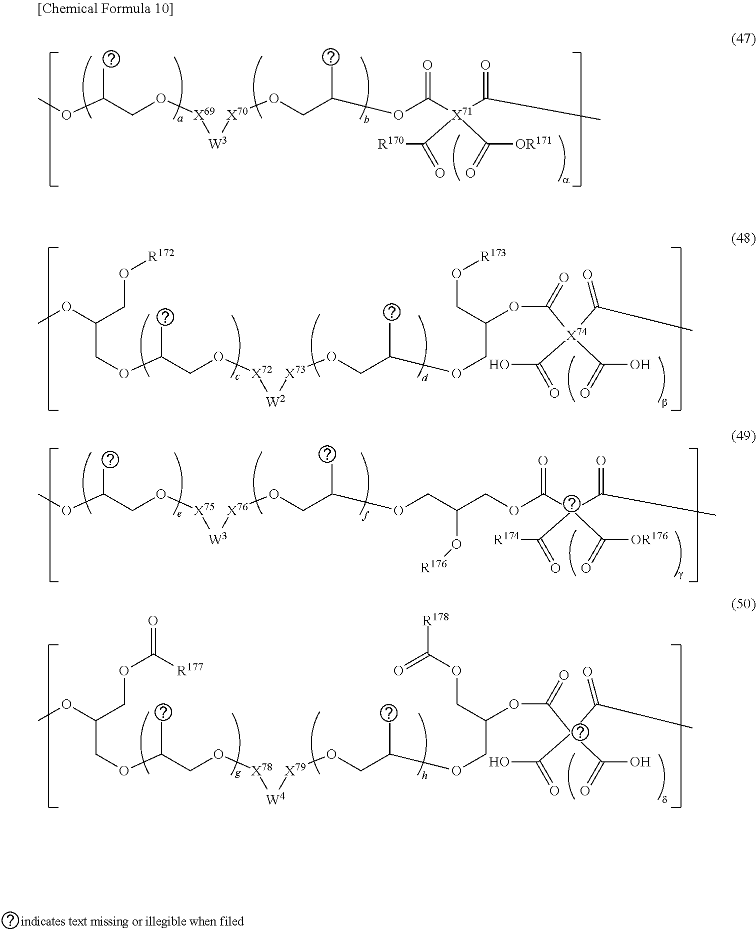

In the general formulas (47) to (50), X⁶⁹, X⁷⁰, X⁷², X⁷³, X⁷⁵, X⁷⁶, X⁷⁸, and X⁷⁹ each independently represent a monocyclic or condensed polycyclic hydrocarbon ring. X⁷¹, X⁷⁴, X⁷⁷, and X⁸⁰ each independently represent a divalent to decavalent organic group of a carboxylic acid residue and/or a derivative residue thereof. W¹ to W⁴ each independently represents a norganic group having two or more aromatic groups. R¹⁶⁰ to R¹⁶⁷ each independently represent hydrogen or an alkyl group having 1 to 6 carbon atoms, and R¹⁷⁰ to R¹⁷⁵, R¹⁷⁷, and R¹⁷⁸ each independently represent hydrogen or an organic group having an ethylenically unsaturated double bond group. R¹⁷⁶ represents hydrogen or an alkyl group having 1 to 10 carbon atoms. a, b, c, d, e, f, g, and h each independently represent an integer of 0 to 10, and α, β, γ, and δ each independently represent 0 or 1.

In the general formulas (47) to (50), X⁶⁹, X⁷⁰, X⁷², X⁷³, X⁷⁵, X⁷⁶, X⁷⁸, and X⁷⁹ each independently preferably represent a divalent to decavalent monocyclic or polycyclic condensed hydrocarbon ring having 6 to 15 carbon atoms. Furthermore, X⁷¹, X⁷⁴, X⁷⁷, and X⁸⁰ each independently preferably represent a divalent to decavalent organic group having one or more selected from an aliphatic structure having 2 to 20 carbon atoms, an alicyclic structure having 4 to 20 carbon atoms, and an aromatic structure having 6 to 30 carbon atoms. Furthermore, W¹ to W⁴ each independently preferably represent a substituent represented by any of the general formulas (51) to (56). Furthermore, R¹⁷⁰ to R¹⁷⁵, R¹⁷⁷ and R¹⁷⁸ each independently preferably represent a substituent represented by general formula (57). The organic groups having an alkyl group, an aliphatic structure, alicyclic structure, an aromatic structure, a monocyclic or polycyclic condensed aromatic hydrocarbon ring, and an ethylenically unsaturated double bond group as described above may have a hetero atom, and may be either unsubstituted or substituted.

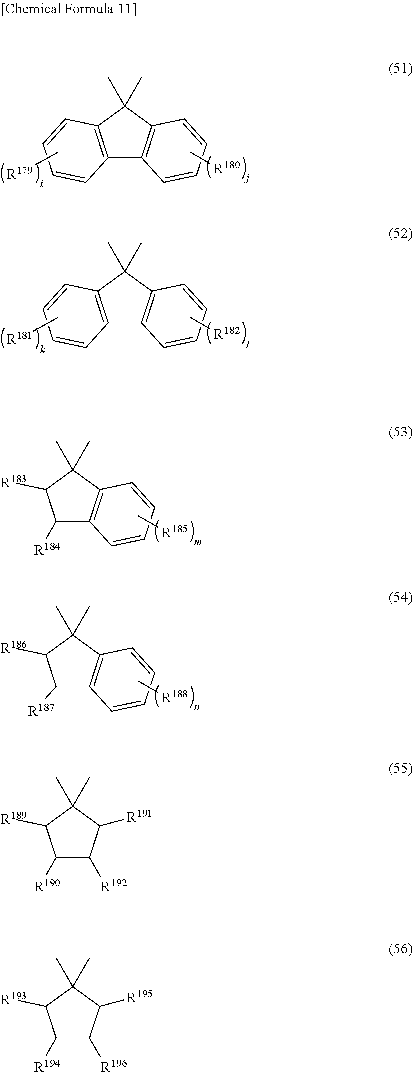

In the general formulas (51) to (56), R¹⁷⁹ to R⁸², R¹⁸⁵, and R¹⁸⁸ each independently represent an alkyl group having 1 to 10 carbon atoms. R¹⁸³, R¹⁸⁴, R¹⁸⁶, R¹⁸⁷, R¹⁸⁹, R¹⁹¹, and R¹⁹³ to R¹⁹⁶ each independently represent hydrogen, an alkyl group having 1 to 10 carbon atoms, a cycloalkyl group having 4 to 10 carbon atoms, or an aryl group having 6 to 15 carbon atoms. R¹⁹⁰ and R¹⁹² each independently represent hydrogen, an alkyl group having 1 to 10 carbon atoms, a cycloalkyl group having 4 to 10 carbon atoms, or an aryl group having 6 to 15 carbon atoms, and R¹⁹⁰ and R¹⁹² may form a ring. Examples of the ring formed by R¹⁹⁰ and R¹⁹² include a benzene ring or a cyclohexane ring. At least one of R¹⁸³ and R¹⁸⁴ represents an aryl group having 6 to 15 carbon atoms. at least one of R¹⁸⁶ and R¹⁸⁷ represents an aryl group having 6 to 15 carbon atoms. at least one of R¹⁸⁹ and R¹⁵⁰ represents an aryl group having 6 to 15 carbon atoms, and at least one of R¹⁹¹ and R¹⁹² represents an aryl group having 6 to 15 carbon atoms, and R¹⁹⁰ and R¹⁹² may form a ring. At least one of R⁹³ and R⁹⁴ represents an aryl group having 6 to 15 carbon atoms, and at least one of R¹⁹⁵ and R¹⁹ represents an aryl group having 6 to 15 carbon atoms. i, j, k, l, m, and n each independently represent an integer of 0 to 4. In the general formulas (51) to (56), R¹⁹⁰ and R¹⁹² each independently preferably represent hydrogen, an alkyl group having 1 to 6 carbon atoms, a cycloalkyl group having 4 to 7 carbon atoms, or an aryl group having 6 to 10 carbon atoms, and the ring formed by R¹⁹⁰ and R¹⁹² is preferably a benzene ring. The above-described alkyl group, cycloalkyl group, and aryl group may be either unsubstituted or substituted.

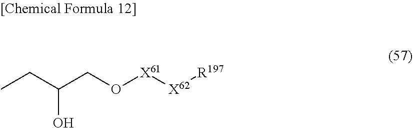

In the general formula (57), X⁸¹ represents a direct bond, an alkylene chain having 1 to 10 carbon atoms, a cycloalkylene chain having 4 to 10 carbon atoms, or an arylene chain having 6 to 15 carbon atoms, and X⁸² represents a direct bond or an arylene chain having 6 to 15 carbon atoms. R¹⁹⁷ represents a vinyl group, an aryl group, or a (meth)acrylic group. In the general formula (57), X⁸¹ preferably represents a direct bond, an alkylene chain having 1 to 6 carbon atoms, a cycloalkylene chain having 4 to 7 carbon atoms, or an arylene chain having 6 to 10 carbon atoms. Furthermore, X⁸² preferably represents a direct bond or an arylene chain having 6 to 10 carbon atoms. The alkylene chain, cycloalkylene chain, arylene chain, vinyl group, aryl group, and (meth)acrylic group described above may be either unsubstituted or substituted.

<Structural Unit Derived from One or More Selected from Tetracarboxylic Acid Having Aromatic Carboxylic Acid and Derivative Thereof, Tetracarboxylic Dianhydride Having Aromatic Group, Tricarboxylic Acid Having Aromatic Group, and Dicarboxylic Acid Having Aromatic Group>

The (A2-2) polycyclic side chain-containing resin for use in the present invention preferably contains a structural unit derived from an aromatic carboxylic acid and a derivative thereof. The (A2-2) polycyclic side chain-containing resin contains a structural unit derived from an aromatic carboxylic acid and a derivative thereof, thereby allowing the heat resistance of the aromatic group to improve the heat resistance of the cured film. As the aromatic carboxylic acid a derivative thereof, one or more selected from a tetracarboxylic acid having an aromatic group, a tetracarboxylic dianhydride having an aromatic group, a tricarboxylic acid having an aromatic group, and a dicarboxylic acid having an aromatic group are preferred.

In addition, in the case of containing, in particular, the (D1) pigment as the (D) colorant described later, the (A2-2) polycyclic side chain-containing resin contains a structural unit derived from an aromatic carboxylic acid and a derivative thereof, thereby allowing the steric hindrance of the aromatic group to improve the dispersion stability of the (D1) pigment. Furthermore, in a case where the (D1) pigment is an (D1-1) organic pigment, the aromatic group in the (A2-2) polycyclic side chain-containing resin interacts with an aromatic group of the (D1-1) organic pigment, thus allowing the dispersion stability of the (D1-1) organic pigment to be improved.

Examples of the aromatic carboxylic acid and derivative thereof include the above-mentioned compounds included in the aromatic tetracarboxylic acid and/or derivative thereof, aromatic tricarboxylic acid and/or derivative thereof, or aromatic dicarboxylic acid and/or derivative thereof.

The content ratio of the structural units derived from aromatic carboxylic acids and/or derivatives thereof to structural units derived from all tetracarboxylic acids and all dicarboxylic acids and derivatives thereof in the (A2-2) polycyclic side chain-containing resin is preferably 10 to 100 mol %, more preferably 20 to 100 mol %, still more preferably 30 to 100 mol %. When the content ratio is 10 to 100 mol %, the heat resistance of the cured film can be improved.

<Acid Group Derived from Carboxylic Acid and Derivative Thereof>

The (A2-2) polycyclic side chain-containing resin for use in the present invention contains a structural unit derived from a carboxylic acid and a derivative thereof, and the (A2-2) polycyclic side chain-containing resin preferably has an acidic group. The (A2-2) polycyclic side chain-containing resin has an acidic group, thereby allowing the patternability with an alkaline developer and the resolution after development to be improved.

As the acidic group, a group that exhibits an acidity of less than pH 6 is preferred. Examples of the group that exhibits an acidity of less than pH 6 include a carboxy group, a carboxylic anhydride group, a sulfonic acid group, a phenolic hydroxyl group, and a hydroxyimide group. From the viewpoint of improving the patternability with an alkaline developer and improving the resolution after development, a carboxy group, a carboxylic anhydride group, or a phenolic hydroxyl group is preferred, and a carboxy group or a carboxylic anhydride group is more preferred.

The content ratio of structural units derived from various types of monomer components in the (A2-2) polycyclic side chain-containing resin can be determined by combining ¹H-NMR, ¹³C-NMR, ²⁹Si-NMR, IR, TOF-MS, elemental analysis, ash measurement, and the like.

### <Specific Examples of (A2-2) Polycyclic Side Chain-Containing Resin>

Examples of the (A2-2) polycyclic side chain-containing resin for use in the present invention include “ADEKA ARKLS” (registered trademark) WR-101 or WR-301 (all manufactured by ADEKA Corporation), OGSOL (registered) trademark) CR-1030, CR-TR1, CR-TR2, CR-TR3, CR-TR4, CR-TR5, CR-TR6, CR-TR7, CR-TR8, CR-TR9, or CR-TR10 (all manufactured by Osaka Gas Chemicals Co., Ltd.), and TR-B201 or TR-B202 (all manufactured by TRONLY).

### <Physical Properties of (A2-2) Polycyclic Side Chain-Containing Resin>

The Mw of the (A2-2) polycyclic side chain-containing resin for use in the present invention is preferably 500 or more, more preferably 1,000 or more, still more preferably 1,500 or more in terms of polystyrene measured by GPC. When the Mw is 500 or more, the resolution after development can be improved. On the other hand, the Mw is preferably 100,000 or less, more preferably 50,000 or less, still more preferably 20,000 or less. When the Mw is 100,000 or less, the leveling property in the case of coating and the patternability with an alkaline developer can be improved.

### <(A2-3) Acid-Modified Epoxy Resin>

Examples of the (A2-3) acid-modified epoxy resin for use in the present invention include the following acid-modified epoxy resins (I) to (VI).

(I) The acid-modified epoxy resin obtained by reacting an epoxy compound with the compound obtained by reacting a polyfunctional phenol compound and a polyfunctional carboxylic anhydride.

(II) The acid-modified epoxy resin obtained by reacting a polyfunctional carboxylic anhydride with the compound obtained by reacting a polyfunctional phenol compound and an epoxy compound.

(III) The acid-modified epoxy resin obtained by reacting an epoxy compound with the compound obtained by reacting a polyfunctional alcohol compound and a polyfunctional carboxylic anhydride.

(IV) The acid-modified epoxy resin obtained by reacting a polyfunctional carboxylic anhydride with the compound obtained by reacting a polyfunctional alcohol compound and an epoxy compound.

(V) The acid-modified epoxy resin obtained by reacting an epoxy compound with the compound obtained by reacting a polyfunctional epoxy compound with a polyfunctional carboxylic acid compound.

(VI) The acid-modified epoxy resin obtained by reacting a polyfunctional carboxylic anhydride with the compound obtained by reacting a polyfunctional epoxy compound and a carboxylic acid compound.

Examples of the phenol compound, alcohol compound, epoxy compound, carboxylic anhydride, and carboxylic acid compound include the compounds described in International Publication No. 2017/057281.

The (A2-3) acid-modified epoxy resin, which is a thermosetting resin, has a highly heat-resistance aromatic ring structure in the epoxy resin skeleton of the main chain. Accordingly, the resin composition contains therein the (A2-3) acid-modified epoxy resin, thereby making it possible improve the heat resistance of the cured film obtained. For that reason, the cured film is suitable in such a case of using the cured film for applications which require heat resistance.

The (A2-3) acid-modified epoxy resin for use in the present invention preferably has an ethylenically unsaturated double bond group. The resin composition contains therein the (A2-3) acid-modified epoxy resin having an ethylenically unsaturated double bond group, thereby making it possible to improve the sensitivity for exposure. In addition, the three-dimensional crosslinked structure to be formed has, as its main component, an alicyclic structure or an aliphatic structure, thus keeping the softening point of the resin from being increased, making it possible to obtain a pattern in a low-taper shape, and making it possible to improve the mechanical characteristic of the cured film obtained. For that reason, the cured film is suitable in such a case of using the cured film for applications which require a mechanical characteristic.

The (A2-3) acid-modified epoxy resin for use in the present invention has a carboxy group and/or a carboxylic anhydride group as an alkali-soluble group. The resin has a carboxy group and/or a carboxylic anhydride group, allowing the resolution after development to be improved.

The (A2-3) acid-modified epoxy resin for use in the present invention preferably contains, from the viewpoint of improving the heat resistance of the cured film, one or more selected from a structural unit represented by general formula (35), a structural unit represented by general formula (36), a structural unit represented by general formula (37), a structural unit represented by general formula (38), a structural unit represented by general formula (41), a structural unit represented by general formula (42), and a structural unit represented by general formula (43). In addition, the (A2-3) acid-modified epoxy resin for use in the present invention preferably has an ethylenically unsaturated double bond group for any one or more of the main chain, the side chain, and the terminal, from the viewpoint of improving the sensitivity for exposure and improving the mechanical characteristic of the cured film.

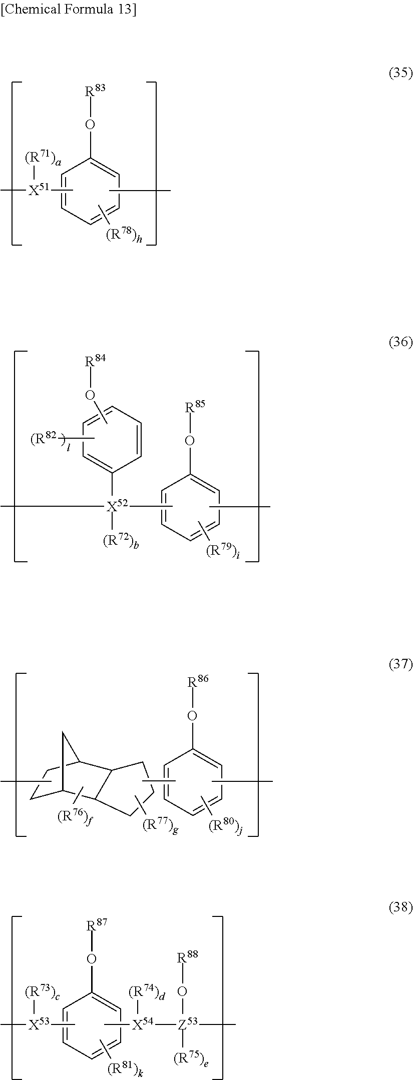

In the general formulas (35) to (38), X⁵¹ to X⁵⁴ each independently represent an aliphatic structure having 1 to 6 carbon atoms. Z⁵³ represents a trivalent to 16-valent aromatic structure having 10 to 25 carbon atoms. R⁷¹ to R⁷⁵ each independently represent an alkyl group having 1 to 10 carbon atoms, a cycloalkyl group having 4 to 10 carbon atoms, or an aryl group having 6 to 15 carbon atoms, and R⁷⁶ and R⁷⁷ each independently represent an alkyl group having 1 to 10 carbon atoms, R⁷⁸ to R⁸² each independently represent halogen, an alkyl group having 1 to 10 carbon atoms, a cycloalkyl group having 4 to 10 carbon atoms, or an aryl group having 6 to 15 carbon atoms, and R⁸³ to R⁸⁸ each independently represent a substituent represented by general formula (39). a, b, c, d, and e each independently represent an integer of 0 to 10, f represents an integer of 0 to 8, g represents an integer of 0 to 6, h, i, j, and k each independently represent an integer of 0 to 3, and 1 represents an integer of 0 to 4. The above-described alkyl group, cycloalkyl group, aryl group, aliphatic structure, and aromatic structure may have a hetero atom, and may be either unsubstituted or substituted.

The aromatic structure for Z⁵³ of the general formula (38) contains one or more selected from the group consisting of a terphenyl structure, a naphthalene structure, an anthracene structure, and a fluorene structure. Other aromatic structures for Z⁵³ of the general formula (38) include a 1,2,3,4-tetrahydronaphthalene structure, a 2,2-diphenylpropane structure, a diphenyl ether structure, a diphenyl ketone structure, and a diphenyl sulfone structure.

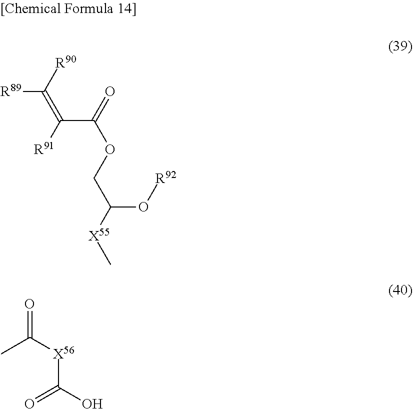

In the general formula (39), X⁵⁵ represents an alkylene chain having 1 to 6 carbon atoms or a cycloalkylene chain having 4 to 10 carbon atoms. R⁸⁹ to R⁹¹ each independently represent hydrogen, an alkyl group having 1 to 10 carbon atoms, or an aryl group having 6 to 15 carbon atoms. R⁹² represents hydrogen or a substituent represented by general formula (40). In the general formula (39), R⁸⁹ and R⁹⁰ each independently preferably represent hydrogen or an alkyl group having 1 to 4 carbon atoms, more preferably hydrogen. R⁹¹ preferably represents hydrogen or an alkyl group having 1 to 4 carbon atoms, more preferably hydrogen or a methyl group. In the general formula (40), X⁵⁶ represents an alkylene chain having 1 to 6 carbon atoms or a cycloalkylene chain having 4 to 10 carbon atoms. In the general formula (40), X⁵⁶ preferably represents an alkylene chain having 1 to 4 carbon atoms or a cycloalkylene chain having 4 to 7 carbon atoms. The alkylene chain, cycloalkylene chain, alkyl group, and aryl group described above may be either unsubstituted or substituted.

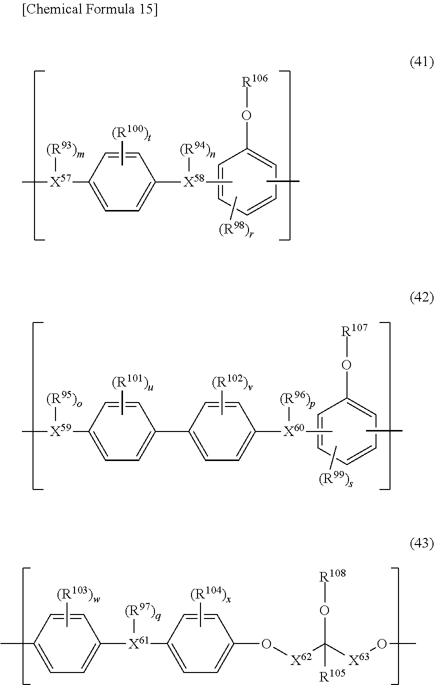

In the general formulas (41) to (43), X⁵⁷ to X⁶¹ each independently represent an aliphatic structure having 1 to 6 carbon atoms, and X⁶² and X⁶³ each independently represent an alkylene chain having 1 to 6 carbon atoms, or a cycloalkylene chain having 4 to 10 carbon atoms. R⁹³ to R⁹⁷ each independently represent an alkyl group having 1 to 10 carbon atoms, a cycloalkyl group having 4 to 10 carbon atoms, or an aryl group having 6 to 15 carbon atoms, and R⁹⁸ to R¹⁰⁴ each independently represent halogen, an alkyl group having 1 to 10 carbon atoms, a cycloalkyl group having 4 to 10 carbon atoms, or an aryl group having 6 to 15 carbon atoms, R¹⁰⁵ represents hydrogen or an alkyl group having 1 to 6 carbon atoms, R¹⁰⁵ and R¹⁰⁷ each independently represent a substituent represented by general formula (39), and R¹⁰⁸ represents hydrogen, a substituent represented by general formula (39), or a substituent represented by general formula (40). m, n, o, p, and q each independently represent an integer of 0 to 10, r and s each independently represent an integer of 0 to 3, and t, u, v, w, and x each independently represent an integer of 0 to 4. The above-mentioned alkylene chain, cycloalkylene chain, alkyl group, cycloalkyl group, aryl group, and aliphatic structure may have a hetero atom, and may be either unsubstituted or substituted.

Among the (A2-3) acid-modified epoxy resins for use in the present invention, as the (A2-3) acid-modified epoxy resin having a structural unit represented by general formula (43), the terminal preferably has a substituent represented by general formula (44) and/or a substituent represented by general formula (45).

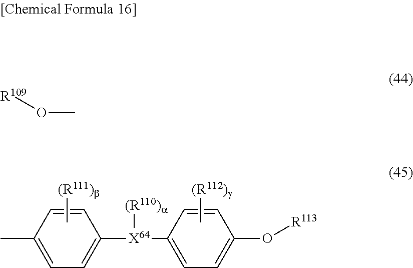

In the general formula (44), R¹⁰⁹ represents a substituent represented by general formula (39). In the general formula (45), X⁶⁴ represents an aliphatic structure having 1 to 6 carbon atoms. R¹⁰ represents an alkyl group having 1 to 10 carbon atoms, a cycloalkyl group having 4 to 10 carbon atoms, or an aryl group having 6 to 15 carbon atoms, and R¹¹¹ and R¹¹² each independently represent halogen, an alkyl group having 1 to 10 carbon atoms, a cycloalkyl group having 4 to 10 carbon atoms, or an aryl group having 6 to 15 carbon atoms. R¹¹³ represents a substituent represented by general formula (39). α represents an integer of 0 to 10. β and γ represent integers of 0 to 4. In the general formula (45), X⁶ preferably represents an aliphatic structure having 1 to 4 carbon atoms. R¹⁰ preferably represents an alkyl group having 1 to 6 carbon atoms, a cycloalkyl group having 4 to 7 carbon atoms, or an aryl group having 6 to 10 carbon atoms, and R¹¹¹ and R¹¹² each independently represent halogen, an alkyl group having 1 to 6 carbon atoms, a cycloalkyl group having 4 to 7 carbon atoms, or an aryl group having 6 to 10 carbon atoms.

<Structural Units Derived from Aromatic Carboxylic Acid and Derivative Thereof>

The (A2-3) acid-modified epoxy resin for use in the present invention preferably contains a structural unit derived from an aromatic carboxylic acid and a derivative thereof. The (A2-3) acid-modified epoxy resin contains a structural unit derived from an aromatic carboxylic acid and a derivative thereof, thereby allowing the heat resistance of the aromatic group to improve the heat resistance of the cured film. As the aromatic carboxylic acid and derivative thereof, one or more selected from a tetracarboxylic acid having an aromatic group, a tricarboxylic acid having an aromatic group, a tricarboxylic anhydride having an aromatic group, a dicarboxylic acid having an aromatic group, and a dicarboxylic anhydride having an aromatic group are preferred.

In addition, in the case of containing, in particular, the (D1) pigment as the (D) colorant described later, the (A2-3) acid-modified epoxy resin contains a structural unit derived from an aromatic carboxylic acid and a derivative thereof, thereby allowing the steric hindrance of the aromatic group to improve the dispersion stability of the (D1) pigment. Furthermore, in a case where the (D1) pigment is an (D1-1) organic pigment, the aromatic group in the (A2-3) acid-modified epoxy resin interacts with an aromatic group of the (D1-1) organic pigment, thus allowing the dispersion stability of the (D1-1) organic pigment to be improved.

Examples of the aromatic carboxylic acid and derivative thereof include the above-described compounds included in the aromatic tetracarboxylic acid and/or derivative thereof, the aromatic tricarboxylic acid and/or derivative thereof, or the aromatic dicarboxylic acid and/or derivative thereof.

The content ratio of the structural units derived from aromatic carboxylic acids and/or derivatives thereof to structural units derived from all carboxylic acids and derivatives thereof in the (A2-3) acid-modified epoxy resin is preferably 10 to 100 mol %, more preferably 20 to 100 mol %, still more preferably 30 to 100 mol %. When the content ratio is 10 to 100 mol %, the heat resistance of the cured film can be improved.

<Acid Group Derived from Carboxylic Acid and Derivative Thereof>

The (A2-3) acid-modified epoxy resin for use in the present invention preferably contains a structural unit derived from a carboxylic acid and a derivative thereof, and the (A2-3) acid-modified epoxy resin preferably has an acidic group. The (A2-3) acid-modified epoxy resin has an acidic group, thereby allowing the patternability with an alkaline developer and the resolution after development to be improved.

As the acidic group, a group that exhibits an acidity of less than pH 6 is preferred. Examples of the group that exhibits an acidity of less than pH 6 include a carboxy group, a carboxylic anhydride group, a sulfonic acid group, a phenolic hydroxyl group, and a hydroxyimide group. From the viewpoint of improving the patternability with an alkaline developer and improving the resolution after development, a carboxy group, a carboxylic anhydride group, or a phenolic hydroxyl group is preferred, and a carboxy group or a carboxylic anhydride group is more preferred.

The content ratio of structural units derived from various types of monomer components in the (A2-3) acid-modified epoxy resin can be determined by combining ¹H-NMR, ¹³C-NMR, ²⁹Si-NMR, IR, TOF-MS, elemental analysis, ash measurement, and the like.

### <Specific Examples of (A2-3) Acid-Modified Epoxy Resin>

Examples of the (A2-3) acid-modified epoxy resin for use in the present invention include “KAYARAD” (registered trademark) PCR-1222H, CCR-1171H, TCR-1348H, ZAR-1494H, ZFR-1401H, ZCR-1798H, ZXR-1807H, ZCR-6002H, or ZCR-8001H (all manufactured by Nippon Kayaku Co., Ltd.) or “NK OLIGO” (registered trademark) EA-6340, EA-7140, or EA-7340 (all manufactured by Shin Nakamura Chemical Co., Ltd.).

### <Physical Properties of (A2-3) Acid-Modified Epoxy Resin>

The Mw of the (A2-3) acid-modified epoxy resin for use in the present invention is preferably 500 or more, more preferably 1,000 or more, still more preferably 1,500 or more in terms of polystyrene measured by GPC. When the Mw falls within the range described above, the resolution after development can be improved. On the other hand, the Mw is preferably 100,000 or less, more preferably 50,000 or less, still more preferably 20,000 or less. When the Mw falls within the range described above, the leveling property in the case of coating and the patternability with an alkaline developer can be improved.

### <(A2-4) Acrylic Resin>

Examples of the (A2-4) acrylic resin for use in the present invention include the acrylic resin obtained by radical copolymerization of one or more selected from a copolymerization component having an acidic group, a copolymerization component derived from a (meth)acrylic ester, and other copolymerization components.

Examples of the copolymerization component having an acidic group, the copolymerization component derived from a (meth)acrylic acid ester, and other copolymer components include the compounds described in International Publication No. 2017/057281.

The (A2-4) acrylic resin for use in the present invention preferably has an ethylenically unsaturated double bond group. The negative photosensitive resin composition contains therein the (A2-4) acrylic resin having an ethylenically unsaturated double bond group, thereby making it possible to improve the sensitivity for exposure. In addition, the three-dimensional crosslinked structure to be formed has, as its main component, an alicyclic structure or an aliphatic structure, thus keeping the softening point of the resin from being increased, making it possible to obtain a pattern in a low-taper shape, and making it possible to improve the mechanical characteristic of the cured film obtained. For that reason, the cured film is suitable in such a case of using the cured film for applications which require a mechanical characteristic.

The (A2-4) acrylic resin for use in the present invention preferably contains a structural unit represented by general formula (61) and/or a structural unit represented by general formula (62), from the viewpoint of improving the sensitivity for exposure and improving the mechanical characteristic of the cured film.

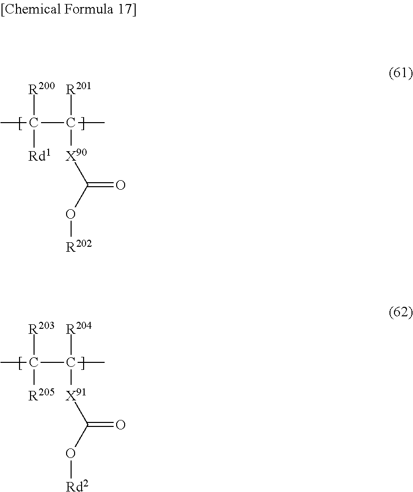

In the general formulas (61) and (62), Rd¹ and Rd² each independently represent an alkyl group having 1 to 10 carbon atoms, a cycloalkyl group having 4 to 15 carbon atoms, or an aryl group having 6 to 15 carbon atoms, which has an ethylenically unsaturated double bond group. R²⁰⁰ to R²⁰⁵ each independently represent hydrogen, an alkyl group having 1 to 10 carbon atoms, a cycloalkyl group having 4 to 10 carbon atoms, or an aryl group having 6 to 15 carbon atoms. X⁹⁰ and X⁹¹ each independently represent a direct bond, an alkylene chain having 1 to 10 carbon atoms, a cycloalkylene chain having 4 to 10 carbon atoms, or an arylene chain having 6 to 15 carbon atoms.

In the general formulas (61) and (62), Rd¹ and Rd² each independently preferably represent an alkyl group having 1 to 6 carbon atoms, a cycloalkyl group having 4 to 10 carbon atoms, or an aryl group having 6 to 10 carbon atoms, which has an ethylenically unsaturated double bond group. In addition, R²⁰⁰ to R²⁰⁵ each independently represent preferably hydrogen, an alkyl group having 1 to 6 carbon atoms, a cycloalkyl group having 4 to 7 carbon atoms, or an aryl group having 6 to 10 carbon atoms. In addition, X⁹⁰ and X⁹¹ each independently preferably represent a direct bond, an alkylene chain having 1 to 6 carbon atoms, a cycloalkylene chain having 4 to 7 carbon atoms, or an arylene chain having 6 to 10 carbon atoms. The alkyl group, cycloalkyl group, aryl group, alkylene chain, cycloalkylene chain, and arylene chain described above may have a hetero atom, and may be either unsubstituted or substituted.

The (A2-4) acrylic resin for use in the present invention is preferably an (A2-4) acrylic resin obtained by radical copolymerization of copolymerization components having acidic groups or other copolymerization components. As the other copolymer components, copolymerization components having aromatic groups or copolymerization components having alicyclic groups are preferred.

<Structural Unit Derived from Copolymerization Component Having Acidic Group>

The (A2-4) acrylic resin for use in the present invention preferably contains a structural unit derived from a copolymerization component having an acidic group, and the (A2-4) acrylic resin preferably has an acidic group. The (A2-4) acrylic resin has an acidic group, thereby allowing the patternability with an alkaline developer and the resolution after development to be improved.

As the acidic group, a group that exhibits an acidity of less than pH 6 is preferred. Examples of the group that exhibits an acidity of less than pH 6 include a carboxy group, a carboxylic anhydride group, a sulfonic acid group, a phenolic hydroxyl group, and a hydroxyimide group. From the viewpoint of improving the patternability with an alkaline developer and improving the resolution after development, a carboxy group, a carboxylic anhydride group, or a phenolic hydroxyl group is preferred, and a carboxy group or a carboxylic anhydride group is more preferred.

As the (A2-4) acrylic resin for use in the present invention, an (A2-4) acrylic resin having no epoxy group is preferred in a case where the (A2-4) acrylic resin has a carboxy group. If the (A2-4) acrylic resin has both a carboxy group and an epoxy group, there is a possibility that the carboxy group and the epoxy group may react during the storage of a coating liquid with the negative photosensitive resin composition. Thus, the reaction causes the storage stability of the coating liquid with the resin composition to be decreased. As the (A2-4) acrylic resin having no epoxy group, an (A2-4) acrylic resin obtained by radical copolymerization of a copolymerization component having a carboxy group or a carboxylic anhydride group and another copolymerization component having no epoxy group is preferred.

<Structural Unit Derived from Copolymerization Component Having Aromatic Group>

The (A2-4) acrylic resin for use in the present invention preferably contains a structural unit derived from a copolymerization component having an aromatic group. The (A2-4) acrylic resin contains a structural unit derived from a copolymerization component having an aromatic group, thereby allowing the heat resistance of the aromatic group to improve the heat resistance of the cured film.

In addition, in the case of containing, in particular, the (D1) pigment as the (D) colorant described later, the (A2-4) acrylic resin contains the structural unit derived from a copolymerization component having an aromatic group, thereby allowing the steric hindrance of the aromatic group to improve the dispersion stability of the (D1) pigment. Furthermore, in a case where the (D1) pigment is an (D1-1) organic pigment, the aromatic group in the (A2-4) acrylic resin interacts with an aromatic group of the (D1-1) organic pigment, thus allowing the dispersion stability of the (D1-1) organic pigment to be improved.

The content ratio of the structural unit derived from the copolymerization component having an aromatic group to structural units derived from all of the copolymerization components in the (A2-4) acrylic resin is preferably 10 mol % or higher, more preferably 20 mol % or higher, still more preferably 30 mol % or higher. When the content ratio is 10 mol % or higher, the heat resistance of the cured film can be improved. On the other hand, the content ratio is preferably 80 mol % or lower, more preferably 75 mol % or lower, still more preferably 70 mol % or lower. When the content ratio is 80 mol % or lower, the sensitivity for exposure can be improved.

<Structural Unit Derived from Copolymerization Component Having Alicyclic Group>

The (A2-4) acrylic resin for use in the present invention preferably contains a structural unit derived from a copolymerization component having an alicyclic group. The (A2-4) acrylic resin contains a structural unit derived from a copolymerization component having an alicyclic group, thereby allowing the heat resistance and transparency of the alicyclic group to improve the heat resistance and transparency of the cured film.

The content ratio of the structural unit derived from the copolymerization component having an alicyclic group to structural units derived from all of the copolymerization components in the (A2-4) acrylic resin is preferably 5 mol % or higher, more preferably 10 mol % or higher, still more preferably 15 mol % or higher. When the content ratio is 5 mol % or higher, the heat resistance and transparency of the cured film can be improved. On the other hand, the content ratio is preferably 90 mol % or lower, more preferably 85 mol % or lower, still more preferably 75 mol % or lower. When the content ratio is 90 mol % or lower, the mechanical characteristic of the cured film can be improved.

As the (A2-4) acrylic resin for use in the present invention, a resin obtained further by the ring-opening addition reaction of an unsaturated compound having an ethylenically unsaturated double bond group and an epoxy group with a resin obtained by radical copolymerization of copolymerization components having an acidic groups or other copolymerization components is preferred. The ring-opening addition reaction of the unsaturated compound having an ethylenically unsaturated double bond group and an epoxy group allows an ethylenically unsaturated double bond group to be introduced into the side chain of the (A2-4) acrylic resin.

The content ratio of structural units derived from various types of copolymerization components in the (A2-4) acrylic resin can be determined by combining ¹H-NMR, ¹³C-NMR, ²⁹Si-NMR, IR, TOF-MS, elemental analysis, ash measurement, and the like.

### <Physical Properties of (A2-4) Acrylic Resin>

The Mw of the (A2-4) acrylic resin for use in the present invention is preferably 1,000 or more, more preferably 3,000 or more, still more preferably 5,000 or more in terms of polystyrene measured by GPC. When the Mw is 1,000 or more, the resolution after development can be improved. On the other hand, the Mw is preferably 100,000 or less, more preferably 70,000 or less, still more preferably 50,000 or less. When the Mw is 100,000 or less, the leveling property in the case of coating and the patternability with an alkaline developer can be improved.

The (A2-4) acrylic resin can be synthesized by known methods. Examples thereof include a method for radical copolymerization of a copolymerization component in the presence of a radical polymerization initiator in air or nitrogen. Examples of the method for radical copolymerization include a method of sufficiently purging the inside of a reaction container with nitrogen in air or by bubbling or degassing under reduced pressure, adding, into a reaction solvent therein, copolymerization components and a radical polymerization initiator, reacting the components at 60 to 110° C. for 30 to 500 minutes. Furthermore, a chain transfer agent such as a thiol compound and/or a polymerization terminator such as a phenol compound may be used, if necessary.

In the negative photosensitive resin composition according to the present invention, the content ratio of the (A1) first resin to 100% by mass of the (A1) first resin and (A2) second resin in total is preferably 25% by mass or higher, more preferably 50% by mass or higher, still more preferably 60% by mass or higher, even more preferably 70% by mass or higher, particularly preferably 80% by mass or higher. When the content ratio is 25% by mass or higher, the heat resistance of the cured film and the reliability of the light emitting element can be improved. In addition, the change in pattern opening width between before and after thermal curing can be suppressed, and the halftone characteristics can be improved. On the other hand, the content ratio of the (A1) first resin is preferably 99% by mass or lower, more preferably 98% by mass or lower, still more preferably 97% by mass or lower, even more preferably 95% by mass or lower, particularly preferably 90% by mass or lower. When the content ratio is 99% by mass or lower, the sensitivity for exposure can be improved, and a pattern in a low-taper shape can be formed after development. In addition, halftone characteristics can be improved.

When the content ratio of the (A1) first resin and (A2) second resin in the negative photosensitive resin composition according to the present invention falls within the above-described preferred range, the heat resistance of the cured film can be improved, and a pattern in a low-taper shape can be obtained. Accordingly, the cured film obtained from the negative photosensitive resin composition according to the present invention is suitable for applications which require high heat resistance and a pattern in a low-taper shape, e.g., an insulation layer such as a pixel defining layer of an organic EL display, a TFT planarization layer, or a TFT protective layer. In particular, in applications in which problems due to heat resistance and pattern shapes are expected, such as element failures or characteristic degradation due to degassing by thermal decomposition, or electrode wiring disconnection due to high-taper pattern shapes, the use of a cured film of the negative photosensitive resin composition according to the present invention makes it possible to manufacture a highly reliable element where the above-described problems are not caused. In addition, the negative photosensitive resin composition according to the present invention contains the (D) colorant described later, thus allowing electrode wiring to be prevented from becoming visible or allowing external light reflection to be reduced, and the contrast in image display can be thus improved.

### <(B) Radical Polymerizable Compound>

The negative photosensitive resin composition according to the present invention preferably further contains a (B) radical polymerizable compound. The (B) radical polymerizable compound refers to a compound having a plurality of ethylenically unsaturated double bond groups in the molecule. During exposure, radicals generated from a (C1) photo initiator to be described later causes radical polymerization of the (B) radical polymerizable compound to proceed, thereby making the exposed part of the film of the resin composition insoluble in an alkaline developer, and then allowing a negative pattern to be formed.

Containing the (B) radical polymerizable compound accelerates UV curing during the exposure, thereby allowing the sensitivity for the exposure to be improved. In addition, the crosslink density after thermal curing is improved, thereby allowing the hardness of the cured film to be improved.

As the (B) radical polymerizable compound, a compound having a (meth)acrylic group is preferred, which facilitates radical polymerization. From the viewpoint of improving the sensitivity for exposure and improving the hardness of the cured film, a compound having two or more (meth)acrylic groups in the molecule is more preferred. The double bond equivalent of the (B) radical polymerizable compound is preferably from 80 to 800 g/mol from the viewpoint of improving the sensitivity for exposure and forming a pattern in a low-taper shape.

Examples of the (B) radical polymerizable compound include, in addition to a (B1) fluorene skeleton-containing radical polymerizable compound and an (B2) indane skeleton-containing radical polymerizable compound to be described later, diethylene glycol di(meth)acrylate, triethylene glycol di(meth)acrylate, tetraethylene glycol di(meth)acrylate, propylene glycol di(meth)acrylate, trimethylolpropane di(meth)acrylate, trimethylolpropane tri(meth)acrylate, ditrimethylolpropane tri(meth)acrylate, ditrimethylolpropane tetra(meth)acrylate, 1,3-butanediol di(meth)acrylate, neopentyl glycol di(meth)acrylate, 1,4-butanediol di(meth)acrylate, 1,6-hexanediol di(meth)acrylate, 1,9-nonane di(meth)acrylate, 1,10-decanediol di(meth)acrylate, dimethylol-tricyclodecane di(meth)acrylate, pentaerythritol tri(meth)acrylate, pentaerythritol tetra(meth)acrylate, dipentaerythritol penta(meth)acrylate, dipentaerythritol hexa(meth)acrylate, tripentaerythritol hepta(meth)acrylate, tripentaerythritol octa(meth)acrylate, tetrapentaerythritol nona(meth)acrylate, tetrapentaerythritol deca(meth)acrylate, pentapentaerythritol undeca(meth)acrylate, pentapentaerythritol dodeca(meth)acrylate, ethoxylated bisphenol A di(meth)acrylate, 2,2-bis[4-(3-(meth)acryloxy-2-hydroxypropoxy) phenyl]propane, 1,3,5-tris((meth)acryloxyethyl) isocyanuric acid, or 1,3-bis((meth)acryloxyethyl) isocyanuric acid, or acid modified products. Furthermore, from the viewpoint of improving the resolution after development, the compound obtained by reacting a compound obtained by the ring-opening addition reaction of a compound having two or more glycidoxy groups in the molecule and an unsaturated carboxylic acid having an ethylenically unsaturated double bond group, with a polybasic carboxylic acid or polybasic carboxylic anhydride is also preferred.

The content of the (B) radical polymerizable compound in the negative photosensitive resin composition according to the present invention is, in a case where the (A) alkali-soluble resin and the (B) radical polymerizable compound are regarded as 100 parts by mass in total, preferably 15 parts by mass or more, more preferably 20 parts by mass or more, still more preferably 25 parts by mass or more, particularly preferably 30 parts by mass or more. When the content is 15 parts by mass or more, the sensitivity for exposure can be improved, and a cured film in pattern in a low-taper shape can be obtained. On the other hand, the content of the (B) radical polymerizable compound is preferably 65 parts by mass or less, more preferably 60 parts by mass or less, still more preferably 55 parts by mass or less, particularly preferably 50 parts by mass or less. When the content is 65 parts by mass or less, the heat resistance of the cured film can be improved, and a low taper pattern shape can be obtained.

### <(B1) Fluorene Skeleton-Containing Radical Polymerizable Compound and (B2) Indane Skeleton-Containing Radical Polymerizable Compound>

The negative photosensitive resin composition according to the present invention preferably further contains, as the (B) radical polymerizable compound, one or more selected from the group consisting of a (B1) fluorene skeleton-containing radical polymerizable compound and an (B2) indane skeleton-containing radical polymerizable compound.

The (B1) fluorene skeleton-containing radical polymerizable compound refers to a compound having a plurality of ethylenically unsaturated double bond groups and a fluorene skeleton in the molecule. The (B2) indane skeleton-containing radical polymerizable compound refers to a compound having a plurality of ethylenically unsaturated double bond groups and an indane skeleton in the molecule.

Containing the (B1) fluorene skeleton-containing radical polymerizable compound or the (B2) indane skeleton-containing radical polymerizable compound makes it possible to improve the sensitivity for exposure and control the pattern shape after development, and makes it possible to form a pattern in a low-taper shape after thermal curing. In addition, since it is possible to form a pattern in a forward tapered shape by controlling the pattern shape after development, the halftone characteristics can be improved. Furthermore, the change in pattern opening width between before and after thermal curing can be suppressed.

Furthermore, in the case of containing, in particular, a (D1a-1a) benzofuranone-based black pigment as the (Da) black colorant described later, a development residue derived from the pigment described above may be generated due to the insufficient alkali resistance of the above-described pigment. In such a case, containing the (B3) flexible chain-containing aliphatic radical polymerizable compound described later and the (B1) fluorene skeleton-containing radical polymerizable compound or (B2) indane skeleton-containing radical polymerizable compound makes it possible to inhibit the development residue generation derived from the pigment described above.

As the (B1) fluorene skeleton-containing radical polymerizable compound and the (B2) indane skeleton-containing radical polymerizable compound, a compound having a (meth)acrylic group is preferred, which facilitates radical polymerization. From the viewpoint of improving the sensitivity for exposure and reducing the residue after development, compounds having two or more (meth)acrylic groups in the molecule are more preferred.

Examples of the (B1) fluorene skeleton-containing radical polymerizable compounds include 9,9-bis[4-(2-(meth)acryloxyethoxy) phenyl]fluorene, 9,9-bis[4-(3-(meta)acryloxypropoxy) phenyl]fluorene, 9,9-bis(4-(meth)acryloxyphenyl)fluorene, 9,9-bis[4-(2-hydroxy-3-(meth)acryloxypropoxy) phenyl]fluorene, or 9,9-bis[3,4-bis(2-(meth)acryloxyethoxy)phenyl]fluorene, or OGSOL (registered trademark) EA-50P, EA-0200, EA-0250P, EA-0300, EA-500, EA-1000, EA-F5510, or GA-5000 (all manufactured by Osaka Gas Chemicals Co., Ltd.).

Examples of the (B2) indane skeleton-containing radical polymerizable compound include 1,1-bis[4-(2-(meth)acryloxyethoxy)phenyl]indane, 1,1-bis(4-(meth)acryloxyphenyl)indane, 1,1-bis[4-(2-hydroxy-3-(meth)acryloxypropoxy) phenyl]indane, 1,1-bis[3,4-bis(2-(meth) acryloxyethoxy)phenyl]indane, 2,2-bis[4-(2-(meth)acryloxyethoxy)phenyl]indane, or 2,2-bis(4-(meth) acryloxyphenyl)indane.

The (B1) fluorene skeleton-containing radical polymerizable compound and the (B2) indane skeleton-containing radical polymerizable compound can be synthesized by known methods. For example, the synthesis method described in International Publication No. 2008/139924 can be mentioned.

The total content of the (B1) fluorene skeleton-containing radical polymerizable compound and (B2) indane skeleton-containing radical polymerizable compound in the negative photosensitive resin composition according to the present invention is, in a case where the (A) alkali-soluble resin and the (B) radical polymerizable compound are regarded as 100 parts by mass in total, preferably 0.5 parts by mass or more, more preferably 1 part by mass or more, still more preferably 2 parts by mass or more, even more preferably 3 parts by mass or more, particularly preferably 5 parts by mass or more. When the content is 0.5 parts by mass or more, the sensitivity for exposure can be improved, and a pattern in a low-taper shape can be formed. In addition, the change in pattern opening width between before and after thermal curing can be suppressed, and the halftone characteristics can be improved. On the other hand, the total content of the (B1) fluorene skeleton-containing radical polymerizable compound and (B2) indane skeleton-containing radical polymerizable compound is preferably 25 parts by mass or less, more preferably 22 parts by mass or less, still more preferably 20 parts by mass or less, even more preferably 18 parts by mass or less, particularly preferably 15 parts by mass or less. When the content is 25 parts by mass or less, the change in pattern opening width between before and after thermal curing can be suppressed, and the residue generation after development can be inhibited.

### <(B3) Flexible Chain-Containing Aliphatic Radical Polymerizable Compound>

The negative photosensitive resin composition according to the present invention preferably further contains the (B3) flexible chain-containing aliphatic radical polymerizable compound as the (B) radical polymerizable compound. The (B3) flexible chain-containing aliphatic radical polymerizable compound refers to a compound having a plurality of ethylenically unsaturated double bond groups and a flexible skeleton such as an aliphatic chain or an oxyalkylene chain in the molecule.

Containing the (B3) flexible chain-containing aliphatic radical polymerizable compound causes UV curing during the exposure to proceed efficiently, thereby allowing the sensitivity for the exposure to be improved. In addition, in the case of containing, in particular, the (D1) pigment as (D) colorant described later, the (D1) pigment is immobilized to the cured part by crosslinking during UV curing of the (B3) flexible chain-containing aliphatic radical polymerizable compound, thus making it possible to inhibit the residue generation after development, which is derived from the (D1) pigment. Furthermore, the change in pattern opening width between before and after thermal curing can be suppressed.

Furthermore, in the case of containing, in particular, a (D1a-1a) benzofuranone-based black pigment as the (Da) black colorant described later, a development residue derived from the pigment described above may be generated due to the insufficient alkali resistance of the pigment as described above. Even in such a case, the generation of the development residue derived from the pigment described above can be inhibited by containing the (B3) flexible chain-containing aliphatic radical polymerizable compound.

As the (B3) flexible chain-containing aliphatic radical polymerizable compound, a compound having a group represented by general formula (24) and three or more groups represented by general formula (25) in the molecule is preferred.

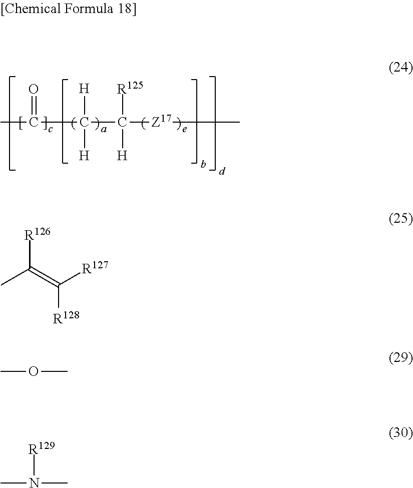

In the general formula (24), R¹²⁵ represents hydrogen or an alkyl group having 1 to 10 carbon atoms. Z¹⁷ represents a group represented by general formula (29) or a group represented by general formula (30). a represents an integer of 1 to 10, b represents an integer of 1 to 4, c represents 0 or 1, d represents an integer of 1 to 4, and e represents 0 or 1. In a case where c is 0, d is 1. In the general formula (25), R¹²⁶ to R¹²⁸ each independently represent hydrogen, an alkyl group having 1 to 10 carbon atoms, or an aryl group having 6 to 15 carbon atoms. In the general formula (30), R¹²⁹ represents hydrogen or an alkyl group having 1 to 10 carbon atoms. In the general formula (24), c preferably represents 1, and e preferably represents 1. In the general formula (25), R¹²⁶ preferably represents hydrogen or an alkyl group having 1 to 4 carbon atoms, more preferably hydrogen or a methyl group. R¹²⁷ and R¹²⁸ each independently preferably represent hydrogen or an alkyl group having 1 to 4 carbon atoms, more preferably hydrogen. In the general formula (30), R¹²⁹ preferably represents hydrogen or an alkyl group having 1 to 4 carbon atoms, more preferably hydrogen or a methyl group. In the general formula (24), in a case where c represents 1, the residue generation after development can be inhibited.

The (B3) flexible chain-containing aliphatic radical polymerizable compound preferably has at least one lactone-modified chain and/or at least one lactam-modified chain. The (B3) flexible chain-containing aliphatic radical polymerizable compound has at least one lactone-modified chain and/or at least one lactam-modified chain, thereby allowing the residue generation after development to be inhibited. When c and e respectively represent 1 and 1 in the general formula (24), the (B3) flexible chain-containing aliphatic radical polymerizable compound has at least one lactone-modified chain and/or at least one lactam-modified chain.

The number of ethylenically unsaturated double bond groups in the molecule of the (B3) flexible chain-containing aliphatic radical polymerizable compound is preferably 2 or more, more preferably 3 or more, still more preferably 4 or more. When the number of ethylenically unsaturated double bond groups is 2 or more, the sensitivity for exposure can be improved. On the other hand, the number of ethylenically unsaturated double bond groups in the molecule of the (B3) flexible chain-containing aliphatic radical polymerizable compound is preferably 12 or less, more preferably 10 or less, still more preferably 8 or less, particularly preferably 6 or less. When the number of ethylenically unsaturated double bond groups is 12 or less, a pattern in a low-taper shape can be formed after thermal curing, and the change in pattern opening width between before and after thermal curing can be suppressed.

Examples of the (B3) flexible chain-containing aliphatic radical polymerizable compound include, as compounds having three or more ethylenically unsaturated double bond groups in the molecules, for example, ethoxylated dipentaerythritol hexa(meth)acrylate, propoxylated dipentaerythritol hexa(meth)acrylate, ε-caprolactone modified dipentaerythritol hexa(meth)acrylate, δ-valerolactone modified dipentaerythritol hexa(meth)acrylate, γ-butyrolactone modified dipentaerythritol hexa(meth)acrylate, S-propiolactone modified dipentaerythritol hexa(meth)acrylate, ε-caprolactam modified dipentaerythritol hexa(meth)acrylate, ε-caprolactone modified dipentaerythritol penta(meth)acrylate, ε-caprolactone modified trimethylolpropane tri(meth)acrylate, ε-caprolactone modified ditrimethylolpropane tetra(meth)acrylate, ε-caprolactone modified glycerin tri(meth)acrylate, ε-caprolactone modified pentaerythritol tri(meth)acrylate, ε-caprolactone modified pentaerythritol tetra(meth)acrylate, or ε-caprolactone modified 1,3,5-tris((meth)acryloxyethyl) isocyanurate, and “KAYARAD” (registered trademark) DPEA-12, DPCA-20, DPCA-30, DPCA-60, or DPCA-120 (all manufactured by Nippon Kayaku Co., Ltd.), or “NK ESTER” (registered trademark) A-DPH-6E, A-DPH-6P, M-DPH-6E, A-9300-1CL, or A-9300-3CL (all manufactured by Shin Nakamura Chemical Co., Ltd.).

Examples of the compound having two ethylenically unsaturated double bond groups in the molecule include ε-caprolactone-modified hydroxypivalate neopentyl glycol di(meth)acrylate, ε-caprolactone modified trimethylolpropane di(meth)acrylate, ε-caprolactone modified ditrimethylolpropane di(meth)acrylate, ε-caprolactone modified glycerin di(meth)acrylate, ε-caprolactone modified pentaerythritol di(meth)acrylate, ε-caprolactone modified dimethylol-tricyclodecane di(meth)acrylate, ε-caprolactone modified 1,3-bis((meth)acryloxyethyl) isocyanuric acid, or ε-caprolactone modified 1,3-bis((meth)acryloxyethyl) isocyanuric acid, or “KAYARAD” (registered trademark) HX-220 or HX-620 (all manufactured by Nippon Kayaku Co., Ltd.).

The (B3) flexible chain-containing aliphatic radical polymerizable compound can be synthesized by known methods.

The content of the (B3) flexible chain-containing aliphatic radical polymerizable compound in the negative photosensitive resin composition according to the present invention is, in a case where the (A) alkali-soluble resin and the (B) radical polymerizable compound are regarded as 100 parts by mass in total, preferably 5 parts by mass or more, more preferably 10 parts by mass or more, still more preferably 15 parts by mass or more, particularly preferably 20 parts by mass or more. When the content is 5 parts by mass or more, the sensitivity for exposure can be improved, and the residue generation after development can be inhibited. In addition, the change in pattern opening width between before and after thermal curing can be suppressed. On the other hand, the content of the (B3) flexible chain-containing aliphatic radical polymerizable compound is preferably 45 parts by mass or less, more preferably 40 parts by mass or less, still more preferably 35 parts by mass or less, particularly preferably 30 parts by mass or less. When the content is 45 parts by mass or less, a cured film in a pattern in a low-taper shape can be obtained.

### <(B4) Alicyclic Group-Containing Radical Polymerizable Compound>

The negative photosensitive resin composition according to the present invention preferably further contains an (B4) alicyclic group-containing radical polymerizable compound as the (B) radical polymerizable compound. The (B4) alicyclic group-containing radical polymerizable compound refers to a compound having a plurality of ethylenically unsaturated double bond groups and an alicyclic group in the molecule.

Containing the (B4) alicyclic group-containing radical polymerizable compound allows the change in pattern opening width between before and after thermal curing to be suppressed. This is presumed to be because an alicyclic group is introduced into the film by UV curing during exposure, thereby improving the heat resistance, and suppressing reflow at the pattern skirt. In addition to the foregoing, a forward tapered shape is allowed by pattern shape control after development, and a pattern in a low-taper shape can be formed after thermal curing. This is presumed to be because the hydrophobicity of the alicyclic group blocks the penetration of the alkaline developer into the film during development, and because the steric hindrance of the alicyclic group inhibits excessive curing during thermal curing.

The negative photosensitive resin composition according to the present invention preferably contains the (B4) alicyclic group-containing radical polymerizable compound and a (F) polyfunctional thiol compound to be described later. The (B4) alicyclic group-containing radical polymerizable compound and the (F) polyfunctional thiol compound are used in combination, thereby making it possible to suppress the change in pattern opening width between before and after thermal curing, and making it possible to form a pattern in a low-taper shape after development. This is believed to be because the (F) polyfunctional thiol compound suppresses oxygen inhibition at the film surface, thereby promoting the UV curing of the (B4) alicyclic group-containing radical polymerizable compound during exposure. More specifically, it is presumed that the heat resistance and hydrophobicity derived from the alicyclic group are improved by the UV curing during exposure. This is believed to be because UV curing is remarkably promoted by the use of the (F) polyfunctional thiol compound in combination, since the (B4) alicyclic group-containing radical polymerizable compound is likely to undergo UV curing inhibited due to the steric hindrance of the alicyclic group in addition to oxygen inhibition at the film surface.

The number of ethylenically unsaturated double bond groups in the molecule of the (B4) alicyclic group-containing radical polymerizable compound is more preferably 2 or more, still more preferably 3 or more. When the number of ethylenically unsaturated double bond groups is 2 or more, the sensitivity for exposure can be improved. On the other hand, the number of ethylenically unsaturated double bond groups in the molecule of the (B4) alicyclic group-containing radical polymerizable compound is preferably 10 or less, more preferably 6 or less. When the number of ethylenically unsaturated double bond groups is 10 or less, a pattern in a low-taper shape can be formed after thermal curing.

The alicyclic group in the molecule of the (B4) alicyclic group-containing radical polymerizable compound is preferably a polycyclic condensed alicyclic skeleton. Having the polycyclic condensed alicyclic skeleton allows the change in pattern opening width between before and after thermal curing to be suppressed. In addition, a forward tapered shape is allowed by pattern shape control after development, and a pattern in a low-taper shape can be formed after thermal curing.

Examples of the polycyclic condensed alicyclic skeleton include a bicyclo[4.3.0]nonane skeleton, a bicyclo[5.4.0]undecane skeleton, a bicyclo[2.2.2]octane skeleton, a tricyclo[5.2.1.0^{2,6}]decane skeleton, a pentacyclopentadecane skeleton, an adamantane skeleton, or a hydroxyadamantane skeleton.

Examples of the (B4) alicyclic group-containing radical polymerizable compound include dimethylol-bicyclo[4.3.0]nonane (meth)acrylate, dimethylol-bicyclo[5.4.0]undecane di(meth)acrylate, dimethylol-bicyclo[2.2.2]octane di(meth)acrylate, dimethylol-tricyclo[5.2.1.0^{2,6}]decane di(meth)acrylate, dimethylol-pentacyclopentadecane di(meth)acrylate, 1,3-adamantane di(meth)acrylate, 1,3,5-adamantane tri(meth)acrylate, or 5-hydroxy-1,3-adamantane di(meth)acrylate.

The (B4) alicyclic group-containing radical polymerizable compound can be synthesized by known methods.

The content of the (B4) alicyclic group-containing radical polymerizable compound in the negative photosensitive resin composition according to the present invention is, in a case where the (A) alkali-soluble resin and the (B) radical polymerizable compound are regarded as 100 parts by mass in total, preferably 1 part by mass or more, more preferably 2 parts by mass or more, still more preferably 3 parts by mass or more, even more preferably 5 parts by mass or more, particularly preferably 10 parts by mass or more. When the content is 1 part by mass or more, the change in pattern opening width between before and after thermal curing can be suppressed. Furthermore, a forward tapered shape is allowed by pattern shape control after development, and a pattern in a low-taper shape can be formed after thermal curing. On the other hand, the content of the (B4) alicyclic group-containing radical polymerizable compound is preferably 30 parts by mass or less, more preferably 27 parts by mass or less, still more preferably 25 parts by mass or less, even more preferably 22 parts by mass or less, particularly preferably 20 part by mass or less. When the content is 30 parts by mass or less, the change in pattern opening width between before and after thermal curing can be suppressed, and the residue generation after development can be inhibited.

### <(C1) Photo Initiator>

The negative photosensitive resin composition according to the present invention further contains the (C1) photo initiator as the (C) photosensitive agent. The (C1) photo initiator refers to a compound that generates radicals through bond cleavage and/or reaction upon exposure. Containing the (C1) photo initiator causes radical polymerization of the above-described (B) radical polymerizable compound to proceed, thereby making the exposed part of the film of the resin composition insoluble in an alkaline developer, and then allowing a negative pattern to be formed. In addition, UV curing during the exposure is accelerated, thereby allowing the sensitivity to be improved.

Further, containing a specific amount of (C1) photo initiator or more allows the change in pattern opening width between before and after thermal curing to be suppressed. This is believed to be due to an increase in radical generation, derived from the (C1) photo initiator during the exposure. More specifically, increasing the radical generation during the exposure is presumed to increase the probability of collision between the generated radicals and the ethylenically unsaturated double bond group in the above-described (B) radical polymerizable compound, thereby accelerating UV curing and then improving the crosslink density, thus suppressing reflow of a pattern taper and a pattern skirt during thermal curing, and thus making it possible to suppress the change in pattern opening width between before and after thermal curing.

As the (C1) photo initiator, for example, a benzyl ketal-based photo initiator, an α-hydroxy ketone-based photo initiator, an α-amino ketone-based photo initiator, an acylphosphine oxide-based photo initiator, a biimidazole-based photo initiator, an oxime ester-based photo initiator, an acridine-based photo initiator, a titanocene-based photo initiator, a benzophenone-based photo initiator, an acetophenone-based photo initiator, an aromatic ketoester-based photo initiator, or a benzoic acid ester-based photo initiator is preferred, and from the viewpoint of improvement in sensitivity at the time of exposure, an α-hydroxy ketone-based photo initiator, an α-amino ketone-based photo initiator, an acylphosphine oxide-based photo initiator, a biimidazole-based photo initiator, an oxime ester-based photo initiator, an acridine-based photo initiator, or a benzophenone-based photo initiator is more preferred, and an α-amino ketone-based photo initiator, an acylphosphine-based photo initiator, a biimidazole-based photo initiator, or an oxime ester-based photo initiator is further preferred.

Examples of the benzyl ketal-based photo initiator include 2,2-dimethoxy-1,2-diphenylethane-1-one.

Examples of the α-hydroxy ketone-based photo initiators include 1-(4-isopropylphenyl)-2-hydroxy-2-methylpropan-1-one, 2-hydroxy-2-methyl-1-phenylpropane-1-one, 1-hydroxycyclohexyl phenyl ketone, 1-[4-(2-hydroxyethoxy)phenyl]-2-hydroxy-2-methylpropane-1-one, or 2-hydroxy-1-[4-[4-(2-hydroxy-2-methylpropionyl)benzyl]phenyl]-2-methylpropan-1-one.

Examples of the α-amino ketone-based photo initiator include 2-methyl-1-[4-(methylthio)phenyl]-2-morpholinopropan-1-one, 2-benzyl-2-dimethylamino-1-(4-morpholinophenyl)-butane-1-one, 2-dimethylamino-2-(4-methylbenzyl)-1-(4-morpholinophenyl)-butane-1-one, or 3,6-bis(2-methyl-2-morpholinopropionyl)-9-octyl-9H-carbazole.

Examples of the acyl phosphine oxide-based photo initiator include 2,4,6-trimethyl benzoyl-diphenyl phosphine oxide, bis(2,4,6-trimethyl benzoyl)-phenyl phosphine oxide, or bis(2,6-dimethoxy benzoyl)-(2,4,4-trimethylpentyl)phosphine oxide.

Examples of the biimidazole-based photo initiator include 2,2′-bis(2-chlorophenyl)-4,4′,5,5′-tetraphenyl-1,2′-biimidazole, 2,2′,5-tris(2-chlorophenyl)-4-(3,4-dimethoxyphenyl)-4′,5′-diphenyl-1,2′-biimidazole, 2,2′,5-tris(2-fluorophenyl)-4-(3,4-dimethoxyphenyl)-4′,5′-diphenyl-1,2′-biimidazole, 2,2′-bis(2,4-dichlorophenyl)-4,4′,5,5′-tetraphenyl-1,2′-biimidazole, or 2,2′-bis(2-methoxyphenyl)-4,4′,5,5′-tetraphenyl-1,2′-biimidazole.

Examples of the oxime ester photo initiator include 1-phenylpropane-1,2-dione-2-(0-ethoxycarbonyl)oxime, 1-phenylbutane-1,2-dione-2-(0-methoxycarbonyl)oxime, 1,3-diphenylpropane-1,2,3-trione-2-(0-ethoxycarbonyl)oxime, 1-[4-(phenylthio)phenyl]octane-1,2-dione-2-(0-benzoyl)oxime, 1-[4-[4-carboxyphenylthio]phenyl]propane-1,2-dione-2-(0-acetyl)oxime, 1-[4-[4-(2-hydroxyethoxy)phenylthio]phenyl]propane-1,2-dione-2-(0-acetyl)oxime, 1-[4-(phenylthio)phenyl]-3-cyclopentylpropane-1,2-dione-2-(0-benzoyl)oxime, 1-[4-(phenylthio)phenyl]-2-cyclopentylethane-1,2-dione-2-(0-acetyl)oxime, 1-[9,9-diethylfluorene-2-yl]propane-1,2-dion-2-(O-acetyl)oxime, 1-[9,9-di-n-propyl-7-(2-methylbenzoyl)-fluoren-2-yl]ethanone-1-(O-acetyl)oxime, 1-[9-ethyl-6-(2-methylbenzoyl)-9H-carbazol-3-yl]ethanone-1-(O-acetyl)oxime, 1-[9-ethyl-6-[2-methyl-4-[1-(2,2-dimethyl-1,3-dioxolan-4-yl)methyloxy]benzoyl]-9H-carbazol-3-yl]ethanone-1-(0-acetyl)oxime, 1-[9-ethyl-6-(2-methylbenzoyl)-9H-carbazol-3-yl]-3-cyclopentylpropan-1-one-1-(O-acetyl)oxime, and 1-(9-ethyl-6-nitro-9H-carbazol-3-yl)-1-[2-methyl-4-(1-methoxypropan-2-yloxy)phenyl]methanone-1-(O-acetyl)oxime.

Examples of the acridine-based photo initiator include 1,7-bis(acridin-9-yl)-n-heptane.

Examples of the titanocene-based photo initiator include bis(η⁵-2,4-cyclopentadien-1-yl)-bis[2,6-difluoro)-3-(1H-pyrrol-1-yl)phenyl] titanium (IV) or bis(η⁵-3-methyl-2,4-cyclopentadien-1-yl)-bis(2,6-difluorophenyl) titanium (IV).

Examples of the benzophenone-based photo initiator include benzophenone, 4,4′-bis(dimethylamino)benzophenone, 4,4′-bis(diethylamino)benzophenone, 4-phenylbenzophenone, 4,4-dichlorobenzophenone, 4-hydroxybenzophenone, alkylated benzophenone, 3,3′,4,4′-tetrakis(t-butylperoxycarbonyl)benzophenone, 4-methylbenzophenone, dibenzyl ketone, or fluorenone.

Examples of the acetophenone-based photo initiator include 2,2-diethoxyacetophenone, 2,3-diethoxyacetophenone, 4-t-butyldichloroacetophenone, benzalacetophenone, or 4-azidobenzalacetophenone.

Examples of the aromatic ketoester-based photo initiator include methyl 2-phenyl-2-oxyacetate.

Examples of the benzoate-based photo initiator include ethyl 4-dimethylaminobenzoate, (2-ethyl)hexyl 4-dimethylaminobenzoate, ethyl 4-diethylaminobenzoate, or methyl 2-benzoylbenzoate.

The content of the (C1) photo initiator in the negative photosensitive resin composition according to the present invention is, in a case where the (A) alkali-soluble resin and the (B) radical polymerizable compound are regarded as 100 parts by mass in total, preferably 0.1 parts by mass or more, more preferably 0.5 parts by mass or more, still more preferably 0.7 parts by mass or more, particularly preferably 1 part by mass or more. When the content is 0.1 parts by mass or more, the sensitivity for exposure can be improved. Furthermore, the content of the (C1) photo initiator is, from the viewpoint of controlling the pattern opening width, preferably 10 parts by mass or more, more preferably 12 parts by mass or more, still more preferably 14 parts by mass or more, particularly preferably 15 parts by mass or more. When the content is 10 parts by mass or more, the change in pattern opening width between before and after thermal curing can be suppressed. On the other hand, the content of the (C1) photo initiator is preferably 30 parts by mass or less, more preferably 25 parts by mass or less, still more preferably 22 parts by mass or less, particularly preferably 20 parts by mass or less. When the content is 30 parts by mass or less, the resolution after development can be improved, and a cured film with a pattern in a low-taper shape can be obtained.

### <(C1-1) Specific Oxime Ester-Based Photo Initiator>

The negative photosensitive resin composition according to the present invention contains, as the (C1) photo initiator, the (C1-1) oxime ester-based photo initiator that has one or more structures selected from the group consisting of (I), (II), and (III) (hereinafter, referred to as “(C1-1) specific oxime ester-based photo initiator”).

(I) one or more structures selected from the group consisting of a naphthalenecarbonyl structure, a trimethylbenzoyl structure, a thiophenecarbonyl structure, and a furancarbonyl structure;

(II) a nitro group, a carbazole structure, and a group represented by the general formula (11);

(III) a nitro group and one or more structures selected from the group consisting of a fluorene structure, a dibenzofuran structure, a dibenzothiophene structure, a naphthalene structure, a diphenylmethane structure, a diphenylamine structure, a diphenyl ether structure, and a diphenyl sulfide structure.

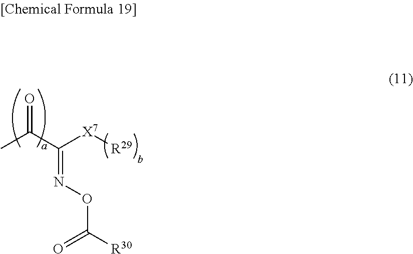

In the general formula (11), X⁷ represents a direct bond, an alkylene group having 1 to 10 carbon atoms, a cycloalkylene group having 4 to 10 carbon atoms, or an arylene group having 6 to 15 carbon atoms. In a case where X⁷ represents a direct bond, an alkylene group having 1 to 10 carbon atoms, or a cycloalkylene group having 4 to 10 carbon atoms, R²⁹ represents hydrogen, an alkyl group having 1 to 10 carbon atoms, or a cycloalkyl group having 4 to 10 carbon atoms, an alkoxy group having 1 to 10 carbon atoms, a haloalkyl group having 1 to 10 carbon atoms, a haloalkoxy group having 1 to 10 carbon atoms, an acyl group having 2 to 10 carbon atoms, or a nitro group. In a case where X⁷ represents an arylene group having 6 to 15 carbon atoms, R²⁹ represents hydrogen, an alkyl group having 1 to 10 carbon atoms, a cycloalkyl group having 4 to 10 carbon atoms, an aryl group having 6 to 15 carbon atoms, a haloalkyl group having 1 to 10 carbon atoms, a haloalkoxy group having 1 to 10 carbon atoms, a heterocyclic group having 4 to 10 carbon atoms, a heterocyclic oxy group having 4 to 10 carbon atoms, an acyl group having 2 to 10 carbon atoms, or a nitro group. R³⁰ represents hydrogen, an alkyl group having 1 to 10 carbon atoms, a cycloalkyl group having 4 to 10 carbon atoms, or an aryl group having 6 to 15 carbon atoms. a represents 0 or 1, and b represents an integer of 0 to 10.

In the general formula (11), X⁷ preferably represents an alkylene group having 1 to 10 carbon atoms from the viewpoint of improving the solubility in a solvent, or an arylene group having 6 to 15 carbon atoms from the viewpoint of improving the sensitivity for exposure. R²⁹ preferably represents a cycloalkyl group having 4 to 10 carbon atoms, a haloalkyl group having 1 to 10 carbon atoms, or a haloalkoxy group having 1 to 10 carbon atoms from the viewpoint of improving the solubility in a solvent. Furthermore, R²⁹ preferably represents, from the viewpoint of improving the sensitivity for exposure and forming a pattern in a low-taper shape after development, a haloalkyl group having 1 to 10 carbon atoms, a haloalkoxy group having 1 to 10 carbon atoms, a heterocyclic ring group having 4 to 10 carbon atoms, a heterocyclic oxy group having 4 to 10 carbon atoms, an acyl group having 2 to 10 carbon atoms, or a nitro group. R³⁰ preferably represents hydrogen or an alkyl group having 1 to 10 carbon atoms, more preferably hydrogen or an alkyl group having 1 to 4 carbon atoms, still more preferably methyl, from the viewpoint of improving the sensitivity for exposure. a preferably represents 0 from the viewpoint of improving the sensitivity for exposure.

The group represented by the general formula (11) is a group having an oxime ester structure, which is a group having a structure that generates a radical through a bond cleavage and/or a reaction by UV for exposure. It is to be noted that the fluorene structure, carbazole structure, dibenzofuran structure, dibenzothiophene structure, naphthalene structure, diphenylmethane structure, diphenylamine structure, diphenyl ether structure, or diphenyl sulfide structure in the above-described (II) or (III) represents a mother skeleton to which the above-described group having the oxime ester structure is bonded. In addition, the naphthalenecarbonyl structure, trimethylbenzoyl structure, thiophenecarbonyl structure, furancarbonyl structure, and nitro group in the above-described (I), (II), or (III) represent a structure or a group that is bonded to the mother skeleton to which the above-described group having the oxime ester structure is bonded.

The (C1-1) specific oxime ester-based photo initiator an oxime ester-based compound having, in the molecule, a specific conjugated structure which increases the absorbance in the ultraviolet area to allow for the promotion of radical curing in the deep part of the film during exposure and radical curing by the increased radical generation during exposure.

Containing the (C1-1) specific oxime ester-based photo initiator allows the sensitivity for exposure to be improved, and allows a pattern in a low-taper shape to be formed after development. In addition, halftone characteristics can be improved. This is presumed to be because radical curing is allowed in the deep part of the film during exposure. In addition, it is believed to be because the specific conjugated structure of the (C1-1) specific oxime ester-based photo initiator interacts with the aromatic groups of the (A1) first resin and (A2) second resin to make the entire film compatible, while the promotion of radical curing into the deep part of the film by the increased radical generation during exposure suppresses the penetration of the developer into the cured film during alkali development, thereby allowing side etching with the developer to be suppressed.

Further, containing the (C1-1) specific oxime ester-based photo initiator allows the change in pattern opening width between before and after thermal curing to be suppressed. This is presumed to be because in addition to the suppression of side etching by the developer during alkali development, which allows the pattern formation in a low-taper shape after development, in the same manner as described above, UV curing during exposure is promoted, thereby increasing the molecular weight of the cured film, and then suppressing reflow of the pattern skirt during thermal curing.

The (C1-1) specific oxime ester-based photo initiator preferably contains, from the viewpoints of improving the sensitivity for exposure, reducing the taper by pattern shape control after development, suppressing the change in pattern opening width between before and after thermal curing, and improving the halftone characteristics, one or more selected from the group consisting of a compound represented by the general formula (12), a compound represented by the general formula (13), and a compound represented by the general formula (14), more preferably a compound represented by the general formula (13).

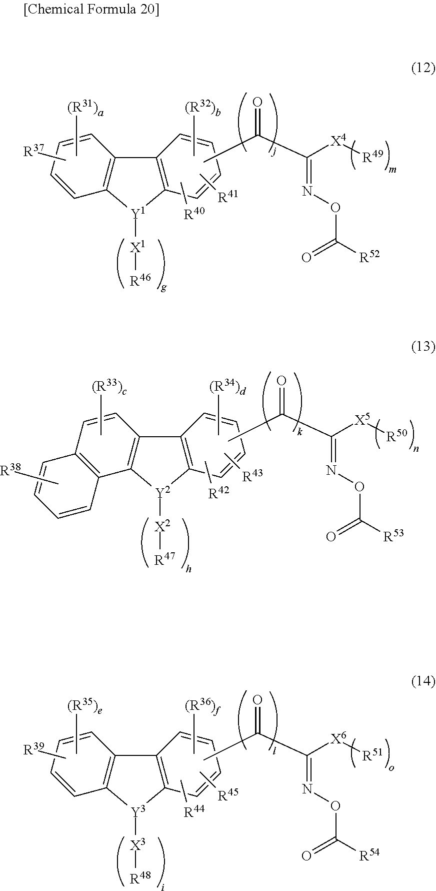

In the general formulas (12) to (14), X¹ to X⁶ each independently represent a direct bond, an alkylene group having 1 to 10 carbon atoms, a cycloalkylene group having 4 to 10 carbon atoms, or an arylene group having 6 to 15 carbon atoms. Y¹ to Y³ each independently represent carbon, nitrogen, oxygen, or sulfur. R³¹ to R³⁶ each independently represent an alkyl group having 1 to 10 carbon atoms, a cycloalkyl group having 4 to 10 carbon atoms, an aryl group having 6 to 15 carbon atoms, an alkoxy group having 1 to 10 carbon atoms, or a hydroxyalkyl group having 1 to 10 carbon atoms. R³⁷ to R³⁹ each independently represent a group represented by the general formula (15), a group represented by the general formula (16), a group represented by the general formula (17), a group represented by the general formula (18), or a nitro group. R⁴⁰ to R⁴⁵ each independently represent hydrogen, an alkyl group having 1 to 10 carbon atoms, a cycloalkyl group having 4 to 10 carbon atoms, an aryl group having 6 to 15 carbon atoms, or a group that forms a ring having 4 to 10 carbon atoms. R⁴⁶ to R⁴⁸ each independently represent hydrogen, an alkyl group having 1 to 10 carbon atoms, a cycloalkyl group having 4 to 10 carbon atoms, an aryl group having 6 to 15 carbon atoms, an alkenyl group having 1 to 10 carbon atoms, an alkoxy group having 1 to 10 carbon atoms, a haloalkyl group having 1 to 10 carbon atoms, a haloalkoxy group having 1 to 10 carbon atoms, or an acyl group having 2 to 10 carbon atoms. R⁴⁹ to R⁵¹ each independently represent hydrogen, an alkyl group having 1 to 10 carbon atoms, a cycloalkyl group having 4 to 10 carbon atoms, an aryl group having 6 to 15 carbon atoms, an alkoxy group having 1 to 10 carbon atoms, a haloalkyl group having 1 to 10 carbon atoms, a haloalkoxy group having 1 to 10 carbon atoms, a heterocyclic group having 4 to 10 carbon atoms, a heterocyclic oxy group having 4 to 10 carbon atoms, an acyl group having 2 to 10 carbon atoms, or a nitro group. R⁵² to R⁵⁴ each independently represent hydrogen, an alkyl group having 1 to 10 carbon atoms, a cycloalkyl group having 4 to 10 carbon atoms, or an aryl group having 6 to 15 carbon atoms. a represents an integer of 0 to 3, b represents 0 or 1, c represents an integer of 0 to 5, d represents 0 or 1, e represents an integer of 0 to 4, f represents an integer of 0 to 2, g, h, and i each independently represent an integer of 0 to 2, j, k, and l each independently represent 0 or 1, and m, n, and o each independently represent an integer of 0 to 10. In a case where Y¹ represents nitrogen, R³⁷ represents a nitro group, and X⁴ represents an arylene group having 6 to 15 carbon atoms, R⁴⁹ represents hydrogen, an alkyl group having 1 to 10 carbon atoms, a cycloalkyl group having 4 to 10 carbon atoms, an aryl group having 6 to 15 carbon atoms, a haloalkyl group having 1 to 10 carbon atoms, a haloalkoxy group having 1 to 10 carbon atoms, a heterocyclic group having 4 to 10 carbon atoms, a heterocyclic oxy group having 4 to 10 carbon atoms, an acyl group having 2 to 10 carbon atoms, or a nitro group.

In the general formulas (12) to (14), X¹ to X⁶ each independently preferably represent an alkylene group having 1 to 10 carbon atoms from the viewpoint of improving the solubility in a solvent, and preferably represent an arylene group having 6 to 15 carbon atoms from the viewpoint of improving the sensitivity for exposure. Examples of the ring having 4 to 10 carbon atoms, formed in R⁴⁰ to R⁴⁵, include a benzene ring or a cyclohexane ring. R⁴⁶ to R⁴⁸ each independently preferably represent, from the viewpoint of improving the solubility in a solvent, an alkyl group having 1 to 10 carbon atoms, a cycloalkyl group having 4 to 10 carbon atoms, a haloalkyl group having 1 to 10 carbon atoms, or a haloalkoxy group having 1 to 10 carbon atoms, and more preferably a fluoroalkyl group having 1 to 10 carbon atoms, or a fluoroalkoxy group having 1 to 10 carbon atoms. Furthermore, R⁴⁶ to R⁴⁸ each independently preferably represent, from the viewpoints of improving the sensitivity for exposure and forming a pattern in a low-taper shape after development, a haloalkyl group having 1 to 10 carbon atoms, a haloalkoxy group having 1 to 10 carbon atoms, or an acyl group having 2 to 10 carbon atoms, more preferably a fluoroalkyl group having 1 to 10 carbon atoms or a fluoroalkoxy group having 1 to 10 carbon atoms. R⁴⁹ to R⁵¹ each independently preferably represent, from the viewpoint of improving the solubility in a solvent, a cycloalkyl group having 4 to 10 carbon atoms, a haloalkyl group having 1 to 10 carbon atoms, or a haloalkoxy group having 1 to 10 carbon atoms, and more preferably a fluoroalkyl group having 1 to 10 carbon atoms, or a fluoroalkoxy group having 1 to 10 carbon atoms. Furthermore, R⁴⁹ to R⁵¹ each independently preferably represent, from the viewpoints of improving the sensitivity for exposure and forming a pattern in a low-taper shape after development, a haloalkyl group having 1 to 10 carbon atoms, a haloalkoxy group having 1 to 10 carbon atoms, a heterocyclic ring group having 4 to 10 carbon atoms, a heterocyclic oxy group having 4 to 10 carbon atoms, or an acyl group having 2 to 10 carbon atoms or a nitro group, more preferably a fluoroalkyl group having 1 to 10 carbon atoms or a fluoroalkoxy group having 1 to 10 carbon atoms. R⁵² to R⁵⁴ each independently preferably represent, from the viewpoint of improving the sensitivity for exposure, hydrogen or an alkyl group having 1 to 10 carbon atoms, more preferably hydrogen or an alkyl group having 1 to 4 carbon atoms, still more preferably methyl. j, k, and l each independently preferably represent 0 from the viewpoint of improving the sensitivity for exposure.

Furthermore, the (C1-1) specific oxime ester-based photo initiator preferably contains, from the viewpoints of improving the sensitivity for exposure, reducing the taper by pattern shape control after development, suppressing the change in pattern opening width between before and after thermal curing, and improving the halftone characteristics, a compound represented by the general formula (12) and/or a compound represented by the general formula (13). In the general formula (12) and the general formula (13), Y¹ and Y² preferably represent carbon or nitrogen. R⁴⁶ and R⁴⁷ preferably include at least an alkenyl group having 1 to 10 carbon atoms, more preferably include an alkenyl group having 1 to 6 carbon atoms. R⁴⁹ and R⁵⁰ preferably include at least an alkenyl group having 1 to 10 carbon atoms, more preferably include an alkenyl group having 1 to 6 carbon atoms. This is believed to be because the inclusion of the alkenyl group can further enhance the compatibility between the resin and the initiator, thereby causing UV curing during exposure to proceed efficiently even in the deep part of the film.

Examples of the alkenyl group include a vinyl group, 1-methylethenyl group, an allyl group, 1-methyl-2-propenyl group, 2-methyl-2-propenyl group, 1-propenyl group, 2-methyl-1-propenyl group, 1-butenyl group, 2-butenyl group, 2-methyl-2-butenyl group, 3-methyl-2-butenyl group, 2,3-dimethyl-2-butenyl group, 3-butenyl group, or a cinnamyl group.

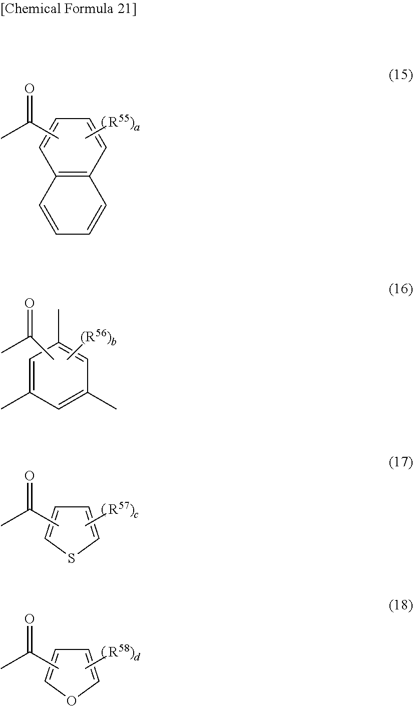

In the general formulas (15) to (18), R⁵⁵ to R⁵⁸ each independently represent an alkyl group having 1 to 10 carbon atoms, a cycloalkyl group having 4 to 10 carbon atoms, an aryl group having 6 to 15 carbon atoms, an alkoxy group having 1 to 10 carbon atoms, a hydroxyalkyl group having 1 to 10 carbon atoms, or a group that forms a ring. Examples of the ring formed for the plurality of R⁵⁵ to R⁵⁸ include a benzene ring, a naphthalene ring, an anthracene ring, a cyclopentane ring, and a cyclohexane ring. a represents an integer of 0 to 7, b represents an integer of 0 to 2, and c and d each independently represent an integer from 0 to 3. The ring formed for the plurality of R⁵⁵ to R⁵⁸ is preferably a benzene ring or a naphthalene ring.

The (C1-1) specific oxime ester-based photo initiator preferably has a halogen-substituted group. The (C1-1) specific oxime ester-based photo initiator has the halogen-substituted group, thereby improving the solubility in a solvent. In addition, the compatibility with the (A1) first resin and the (A2) second resin is improved, thereby allowing the sensitivity for exposure to be improved, and allowing a pattern in a low-taper shape to be formed after development. In addition, the change in pattern opening width between before and after thermal curing can be suppressed, and the halftone characteristics can be improved. As the halogen, fluorine is preferred. This is believed to be because in a case where the (C1-1) specific oxime ester-based photo initiator has a fluorine-substituted group, one or more selected from the (A1-1) polyimide, the (A1-2) polyimide precursor, the (A1-3) polybenzoxazole, and the (A1-4) polybenzoxazole precursor as the (A1) first resin contain a structural unit having a fluorine atom, thus allowing the compatibility between the resin and the initiator to be further enhanced, and then causing V curing during exposure to proceed efficiently even in the deep part of the film.

Examples of the halogen-substituted group include a fluoromethyl group, a fluoroethyl group, a chloroethyl group, a bromoethyl group, an iodoethyl group, a trifluoromethyl group, a trifluoropropyl group, a trichloropropyl group, a tetrafluoropropyl group, a trifluoropentyl group, a tetrafluoropentyl group, a pentafluoropentyl group, a heptafluoropentyl group, a heptafluorodecyl group, a fluorocyclopentyl group, a tetrafluorocyclopentyl group, a fluorophenyl group, a pentafluorophenyl group, a trifluoromethoxy group, a trifluoropropoxy group, a tetrafluoropropoxy group, a trifluoropentyloxy group, a pentafluoropentyloxy group, a tetrafluorocyclopentyloxy group, or a pentafluorophenoxy group.

Examples of the (C1-1) specific oxime ester-based photo initiator include compounds that have the structures shown below (OXL-1 to OXL-102).

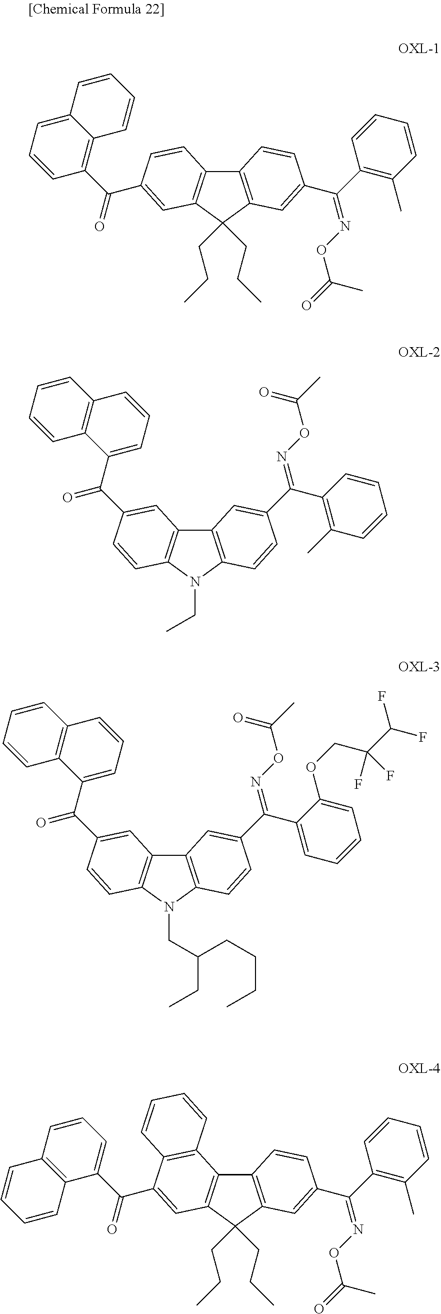 [SMILES: CCCC1(CCC)c2cc(C(=O)c3cccc4ccccc34)ccc2-c2ccc(/C(=N\OC(C)=O)c3ccccc3C)cc21 ; CCn1c2ccc(C(=O)c3cccc4ccccc34)cc2c2cc(/C(=N/OC(C)=O)c3ccccc3C)ccc21 ; CCCCC(CC)Cn1c2ccc(C(=O)c3cccc4ccccc34)cc2c2cc(/C(=N/OC(C)=O)c3ccccc3OCC(F)(F)C(F)F)ccc21 ; CCCC1(CCC)c2cc(/C(=N\OC(C)=O)c3ccccc3C)ccc2-c2c1cc(C(=O)c1cccc3ccccc13)c1ccccc21]

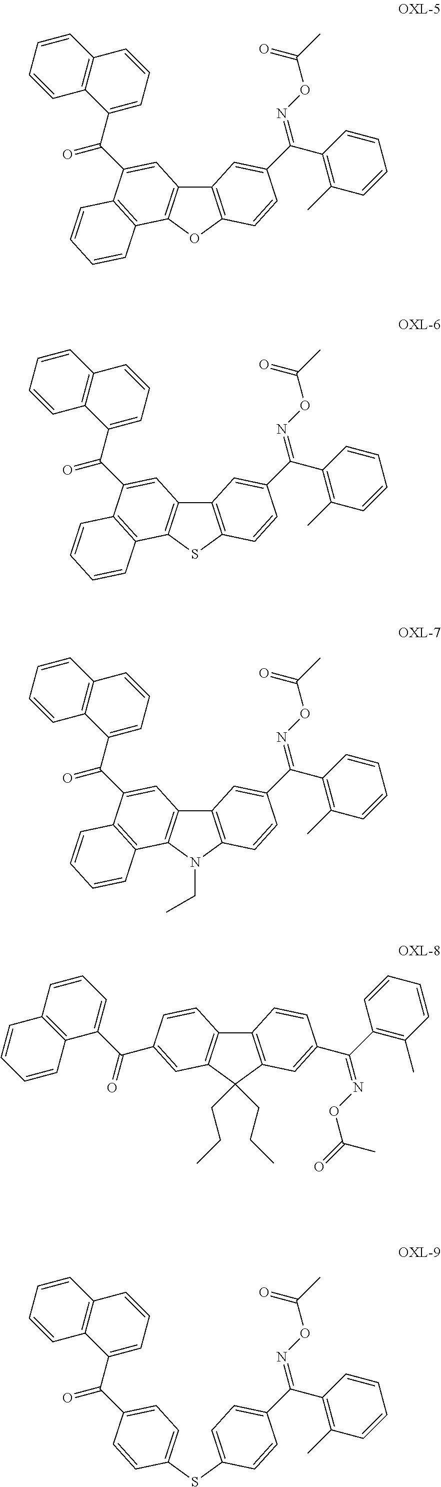 [SMILES: CC(=O)O/N=C(/c1ccc2oc3c4ccccc4c(C(=O)c4cccc5ccccc45)cc3c2c1)c1ccccc1C ; CC(=O)O/N=C(/c1ccc2sc3c4ccccc4c(C(=O)c4cccc5ccccc45)cc3c2c1)c1ccccc1C ; CCn1c2ccc(/C(=N/OC(C)=O)c3ccccc3C)cc2c2cc(C(=O)c3cccc4ccccc34)c3ccccc3c21 ; CCCC1(CCC)c2cc(C(=O)c3cccc4ccccc34)ccc2-c2ccc(/C(=N\OC(C)=O)c3ccccc3C)cc21 ; CC(=O)O/N=C(/c1ccc(Sc2ccc(C(=O)c3cccc4ccccc34)cc2)cc1)c1ccccc1C]

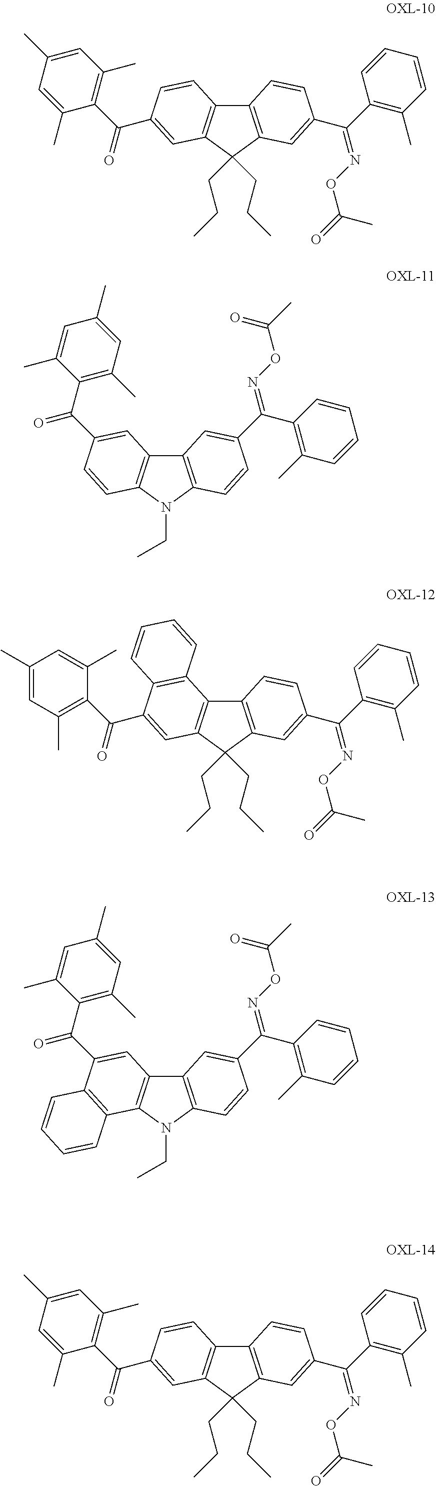 [SMILES: CCCC1(CCC)c2cc(C(=O)c3c(C)cc(C)cc3C)ccc2-c2ccc(/C(=N\OC(C)=O)c3ccccc3C)cc21 ; CCn1c2ccc(C(=O)c3c(C)cc(C)cc3C)cc2c2cc(/C(=N/OC(C)=O)c3ccccc3C)ccc21 ; CCCC1(CCC)c2cc(/C(=N\OC(C)=O)c3ccccc3C)ccc2-c2c1cc(C(=O)c1c(C)cc(C)cc1C)c1ccccc21 ; CCn1c2ccc(/C(=N/OC(C)=O)c3ccccc3C)cc2c2cc(C(=O)c3c(C)cc(C)cc3C)c3ccccc3c21 ; CCCC1(CCC)c2cc(C(=O)c3c(C)cc(C)cc3C)ccc2-c2ccc(/C(=N\OC(C)=O)c3ccccc3C)cc21]

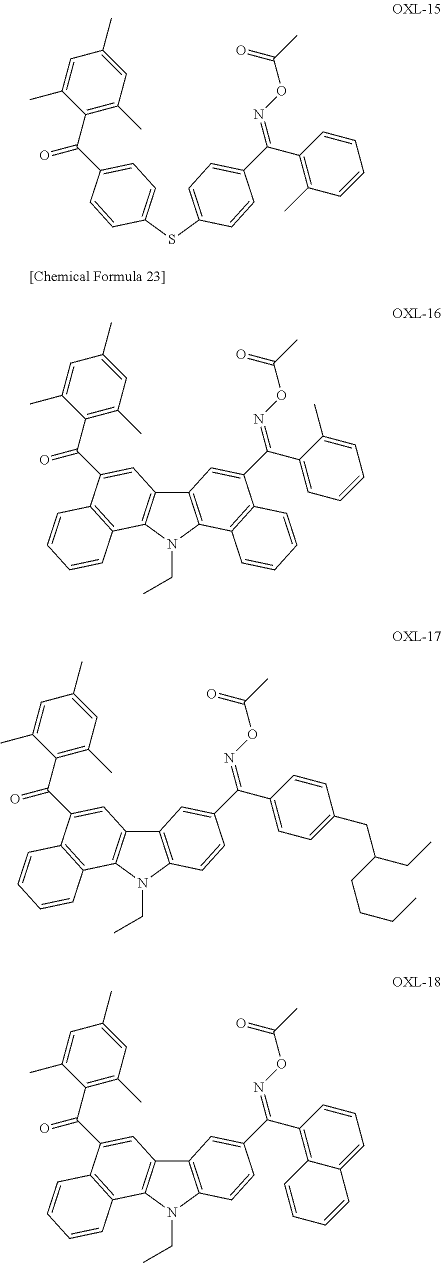

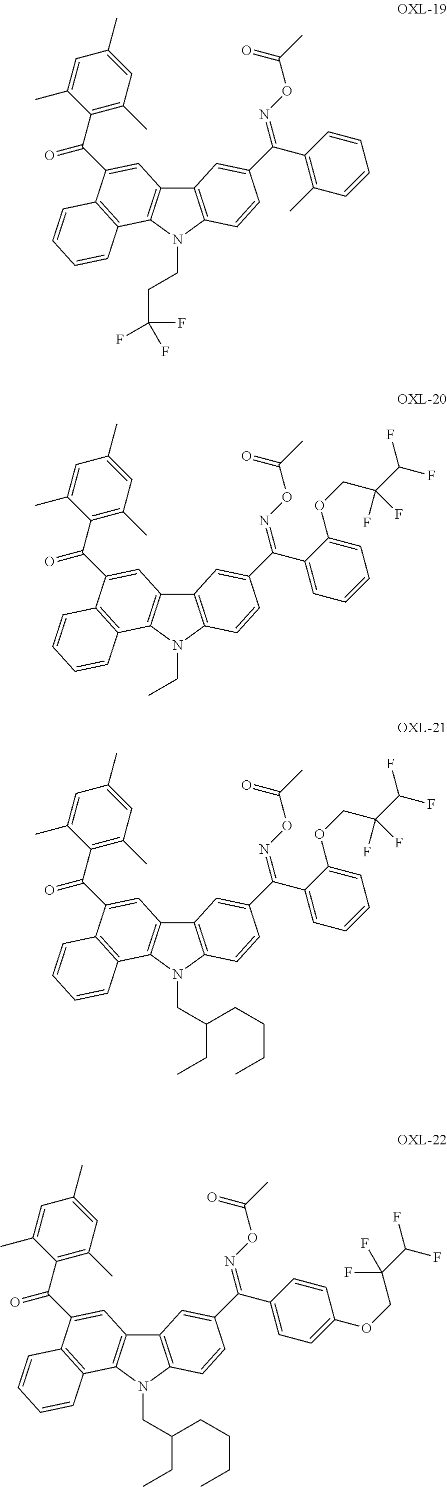 [SMILES: CC(=O)O/N=C(/c1ccc2c(c1)c1cc(C(=O)c3c(C)cc(C)cc3C)c3ccccc3c1n2CCC(F)(F)F)c1ccccc1C ; CCn1c2ccc(/C(=N/OC(C)=O)c3ccccc3OCC(F)(F)C(F)F)cc2c2cc(C(=O)c3c(C)cc(C)cc3C)c3ccccc3c21 ; CCCCC(CC)Cn1c2ccc(/C(=N/OC(C)=O)c3ccccc3OCC(F)(F)C(F)F)cc2c2cc(C(=O)c3c(C)cc(C)cc3C)c3ccccc3c21 ; CCCCC(CC)Cn1c2ccc(/C(=N/OC(C)=O)c3ccc(OCC(F)(F)C(F)F)cc3)cc2c2cc(C(=O)c3c(C)cc(C)cc3C)c3ccccc3c21]

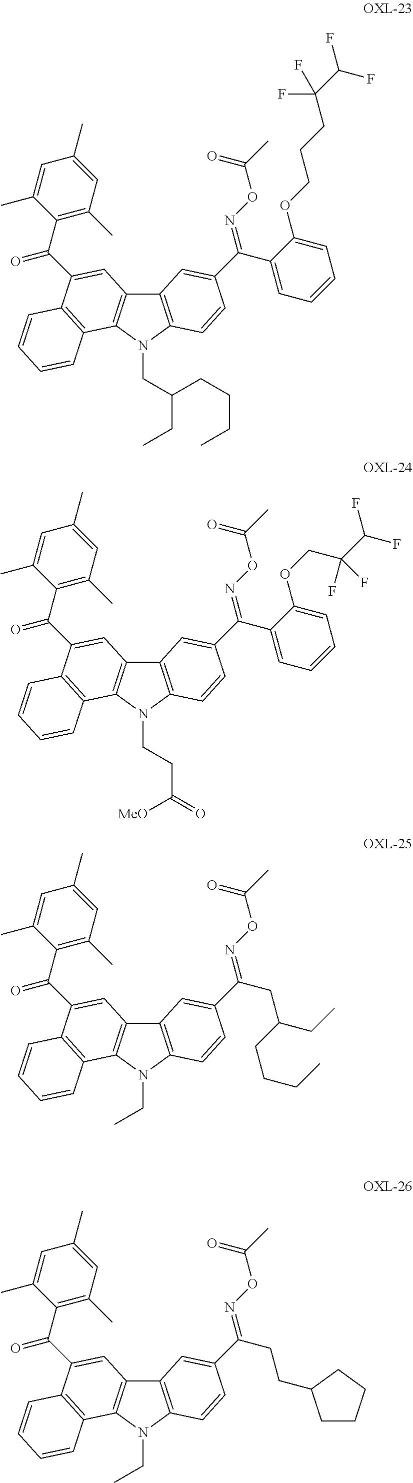

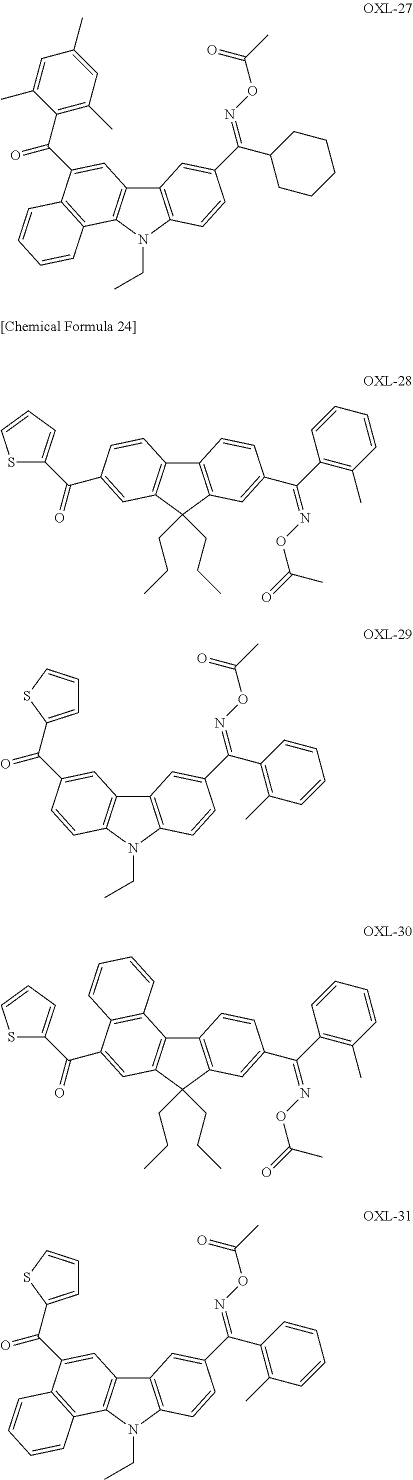 [SMILES: CCn1c2ccc(/C(=N/OC(C)=O)C3CCCCC3)cc2c2cc(C(=O)c3c(C)cc(C)cc3C)c3ccccc3c21 ; CCCC1(CCC)c2cc(C(=O)c3cccs3)ccc2-c2ccc(/C(=N\OC(C)=O)c3ccccc3C)cc21 ; CCn1c2ccc(C(=O)c3cccs3)cc2c2cc(/C(=N/OC(C)=O)c3ccccc3C)ccc21 ; CCCC1(CCC)c2cc(/C(=N\OC(C)=O)c3ccccc3C)ccc2-c2c1cc(C(=O)c1cccs1)c1ccccc21 ; CCn1c2ccc(/C(=N/OC(C)=O)c3ccccc3C)cc2c2cc(C(=O)c3cccs3)c3ccccc3c21]

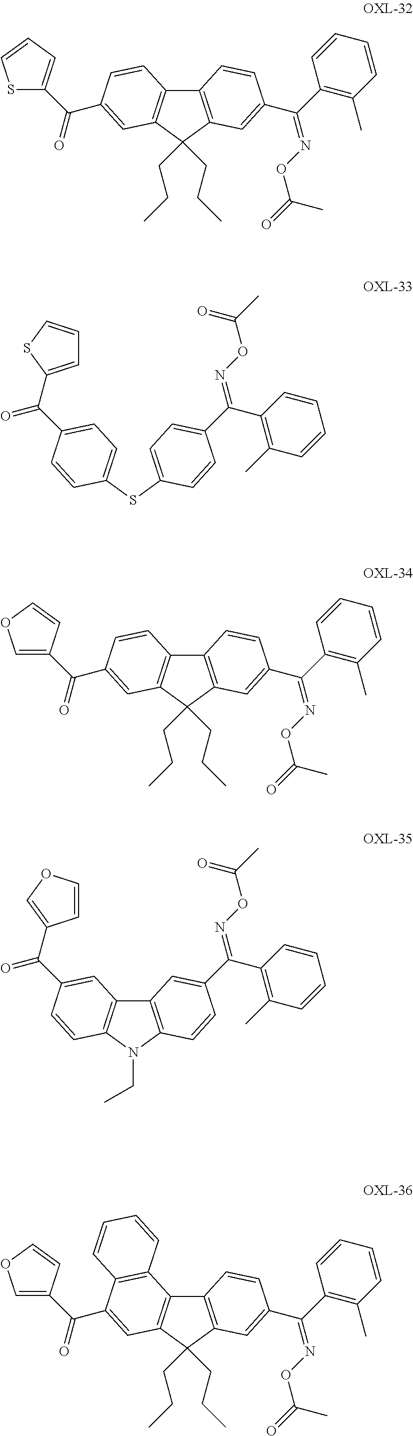 [SMILES: CCCC1(CCC)c2cc(C(=O)c3cccs3)ccc2-c2ccc(/C(=N\OC(C)=O)c3ccccc3C)cc21 ; CC(=O)O/N=C(/c1ccc(Sc2ccc(C(=O)c3cccs3)cc2)cc1)c1ccccc1C ; CCCC1(CCC)c2cc(C(=O)c3ccoc3)ccc2-c2ccc(/C(=N\OC(C)=O)c3ccccc3C)cc21 ; CCn1c2ccc(C(=O)c3ccoc3)cc2c2cc(/C(=N/OC(C)=O)c3ccccc3C)ccc21 ; CCCC1(CCC)c2cc(/C(=N\OC(C)=O)c3ccccc3C)ccc2-c2c1cc(C(=O)c1ccoc1)c1ccccc21]

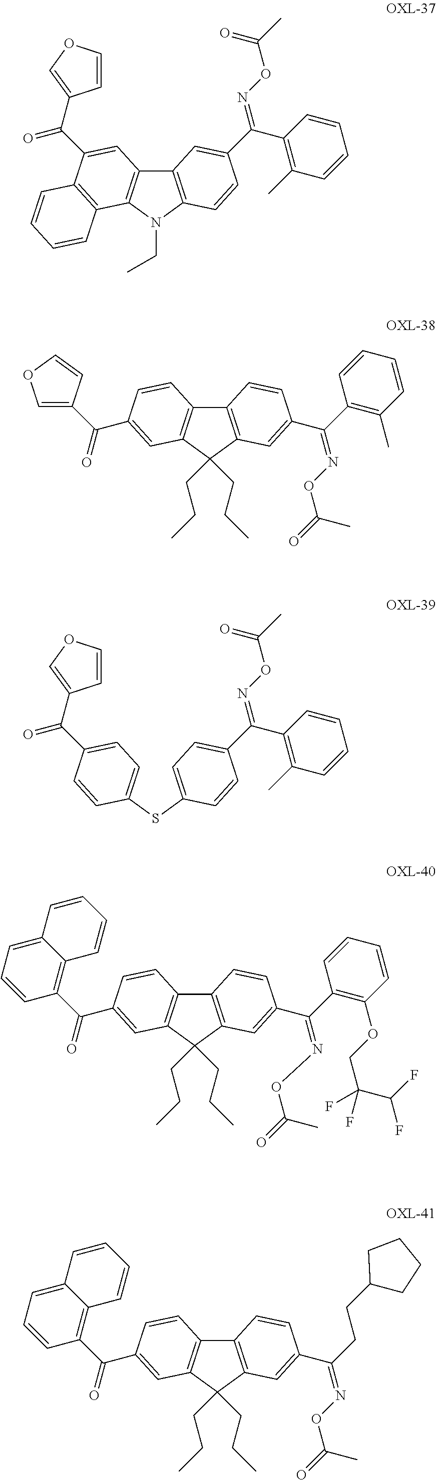 [SMILES: CCn1c2ccc(/C(=N/OC(C)=O)c3ccccc3C)cc2c2cc(C(=O)c3ccoc3)c3ccccc3c21 ; CCCC1(CCC)c2cc(C(=O)c3ccoc3)ccc2-c2ccc(/C(=N\OC(C)=O)c3ccccc3C)cc21 ; CC(=O)O/N=C(/c1ccc(Sc2ccc(C(=O)c3ccoc3)cc2)cc1)c1ccccc1C ; CCCC1(CCC)c2cc(C(=O)c3cccc4ccccc34)ccc2-c2ccc(/C(=N\OC(C)=O)c3ccccc3OCC(F)(F)C(F)F)cc21 ; CCCC1(CCC)c2cc(C(=O)c3cccc4ccccc34)ccc2-c2ccc(/C(CCC3CCCC3)=N\OC(C)=O)cc21]

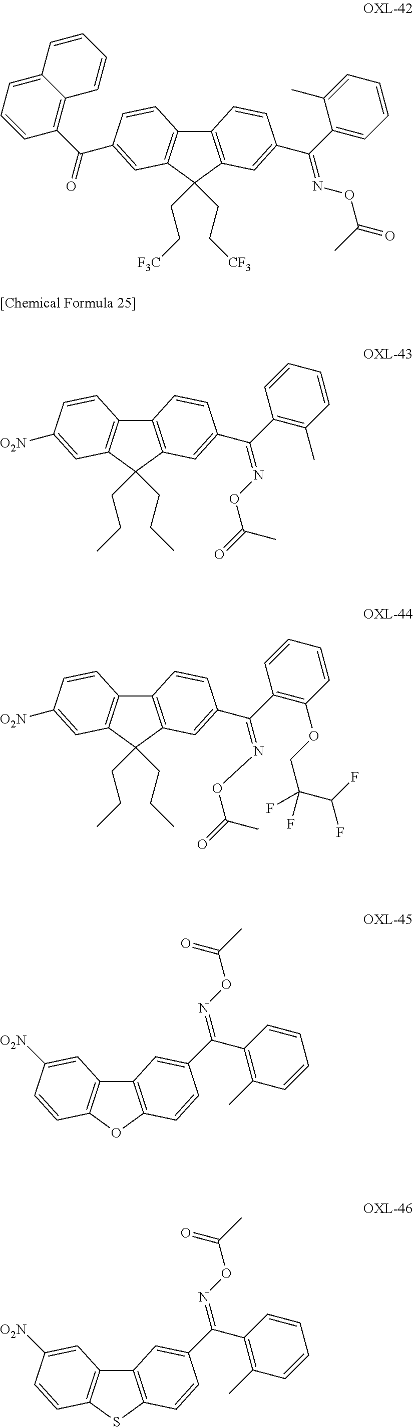 [SMILES: CC(=O)O/N=C(/c1ccc2c(c1)C(CCC(F)(F)F)(CCC(F)(F)F)c1cc(C(=O)c3cccc4ccccc34)ccc1-2)c1ccccc1C ; CCCC1(CCC)c2cc(/C(=N\OC(C)=O)c3ccccc3C)ccc2-c2ccc([N+](=O)[O-])cc21 ; CCCC1(CCC)c2cc(/C(=N\OC(C)=O)c3ccccc3OCC(F)(F)C(F)F)ccc2-c2ccc([N+](=O)[O-])cc21 ; CC(=O)O/N=C(/c1ccc2oc3ccc([N+](=O)[O-])cc3c2c1)c1ccccc1C ; CC(=O)O/N=C(/c1ccc2sc3ccc([N+](=O)[O-])cc3c2c1)c1ccccc1C]

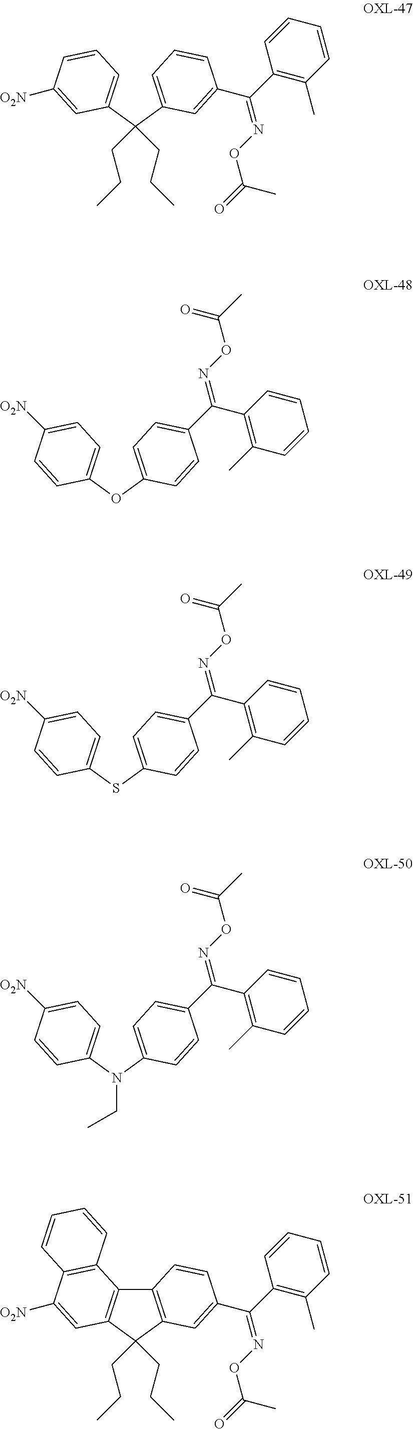 [SMILES: CCCC(CCC)(c1cccc(/C(=N\OC(C)=O)c2ccccc2C)c1)c1cccc([N+](=O)[O-])c1 ; CC(=O)O/N=C(/c1ccc(Oc2ccc([N+](=O)[O-])cc2)cc1)c1ccccc1C ; CC(=O)O/N=C(/c1ccc(Sc2ccc([N+](=O)[O-])cc2)cc1)c1ccccc1C ; CCN(c1ccc(/C(=N/OC(C)=O)c2ccccc2C)cc1)c1ccc([N+](=O)[O-])cc1 ; CCCC1(CCC)c2cc(/C(=N\OC(C)=O)c3ccccc3C)ccc2-c2c1cc([N+](=O)[O-])c1ccccc21]

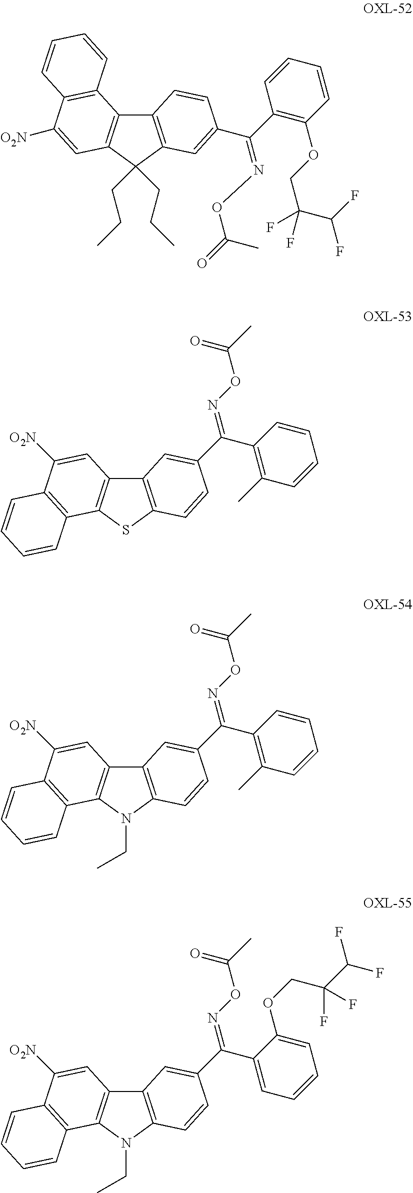 [SMILES: CCCC1(CCC)c2cc(/C(=N\OC(C)=O)c3ccccc3OCC(F)(F)C(F)F)ccc2-c2c1cc([N+](=O)[O-])c1ccccc21 ; CC(=O)O/N=C(/c1ccc2sc3c4ccccc4c([N+](=O)[O-])cc3c2c1)c1ccccc1C ; CCn1c2ccc(/C(=N/OC(C)=O)c3ccccc3C)cc2c2cc([N+](=O)[O-])c3ccccc3c21 ; CCn1c2ccc(/C(=N/OC(C)=O)c3ccccc3OCC(F)(F)C(F)F)cc2c2cc([N+](=O)[O-])c3ccccc3c21]

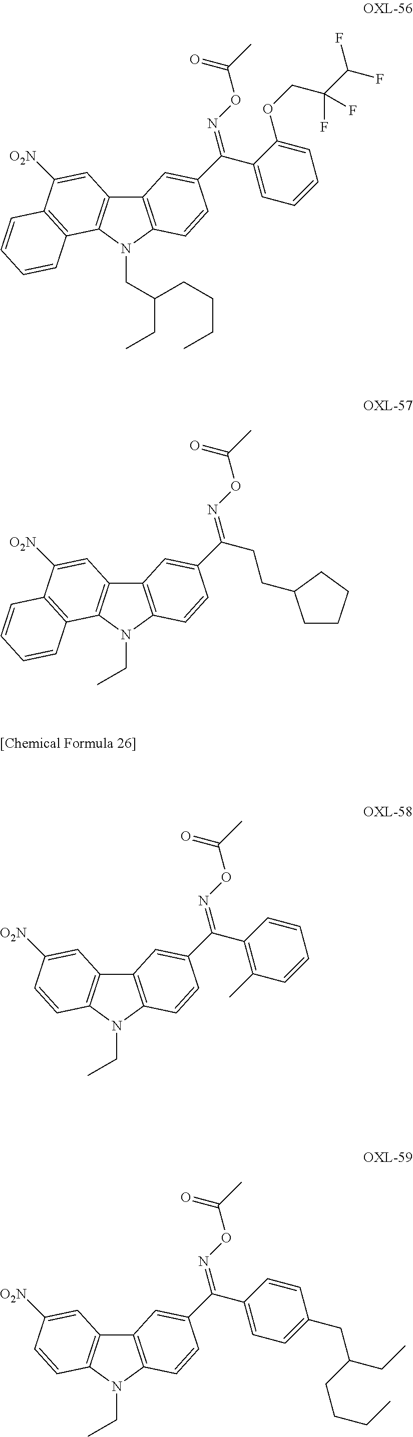 [SMILES: CCCCC(CC)Cn1c2ccc(/C(=N/OC(C)=O)c3ccccc3OCC(F)(F)C(F)F)cc2c2cc([N+](=O)[O-])c3ccccc3c21 ; CCn1c2ccc(/C(CCC3CCCC3)=N/OC(C)=O)cc2c2cc([N+](=O)[O-])c3ccccc3c21 ; CCn1c2ccc(/C(=N/OC(C)=O)c3ccccc3C)cc2c2cc([N+](=O)[O-])ccc21 ; CCCCC(CC)Cc1ccc(/C(=N\OC(C)=O)c2ccc3c(c2)c2cc([N+](=O)[O-])ccc2n3CC)cc1]

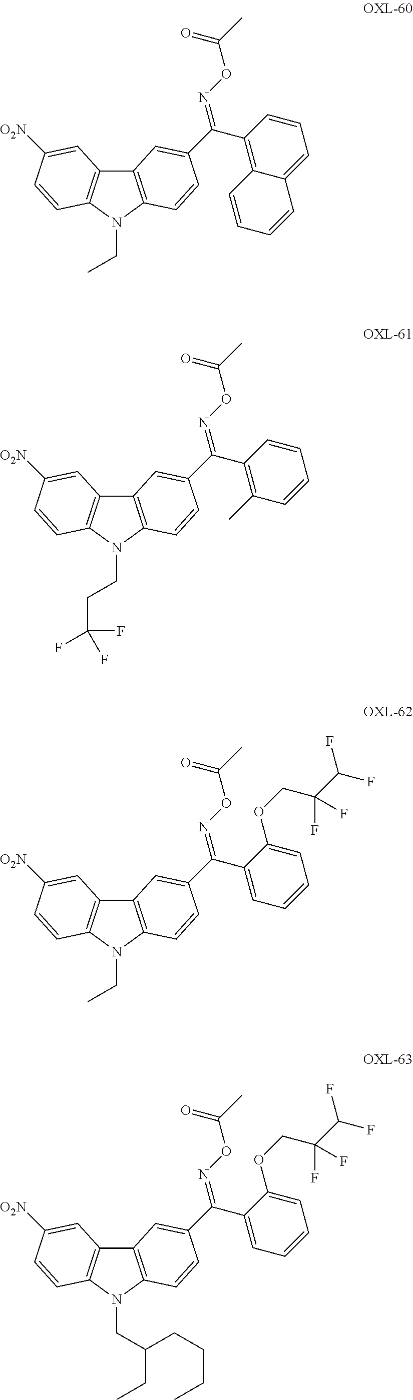 [SMILES: CCn1c2ccc(/C(=N/OC(C)=O)c3cccc4ccccc34)cc2c2cc([N+](=O)[O-])ccc21 ; CC(=O)O/N=C(/c1ccc2c(c1)c1cc([N+](=O)[O-])ccc1n2CCC(F)(F)F)c1ccccc1C ; CCn1c2ccc(/C(=N/OC(C)=O)c3ccccc3OCC(F)(F)C(F)F)cc2c2cc([N+](=O)[O-])ccc21 ; CCCCC(CC)Cn1c2ccc(/C(=N/OC(C)=O)c3ccccc3OCC(F)(F)C(F)F)cc2c2cc([N+](=O)[O-])ccc21]

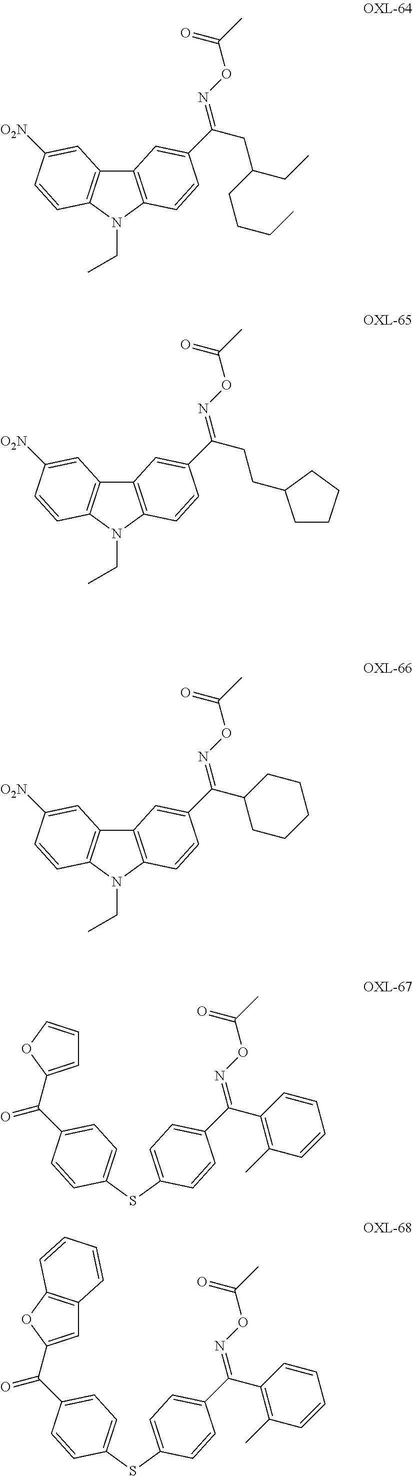 [SMILES: CCCCC(CC)C/C(=N\OC(C)=O)c1ccc2c(c1)c1cc([N+](=O)[O-])ccc1n2CC ; CCn1c2ccc(/C(CCC3CCCC3)=N/OC(C)=O)cc2c2cc([N+](=O)[O-])ccc21 ; CCn1c2ccc(/C(=N/OC(C)=O)C3CCCCC3)cc2c2cc([N+](=O)[O-])ccc21 ; CC(=O)O/N=C(/c1ccc(Sc2ccc(C(=O)c3ccco3)cc2)cc1)c1ccccc1C ; CC(=O)O/N=C(/c1ccc(Sc2ccc(C(=O)c3cc4ccccc4o3)cc2)cc1)c1ccccc1C]

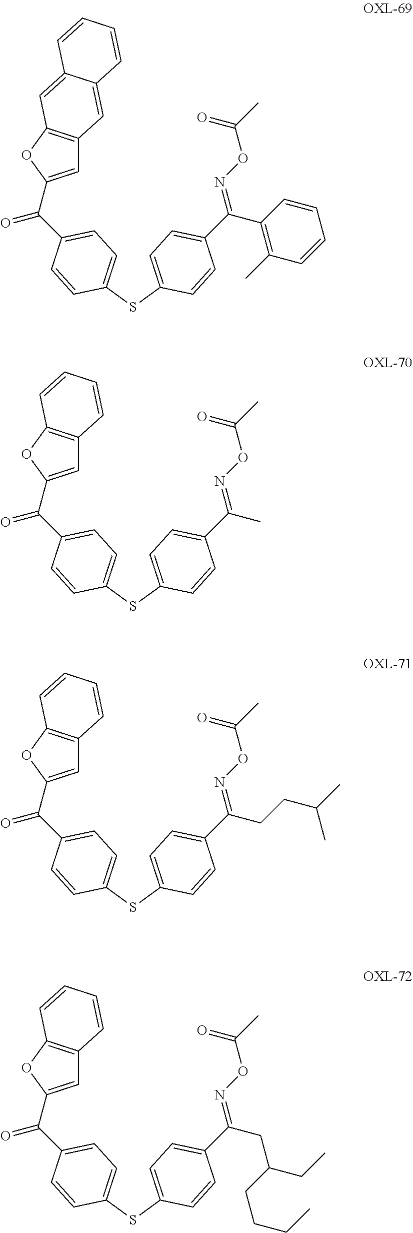 [SMILES: CC(=O)O/N=C(/c1ccc(Sc2ccc(C(=O)c3cc4cc5ccccc5cc4o3)cc2)cc1)c1ccccc1C ; CC(=O)O/N=C(\C)c1ccc(Sc2ccc(C(=O)c3cc4ccccc4o3)cc2)cc1 ; CC(=O)O/N=C(\CCC(C)C)c1ccc(Sc2ccc(C(=O)c3cc4ccccc4o3)cc2)cc1 ; CCCCC(CC)C/C(=N\OC(C)=O)c1ccc(Sc2ccc(C(=O)c3cc4ccccc4o3)cc2)cc1]

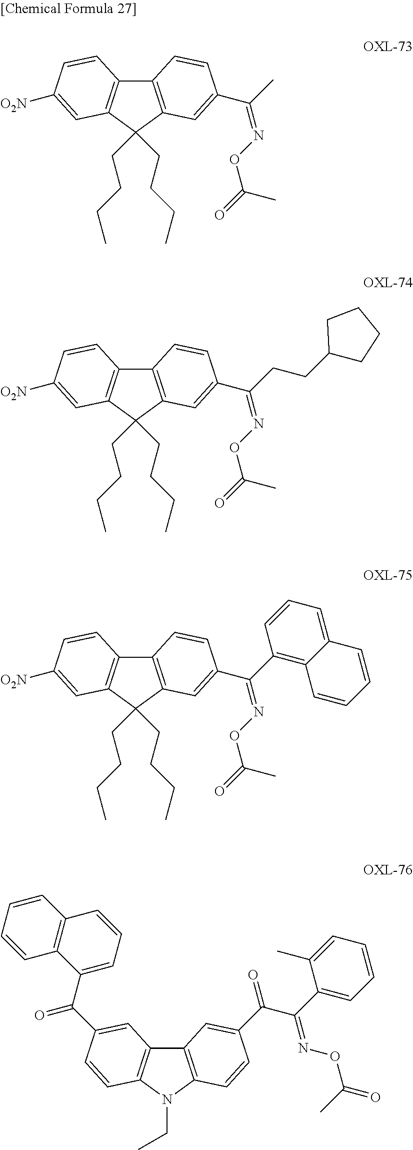 [SMILES: CCCCC1(CCCC)c2cc(/C(C)=N\OC(C)=O)ccc2-c2ccc([N+](=O)[O-])cc21 ; CCCCC1(CCCC)c2cc(/C(CCC3CCCC3)=N\OC(C)=O)ccc2-c2ccc([N+](=O)[O-])cc21 ; CCCCC1(CCCC)c2cc(/C(=N\OC(C)=O)c3cccc4ccccc34)ccc2-c2ccc([N+](=O)[O-])cc21 ; CCn1c2ccc(C(=O)/C(=N/OC(C)=O)c3ccccc3C)cc2c2cc(C(=O)c3cccc4ccccc34)ccc21]

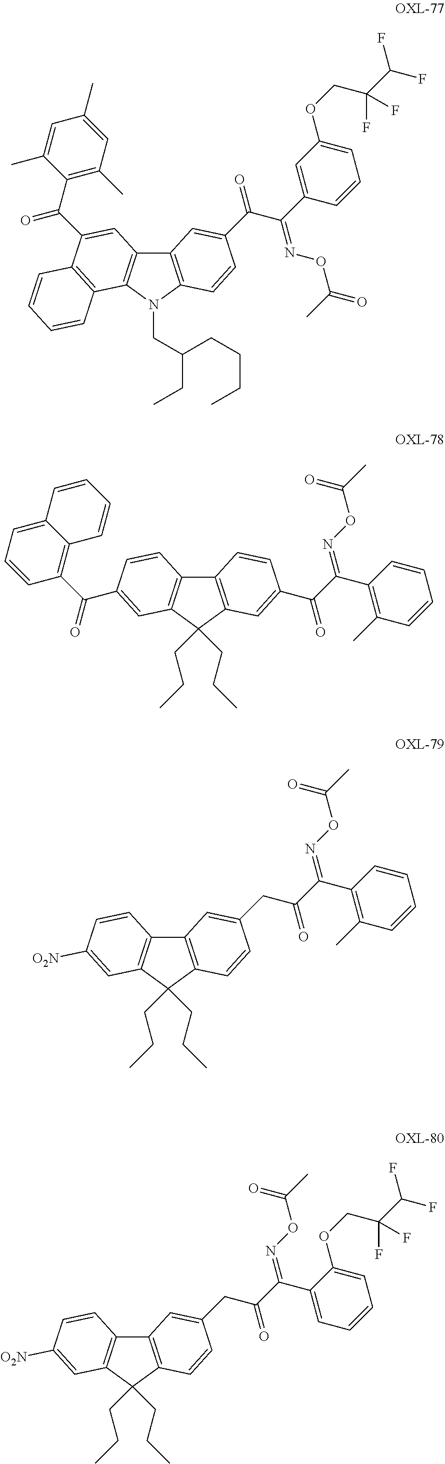 [SMILES: CCCCC(CC)Cn1c2ccc(C(=O)/C(=N/OC(C)=O)c3cccc(OCC(F)(F)C(F)F)c3)cc2c2cc(C(=O)c3c(C)cc(C)cc3C)c3ccccc3c21 ; CCCC1(CCC)c2cc(C(=O)/C(=N/OC(C)=O)c3ccccc3C)ccc2-c2ccc(C(=O)c3cccc4ccccc34)cc21 ; CCCC1(CCC)c2ccc(CC(=O)/C(=N/OC(C)=O)c3ccccc3C)cc2-c2ccc([N+](=O)[O-])cc21 ; CCCC1(CCC)c2ccc(CC(=O)/C(=N/OC(C)=O)c3ccccc3OCC(F)(F)C(F)F)cc2-c2ccc([N+](=O)[O-])cc21]

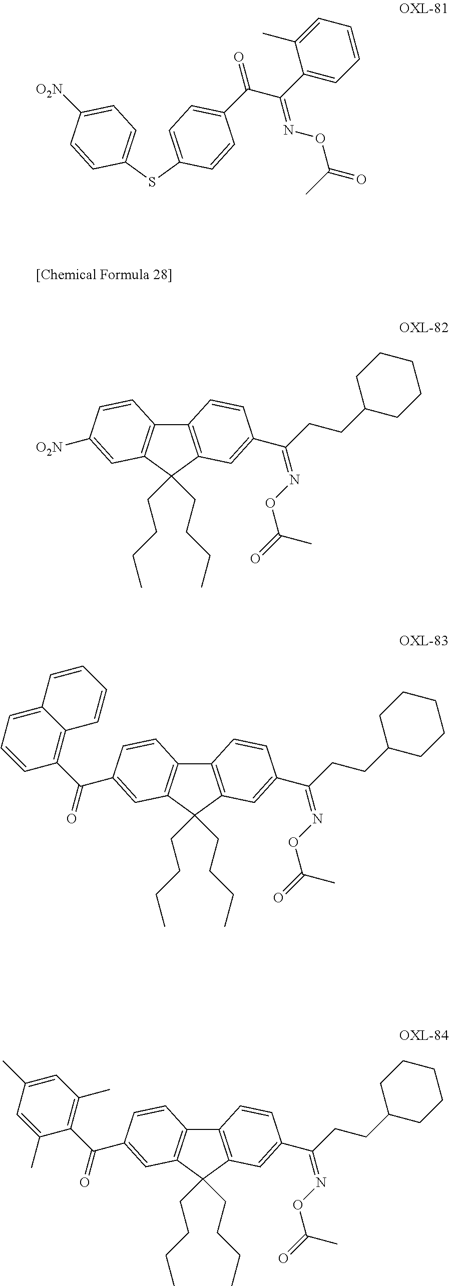 [SMILES: CC(=O)O/N=C(/C(=O)c1ccc(Sc2ccc([N+](=O)[O-])cc2)cc1)c1ccccc1C ; CCCCC1(CCCC)c2cc(/C(CCC3CCCCC3)=N\OC(C)=O)ccc2-c2ccc([N+](=O)[O-])cc21 ; CCCCC1(CCCC)c2cc(C(=O)c3cccc4ccccc34)ccc2-c2ccc(/C(CCC3CCCCC3)=N\OC(C)=O)cc21 ; CCCCC1(CCCC)c2cc(C(=O)c3c(C)cc(C)cc3C)ccc2-c2ccc(/C(CCC3CCCCC3)=N\OC(C)=O)cc21]

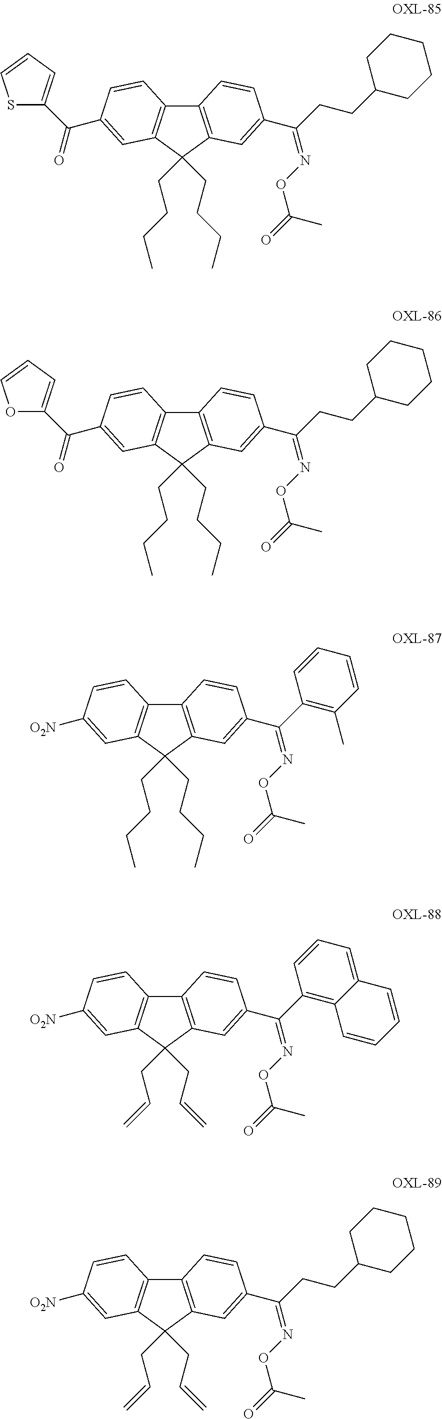 [SMILES: CCCCC1(CCCC)c2cc(C(=O)c3cccs3)ccc2-c2ccc(/C(CCC3CCCCC3)=N\OC(C)=O)cc21 ; CCCCC1(CCCC)c2cc(C(=O)c3ccco3)ccc2-c2ccc(/C(CCC3CCCCC3)=N\OC(C)=O)cc21 ; CCCCC1(CCCC)c2cc(/C(=N\OC(C)=O)c3ccccc3C)ccc2-c2ccc([N+](=O)[O-])cc21 ; C=CCC1(CC=C)c2cc(/C(=N\OC(C)=O)c3cccc4ccccc34)ccc2-c2ccc([N+](=O)[O-])cc21 ; C=CCC1(CC=C)c2cc(/C(CCC3CCCCC3)=N\OC(C)=O)ccc2-c2ccc([N+](=O)[O-])cc21]

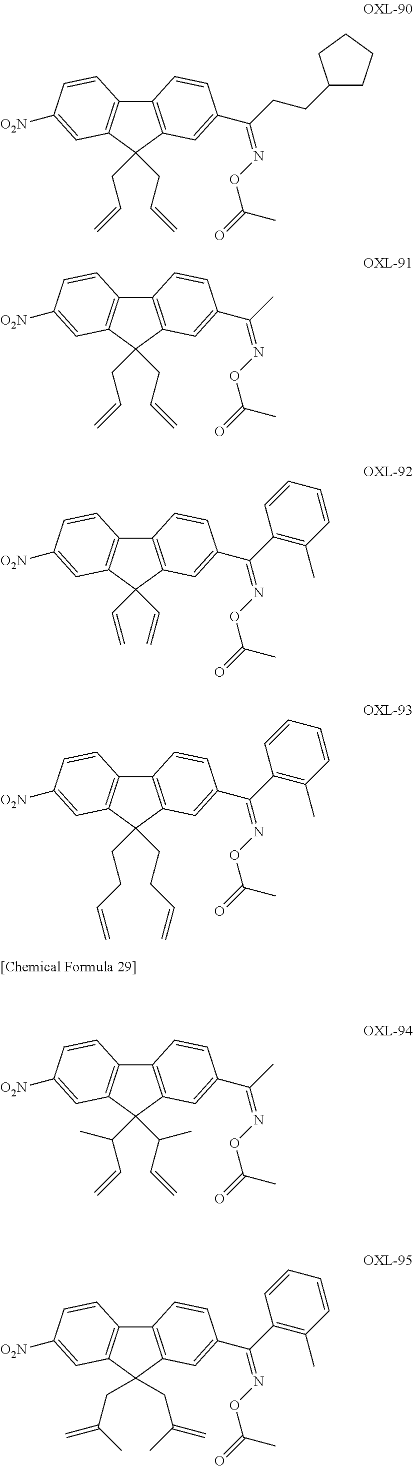

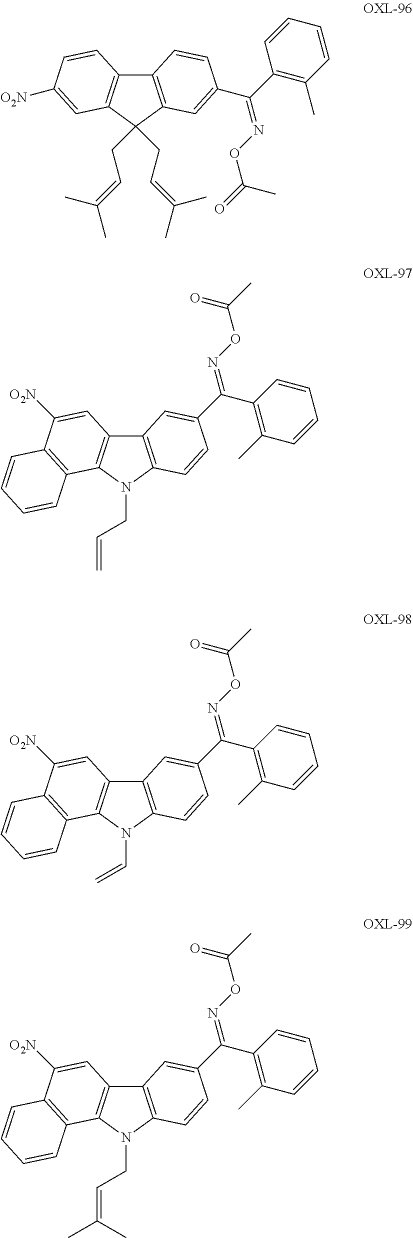 [SMILES: CC(=O)O/N=C(\c1ccc2c(c1)C(CC=C(C)C)(CC=C(C)C)c1cc([N+](=O)[O-])ccc1-2)c1ccccc1C ; C=CCn1c2ccc(/C(=N/OC(C)=O)c3ccccc3C)cc2c2cc([N+](=O)[O-])c3ccccc3c21 ; C=Cn1c2ccc(/C(=N/OC(C)=O)c3ccccc3C)cc2c2cc([N+](=O)[O-])c3ccccc3c21 ; CC(=O)O/N=C(/c1ccc2c(c1)c1cc([N+](=O)[O-])c3ccccc3c1n2CC=C(C)C)c1ccccc1C]

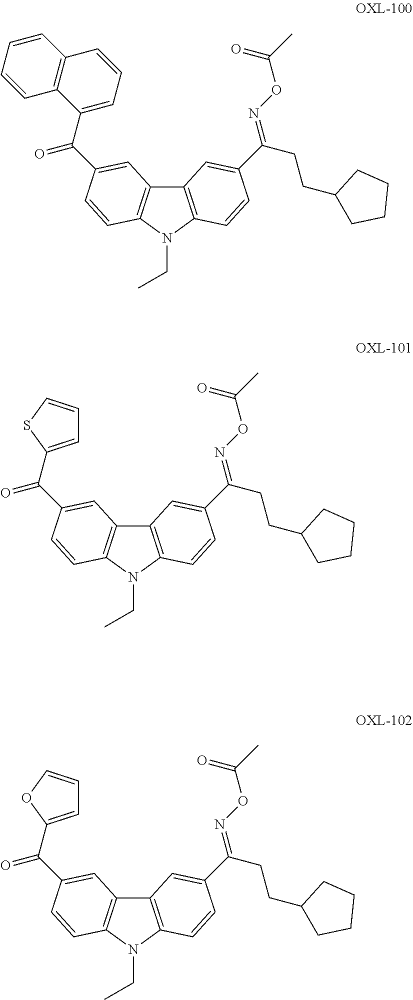 [SMILES: CCn1c2ccc(C(=O)c3cccc4ccccc34)cc2c2cc(/C(CCC3CCCC3)=N/OC(C)=O)ccc21 ; CCn1c2ccc(C(=O)c3cccs3)cc2c2cc(/C(CCC3CCCC3)=N/OC(C)=O)ccc21 ; CCn1c2ccc(C(=O)c3ccco3)cc2c2cc(/C(CCC3CCCC3)=N/OC(C)=O)ccc21]

The (C1-1) specific oxime ester-based photo initiator can be synthesized by known methods. The methods include the synthesis methods described in JP 2013-190459 A, JP 2016-191905 A, and International Publication No. 2014/500852.

The maximum absorption wavelength of the (C1-1) specific oxime ester-based photo initiator is preferably 330 nm or more, more preferably 340 nm or more, still more preferably 350 nm or more. When the maximum absorption wavelength is 330 nm or more, the sensitivity for exposure can be improved, and a pattern in a low-taper shape can be formed after development. In addition, the change in pattern opening width between before and after thermal curing can be suppressed, and the halftone characteristics can be improved. On the other hand, the maximum absorption wavelength of the (C1-1) specific oxime ester-based photo initiator is preferably 410 nm or less, more preferably 400 nm or less, still more preferably 390 nm or less, particularly preferably 380 nm or less. When the maximum absorption wavelength is 410 nm or less, the sensitivity for exposure can be improved, and a pattern in a low-taper shape can be formed after development. In addition, the change in pattern opening width between before and after thermal curing can be suppressed, and the halftone characteristics can be improved.

Furthermore, in a case where the maximum transmission wavelength of the (Da) black colorant described later is 330 to 410 nm, when the maximum absorption wavelength of the (C1-1) specific oxime ester-based photo initiator is 330 to 410 nm, the sensitivity for exposure can be improved, and a pattern in a low-taper shape can be formed after development. In addition, the change in pattern opening width between before and after thermal curing can be suppressed, and the halftone characteristics can be improved. This is believed to be because UV light for exposure is capable of reaching the deep part of the film, thus causing UV curing to proceed efficiently even in the deep part of the film.

It is to be noted that the maximum absorption wavelength and the maximum transmission wavelength respectively refer to a wavelength at which the maximum absorption is exhibited and a wavelength at which the maximum transmission is exhibited in the absorption spectrum and transmission spectrum within a wavelength range of 300 to 800 nm.

The absorbance at a wavelength of 360 nm in a 0.01 g/L propylene glycol monomethyl ether acetate solution of the (C1-1) specific oxime ester-based photo initiator is preferably 0.20 or more, more preferably 0.25 or more, still more preferably 0.30 or more, even more preferably 0.35 or more, particularly preferably 0.40 or more, most preferably 0.45 or more. When the absorbance is 0.20 or more, the sensitivity for exposure can be improved, and a pattern in a low-taper shape can be formed after development. In addition, the change in pattern opening width between before and after thermal curing can be suppressed, and the halftone characteristics can be improved. On the other hand, the absorbance at a wavelength of 360 nm in a 0.01 g/L propylene glycol monomethyl ether acetate solution of the (C1-1) specific oxime ester-based photo initiator is preferably 1.00 or less. When the absorbance is 1.00 or less, the residue generation after development can be inhibited, and the resolution after development can be improved.

The content of the (C1-1) specific oxime ester-based photo initiator in the negative photosensitive resin composition according to the present invention is, in a case where the (A) alkali-soluble resin and the (B) radical polymerizable compound are regarded as 100 parts by mass in total, preferably 0.1 parts by mass or more, more preferably 0.5 parts by mass or more, still more preferably 0.7 parts by mass or more, particularly preferably 1 part by mass or more. When the content is 0.5 parts by mass or more, the sensitivity for exposure can be improved. Furthermore, the content of the (C1-1) specific oxime ester-based photo initiator is, from the viewpoint of pattern shape control after development and after thermal curing, preferably 3 parts by mass or more, more preferably 4 parts by mass or more, still more preferably 5 parts by mass or more, particularly preferably 7 parts by mass or more. When the content is 3 parts by mass or more, a pattern in a low-taper shape can be formed after development, and the change in pattern opening width between before and after thermal curing can be suppressed. In addition, halftone characteristics can be improved. On the other hand, the content of the (C1-1) specific oxime ester-based photo initiator is preferably 25 parts by mass or less, more preferably 22 parts by mass or less, still more preferably 20 parts by mass or less, particularly preferably 15 parts by mass or less. When the content is 25 parts by mass or less, the resolution after development can be improved, and a cured film with a pattern in a low-taper shape can be obtained. Furthermore, the content of the (C1-1) specific oxime ester-based photo initiator is, from the viewpoint of pattern shape control after development, preferably 20 parts by mass or less, more preferably 17 parts by mass or less, still more preferably 15 parts by mass or less, particularly preferably 13 parts by mass or less. When the content is 20 parts by mass or less, a pattern in a low-taper shape can be formed after development, and the change in pattern opening width between before and after thermal curing can be suppressed.

### <(C1-2) α-Aminoketone-Based Photo Initiator, (C1-3) Acylphosphine Oxide-Based Photo Initiator, and (C1-4) Biimidazole-Based Photo Initiator>

The negative photosensitive resin composition according to the present invention preferably further contains, as the (C1) photo initiator, one or more selected from the group consisting of the (C1-2) α-aminoketone-based photo initiator, the (C1-3) acylphosphine oxide-based photo initiator, and the (C1-4) biimidazole photo initiator.

Containing one or more selected from the group consisting of the (C1-2) α-aminoketone-based photo initiator, the (C1-3) acylphosphine oxide-based photo initiator, and the (C1-4) biimidazole photo initiator allows the sensitivity for exposure to be improved. This is believed to be because these photo initiators have an absorption wavelength in a wavelength region that is different from the absorption wavelength of the above-described (C1-1) specific oxime ester-based photo initiator, thereby allowing UV light for exposure to be more efficiently used for radical curing. In addition to the foregoing, the change in pattern opening width between before and after thermal curing can be suppressed. This is presumed to be for the following reason, in addition to radical curing proceeding more efficiently depending on the difference in absorption wavelength as well. It is believed that since the (C1-2) α-aminoketone-based photo initiator, the (C1-3) acylphosphine oxide-based photo initiator, and the (C1-4) biimidazole-based photo initiator contain nitrogen or phosphorus in the molecules, amine or phosphine is produced by photolysis during exposure and/or thermal decomposition during thermal curing, and these products act as a cross-linking catalyst during thermal curing, thereby suppressing reflow at the pattern skirt.

In the negative photosensitive resin composition according to the present invention, the content ratio of the (C1-1) specific oxime ester-based photo initiator in the (C1) photo initiator is preferably 55% by mass or higher, more preferably 60% by mass or higher, still more preferably 65% by mass or higher, even more preferably 70% by mass or higher, particularly preferably 75% by mass or higher. When the content ratio is 55% by mass or higher, the sensitivity for exposure can be improved, and the change in pattern opening width between before and after thermal curing can be suppressed. On the other hand, the content ratio of the (C1-1) specific oxime ester-based photo initiator is preferably 95% by mass or lower, more preferably 93% by mass or lower, still more preferably 90% by mass or lower, still more preferably 88% by mass or lower, particularly preferably 85% by mass or lower. When the content ratio is 95% by mass or lower, the sensitivity for exposure can be improved, and the change in pattern opening width between before and after thermal curing can be suppressed.

In the negative photosensitive resin composition according to the present invention, the total content ratio of the (C1-2) α-aminoketone-based photo initiator, (C1-3) acylphosphine oxide-based photo initiator, and (C1-4) biimidazole-based photo initiator in the (C1) photo initiator is preferably 5% by mass or higher, more preferably 7% by mass or higher, still more preferably 10% by mass or higher, even more preferably 12% by mass or higher, particularly preferably 15% by mass or higher. When the content ratio is 5% by mass or higher, the sensitivity for exposure can be improved, and the change in pattern opening width between before and after thermal curing can be suppressed. On the other hand, the content ratio of the (C1-1) specific oxime ester-based photo initiator is preferably 45% by mass or lower, more preferably 40% by mass or lower, still more preferably 35% by mass or lower, even more preferably 30% by mass or lower, particularly preferably 25% by mass or lower. When the content ratio is 45%. by mass or lower, the sensitivity for exposure can be improved, and the change in pattern opening width between before and after thermal curing can be suppressed.

### <(C2) Photo Acid Generator>

The negative photosensitive resin composition according to the present invention may further contain a (C2) photo acid generator as the (C) photosensitive agent. The (C2) photo acid generator refers to a compound that causes bond cleavage upon exposure to generate an acid. Containing the (C2) photo acid generator accelerates UV curing during the exposure, allowing the sensitivity to be improved. Furthermore, the crosslink density after thermal curing of the resin composition is improved, thereby allowing the chemical resistance of the cured film to be improved. Examples of the (C2) photo acid generator include ionic compounds and non-ionic compounds.

As the (C2) photo acid generator of an ionic compound, compounds containing no heavy metal or halogen ion are preferred, and triorganosulfonium salt-based compounds are more preferred. Examples of the triorganosulfonium salt-based compound include methanesulfonate, trifluoromethanesulfonate, camphorsulfonate, or 4-toluenesulfonate of triphenylsulfonium; methanesulfonate, trifluoromethanesulfonate, camphorsulfonate, or 4-toluenesulfonate of dimethyl-1-naphthylsulfonium; methanesulfonate, trifluoromethanesulfonate, camphorsulfonate, or 4-toluenesulfonate of dimethyl(4-hydroxy-1-naphthyl) sulfonium; methanesulfonate, trifluoromethanesulfonate, camphorsulfonate, or 4-toluenesulfonate of dimethyl(4,7-dihydroxy-1-naphthyl) sulfonium; methanesulfonate, trifluoromethanesulfonate, camphorsulfonate, or 4-toluenesulfonate of diphenyliodonium.

Examples of the (C2) photo acid generator of a non-ionic compound include halogen-containing compounds, diazomethane compounds, sulfone compounds, sulfonate ester compounds, carboxylic acid ester compounds, sulfonimide compounds, phosphate ester compounds, and sulfone benzotriazoles compounds. Among these (C2) photo acid generators, the non-ionic compounds are more preferred than the ionic compounds from the viewpoints of the solubility and the insulation properties of the cured film. From the viewpoint of the strength of the acid generated, those that generate benzenesulfonic acid, 4-toluenesulfonic acid, perfluoroalkylsulfonic acid, or phosphoric acid are more preferred. From the viewpoints of the high sensitivity due to the high quantum yield for j-lines (wavelength: 313 nm), i-lines (wavelength: 365 nm), h-lines (wavelength: 405 nm), or g-lines (wavelength: 436 nm), and the transparency of the cured film, a sulfonic acid ester compound, a sulfonimide compound, or an imino sulfonic acid ester compound is still more preferred.

The content of the (C2) photo acid generator in the negative photosensitive resin composition according to the present invention is, in a case where the (A) alkali-soluble resin and the (B) radical polymerizable compound are regarded as 100 parts by mass in total, preferably 0.1 parts by mass or more, more preferably 0.5 parts by mass or more, still more preferably 0.7 parts by mass or more, particularly preferably 1 part by mass or more. When the content is 0.1 parts by mass or more, the sensitivity for exposure can be improved. On the other hand, the content of the (C2) photo acid generator is preferably 25 parts by mass or less, more preferably 20 parts by mass or less, still more preferably 17 parts by mass or less, particularly preferably 15 parts by mass or less. When the content is 25 parts by mass or less, the resolution after development can be improved, and a low-taper pattern shape can be obtained.

### <(D) Colorant, (Da) Black Colorant, and (Db) Non-Black Colorant>

The negative photosensitive resin composition according to the present invention further contains, as the (D) colorant, the (Da) black colorant. The colorant (D) refers to a compound that absorbs light of a specific wavelength, in particular, a compound which is colored by absorbing light with a wavelength of visible light (380 to 780 nm). Containing the (D) colorant makes it possible to color a film obtained from the negative photosensitive resin composition, and makes it possible to impart colorability of coloring the light transmitted through the resin composition film or the light reflected from the resin composition film into a desired color. Furthermore, it is possible to impart a light-blocking property of blocking light with a wavelength absorbed by the (D) colorant, from light transmitted through the resin composition film or light reflected from the resin composition film.

Examples of the colorant (D) include compounds that absorb light with a wavelength of visible light and are colored in red, orange, yellow, green, blue, or purple. Two or more of these colorants are combined, thereby making it possible to improve the toning property of toning light transmitted through the resin composition film or reflected from the resin composition film to desired color coordinates.

The negative photosensitive resin composition according to the present invention contains, as the (D) colorant, the (Da) black colorant as an essential component. The (Da) black colorant refers to a compound which is colored in black by absorbing light with a wavelength of visible light. Containing the (Da) black colorant makes it possible to improve the light blocking property of blocking the light transmitted through the resin composition film or the light reflected from the resin composition film, because the resin composition film is blackened. For this reason, the composition is suitable for applications such as a pixel defining layer, an electrode insulation layer, a wiring insulation layer, an interlayer insulation layer, a TFT planarization layer, an electrode planarization layer, a wiring planarization layer, a TFT protective layer, an electrode protective layer, a wiring protective layer, a gate insulation layer, a color filter, a black matrix, or a black column spacer. The composition is preferred as in particular, a light-blocking pixel defining layer, electrode insulation layer, wiring insulation layer, interlayer insulation layer, TFT planarization layer, electrode planarization layer, wiring planarization layer, TFT protective layer, electrode protective layer, wiring protective layer, or gate insulating layer of an organic EL display protective layer, and suitable for applications which require contrast increased by suppression of external light reflection, such as a light-blocking pixel defining layer, interlayer insulation layer, TFT planarization layer, or TFT protective layer.

The black color in the colorant refers to a color with Colour Index Generic Name (hereinafter a “C.I. number”) including “BLACK” therein. The color assigned with no C.I. number refers to a black color in the case of the composition as a cured film. The black color in a mixture of (D) colorants of two or more colors with non-black C.I. numbers, and a mixture of (D) colorants of two or more colors, including at least one (D) colorant assigned with no C.I. number refers to a black color in the case of the composition as a cured film. The black color in the case of the composition a cured film means that in the transmission spectrum of the cured film of the resin composition containing the (D) colorant, based on the Lambert Beer formula, the transmittance per 1.0 μm of the film thickness at a wavelength of 550 nm is converted with the film thickness within the range of 0.1 to 1.5 μm such that the transmittance at a wavelength of 550 nm is 10%, the transmittance at a wavelength of 450 to 650 nm in the converted transmission spectrum is 25% or less.

The transmission spectrum of the cured film can be obtained by the following method. A resin composition containing at least an arbitrary binder resin and the (D) colorant is prepared such that the content ratio of the (D) colorant in the total solid content of the resin composition is 35% by mass. After a film of the resin composition is applied onto a Tempax glass substrate (manufactured by AGC TECHNO GLASS CO., LTD.), the film is prebaked at 110° C. for 2 minutes to form a film, thereby providing a prebaked film. Next, with the use of a high-temperature inert gas oven (INH-9CD-S; manufactured by Koyo Thermo Systems Co., Ltd.), the film is subjected to thermal curing at 250° C. for 60 minutes under a nitrogen atmosphere, thereby preparing a cured film of 1.0 μm in film thickness from the resin composition containing the (D) colorant (hereinafter, “colorant-containing cured film”). In addition, a resin composition containing the binder resin and containing no (D) colorant is prepared, and applied onto a Tempax glass substrate, and prebaked and subjected to thermal curing by the same manner as mentioned above, thereby preparing a cured film of 1.0 μm in film thickness from the resin composition containing no (D) colorant (hereinafter, a “blank cured film”. With the use of a ultraviolet-visible spectrophotometer (MultiSpec-1500; manufactured by Shimadzu Corporation), first, the Tempax glass substrate with the blank cured film formed to have the thickness of 1.0 μm is measured, and the ultraviolet-visible absorption spectrum is regarded as a blank. Next, the Tempax glass substrate with the prepared colorant-containing cured film formed is measured with a single beam, thereby measuring the transmittance per 1.0 μm of the film thickness at a wavelength of 450 to 650 nm, and calculating the transmittance of the colorant-containing cured film from the difference from the blank.

As the (Da) black colorant, a compound which is colored in black by absorbing light of all wavelengths of visible light is preferred from the viewpoint of the light-blocking property. Also preferred is a mixture of two or more (D) colorants selected from red, orange, yellow, green, blue, or purple colorants. Two or more of these (D) colorants are combined, thereby allowing pseudo-coloring in black, and allowing the light-blocking property to be improved.

The maximum transmission wavelength of the (Da) black colorant is preferably 330 nm or more, more preferably 340 nm or more, still more preferably 350 nm or more. When the maximum transmission wavelength is 330 nm or more, the sensitivity for exposure can be improved, and a pattern in a low-taper shape can be formed after development. In addition, the change in pattern opening width between before and after thermal curing can be suppressed, and the halftone characteristics can be improved. On the other hand, the maximum transmission wavelength of the (Da) black colorant is preferably 410 nm or less, more preferably 400 nm or less, still more preferably 390 nm or less, particularly preferably 380 nm or less. When the maximum transmission wavelength is 410 nm or less, the sensitivity for exposure can be improved, and a pattern in a low-taper shape can be formed after development. In addition, the change in pattern opening width between before and after thermal curing can be suppressed, and the halftone characteristics can be improved.

Further, as described above, in a case where the maximum transmission wavelength of the (Da) black colorant is 330 to 410 nm, the maximum absorption wavelength of the above-described (C1-1) specific oxime ester-based photo initiator is preferably 330 to 410 nm.

The maximum transmission wavelength in the (D) colorant can be calculated by, in the same way as the above-described method for measuring the transmission spectrum of the cured film, measuring the transmittance per 1.0 μm of the film thickness at a wavelength of 300 to 800 nm, and within the wavelength range of 300 to 800 nm, determining a wavelength at which the maximum transmission in the transmission spectrum is exhibited.

As the negative photosensitive resin composition according to the present invention, the (Da) black colorant described above preferably contains one or more selected from a (D1a) black pigment, a (D2a-1) black dye, and a (D2a-2) dye mixture of two or more colors to be described later, and from the viewpoint of light-blocking property, more preferably contains the black pigment (D1a) described later.

The (Db) non-black colorant refers to a compound which is colored by absorbing light with a wavelength of visible light. More specifically, the (Db) non-black colorant is the above-described colorant which is colored in red, orange, yellow, green, blue, or purple, excluding black. Containing the (Da) black colorant and the (Db) non-black colorant makes it possible to impart a light-blocking property as well as colorability and/or a toning property to the resin composition film.

As the negative photosensitive resin composition according to the present invention, the above-described (Db) non-black colorant preferably contains a (D1b) non-black pigment and/or a (D2b) non-black dye, which will be described later, and from the viewpoints of the light-blocking property, and heat resistance or weather resistance, preferably contains the (D1b) non-black pigment, which will be described later.

In the negative photosensitive resin composition according to the present invention, the content ratio of the (D) colorant to 100% by mass in total of the (A) alkali-soluble resin, (D) colorant, and (E) dispersant described later is preferably 15% by mass or higher, more preferably 20% by mass or higher, still more preferably 25% by mass or higher, particularly preferably 30% by mass or higher. When the content ratio is 15% by mass or higher, the light-blocking property, the colorability, or the toning property can be improved. On the other hand, the content ratio of the (D) colorant is preferably 80% by mass or lower, more preferably 75% by mass or lower, still more preferably 70% by mass or lower, particularly preferably 65% by mass or lower. When the content ratio is 80% by mass or lower, the sensitivity during the exposure can be improved.

Furthermore, the content ratio of the (D) colorant to the total solid content of the negative photosensitive resin composition according to the present invention, excluding the solvent, is preferably 5% by mass or higher, more preferably 10% by mass or higher, still more preferably 15% by mass or higher, particularly preferably 20% by mass or higher. When the content ratio is 5% by mass or higher, the light-blocking property, the colorability, or the toning property can be improved. On the other hand, the content ratio of the (D) colorant is preferably 70% by mass or lower, more preferably 65% by mass or lower, still more preferably 60% by mass or lower, even more preferably 55%. by mass or lower, particularly preferably 50% by mass or lower. When the content ratio is 70% by mass or lower, the sensitivity for exposure can be improved.

In the negative photosensitive resin composition according to the present invention, the preferred content ratio of the (Da) black colorant is the same as the preferred content ratio of the (D) colorant described above.

### <(D1) Pigment, (D1-1) Organic Pigment, and (D1-2) Inorganic Pigment>

In the negative photosensitive resin composition according to the present invention, the above-mentioned (D) colorant preferably contains the (D1) pigment. As an aspect in which the above-described (D) colorant contains the (D1) pigment, the above-described (Da) black colorant is necessarily contained, and the (Db) non-black colorant can be optionally contained. The (D1) pigment refers to a compound that colors an object with the (D1) pigment physically adsorbed on the surface of the object, or with the interaction between the (D1) pigment and the surface of the object, and typically, the (D1) pigment is insoluble in solvents. In addition, coloring with (D1) pigment has high hiding power, and fading due to ultraviolet rays or the like is less likely to be caused. Containing the (D1) pigment allows coloring in a color with excellent hiding power, and then allows the light-blocking property and weather resistance of the resin composition film to be improved.

The number average particle size of the (D1) pigment is preferably 1 to 1,000 nm, more preferably 5 to 500 nm, still more preferably 10 to 200 nm. When the number average particle size of the (D1) pigment is 1 to 1,000 nm, the light-blocking property of the resin composition film and the dispersion stability of the (D1) pigment can be improved. In this regard, the number average particle size of the (D1) pigment can be determined by measuring laser scattering (dynamic light scattering method) due to the Brownian motion of the (D1) pigment in the solution, with the use of a submicron particle size distribution measurement device (N4-PLUS; manufactured by Beckman Coulter) or a zeta potential/particle size/molecular weight measurement device (Zeta Sizer Nano ZS; Sysmex Corporation) Furthermore, the number average particle size of the (D1) pigment in the cured film obtained from the resin composition can be determined by measurement with the use of a scanning electron microscope (hereinafter “SEM”) and a transmission electron microscope (hereinafter “TEM”). At the magnification of 50,000 to 200,000 times, the number average particle size of the (D1) pigment is directly measured. When the (D1) pigment has a true sphere, the diameter of the true sphere is measured, and regarded as the number average particle size. When the (D1) pigment is not a true sphere, the longest diameter (hereinafter, referred to as a “long axis diameter”) and the longest diameter (hereinafter, referred to as a “short axis diameter”) in a direction perpendicular to the long axis diameter are measured, and the biaxial average diameter obtained by averaging the long axis diameter and the short axis diameter is regarded as the number average particle size.

Examples of the (D1) pigment include the (D1-1) organic pigment and the (D1-2) inorganic pigment. Examples of the (D1-1) organic pigment include phthalocyanine-based pigments, anthraquinone-based pigments, quinacridone-based pigments, dioxazine-based pigments, thioindigo-based pigments, diketopyrrolopyrrole-based pigments, selenium-based pigments, indoline-based pigments, benzofuranone-based pigments. perylene-based pigments, aniline-based pigments, azo-based pigments, condensed azo-based pigments, and carbon black. Examples of the (D1-2) inorganic pigment include graphite or silver-tin alloys, or fine particles of metals such as titanium, copper, iron, manganese, cobalt, chromium, nickel, zinc, calcium, or silver, or oxides, composite oxides, sulfides, sulfates, nitrates, carbonates, nitrides, carbides, or oxynitrides of the metals.

The preferred content ratio of the (D1) pigment, (D1-1) organic pigment, and (D1-2) inorganic pigment to the total solid content of the negative photosensitive resin composition according to the present invention, excluding the solvent, is the same as the preferred content ratio of the (D) colorant.

### <(D1a) Black Pigment and (D1b) Non-Black Pigment>

In the negative photosensitive resin composition according to the present invention, the (D1) pigment described above preferably contains the (D1a) black pigment, or contains the (D1a) black pigment and the (D1b) non-black pigment.

The (D1a) black pigment refers to a pigment which is colored in black by absorbing light with a wavelength of visible light. Containing the (D1a) black pigment makes the resin composition film blackened, and provides excellent hiding power, thus allowing the light-blocking property of the resin composition film to be improved.

As the negative photosensitive resin composition according to the present invention, the above-described (Da) black colorant is preferably the (D1a) black pigment, and the (D1a) black pigment is preferably one or more selected from a (D1a-1) black organic pigment, a (D1a-2) black inorganic pigment, and a (D1a-3) coloring pigment mixture of two or more colors which will be described later.

The (D1b) non-black pigment refers to a pigment which is colored in purple, blue, green, yellow, orange, or red, excluding black, by absorbing light with a wavelength of visible light. Containing the (Dib) non-black pigment allows the resin composition film to be colored, and thereby allowing colorability or a toning property to be imparted. Two or more (D1b) non-black pigments are combined, thereby allowing the resin composition film to be toned to desired color coordinates, and then allows the toning property to be improved. Examples of the (D1b) non-black pigment include pigments that are colored in red, orange, yellow, green, blue, or purple, excluding black, which will be described later.

As the negative photosensitive resin composition according to the present invention, the (D1b) non-black pigment described above is preferably a (D1b-1) non-black organic pigment and/or a (D1b-2) non-black inorganic pigment, which will be described later.

### <(D1a-1) Black Organic Pigment, (D1a-2) Black Inorganic Pigment, and (D1a-3) Pigment Mixture of Two or More Colors>

As the negative photosensitive resin composition according to the present invention, the above-described (D1a) black pigment is preferably one or more selected from the (D1a-1) black organic pigment, the (D1a-2) black inorganic pigment, and the (D1a-3) coloring pigment mixture of two or more colors.

The (D1a-1) black organic pigment refers to an organic pigment which is colored in black by absorbing light with a wavelength of visible light. Containing the (D1a-1) black organic pigment makes the resin composition film blackened, and provides excellent hiding power, thus allowing the light-blocking property of the resin composition film to be improved. Furthermore, since the pigment is an organic substance, the chemical structure change or functionality transformation adjusts the transmission spectrum or absorption spectrum of the resin composition film, such as transmitting or blocking light with a desired specific wavelength, thereby making it possible to improve the toning property. In addition, since the (D1a-1) black organic pigment is superior in insulation properties and low dielectric properties, as compared with common inorganic pigments, containing the (D1a-1) black organic pigment is capable of improving the resistance value of the film. In particular, in the case of use as an insulation layer such as a pixel defining layer of an organic EL display, a TFT planarization layer, or a TFT protective layer, defective light emissions can be suppressed, thereby improving reliability.

Examples of the (D1a-1) black organic pigment include anthraquinone-based black pigments, benzofuranone-based black pigments, perylene-based black pigments, aniline-based black pigments, azo-based black pigments, azomethine-based black pigments, and carbon black. Examples of the carbon black include channel black, furnace black, thermal black, acetylene black, and lamp black. From the viewpoint of light-blocking properties, channel black is preferred.

The (D1a-2) black inorganic pigment refers to an inorganic pigment which is colored in black by absorbing light with a wavelength of visible light. Containing the (D1a-2) black inorganic pigment makes the resin composition film blackened, and provides excellent hiding power, thus allowing the light-blocking property of the resin composition film to be improved. Furthermore, since the pigment, which is an inorganic substance, is superior in heat resistance and weather resistance, the heat resistance and weather resistance of the resin composition film can be improved.

Examples of the (D1a-2) black inorganic pigment include graphite, or fine particles, oxides, composite oxides, sulfides, sulfates, nitrates, carbonates, nitrides, carbides, or oxynitrides of metals such as titanium, copper, iron, manganese, cobalt, chromium, nickel, zinc, calcium, or silver. From the viewpoint of improving the light-blocking property, fine particles, oxides, composite oxides, sulfides, nitrides, carbides, or oxynitrides of titanium or silver are preferred, and nitrides or oxynitrides of titanium are more preferred as the (D1a-2) black inorganic pigment.

The (D1a-3) pigment mixture of two or more colors refers to a pigment mixture which is colored in pseudo black by combining two or more pigments selected from red, orange, yellow, green, blue, or purple pigments. Containing the (D1a-3) pigment mixture of two or more colors makes the resin composition film blackened, and provides excellent hiding power, thus allowing the light-blocking property of the resin composition film to be improved. Furthermore, since the two or more pigments are mixed, the adjustment of the transmission spectrum or absorption spectrum of the resin composition film, such as transmitting or blocking light with a desired specific wavelength, makes it possible to improve the toning property.

As the black organic pigment, the black inorganic pigment, the red pigment, the orange pigment, the yellow pigment, the green pigment, the blue pigment, and the purple pigment, known pigments can be used.

### <(D1b-1) Non-Black Organic Pigment, (D1b-2) Non-Black Inorganic Pigment>

As the negative photosensitive resin composition according to the present invention, the non-black pigment (D1b) described above is preferably the (D1b-1) non-black organic pigment and/or the (D1b-2) non-black inorganic pigment.

The (D1b-1) non-black organic pigment refers to an organic pigment which is colored in red, orange, yellow, green, blue, or purple, excluding black, by absorbing light with a wavelength of visible light. Furthermore, since the (D1b-1) non-black organic pigment is an organic substance, the chemical structure change or functionality transformation adjusts the transmission spectrum or absorption spectrum of the resin composition film, such as transmitting or blocking light with a desired specific wavelength, thereby making it possible to improve the toning property.

The (D1b-2) non-black inorganic pigment refers to an inorganic pigment which is colored in red, orange, yellow, green, blue, or purple, excluding black, by absorbing light with a wavelength of visible light. Furthermore, since the (D1b-2) non-black organic pigment, which is an inorganic substance, is superior in heat resistance and weather resistance, the heat resistance and weather resistance of the resin composition film can be improved.

As the negative photosensitive resin composition according to the present invention, the (D1b-1) non-black organic pigment is preferably one or more selected from the group consisting of a blue pigment, a red pigment, a yellow pigment, a purple pigment, an orange pigment, and a green pigment. The (D1a-3) coloring pigment mixture of two or more colors as the (D1a) black pigment described above is, however, excluded. When the (D1b-1) non-black organic pigment is one or more selected from the group consisting of a blue pigment, a red pigment, a yellow pigment, a purple pigment, an orange pigment, and a green pigment, the transmittance of wavelengths in the ultraviolet area can be increased while maintaining the light-blocking property of the resin composition film, thus allowing the sensitivity for exposure to be improved, and allowing a pattern in a low-taper shape to be formed after development. In addition, halftone characteristics can be improved.

Examples of a pigment that is colored in blue include, pigment blue 15, 15:3, 15:4, 15:6, 22, 60, or 64 (the numerical values all refer to C.I. numbers). Examples of a pigment that is colored in red include pigment red 9, 48, 97, 122, 123, 144, 149, 166, 168, 177, 179, 180, 190, 192, 209, 215, 216, 217, 220, 223, 224, 226, 227, 228, 240, or 250 (the numerical values all refer to C.I. numbers). Examples of a pigment that is colored in yellow include pigment yellow 12, 13, 17, 20, 24, 83, 86, 93, 95, 109, 110, 117, 120, 125, 129, 137, 138, 139, 147, 148, 150, 151, 153, 154, 166, 168, 175, 180, 181, 185, 192, or 194 (the numerical values all refer to C.I. numbers). Examples of a pigment that is colored in violet include pigment violet 19, 23, 29, 30, 32, 37, 40, or 50 (the numerical values all refer to C.I. numbers). Examples of a pigment that is colored in orange include pigment orange 12, 36, 38, 43, 51, 55, 59, 61, 64, 65, 71, or 72 (the numerical values all refer to C.I. numbers). Examples of a pigment that is colored in green include pigment green 7, 10, 36, or 58 (the numerical values all refer to C.I, numbers).

From the viewpoint of improving the sensitivity for exposure, reducing the taper by pattern shape control after development, and improving halftone characteristics, as the (D1b-1) non-black organic pigment, the above-described blue pigment is preferably one or more selected from the group consisting of the C.I. pigment blue 15:4, the C.I. pigment blue 15:6, and the C.I. pigment blue 60, the above-described red pigment is preferably one or more selected from the group consisting of the C.I. pigment red 123, the C.I. pigment red 149, the C.I. pigment red 177, the C.I. pigment red 179, and the C.I. pigment red 190, the above-described yellow pigment is preferably one or more selected from the group consisting of the C.I. pigment yellow 120, the C.I. pigment yellow 151, the C.I. pigment yellow 175, the C.I. pigment yellow 180, the C.I. pigment yellow 181, the C.I. pigment yellow 192, and the C.I. pigment yellow 194, the above-described purple pigment is preferably one or more selected from the group consisting of the C.I. pigment violet 19, the C.I. pigment violet 29, and the C.I. pigment violet 37, and the above-described orange pigment is preferably one or more selected from the group consisting of the C.I. pigment orange 43, the C.I. pigment orange 64, and the C.I. pigment orange 72.

Furthermore, when the (D1b-1) non-black organic pigment is composed as described above, the pigment has excellent heat resistance, and the halogen content derived from the pigment in the resin composition can be reduced, thereby providing excellent insulation properties and low dielectric properties. Thus, in particular, in the case of use as an insulation layer such as a pixel defining layer of an organic EL display, a TFT planarization layer, or a TFT protective layer, defective light emissions can be suppressed, thereby improving reliability.

The content ratio of the (D1b-1) non-black organic pigment to the total solid content of the negative photosensitive resin composition according to the present invention, excluding the solvent, is preferably 1. by mass or higher, more preferably 3% by mass or higher, still more preferably 5% by mass or higher, particularly preferably 7% by mass or higher. When the content ratio is 1% by mass or higher, the sensitivity for exposure can be improved, and a pattern in a low-taper shape can be formed after development. In addition, halftone characteristics can be improved. On the other hand, the content ratio of the (D1b-1) non-black organic pigment is preferably 25% by mass or lower, more preferably 22% by mass or lower, still more preferably 20. by mass or lower, even more preferably 17% by mass or lower, particularly preferably 15% by mass or lower. When the content ratio is 25% by mass or lower, the light-blocking property and the toning property can be improved.

### <(D1a-1a) Benzofuranone-Based Black Pigment, (D1a-1b) Perylene-Based Black Pigment, and (D1a-1c) Azo-Based Black Pigment>

As the negative photosensitive resin composition according to the present invention, from the viewpoints of improving the sensitivity for exposure, reducing the taper by pattern shape control after development, suppressing the change in pattern opening width between before and after thermal curing, and improving the halftone characteristics, the (D1a-1) black organic pigment described above is preferably one or more selected from the group consisting of a (D1a-1a) benzofuranone-based black pigment, a (D1a-1b) perylene-based black pigment, and an (D1a-1c) azo-based black pigment, more preferably a (D1a-1a) benzofuranone-based black pigment.

Containing one or more selected from the group consisting of the (D1a-1a) benzofuranone-based black pigment, the (D1a-1b) perylene-based black pigment, and the (D1a-1c) azo-based black pigment makes the resin composition film blackened, and provides excellent hiding power, thus allowing the light-blocking property of the resin composition film to be improved. In particular, as compared with common organic pigments, the light-blocking property per unit content ratio of the pigment in the resin composition is excellent, thus allowing the same light-blocking property to be imparted with a low content ratio. Accordingly, the light-blocking property of the film can be improved, and the sensitivity for exposure can be improved. Furthermore, since the pigment is an organic substance, the chemical structure change or functionality transformation adjusts the transmission spectrum or absorption spectrum of the resin composition film, such as transmitting or blocking light with a desired specific wavelength, thereby making it possible to improve the toning property. In particular, since the transmittance of wavelengths in the near-infrared area (for example, 700 nm or more) can be improved, the composition has a light-blocking property, and the composition is suitable for applications which use light with wavelengths in the near-infrared area. Moreover, as compared with common organic pigments and inorganic pigments, the pigment is excellent in insulation properties and low dielectric properties, the resistance value of a film can be improved. In particular, in the case of use as an insulation layer such as a pixel defining layer of an organic EL display, a TFT planarization layer, or a TFT protective layer, defective light emissions can be suppressed, thereby improving reliability.

In addition, the (D1a-1a) benzofuranone-based black pigment absorbs light with a wavelength of visible light, and at the same time, has a high transmittance for wavelengths in the ultraviolet area (for example, 400 nm or less), and thus, containing the (D1a-1a) benzofuranone-based black pigment allows the sensitivity for exposure to be improved, and allows a pattern in a low-taper shape to be formed after development.

On the other hand, in the case of containing a (D1a-1a) benzofuranone-based black pigment, a development residue derived from the pigment described above may be generated due to the insufficient alkali resistance of the pigment as described above. More specifically, when the surface of the (D1a-1a) benzofuranone-based black pigment described above is exposed to an alkaline developer during development, a part of the surface may be decomposed or dissolved, thereby remaining on the substrate as a development residue derived from the pigment described above. In such a case, as described above, containing the (B3) flexible chain-containing aliphatic radical polymerizable compound and the (B1) fluorene skeleton-containing radical polymerizable compound or (B2) indane skeleton-containing radical polymerizable compound makes it possible to inhibit the development residue generation derived from the pigment described above.

The (D1a-1a) benzofuranone-based black pigment refers to a compound with a benzofuran-2(3H)-one structure or a benzofuran-3(2H)-one structure in the molecule, which is colored in black by absorbing light with a wavelength of visible light. The (D1a-1a) benzofuranone-based black pigment is preferably a benzofuranone compound represented by any of the general formulas (63) to (68), a geometric isomer thereof, a salt thereof, or a salt of the geometric isomer.

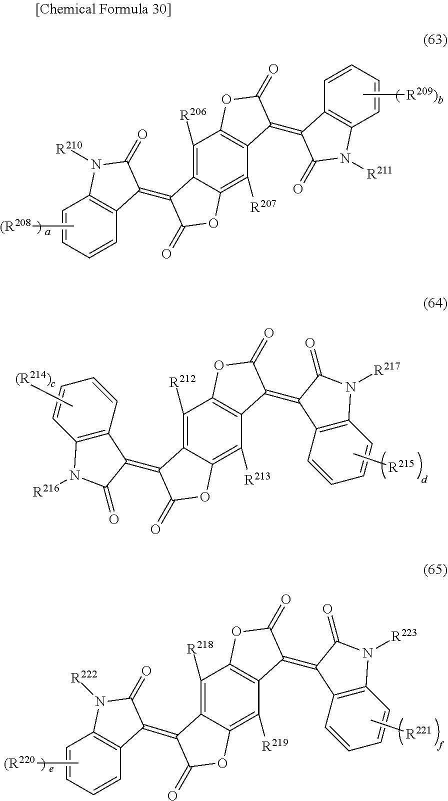

In general formulas (63) to (65), R²⁰⁶, R²⁰⁷, R²¹², R²¹³, R²¹⁸, and R²¹⁹ each independently represent hydrogen, a halogen atom, an alkyl group having 1 to 10 carbon atoms, or an alkyl group having 1 to 10 carbon atoms with 1 to 20 fluorine atoms. R²⁰⁸, R²⁰⁹, R²¹⁴, R²¹⁵, R²²⁰, and R²²¹ each independently represent hydrogen, a halogen atom, R²⁵, COOH, COOR²⁵¹, COO⁻, CONH₂, CONHR²⁵¹, CONR²⁵¹R²⁵², CN, OH, OR²⁵¹, OCOR²⁵¹, OCONH₂, OCONHR²⁵¹, OCONR²⁵¹R²⁵², NO₂, NH₂, NHR²⁵¹, NR²⁵¹R²⁵², NHCOR²⁵¹, NR²⁵¹COR²⁵², N═CH₂, N═CHR²⁵¹, N═CR²⁵¹R²⁵², SH, SR²⁵¹, SOR²⁵¹, SO₂R²⁵¹, SO₃R²⁵¹, SO₃H, SO₃⁻, SO₂NH₂, SO₂NHR²⁵¹, or SO₂NR²⁵¹R²⁵², and R²⁵¹ and R²⁵² each independently represent an alkyl group having 1 to 10 carbon atoms, a cycloalkyl group having 4 to 10 carbon atoms, an alkenyl group having 2 to 10 carbon atoms, a cycloalkenyl group having 4 to 10 carbon atoms, or an alkynyl group of 2 to 10 carbon atoms. More than one R²⁰⁸, R²⁰⁹, R²¹⁴, R²¹⁵, R²²⁰, or R²²¹ may form a ring with a direct bond, or an oxygen atom bridge, a sulfur atom bridge, an NH bridge, or an NR²⁵¹ bridge. R²¹⁰, R²¹¹, R²¹⁶, R²¹⁷, R²²², and R²²³ each independently represent hydrogen, an alkyl group having 1 to 10 carbon atoms, or an aryl group having 6 to 15 carbon atoms. a, b, c, d, e, and f each independently represent an integer of 0 to 4. The alkyl group, cycloalkyl group, alkenyl group, cycloalkenyl group, alkynyl group, and aryl group described above may have a hetero atom, and may be either unsubstituted or substituted.

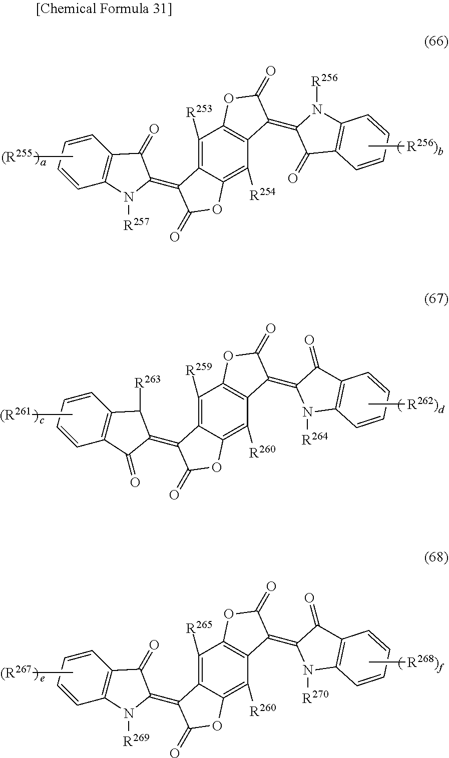

In general formulas (66) to (68), R²⁵³, R²⁵⁴, R²⁵⁹, R²⁶⁰, R²⁶⁵, and R²⁶⁶ each independently represent hydrogen, a halogen atom, an alkyl group having 1 to 10 carbon atoms, or an alkyl group having 1 to 10 carbon atoms with 1 to 20 fluorine atoms. R²⁵⁵, R²⁵⁶, R²⁶¹, R²⁶², R²⁶⁷, and R²⁶⁸ each independently represent hydrogen, a halogen atom, R²⁷¹, COOH, COOR²⁷¹, COO⁻, CONH₂, CONHR²⁷¹, CONR²⁷¹R²⁷¹, CN, OH, OR²⁷¹, OCOR²⁷¹, OCONH₂, OCONHR²⁷¹, OCONR²⁷¹R²⁷², NO₂, NH₂, NHR²⁷¹, NR²⁷¹R²⁷¹, NHCOR²⁷¹, NR²⁷¹COR²⁷², N═CH₂, N═CHR²⁷¹, N═CR²⁷¹R²⁷¹, SH, SR²⁷¹, SOR²⁷¹, SO₂R²⁷¹, SO₃R²⁷¹, SO₃H, SO₃⁻, SO₂NH₂, SO₂NHR²⁷¹, or SO₂NR²⁷¹R²⁷², and R²⁷¹ and R²⁷² each independently represent an alkyl group having 1 to 10 carbon atoms, a cycloalkyl group having 4 to 10 carbon atoms, an alkenyl group having 2 to 10 carbon atoms, a cycloalkenyl group having 4 to 10 carbon atoms, or an alkynyl group of 2 to 10 carbon atoms. More than one R²⁵⁵, R²⁵⁶, R²⁶¹, R²⁶², R²⁶⁷, or R²⁶⁸ may form a ring with a direct bond, or an oxygen atom bridge, a sulfur atom bridge, an NH bridge, or an NR²⁷¹ bridge. R²⁵⁷, R²⁵⁸, R²⁶³, R²⁶⁴, R²⁶⁹, and R²⁷¹ each independently represent hydrogen, an alkyl group having 1 to 10 carbon atoms, or an aryl group having 6 to 15 carbon atoms. a, b, c, d, e, and f each independently represent an integer of 0 to 4. The alkyl group, cycloalkyl group, alkenyl group, cycloalkenyl group, alkynyl group, and aryl group described above may have a hetero atom, and may be either unsubstituted or substituted.

Examples of the (D1a-1a) benzofuranone-based black pigment include “IRGAPHOR” (registered trademark) BLACK S0100CF (manufactured by BASF), the black pigment described in International Publication No. 2010/081624, or the black pigment described in International Publication No. 2010/081756.

The (D1a-1b) perylene-based black pigment refers to a compound with a perylene structure in the molecule, which is colored in black by absorbing light with a wavelength of visible light. The (D1a-1b) perylene-based black pigment is preferably a perylene compound represented by any of the general formulas (69) to (71), a geometric isomer thereof, a salt thereof, or a salt of the geometric isomer.

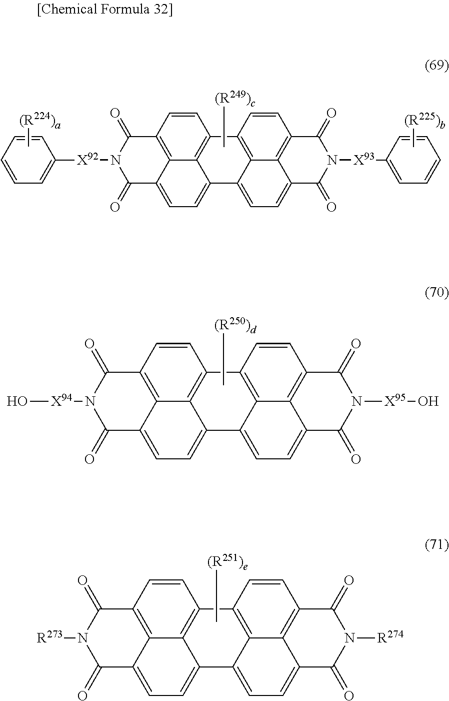

In the general formulas (69) to (71), X⁹², X⁹³, X⁹⁴, and X⁹⁵ each independently represent an alkylene chain having 1 to 10 carbon atoms. R²²⁴ and R²²⁵ each independently represent hydrogen, a hydroxy group, an alkoxy group having 1 to 6 carbon atoms, or an acyl group having 2 to 6 carbon atoms. R²⁴⁹, R²⁵⁰, and R²⁵¹ each independently represent hydrogen, a halogen atom, an alkyl group having 1 to 10 carbon atoms, or an alkyl group having 1 to 10 carbon atoms with 1 to 20 fluorine atoms. R²⁷³ and R²⁷⁴ each independently represent hydrogen or an alkyl group having 1 to 10 carbon atoms. a and b each independently represent an integer of 0 to 5. The alkylene chain, alkoxy group, acyl group, and alkyl group described above may have a hetero atom, and may be either unsubstituted or substituted.

Examples of the (D1a-1b) perylene-based black pigment include pigment black 31 or 32 (the numerical values are both C.I. numbers). The examples include, besides the pigments described above, “PALIOGEN” (registered trademark) BLACK S0084, K0084, L0086, K0086, EH0788, or FK4281 (all manufactured by BASF).

The (D1a-1c) azo-based black pigment refers to a compound with an azo group in the molecule, which is colored in black by absorbing light with a wavelength of visible light. The (D1a-1c) azo-based black pigment is preferably an azo compound represented by general formula (72).

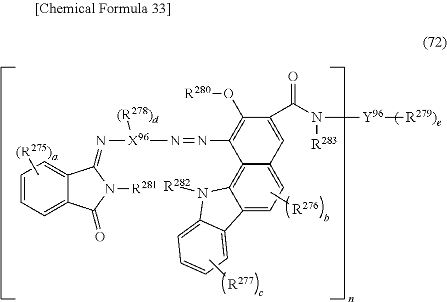

In the general formula (72), X⁹⁶ represents an arylene chain having 6 to 15 carbon atoms. Y⁹⁶ represents an arylene chain having 6 to 15 carbon atoms. R²⁷⁵, R²⁷⁶, and R²⁷⁷ each independently represent a halogen or an alkyl group having 1 to 10 carbon atoms. R²⁷⁸ represents halogen, an alkyl group having 1 to 10 carbon atoms, an alkoxy group having 1 to 6 carbon atoms, or a nitro group. R²⁷⁹ represents halogen, an alkyl group having 1 to 10 carbon atoms, an alkoxy group having 1 to 6 carbon atoms, an acylamino group having 2 to 10 carbon atoms, or a nitro group. R²⁸⁰, R²⁸¹, R²⁸², and R²⁸³ each independently represent hydrogen or an alkyl group having 1 to 10 carbon atoms. a represents an integer of 0 to 4, b represents an integer of 0 to 2, c represents an integer of 0 to 4, d and e each independently represent an integer of 0 to 8, and n represents an integer of 1 to 4. The above-mentioned arylene chain, alkyl group, alkoxy group, and acylamino group may have a hetero atom, and may be either unsubstituted or substituted.

Examples of the (D1a-1c) azo-based black pigment include “CHROMOFINE” (registered trademark) BLACK A1103 (manufactured by Dainichiseika Color & Chemicals Mfg. Co., Ltd.), the black pigment described in JP 01-170601 A, or the black pigment described in JP 02-034664 A.

The content ratio of one or more selected from the group consisting of the (D1a-1a) benzofuranone-based black pigment, the (D1a-1b) perylene-based black pigment, and the (D1a-1c) azo-based black pigment in the total solid content of the negative photosensitive resin composition according to the present invention, excluding the solvent, is preferably 5% by mass or higher, more preferably 10% by mass or higher, still more preferably 15% by mass or higher, particularly preferably 20% by mass or higher. When the content ratio is 5% by mass or higher, the light-blocking property and the toning property can be improved. On the other hand, content ratio of one or more selected from the group consisting of the (D1a-1a) benzofuranone-based black pigment, the (D1a-1b) perylene-based black pigment, and the (D1a-1c) azo-based black pigment is preferably 70% by mass or lower, more preferably 65% by mass or lower, still more preferably 60% by mass or lower, even more preferably 55% by mass or lower, particularly preferably 50% by mass or lower. When the content ratio is 70. by mass or lower, the sensitivity for exposure can be improved.

<(D1a-3a) Coloring Pigment Mixture Including Blue Pigment, Red Pigment, and Yellow Pigment, (D1a-3b) Coloring Pigment Mixture Including Purple Pigment and Yellow Pigment, (D1a-3c) Coloring Pigment Mixture Including Blue Pigment, Red Pigment, and Orange Pigment, and (D1a-3d) Coloring Pigment Mixture Including Blue Pigment, Purple Pigment, and Orange Pigment>

As the negative photosensitive resin composition according to the present invention, the above-mentioned (D1a-3) pigment mixture of two or more colors is preferably a (D1a-3a) coloring pigment mixture including a blue pigment, a red pigment, and a yellow pigment, a (D1a-3b) coloring pigment mixture including a purple pigment and a yellow pigment, a (D1a-3c) coloring pigment mixture including a blue pigment, a red pigment, and an orange pigment, or a (D1a-3d) coloring pigment mixture including a blue pigment, a purple pigment, and an orange pigment.

When the (D1a-3) pigment mixture of two or more colors is composed as described above, the resin composition film is blackened, and excellent hiding power is provided. Thus, the light-blocking property of the resin composition film can be improved. In addition, the transmittance of wavelengths in the ultraviolet area can be increased, thus allowing the sensitivity for exposure to be improved, and allowing a pattern in a low-taper shape to be formed after development. In addition, the change in pattern opening width between before and after thermal curing can be suppressed, and the halftone characteristics can be improved. In addition, since the transmittance of wavelengths in the near-infrared area can be improved, the composition has a light-blocking property, and the composition is suitable for applications which use light with wavelengths in the near-infrared area.

Examples of a pigment that is colored in blue include, pigment blue 15, 15:3, 15:4, 15:6, 22, 60, or 64 (the numerical values all refer to C.I. numbers). Examples of a pigment that is colored in red include pigment red 9, 48, 97, 122, 123, 144, 149, 166, 168, 177, 179, 180, 190, 192, 209, 215, 216, 217, 220, 223, 224, 226, 227, 228, 240, or 250 (the numerical values all refer to C.I. numbers). Examples of a pigment that is colored in yellow include pigment yellow 12, 13, 17, 20, 24, 83, 86, 93, 95, 109, 110, 117, 120, 125, 129, 137, 138, 139, 147, 148, 150, 151, 153, 154, 166, 168, 175, 180, 181, 185, 192, or 194 (the numerical values all refer to C.I. numbers). Examples of a pigment that is colored in violet include pigment violet 19, 23, 29, 30, 32, 37, 40, or 50 (the numerical values all refer to C.I. numbers). Examples of a pigment that is colored in orange include pigment orange 12, 36, 38, 43, 51, 55, 59, 61, 64, 65, 71, or 72 (the numerical values all refer to C.I. numbers). Examples of a pigment that is colored in green include pigment green 7, 10, 36, or 58 (the numerical values all refer to C.I, numbers).

As the negative photosensitive resin composition according to the present invention, in the above-described (D1a-3) pigment mixture of two or more colors, the above-described blue pigment is preferably one or more selected from the group consisting of the C.I. pigment blue 15:4, the C.I. pigment blue 15:6, and the C.I. pigment blue 60, the above-described red pigment is preferably one or more selected from the group consisting of the C.I. pigment red 123, the C.I. pigment red 149, the C.I. pigment red 177, the C.I. pigment red 179, and the C.I. pigment red 190, the above-described yellow pigment is preferably one or more selected from the group consisting of the C.I. pigment yellow 120, the C.I. pigment yellow 151, the C.I. pigment yellow 175, the C.I. pigment yellow 180, the C.I. pigment yellow 181, the C.I. pigment yellow 192, and the C.I. pigment yellow 194, the above-described purple pigment is preferably one or more selected from the group consisting of the C.I. pigment violet 19, the C.I. pigment violet 29, and the C.I. pigment violet 37, and the above-described orange pigment is preferably one or more selected from the group consisting of the C.I. pigment orange 43, the C.I. pigment orange 64, and the C.I. pigment orange 72.

When the (D1a-3) pigment mixture of two or more colors is composed as described above, the pigment has excellent alkali resistance, thus suppressing the decomposition or dissolution of the pigment surface during alkali development, and allowing the development residue generation due to the pigment to be inhibited. Furthermore, the pigment has excellent heat resistance, and the halogen content derived from the pigment in the resin composition can be reduced, thereby providing excellent insulation properties and low dielectric properties. Thus, the resistance value of the film can be improved. In particular, in the case of use as an insulation layer such as a pixel defining layer of an organic EL display, a TFT planarization layer, or a TFT protective layer, defective light emissions can be suppressed, thereby improving reliability.

The content ratio of the (D1a-3) pigment mixture of two or more colors to the total solid content of the negative photosensitive resin composition according to the present invention, excluding the solvent, is preferably 5% by mass or higher, more preferably 10% by mass or higher, still more preferably 15% by mass or higher, particularly preferably 20% by mass or higher. When the content ratio is 5% by mass or higher, the light-blocking property and the toning property can be improved. On the other hand, the content ratio of the (D1a-3) pigment mixture of two or more colors is preferably 70% by mass or lower, more preferably 65. by mass or lower, still more preferably 60. by mass or lower, even more preferably 55% by mass or lower, particularly preferably 50% by mass or lower. When the content ratio is 70% by mass or lower, the sensitivity for exposure can be improved.

### <(DC) Covering Layer>

As the negative photosensitive resin composition according to the present invention, the (D1a-1) black organic pigment preferably further contains a (DC) covering layer. The (DC) covering layer refers to a layer covering a pigment surface, which is formed by a treatment such as a surface treatment with a silane coupling agent, a surface treatment with a silicate, a surface treatment with a metal alkoxide, or a covering treatment with a resin, for example.

Containing the (DC) covering layer makes it possible to modify the surface condition of the particles, such as acidifying or basifying the particle surfaces of the (D1a-1) black organic pigment or making the particle surfaces hydrophilic or hydrophobic, and then makes it possible to improve the acid resistance, the alkali resistance, the solvent resistance, the dispersion stability, the heat resistance, and the like. Thus, the development residue generation derived from a pigment can be inhibited. In addition, side etching during development is suppressed, a pattern in a low-taper shape can be formed after development, and reflow of the pattern skirt during thermal curing is suppressed, and thus, the change in pattern opening width between before and after thermal curing can be suppressed. In addition, since it is possible to form a pattern in a low-taper shape by controlling the pattern shape after development, the halftone characteristics can be improved. In addition, an insulating covering layer is formed on the particle surfaces to improve the insulation properties of the cured film for reduction in leakage current and the like, thereby allowing display reliability and the like to be improved.

In the case of containing, in particular, the (D1a-1a) benzofuranone-based black pigment as the (D1a-1) black organic pigment, the (D1a-1a) benzofuranone-based black pigment contains therein the (DC) covering layer, thereby allowing the alkali resistance of the pigment to be improved, and then allowing the development residue generation derived from the pigment to be inhibited.

The average coverage of the (DC) covering layer with respect to the (D1a-1) black organic pigment is preferably 50% or higher, preferably 70% or higher, more preferably 80% or higher, still more preferably 90% or higher. When the average coverage of the (DC) covering layer is 80% or higher, the residue generation during development can be inhibited.

For the average coverage of the (DC) covering layer with respect to the (D1a-1) black organic pigment, a cross section is observed at a magnification of 50,000 to 200,000 times under the condition of an acceleration voltage of 300 kV with the use of a transmission electron microscope (H9500; manufactured by Hitachi High-Technologies Corporation), and for 100 black pigment particles randomly selected, the average coverage N (%) can be determined by determining the coverage M (%) for each black pigment from the following formula, and calculating the number average value.

Coverage M (%)={L1/(L1+L2)}×100

L1: the total length (nm) of a site of the outer periphery of a particle, covered with the covering layer

L2: the total length (nm) of a site of the outer periphery of the particle, covered with no covering layer (the site with the interface and the embedded resin in direct contact)

L1+L2: the outer peripheral length (nm) of a particle

### <(DC-1) Silica Covering Layer, (DC-2) Metal Oxide Covering Layer, and (DC-3) Metal Hydroxide Covering Layer>

The (DC) covering layer preferably contains one selected from the group consisting of a (DC-1) silica covering layer, a (DC-2) metal oxide covering layer, and a (DC-3) metal hydroxide covering layer. The silica, the metal oxide, and the metal hydroxide have the function of imparting alkali resistance to the pigment, thus the development residue generation derived from the pigment to be inhibited.

The silica included in the (DC-1) silica covering layer refers to a general term for silicon dioxide and hydrates thereof. The metal oxide included in the (DC-2) metal oxide covering layer refers to a general term for metal oxides and hydrates thereof. Examples of the metal oxide include alumina as an example, and include alumina (Al₂O₃) or an alumina hydrate (Al₂O₃.nH₂O), for example. Examples of the metal hydroxide contained in the (DC-3) metal hydroxide covering layer include an aluminum hydroxide (Al(OH)₃). Because silica has a low dielectric constant, the dielectric constant of the pixel defining layer can be kept from being increased, even in a case where the content of the (DC) covering layer of the (D1a-1) black organic pigment is high.

The (DC-1) silica covering layer, (DC-2) metal oxide covering layer, and (DC-3) metal hydroxide covering layer of the (DC) covering layer can be analyzed by, for example, an X-ray diffraction method. Examples of the X-ray diffractometer include a powder X-ray diffractometer (manufactured by Mac Science). The mass of the silicon atoms or metal atoms contained in the (DC-1) silica covering layer, (DC-2) metal oxide covering layer, and (DC-3) metal hydroxide covering layer is rounded to one decimal place to calculate the value down to the first decimal place. In addition, the mass of the pigment particles, excluding the (DC) covering layer, contained in the (D1a-1) black organic pigment including the (DC) covering layer can be determined by the following method, for example. After the operation of putting the pigment with the mass measured in a mortar, grinding the pigment with a pestle for the removal of the (DC) covering layer, then dissolving only the pigment particles by immersion in an amide-based solvent such as N,N-dimethylformamide, and removing the particles as filtrate, is repeated until the filter cake completely loses the blackness, the mass of the filter cake is measured, and the mass of the pigment particles is calculated from the difference from the pigment mass.

The metal oxide or metal hydroxide contained in the (DC-2) metal oxide covering layer or (DC-3) metal hydroxide covering layer preferably has both chemical durability such as alkali resistance, heat resistance and light resistance, and physical durability such as Vickers hardness that can withstand mechanical energy input appropriately optimized in the dispersion step, and wear resistance. Examples of the metal oxide and metal hydroxide include alumina, zirconia, zinc oxides, titanium oxides, and ferric oxides. Alumina or zirconia is preferred from the viewpoint of insulation properties, and ultraviolet transmittance and near-infrared transmittance, and alumina is more preferred from the viewpoint of dispersibility in alkali-soluble resins and solvents. The metal oxide and the metal hydroxide may be surface-modified with a group including an organic group.

In a case where the (DC) covering layer contains the (DC-1) silica covering layer, an alumina covering layer is formed as the (DC-2) metal oxide covering layer on the surface of the (DC-1) silica covering layer, thereby a decrease in pattern linearity to be suppressed. Since alumina is effective for dispersibility improvement in an aqueous pigment suspension even in the pigment granulation step performed after the pigment surface treatment step, the secondary aggregation particle diameter can be adjusted to a desired range, and furthermore, the productivity and quality stability can be improved. As the (DC-2) metal oxide covering layer contained in the (DC) covering layer, the covering amount of the alumina covering layer is preferably 10 parts by mess or more, more preferably 20 parts by weight or more, in a case where the silica contained in the (DC-1) silica covering layer is regarded as 100 parts by mass.

In the case of the (DC) covering layer containing the (DC-1) silica covering layer, the silica content is preferably 1 part by mass or more, more preferably 2 parts by mass or more, still more preferably 5 parts by mass or more, in a case where the pigment particles are regarded as 100 parts by mass. The content is adjusted to 1 part by mass or more, thereby making it possible to increase the coverage on the pigment particle surface and inhibit the development residue generation derived from the pigment. On the other hand, the content of the silica is preferably 20 parts by mass or less, more preferably 10 parts by mass or less. The content is adjusted to 20 parts by mass or less, thereby allowing the pattern linearity of the pixel defining layer to be improved.

In the case of the (DC) covering layer containing the (DC-2) metal oxide covering layer and/or the (DC-3) metal hydroxide covering layer, the total content of metal oxide and metal hydroxide is preferably 0.1 parts by mass or more, more preferably 0.5 parts by mass or more, in a case where the pigment particles are regarded as 100 parts by mass. The total content is adjusted to 0.1 parts by mass or more, the dispersibility and the pattern linearity can be improved. On the other hand, the total content of the metal oxide and metal hydroxide is preferably 15 parts by mass or less, more preferably 10 parts by mass or less. The total content is adjusted to 15 parts by mass or less, thereby making it possible to keep the concentration gradient of the pigment from being generated, and improve the storage stability of the coating liquid, in the photosensitive composition according to the present invention, which is designed to make the viscosity lower, preferably, provide a viscosity of 15 mPa s or lower.

It is to be noted that the content of the silica refers to the silicon dioxide equivalent value calculated from the content of silicon atoms, which refers to a SiO₂ equivalent value, including cases where there is not only a single component in the (DC) covering layer and at the surface layer, and cases where the amount of dehydration varies due to thermal history. The contents of the metal oxide and metal hydroxide refer to the metal oxide and metal hydroxide equivalent values calculated from the metal atom content. More specifically, in the case of alumina, zirconia, and titanium oxide, the contents respectively refer to an Al₂O₃ equivalent value, a ZrO₂ equivalent value, and a TiO₂ equivalent value. In addition, the total content of the metal oxide and metal hydroxide refers to the content in the case of containing either the metal oxide or the metal hydroxide, or refers to the total content in the case of containing the both.

The (DC) covering layer may be surface-modified with an organic group by using a silane coupling agent, with, as a reactive site, hydroxy at the surface of the silica, metal oxide, or metal hydroxide contained in the (DC-1) silica covering layer, (DC-2) metal oxide covering layer, or (DC-3) metal hydroxide covering layer. As the organic group, an ethylenically unsaturated double bond group is preferred. The surface modification with a silane coupling agent having an ethylenically unsaturated double bond group is capable of imparting radical polymerizability to the (D1a-1) black organic pigment, and suppressing film peeling at the cured part, thereby inhibiting the development residue generation derived from the pigment at the unexposed part.

As the (D1a-1) black organic pigment including the (DC) covering layer, the outermost layer may be further subjected to a surface treatment with an organic surface treatment agent. The outermost layer is subjected to the surface treatment, thereby allowing the wettability to the resin or the solvent to be improved. The (DC) covering layer may further contain a resin covering layer formed by a covering treatment with a resin. Containing the resin covering layer provides particle surfaces covered with an insulating resin with low conductivity, thereby allowing the particle surface condition to be modified, and allowing the light-blocking and insulating properties of the cured film to be improved.

### <(D2) Dye>

In the negative photosensitive resin composition according to the present invention, the above-mentioned (D) colorant preferably contains a (D2) pigment. As an aspect in which the (D) colorant described above contains the (D2) dye, it is preferable to contain the (D2) dye as the colorant other than the (Da) black colorant and/or (Db) non-black colorant described above.

The (D2) dye refers to a compound that colors an object by chemical adsorption or strong interaction of a substituent such as an ionic group or a hydroxy group in the (D2) dye on or with the surface structure of the object, and the compound is typically soluble in solvents and the like. In addition, coloring with the (D2) dye is high in coloring power and high in coloring efficiency, because each molecule is adsorbed to an object.

Containing the (D2) dye allows coloring in a color which is excellent in coloring power, and then allows the colorability and toning property of the resin composition film to be improved. Examples of the (D2) dye include direct dyes, reactive dyes, sulfur dyes, vat dyes, acid dyes, metal-containing dyes, metal-containing acid dyes, basic dyes, mordant dyes, acid mordant dyes, dispersive dyes, and cationic dyes, and fluorescent whitening dyes. In this regard, the dispersive dye refers to a dye that is insoluble or poorly soluble in water, without having an anionic ionization group such as a sulfonic acid group or a carboxy group.

Examples of the (D2) dye include anthraquinone-based dyes, azo-based dyes, azine-based dyes, phthalocyanine-based dyes, methine-based dyes, oxazine-based dyes, quinoline-based dyes, indigo-based dyes, indigoid-based dyes, carbonium-based dyes, selenium-based dyes, perinone-based dyes, perylene-based dyes, triarylmethane-based dyes, and xanthene-based dyes. Anthraquinone-based dyes, azo-based dyes, azine-based dyes, methine-based dyes, triarylmethane-based dyes, and xanthene-based dyes are preferred from the viewpoints of solubility in solvents to be described later and heat resistance.

As the negative photosensitive resin composition according to the present invention, the above-described (D2) dye preferably contains one or more selected from the (D2a-1) black dye, the (D2a-2) dye mixture of two or more colors, and the (D2b) non-black dye, which will be described later.

The content ratio of the (D2) dye to the total solid content of the negative photosensitive resin composition according to the present invention, excluding the solvent, is preferably 0.01% by mass or higher, more preferably 0.05% by mass or higher, still more preferably 0.1% by mass or higher. When the content ratio is 0.01% by mass or higher, the colorability or the toning property can be improved. On the other hand, the content ratio of the (D2) dye is preferably 50% by mass or lower, more preferably 45% by mass or lower, still more preferably 40% by mass or lower. When the content ratio is 50% by mass or lower, the heat resistance of the cured film can be improved.

### <(D2a-1) Black Dye, (D2a-2) Dye Mixture of Two or More Colors, and (D2b) Non-Black Dye>

As the negative photosensitive resin composition according to the present invention, the (D2) dye described above preferably contains one or more selected from the (D2a-1) black dye, the (D2a-2) dye mixture of two or more colors, and the (D2b) non-black dye.

The (D2a-1) black dye refers to a dye which is colored in black by absorbing light with a wavelength of visible light. Containing the (D2a-1) black dye makes the resin composition film blackened, and provides excellent colorability, thus allowing the light-blocking property of the resin composition film to be improved.

The (D2a-2) dye mixture of two or more colors refers to a dye mixture which is colored in pseudo black by combining two or more dyes selected from white, red, orange, yellow, green, blue, or purple dyes. Containing the (D2a-2) dye mixture of two or more colors makes the resin composition film blackened, and provides excellent colorability, thus allowing the light-blocking property of the resin composition film to be improved. Furthermore, since the two o more dyes are mixed, the adjustment of the transmission spectrum or absorption spectrum of the resin composition film, such as transmitting or blocking light with a desired specific wavelength, makes it possible to improve the toning property. As the black dye, the red dye, the orange dye, the yellow dye, the green dye, the blue dye, and the purple dye, known dyes can be used.

The (D2b) non-black dye refers to a dye that is colored in white, red, orange, yellow, green, blue, or purple, excluding black, by absorbing light with a wavelength of visible light. Containing the (D2b) non-black dye allows the resin composition film to be colored, and thereby allowing colorability or a toning property to be imparted. Two or more (D2b) non-black dyes are combined, thereby allowing the resin composition film to be toned to desired color coordinates, and then allows the toning property to be improved. Examples of the (D2b) non-black dye include dyes that are colored in white, red, orange, yellow, green, blue, or purple, excluding black, which are described above.

The cured film obtained by curing the negative photosensitive resin composition according to the present invention preferably has an optical density of 0.3 or more, more preferably 0.5 or more, even more preferably 0.7 or more, particularly preferably 1.0 or more, per film thickness of 1 μm in the visible wavelength region. The visible wavelength region is approximately 400 to 700 nm in wavelength. When the optical density per film thickness of 1 μm is 0.3 or more, the cured film allows the light-blocking property to be improved, thus making it possible to prevent electrode wirings form being made visible or reduce external light reflection, and then allowing the contrast in image display to be improved, in display devices such as an organic EL display or a liquid crystal display. For this reason, the composition is suitable for applications such as a pixel defining layer, an electrode insulation layer, a wiring insulation layer, an interlayer insulation layer, a TFT planarization layer, an electrode planarization layer, a wiring planarization layer, a TFT protective layer, an electrode protective layer, a wiring protective layer, a gate insulation layer, a color filter, a black matrix, or a black column spacer. The composition is preferred as in particular, a light-blocking pixel defining layer, electrode insulation layer, wiring insulation layer, interlayer insulation layer, TFT planarization layer, electrode planarization layer, wiring planarization layer, TFT protective layer, electrode protective layer, wiring protective layer, or gate insulating layer of an organic EL display protective layer, and suitable for applications which require contrast increased by suppression of external light reflection, such as a light-blocking pixel defining layer, interlayer insulation layer, TFT planarization layer, or TFT protective layer. On the other hand, the optical density per film thickness of 1 μm is preferably 5.0 or less, more preferably 4.0 or less, still more preferably 3.0 or less. When the optical density per film thickness of 1 μm is 5.0 or less, the sensitivity for exposure can be improved, and a cured film in a pattern in a low-taper shape can be obtained. The optical density of the cured film per film thickness of 1 μm can be adjusted depending on the composition and content ratio of the colorant (D) described above.

### <(E) Dispersant>

The negative photosensitive resin composition according to the present invention preferably further contains the (E) dispersant. The (E) dispersant refers to a compound having a surface affinity group that interacts with the surface of a dispersive dye or the like as the (D1) pigment and/or the (D2) dye described above, and a dispersion-stabilization structure that improves the dispersion stability of a dispersive dye as the (D1) pigment and/or the (D2) dye. Examples of the dispersion stabilization structure of the (E) dispersant include a polymer chain and/or a substituent with an electrostatic charge.

Containing the (E) dispersant allows, in a case where the negative photosensitive resin composition contains a dispersive dye as the (D1) pigment and/or the (D2) dye, the dispersion stability to be improved, and then allows the resolution after development to be improved. In particular, for example, in a case where the (D1) pigment has particles crushed to a number average particle size of 1 μm or less, the particle surface area of the (D1) pigment is increased, thus making particle aggregation of the (D1) pigment more likely to be caused. On the other hand, in the case of containing the (E) dispersant, the interaction between the surface of the crushed (D1) pigment and the surface affinity group of the (E) dispersant, and the steric hindrance and/or electrostatic repulsion due to the dispersion-stabilization structure of the (E) dispersant make it possible to inhibit the particle aggregation of the (D1) pigment, thereby improving the dispersion stability.

Examples of the (E) dispersant having a surface affinity group include a (E) dispersant having only a basic group, a (E) dispersant having a basic group and an acidic group, and a (E) dispersant having only an acidic group, and a (E) dispersant having neither a basic group nor an acidic group. From the viewpoint of improving the dispersion stability of the particles of the (D1) pigment, the (E) dispersant having only a basic group and the (E) dispersant having a basic group and an acidic group are preferred. In addition, the basic group and/or the acidic group which serve as surface affinity group(s) also preferably have a structure that forms a salt with an acid and/or base.

Examples of the basic group of the (E) dispersant or the structure thereof that forms a salt include a tertiary amino group or a quaternary ammonium salt structure, or nitrogen-containing ring skeletons such as a pyrrolidine skeleton, a pyrrole skeleton, an imidazole skeleton, a pyrazole skeleton, a triazole skeleton, a tetrazole skeleton, an imidazoline skeleton, an oxazole skeleton, an isoxazole skeleton, an oxazoline skeleton, an isoxazoline skeleton, a thiazole skeleton, an isothiazole skeleton, a thiazoline skeleton, an isothiazoline skeleton, a thiazine skeleton, a piperidine skeleton, a piperazine skeleton, a morpholine skeleton, a pyridine skeleton, a pyridazine skeleton, a pyrimidine skeleton, a pyrazine skeleton, a triazine skeleton, an isocyanuric acid skeleton, an imidazolidinone skeleton, a propylene urea skeleton, a butylene urea skeleton, a hydantoin skeleton, a barbituric acid skeleton, an alloxan skeleton, or a glycoluril skeleton.

From the viewpoint of improving the dispersion stability and the resolution after development, a tertiary amino group or a quaternary ammonium salt structure, or a nitrogen ring-containing skeleton such as or a pyrrole skeleton, an imidazole skeleton, a pyrazole skeleton, a pyridine skeleton, a pyridazine skeleton, a pyrimidine skeleton, a pyrazine skeleton, a triazine skeleton, an isocyanuric acid skeleton, an imidazolidinone skeleton, a propylene urea skeleton, a butylene urea skeleton, a hydantoin skeleton, a barbituric acid skeleton, an alloxan skeleton, or a glycoluril skeleton is preferred as the basic group or the structure thereof that forms a salt.

Examples of the (E) dispersant having only a basic group include “DISPERBYK” (registered trademark) -108, -160, -167, -182, -2000, or -2164 and “BYK” (registered trademark) -9075, -LP-N6919, or -LP-N21116 (all manufactured by BYK-Chemie Japan), “EFKA” (registered trademark) 4015, 4050, 4080, 4300, 4400, or 4800 (all manufactured by BASF), “Ajisper” (registered trademark) PB711 (manufactured by Ajinomoto Fine-Techno Co., Inc.), and “SOLSPERSE” (registered trademark) 13240, 20000 or 71000 (all manufactured by Lubrizol).

Examples of the (E) dispersant having a basic group and an acidic group include “ANTI-TERRA” (registered trademark) -U100 or -204, “DISPERBYK” (registered trademark) -106, -140, -145, -180, -191, -2001, or -2020, and “BYK” (registered trademark) -9076 (manufactured by BYK-Chemie Japan), “Ajisper” (registered trademark) PB821 or PB881 (all manufactured by Ajinomoto Fine-Techno Co., Inc.), and “SOLSPERSE” (registered trademark) 9000, 13650, 24000, 33000, 37500, 39000, 56000, or 76500 (all manufactured by Lubrizol).

Examples of the (E) dispersant having only an acidic group include “DISPERBYK” (registered trademark) -102, -118, -170 or -2096, “BYK” (registered trademark) -P104 or -220S. (all manufactured by BYK-Chemie Japan), and “SOLSPERSE” (registered trademark) 3000, 16000, 21000, 36000, or 55000 (all manufactured by Lubrizol).

Examples of the dispersant (E) having neither a basic group nor an acidic group include “DISPERBYK” (registered trademark) -103, -192, -2152, or -2200 (all manufactured by BYK-Chemie Japan), and “SOLSPERSE” (registered trademark) 27000, 54000, or X300 (all manufactured by Lubrizol).

The amine value of the (E) dispersant is preferably 5 mgKOH/g or more, more preferably 8 mgKOH/g or more, and still more preferably 10 mgKOH/g or more. When the amine value is 5 mgKOH/g or more, the dispersion stability of the (D1) pigment can be improved. On the other hand, the amine value is preferably 150 mgKOH/g or less, more preferably 120 mgKOH/g or less, still more preferably 100 mgKOH/g or less. When the amine value is 150 mgKOH/g or less, the storage stability of the resin composition can be improved.

The amine value herein refers to the weight of potassium hydroxide that is equivalent to an acid that reacts with per 1 g of the (E) dispersant, and the unit is mgKOH/g. The amine value can be determined by neutralization of 1 g of the (E) dispersant with an acid, and then titration with an aqueous potassium hydroxide solution. From the amine value, the amine equivalent (unit: g/mol), which refers to the resin weight per 1 mol of basic groups such as amino groups, can be calculated, and the number of basic groups such as amino groups in the (E) dispersant can be determined.

The acid value of the (E) dispersant is preferably 5 mgKOH/g or more, more preferably 8 mgKOH/g or more, and still more preferably 10 mgKOH/g or more. When the acid value is 5 mgKOH/g or more, the dispersion stability of the (D1) pigment can be improved. On the other hand, the acid value is preferably 200 mgKOH/g or less, more preferably 170 mgKOH/g or less, still more preferably 150 mgKOH/g or less. When the acid value is 200 mgKOH/g or less, the storage stability of the resin composition can be improved.

The acid value herein refers to the weight of potassium hydroxide that reacts with 1 g of the (E) dispersant, and the unit is mgKOH/g. The acid value can be determined by titrating 1 g of the (E) dispersant with an aqueous potassium hydroxide solution. From the acid value, the acid equivalent (unit: g/mol), which refers to the resin weight per 1 mol of acidic groups, can be calculated, and the number of acidic groups in the (E) dispersant can be determined.

Examples of the (E) dispersant having a polymer chain, acrylic resin-based dispersants, polyoxyalkylene ether-based dispersants, polyester-based dispersants, polyurethane-based dispersants, polyol-based dispersants, polyethyleneimine-based dispersants, and polyallylamine-based dispersants. From the viewpoint of patternability with an alkaline developer, acrylic resin-based dispersants, polyoxyalkylene ether-based dispersants, polyester-based dispersants, polyurethane-based dispersants, and polyol-based dispersants are preferred.

In a case where the negative photosensitive resin composition according to the present invention contains a dispersive dye as the (D1) pigment and/or the (D2) dye, the content ratio of the (E) dispersant in the negative photosensitive resin composition according to the present invention is, in a case where the total of the (D1) pigment and/or dispersive dye and the (E) dispersant is regarded as 100% by mass, preferably 1% by mass or higher, more preferably 5% by mass or higher, still more preferably 10% by mass or higher. When the content ratio is 1% by mass or higher, the dispersion stability of the (D1) pigment and/or dispersive dye can be improved, and the resolution after development can be improved. On the other hand, the content ratio of the (E) dispersant is preferably 60% by mass or lower, more preferably 55% by mass or lower, still more preferably 50% by mass or lower. When the content ratio is 60% by mass or lower, the heat resistance of the cured film can be improved.

### <(F) Polyfunctional Thiol Compound>

The negative photosensitive resin composition according to the present invention preferably further contains a chain transfer agent. The chain transfer agent refers to a compound capable of receiving a radical from a polymer growth terminal of the polymer chain obtained by radical polymerization during exposure and mediating radical transfer to another polymer chain.

It is preferable to contain the (F) polyfunctional thiol compound as a chain transfer agent. Containing the (F) polyfunctional thiol compound allows the sensitivity for exposure to be improved. This is presumed to be because the radicals generated by exposure are transferred to other polymer chains, thereby causing radical crosslinking even to the deep part of the film. In particular, for example, in a case where the resin composition contains the (Da) black colorant as the (D) colorant described above, the light from exposure is absorbed by the (Da) black colorant, and thus, no light may reach the deep part of the film. On the other hand, in the case of containing the (F) polyfunctional thiol compound, the radical transfer causes radical crosslinking even to the deep part of the film, thus allowing the sensitivity for exposure to be improved.

In addition, containing the (F) polyfunctional thiol compound allows a cured film in a low-taper pattern shape to be obtained. This is presumed to be because the radical transfer is capable of controlling the molecular weight of the polymer chain obtained by radical polymerization during exposure. More specifically, containing the (F) polyfunctional thiol compound inhibits the production of a remarkably high-molecular-weight polymer chain due to excessive radical polymerization during exposure, thereby keeping the softening point of the obtained film from being increased. Thus, it is believed that the reflow property of the pattern during the thermal curing is improved, thereby providing a low-taper pattern shape.

Moreover, the negative photosensitive resin composition according to the present invention contains the above-mentioned (C1-1) specific oxime ester-based photo initiator and (F) polyfunctional thiol compound, thereby allowing the residue generation during development to be inhibited, and allowing the change in pattern opening width between before and after thermal curing to be suppressed. This is presumed to be because the (G) polyfunctional thiol compound suppresses oxygen inhibition at the film surface, thereby promoting UV curing during exposure. More specifically, it is believed to be because the UV curing by the (C1-1) specific oxime ester-based photo initiator is remarkably promoted. In the case of containing, in particular, the (D1) pigment as the (D) colorant, through the suppression of oxygen inhibition at the film surface, the (D1) pigment is immobilized to the cured part by crosslinking during UV curing, thus making it possible to inhibit the residue generation after development, which is derived from the (D1) pigment. In particular, in the case of containing, in particular, a (D1a-1a) benzofuranone-based black pigment as the (Da) black colorant, a development residue derived from the pigment described above may be generated due to the insufficient alkali resistance of the above-described pigment. Even in such a case, containing the (G) polyfunctional thiol compound promotes UV curing through the suppression of oxygen inhibition at the film surface, thereby allowing the development residue generation derived from the pigment to be suppressed.

Furthermore, as described above, the negative photosensitive resin composition according to the present invention preferably contains the above-described (B4) alicyclic group-containing radical polymerizable compound and (F) polyfunctional thiol compound, from the viewpoints of suppressing the change in pattern opening width between before and after thermal curing and forming a pattern in a low-taper shape after development.

As the (F) polyfunctional thiol compound, it is preferable to contain one or more selected from the group consisting of a compound represented by the general formula (83), a compound represented by the general formula (85), and a compound represented by the general formula (85).

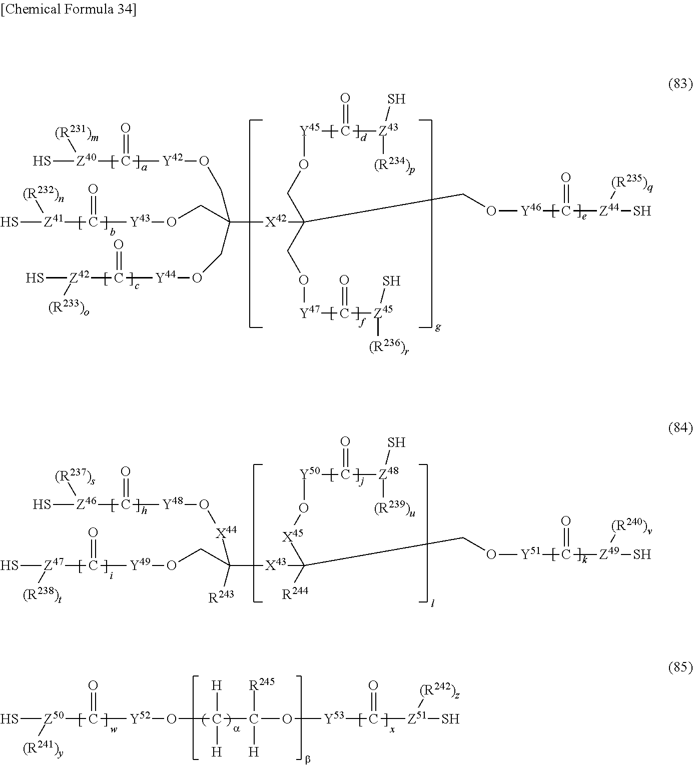

In the general formulas (83) to (85), X⁴² and X⁴³ represent a divalent organic group. X⁴⁴ and X⁴⁵ each independently represent a direct bond or an alkylene chain having 1 to 10 carbon atoms. Y⁴² to Y⁵³ each independently represent a direct bond, an alkylene chain having 1 to 10 carbon atoms, or a group represented by general formula (86). Z⁴⁰ to Z⁵¹ each independently represent a direct bond or an alkylene chain having 1 to 10 carbon atoms. R²³¹ to R²⁴² each independently represent an alkylene chain having 1 to 10 carbon atoms. R²⁴³ to R²⁴⁵ each independently represent hydrogen or an alkyl group having 1 to 10 carbon atoms. a, b, c, d, e, f, h, i, j, k, w, and x each independently represent 0 or 1. g and l each independently represent an integer of 0 to 10. m, n, o, p, q, r, s, t, u, v, y, and z each independently represent an integer of 0 to 10. α and β each independently represent an integer of 1 to 10. In the general formulas (83) to (85), X⁴² and X⁴³ each independently preferably represent a divalent organic group having one or more selected from an aliphatic structure having 1 to 10 carbon atoms, an alicyclic structure having 4 to 20 carbon atoms, and an aromatic structure having 6 to 30 carbon atoms. a, b, c, d, e, f, h, i, j, k, w, and x each independently preferably represent 1. g and l each independently preferably represent an integer of 0 to 5. m, n, o, p, q, r, s, t, u, v, y, and z each independently preferably represent 0. α and β each independently preferably represent an integer of 1 to 5, more preferably 1 or 2, still more preferably 1. The above-described alkyl group, alkylene chain, aliphatic structure, alicyclic structure, and aromatic structure may have a hetero atom, and may be either unsubstituted or substituted.

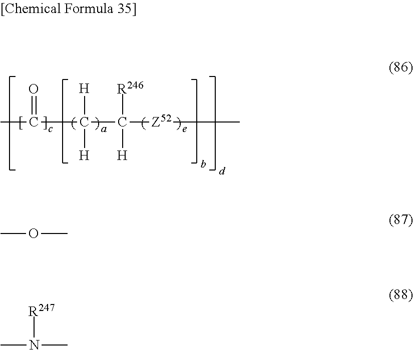

In the general formula (86), R²⁴⁶ represents hydrogen or an alkyl group having 1 to 10 carbon atoms. Z⁵² represents a group represented by general formula (87) or a group represented by general formula (88). a represents an integer of 1 to 10, b represents an integer of 1 to 4, c represents 0 or 1, d represents an integer of 1 to 4, and e represents 0 or 1. In a case where c is 0, d represents 1. In the general formula (88), R²⁴⁷ represents hydrogen or an alkyl group having 1 to 10 carbon atoms. In the general formula (86), R²⁴⁶ represents hydrogen or an alkyl group having 1 to 10 carbon atoms. Z⁵² represents a group represented by general formula (87) or a group represented by general formula (88). a represents an integer of 1 to 10, b represents an integer of 1 to 4, c represents 0 or 1, d represents an integer of 1 to 4, and e represents 0 or 1. In a case where c is 0, d represents 1. In the general formula (88), R²⁴⁷ represents hydrogen or an alkyl group having 1 to 10 carbon atoms. In the general formula (86), c preferably represents 1, and e preferably represents 1. In the general formula (88), R²⁴⁷ preferably represents hydrogen or an alkyl group having 1 to 4 carbon atoms, more preferably hydrogen or a methyl group.

Examples of the (F) polyfunctional thiol compound include β-mercaptopropionic acid, methyl β-mercaptopropionate, 2-ethylhexyl S-mercaptopropionate, stearyl S-mercaptopropionate, methoxybutyl β-mercaptopropionate, β-mercaptobutanoic acid, methyl β-mercaptobutanoate, methyl thioglycolate, n-octyl thioglycolate, methoxybutyl thioglycolate, 1,4-bis(3-mercaptobutanoyloxy) butane, 1,4-bis(3-mercaptopropionyloxy) butane, 1,4-bis(thioglycoloyloxy) butane, ethylene glycol bis(thioglycolate), ethylene glycol bis(3-mercaptopropionate), ethylene glycol bis(2-mercaptopropionate), ethylene glycol bis(3-mercaptobutyrate), diethylene glycol bis(3-mercaptopropionate), tetraethylene glycol bis(3-mercaptopropionate), ethylene glycol bis(2-mercaptoethyl) ether, ethylene glycol bis(3-mercaptopropyl) ether, ethylene glycol bis(2-mercaptopropyl) ether, ethylene glycol bis(3-mercaptobutyl) ether, diethylene glycol bis(2-mercaptoethyl) ether, tetraethylene glycol bis(2-mercaptoethyl) ether, propylene glycol bis(2-mercaptoethyl) ether, butylene glycol bis(2-mercaptoethyl) ether, 1,2-bis(2-mercaptoethylthio) ethane, 1,2-bis(3-mercaptopropylthio) ethane, diethylene thioglycol bis(2-mercaptoethyl) ether, tetraethylene thioglycol bis(2-mercaptoethyl) ether, 1,3-bis(2-mercaptoethylthio) propane, 1,4-bis(2-mercaptoethylthio) butane, trimethylolethane tris(3-mercaptopropionate), trimethylolethane tris(3-mercaptobutyrate), trimethylolpropane tris(3-mercaptopropionate), trimethylolpropane tris(3-mercaptobutyrate), trimethylolpropane tris(thioglycolate), 1,3,5-tris[(3-mercaptopropionyloxy)ethyl] isocyanuric acid, 1,3,5-tris[(3-mercaptobutanoyloxy)ethyl] isocyanuric acid, pentaerythritol tetrakis(3-mercaptopropionate), pentaerythritol tetrakis(3-mercaptobutyrate), pentaerythritol tetrakis(thioglycolate), dipentaerythritol hexakis(3-mercaptopropionate), or dipentaerythritol hexakis(3-mercaptobutyrate).

From the viewpoints of sensitivity improvement for exposure, pattern formation in a low-taper shape, and residue control after development, ethylene glycol bis(thioglycolate), ethylene glycol bis(3-mercaptopropionate), ethylene glycol bis(2-mercaptopropionate), ethylene glycol bis(3-mercaptobutyrate), ethylene glycol bis(2-mercaptoethyl) ether, ethylene glycol bis(3-mercaptopropyl) ether, ethylene glycol bis(2-mercaptopropyl) ether, ethylene glycol bis(3-mercaptobutyl) ether, 1,2-bis(2-mercaptoethylthio) ethane, 1,2-bis(3-mercaptopropylthio) ethane, trimethylolethane tris(3-mercaptopropionate), trimethylolethane tris(3-mercaptobutyrate), trimethylolpropane tris(3-mercaptopropionate), trimethylolpropane tris(3-mercaptobutyrate), trimethylolpropane tris(thioglycolate), 1,3,5-tris[(3-mercaptopropionyloxy)ethyl] isocyanuric acid, 1,3,5-tris[(3-mercaptobutanoyloxy)ethyl] isocyanuric acid, pentaerythritol tetrakis(3-mercaptopropionate), pentaerythritol tetrakis(3-mercaptobutyrate), pentaerythritol tetrakis(thioglycolate), dipentaerythritol hexakis(3-mercaptopropionate), or dipentaerythritol hexakis(3-mercaptobutyrate) is preferred. Furthermore, from the viewpoint of residue control after development, ethylene glycol bis(2-mercaptoethyl) ether, ethylene glycol bis(3-mercaptopropyl) ether, ethylene glycol bis(2-mercaptopropyl) ether, ethylene glycol bis(3-mercaptobutyl) ether, 1,2-bis(2-mercaptoethylthio) ethane, or 1,2-bis(3-mercaptopropylthio) ethane is more preferred.

The content of the (F) polyfunctional thiol compound in the negative photosensitive resin composition according to the present invention is, in a case where the total of the (A) alkali-soluble resin and (B) radical polymerizable compound is regarded as 100 parts by mass, preferably 0.01 parts by mass or more, more preferably 0.1 parts by mass or more, still more preferably 0.3 parts by mass or more, even more preferably 0.5 parts by mass or more, particularly preferably 1 part by mass or more. When the content is 0.01 parts by mass or more, the sensitivity for exposure can be improved, and a cured film in a low-taper pattern shape can be obtained. In addition, the residue generation during development can be inhibited. On the other hand, the content of the (F) polyfunctional thiol compound is preferably 15 parts by mass or less, more preferably 13 parts by mass or less, still more preferably 10 parts by mass or less, even more preferably 8 parts by mass or less, particularly preferably 5 parts by mass or less. When the content is 15 parts by mass or less, a pattern in a low-taper shape can be formed, the residue generation after development can be inhibited, and the heat resistance of the cured film can be improved.

### <Sensitizer>

The negative photosensitive resin composition according to the present invention preferably further contains a sensitizer. The sensitizer refers to a compound capable of absorbing exposure energy, generating excited triplet electrons by internal conversion and intersystem crossing, and mediating energy transfer to the above-described (C1) photo initiator or the like.

Containing the sensitizer allows the sensitivity for exposure to be improved. This is presumed to be because the sensitizer absorbs long-wavelength light which is not absorbed by the (C1) photo initiator or the like, then allowing the photoreaction efficiency to be improved by energy transfer of the light from the sensitizer to the (C1) photo initiator or the like.

As the sensitizer, a thioxanthone-based sensitizer is preferred. Examples of the thioxanthone-based sensitizer include thioxanthone, 2-methylthioxanthone, 2-chlorothioxanthone, 2-isopropylthioxanthone, 2,4-dimethylthioxanthone, 2,4-diethylthioxanthone, and 2,4-dichlorothioxanthone.

The content of the sensitizer in the negative photosensitive resin composition according to the present invention is, in a case where the (A) alkali-soluble resin and the (B) radical polymerizable compound are regarded as 100 parts by mass in total, preferably 0.01 parts by mass or more, more preferably 0.1 parts by mass or more, still more preferably 0.5 parts by mass or more, particularly preferably 1 part by mass or more. When the content is 0.01 parts by mass or more, the sensitivity for exposure can be improved. On the other hand, the content of the sensitizer is preferably 15 parts by mass or less, more preferably 13 parts by mass or less, still more preferably 10 parts by mass or less, particularly preferably 8 parts by mass or less. When the content is 15 parts by mass or less, the resolution after development can be improved, and a cured film in a low-taper pattern shape can be obtained.

### <Polymerization Terminator>

The negative photosensitive resin composition according to the present invention preferably further contains a polymerization terminator. The polymerization terminator refers to a compound capable of terminating radical polymerization by capturing a radical generated at the time of exposure, or a radical at the polymer growth terminal of the polymer chain obtained by radical polymerization at the time of exposure, and holding the radical as a stable radical.

Containing the polymerization terminator in an appropriate amount makes it possible to inhibit the generation of residues after development and improve the resolution after the development. This is presumed to be because the polymerization terminator captures an excessive amount of radical generated at the time of exposure or a radical at the growth terminal of the high-molecular-weight polymer chain, thereby keeping the radical polymerization from proceeding excessively.

As the polymerization terminator, a phenolic polymerization terminator is preferred. Examples of the phenolic polymerization terminator include 4-methoxyphenol, 1,4-hydroquinone, 1,4-benzoquinone, 2-t-butyl-4-methoxyphenol, 3-t-butyl-4-methoxyphenol, 4-t-butylcatechol, 2,6-di-t-butyl-4-methylphenol, 2,5-di-t-butyl-1,4-hydroquinone, or 2,5-di-t-amyl-1,4-hydroquinone, or IRGANOX (registered trademark) 245, 259, 565, 1010, 1035, 1076, 1098, 1135, 1330, 1425, 1520, 1726, 3114 (all manufactured by BASF).

The content of the polymerization terminator in the negative photosensitive resin composition according to the present invention is, in a case where the (A) alkali-soluble resin and the (B) radical polymerizable compound are regarded as 100 parts by mass in total, preferably 0.01 parts by mass or more, more preferably 0.03 parts by mass or more, still more preferably 0.05 parts by mass or more, particularly preferably 0.1 parts by mass or more. When the content is 0.01 parts by mass or more, the resolution after development and the heat resistance of the cured film can be improved. On the other hand, the content of the polymerization terminator is preferably 10 parts by mass or less, more preferably 8 parts by mass or less, still more preferably 5 parts by mass or less, particularly preferably 3 parts by mass or less. When the content is 10 parts by mass or less, the sensitivity for exposure can be improved.

### <Cross-Linking Agent>

The negative photosensitive resin composition according to the present invention preferably further contains a cross-linking agent. The cross-linking agent refers to a compound having a cross-linkable group capable of binding to the resin. Containing the cross-linking agent allows the hardness and chemical resistance of the cured film to be improved. This is presumed to be because the cross-linking agent is capable of introducing a new cross-linked structure into the cured film of the resin composition, thus improving the crosslink density.

In addition, containing the cross-linking agent makes it possible to form a pattern in a low-taper shape after thermal curing. This is believed to be because the (F) cross-linking agent forms a cross-linked structure between the polymers, thereby inhibiting the tight orientation of the polymer chains, and then making it possible to maintain the reflow property of the pattern during thermal curing, and thus allowing a pattern in a low-taper shape to be formed. As the cross-linking agent, a compound having two or more thermal crosslinkable properties in the molecule, is preferred, such as an alkoxymethyl group, a methylol group, an epoxy group, or an oxetanyl group.

Examples of the compound having two or more alkoxymethyl groups or methylol groups in the molecule include DML-PC, DML-OC, DML-PTBP, DML-PCHP, DML-MBPC, DML-MTrisPC, DMOM-PC, DMOM-PTBP, TriML-P, TriML-35XL, TML-HQ, TML-BPA, TML-BPAF, TML-BPAP, TMOM-BPA, TMOM-BPAF, TMOM-BPAP, HML-TPHAP, or HMOM-TPHAP (all manufactured by Honshu Chemical Industry Co., Ltd.), and “NIKALAC” (registered trademark) MX-290, MX-280, MX-270, MX-279, MW-100LM, MW-30HM, MW-390, or MX-750LM (manufactured by SANWA CHEMICAL CO., LTD.).

Examples of the compound having two or more epoxy groups in the molecule include “Epolite” (registered trademark) 40E, 100E, 400E, 70P, 1500NP, 80MF, 3002, or 4000 (all manufactured by Kyoeisha Chemical Co., Ltd.), “Denacol” (registered trademark) EX-212L, EX-216L, EX-321L, or EX-850L (all manufactured by Nagase ChemteX Corporation), “jER” (registered trademark) 828, 1002, 1750, YX8100-BH30, E1256, or E4275 (all manufactured by Mitsubishi Chemical Corporation), GAN, GOT, EPPN-502H, NC-3000, or NC-6000 (all manufactured by Nippon Kayaku Co., Ltd.), “EPICLON” (registered trademark) EXA-9583, HP4032, N695, or HP7200 (all manufactured by Dainippon Ink and Chemicals Inc.), “TECHMORE” (registered trademark) VG-3101L (PRINTEC, INC.), “TEPIC” (registered trademark) S, G, or P (all manufactured by Nissan Chemical Corporation), and “Epototo” (registered trademark) YH-434L (manufactured by Tohto Kasei Co., Ltd.).

Examples of the compound having two or more oxetanyl groups in the molecule include “ETERNACOLL” (registered trademark) EHO, OXBP, OXTP, or OXMA (all manufactured by Ube Industries, Ltd.), and oxetanized phenol novolac.

The content of the cross-linking agent in the negative photosensitive resin composition according to the present invention is, in a case where the total of the (A) alkali-soluble resin and (B) radical polymerizable compound is regarded as 100 parts by mass, preferably 0.5 parts by mass or more, more preferably 1 part by mass or more, still more preferably 2 parts by mass or more, even more preferably 3 parts by mass or more, particularly preferably 5 parts by mass or more. When the content is 0.5 parts by mass or more, the hardness and chemical resistance of the cured film can be improved, and a pattern in a low-taper shape can be formed after thermal curing. On the other hand, the content of the cross-linking agent is preferably 50 parts by mass or less, more preferably 40 parts by mass or less, still more preferably 30 parts by mass or less, even more preferably 25 parts by mass or less, particularly preferably 20 parts by mass or less. When the content is 50 parts by mass or less, the hardness and chemical resistance of the cured film can be improved, and a pattern in a low-taper shape can be formed after thermal curing.

### <Silane Coupling Agent>

The negative photosensitive resin composition according to the present invention preferably further contains a silane coupling agent. The silane coupling agent refers to a compound having a hydrolyzable silyl group or silanol group. Containing the silane coupling agent makes it possible to increase the interaction between the cured film of the resin composition and the underlying substrate interface, thereby allowing the adhesion property to the underlying substrate and the chemical resistance of the cured film to be improved. As the silane coupling agent, a trifunctional organosilane, a tetrafunctional organosilane, or a silicate compound is preferred.

Examples of the trifunctional organosilane include methyltrimethoxysilane, cyclohexyltrimethoxysilane, vinyltrimethoxysilane, 3-acryloxypropyltrimethoxysilane, phenyltrimethoxysilane, 4-hydroxyphenyltrimethoxysilane, 1-naphthyltrimethoxysilane, 4-styryltrimethoxysilane, 3-glycidoxypropyltrimethoxysilane, 3-trimethoxysilylpropyl succinic acid, 3-trimethoxysilylpropyl succinic anhydride, 3,3,3-trifluoropropyltrimethoxysilane, 3-[(3-ethyl-3-oxetanyl)methoxy]propyltrimethoxysilane, 3-aminopropyltrimethoxysilane, 3-(4-aminophenyl)propyltrimethoxysilane, 1-(3-trimethoxysilylpropyl) urea, 3-triethoxysilyl-N-(1,3-dimethylbutylidene)propylamine, 3-mercaptopropyltrimethoxysilane, 3-isocyanatopropyltriethoxysilane, 1,3,5-tris(3-trimethoxysilylpropyl) isocyanurate, or N-t-butyl-2-(3-trimethoxysilylpropyl)succinimide. Examples of the tetrafunctional organosilane or silicate compound include an organosilane represented by general formula (73).

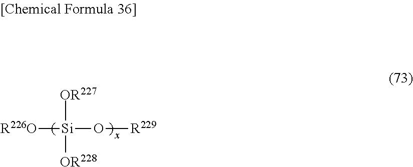

In the general formula (73), R²²⁶ to R²²⁹ each independently represents hydrogen, an alkyl group, an acyl group, or an aryl group, and x represents an integer of 1 to 15. In the general formula (73), R²²⁶ to R²²⁹ each independently preferably hydrogen, an alkyl group having 1 to 6 carbon atoms, an acyl group having 2 to 6 carbon atoms, or an aryl group having 6 to 15 carbon atoms, more p, an alkyl group having 1 to 4 carbon atoms, an acyl group having 2 to 4 carbon atoms, or an aryl group having 6 to 10 carbon atoms. The above-described alkyl group, acyl group, and aryl group may be either unsubstituted or substituted.

Examples of the organosilane represented by general formula (73) include a tetrafunctional organosilane such as tetramethoxysilane, tetraethoxysilane, tetra-n-propoxysilane, tetraisopropoxysilane, tetra-n-butoxysilane, or tetraacetoxysilane, or a silicate compound such as methyl silicate 51 (manufactured by FUSO CHEMICAL CO., LTD.), M silicate 51, silicate 40, or silicate 45 (all manufactured by TAMA CHEMICALS CO., LTD.), or methyl silicate 51, methyl silicate 53A, ethyl silicate 40, or ethyl silicate 48 (all manufactured by COLCOAT CO., LTD.).

The content of the silane coupling agent in the negative photosensitive resin composition according to the present invention is, in a case where the (A) alkali-soluble resin and the (B) radical polymerizable compound are regarded as 100 parts by mass in total, preferably 0.01 parts by mass or more, more preferably 0.1 parts by mass or more, still more preferably 0.5 parts by mass or more, particularly preferably 1 part by mass or more. When the content is 0.01 parts by mass or more, the adhesion property to the underlying substrate and the chemical resistance of the cured film can be improved. On the other hand, the content of the silane coupling agent is preferably 15 parts by mass or less, more preferably 13 parts by mass or less, still more preferably 10 parts by mass or less, particularly preferably 8 parts by mass or less. When the content is 15 parts by mass or less, the resolution after development can be improved.

### <Surfactant>

The negative photosensitive resin composition according to the present invention may further contain a surfactant. The surfactant refers to a compound that has a hydrophilic structure and a hydrophobic structure. Containing the surfactant in an appropriate amount allows the surface tension of the resin composition to be adjusted arbitrarily, thereby improving the leveling property for coating, and then allowing the film thickness uniformity of the coating film to be improved. As the surfactant, a fluororesin-based surfactant, a silicone-based surfactant, a polyoxyalkylene ether-based surfactant, or an acrylic resin-based surfactant is preferable.

The content ratio of the surfactant in the negative photosensitive resin composition according to the present invention is preferably 0.001% by mass or higher, more preferably 0.005% by mass or higher, still more preferably 0.01% by mass or higher, based on the whole negative photosensitive resin composition. When the content ratio is 0.001% by mass or higher, the leveling property for coating can be improved. On the other hand, the content ratio of the surfactant is preferably 1% by mass or lower, more preferably 0.5% by mass or lower, still more preferably 0.03% by mass or lower. When the content ratio is 1% by mass or lower, the leveling property for coating can be improved.

### <Solvent>

The negative photosensitive resin composition according to the present invention preferably further contains a solvent. The solvent refers to a compound capable of dissolving various resins and various additives to be contained in the resin composition. Containing the solvent makes it possible to uniformly dissolve various resins and various additives to be contained in the resin composition, thereby improving the transmittance of the cured film. Furthermore, the viscosity of the resin composition can be adjusted arbitrarily, and a film with a desired film thickness can be formed on the substrate. In addition, the surface tension of the resin composition or the drying speed thereof for coating can be adjusted arbitrarily, and the leveling property for coating and the film thickness uniformity of the coating film can be improved.

As the solvent, a compound having an alcoholic hydroxyl group, a compound having a carbonyl group, or a compound having three or more ether bonds is preferred from the viewpoint of the solubility of various resins and various additives. In addition, a compound having a boiling point of 110 to 250° C. under atmospheric pressure is more preferred. The boiling point is adjusted to 110° C. or higher, thereby causing the solvent to evaporate appropriately for coating, and then causing drying of the coating film to proceed, and thus, coating unevenness can be suppressed, and the film thickness uniformity can be improved. On the other hand, the boiling point is adjusted to 250° C. or lower, thereby allowing the amount of the solvent remaining in the coating film to be reduced. Accordingly, the amount of film shrinkage during thermal curing can be reduced, the flatness of the cured film can be improved, and the film thickness uniformity can be improved.

Examples of the compound having an alcoholic hydroxyl group and a boiling point of 110 to 250° C. under atmospheric pressure include diacetone alcohol, ethyl lactate, ethylene glycol monomethyl ether, propylene glycol monomethyl ether, diethylene glycol monomethyl ether, dipropylene glycol monomethyl ether, 3-methoxy-1-butanol, 3-methoxy-3-methyl-1-butanol, and tetrahydrofurfuryl alcohol.

Examples of the compound having a carbonyl group and a boiling point of 110 to 250° C. under atmospheric pressure include 3-methoxy-n-butyl acetate, 3-methyl-3-n-butyl acetate, propylene glycol monomethyl ether acetate, dipropylene glycol monomethyl ether acetate, and γ-butyrolactone.

Examples of the compound having three or more ether bonds and a boiling point of 110 to 250° C. under atmospheric pressure include diethylene glycol dimethyl ether, diethylene glycol ethyl methyl ether, and dipropylene glycol dimethyl ether.

The content ratio of the solvent in the negative photosensitive resin composition according to the present invention can be adjusted appropriately depending on the coating method and the like. For example, in the case of forming a coating film by spin coating, it is common to adjust the ratio to 50 to 95% by mass of the whole negative photosensitive resin composition.

In the case of containing, as the (D) colorant, a dispersive dye as the (D1) pigment and/or (D2) dye, the solvent is preferably a solvent having a carbonyl group or an ester bond. Containing the solvent having a carbonyl group or an ester bond allows the dispersion stability of the dispersive dye to be improved as the (D1) pigment and/or (D2) dye. Furthermore, from the viewpoint of dispersion stability, the solvent is more preferably a solvent having an acetate bond. Containing the solvent having an acetate bond allows the dispersion stability of the dispersive dye to be improved as the (D1) pigment and/or (D2) dye.

Examples of the solvent having an acetate bond include 3-methoxy-n-butyl acetate, 3-methyl-3-methoxy-n-butyl acetate, ethylene glycol monomethyl ether acetate, propylene glycol monomethyl ether acetate, diethylene glycol monomethyl ether acetate, diethylene glycol monoethyl ether acetate, diethylene glycol mono-n-butyl ether acetate, dipropylene glycol monomethyl ether acetate, cyclohexanol acetate, propylene glycol diacetate, and 1,4-butanediol diacetate.

In the negative photosensitive resin composition according to the present invention, the content ratio of the solvent having a carbonyl group or an ester bond in the solvent is preferably 30 to 100. by mass, more preferably 50 to 100% by mass, still more preferably 70 to 100% by mass. When the content ratio is 30 to 100% by mass, the dispersion stability of the (D1) pigment can be improved.

### <Other Additives>

The negative photosensitive resin composition according to the present invention may further contain other resins or precursors thereof. Examples of the other resins or precursors thereof include polyamide, polyamideimide, epoxy resins, novolac resins, urea resins, and polyurethane, and precursors thereof.

### <Method for Manufacturing Negative Photosensitive Resin Composition According to the Present Invention>

A typical method for manufacturing the negative photosensitive resin composition according to the present invention will be described. In the case of containing the (D1) pigment including the (Da) black colorant as the (D) colorant, the (E) dispersant is added to a solution of the (A1) first resin and (A2) second resin, and with the use of a disperser, the pigment (D1) is dispersed in this mixed solution to prepare a pigment dispersion. Next, this pigment dispersion with the (B) radical polymerizable compound, the (C1) photo initiator, the other additives, and an optional solvent added thereto, is stirred for 20 minutes to 3 hours to provide a uniform solution. After the stirring, the obtained solution is filtered, thereby providing the negative photosensitive resin composition according to the present invention.

Examples of the disperser include a ball mill, a bead mill, a sand grinder, a triple roll mill, and a high-speed impact mill. From the viewpoints of more efficient dispersion and finer dispersion, a bead mill is preferred. Examples of the bead mill include a co-ball mill, a basket mill, a pin mill, or a DYNO mill. Examples of the beads of the bead mill include titania beads, zirconia beads, or zircon beads. The bead diameter of the bead mill is preferably 0.01 to 6 mm, more preferably 0.015 to 5 mm, still more preferably 0.03 to 3 mm. In a case where the primary particle size of the (D1) pigment and the particle size of the secondary particle formed by aggregation of the primary particles are equal to or smaller than several hundred nanometers (nm), fine beads of 0.015 to 0.1 mm are preferred. In this case, a bead mill is preferred which is provided with a separator capable of separating minute beads and a pigment dispersion by a centrifugal separation method. On the other hand, in a case where the (D1) pigment contains coarse particles equal to or larger than several hundred nanometers (nm), beads of 0.1 to 6 mm are preferred from the viewpoint of more efficient dispersion.

### <Cured Pattern in Low Taper Pattern Shape>

The negative photosensitive resin composition according to the present invention is capable of providing a cured film including a cured pattern in a low-taper pattern shape. The taper angle of the inclined side in a cross section of the cured pattern included in the cured film, obtained from the negative photosensitive resin composition according to the present invention is preferably 1° or more, more preferably 5° or more, still more preferably 10° or more, even more preferably 12° or more, particularly preferably 15° or more. When the taper angle is 1° or more, light-emitting elements can be integrated and arranged at high density, and the resolution of the display device can be thus improved. On the other hand, the taper angle of the inclined side in the cross section of the cured pattern included in the cured film is preferably 60° or less, more preferably 55° or less, still more preferably 50° or less, even more preferably 45° or less, particularly preferably 40° or less. When the taper angle is 60° or less, disconnection can be prevented in forming an electrode such as a transparent electrode or a reflective electrode. Furthermore, the electric field concentration at the edge of the electrode can be suppressed, and degradation of the light emitting elements can be thus suppressed.

<Curing Pattern with Step Shape>

The negative photosensitive resin composition according to the present invention is capable of forming a cured pattern that has a step shape with a sufficient difference in film thickness between a thick film part and a thin film part, and has a low-taper pattern shape, while maintaining a high sensitivity. In addition, it is possible to reduce the taper by pattern shape control after development, and it is possible to suppress the change in pattern opening width between before and after thermal curing. Thus, the step shape formed after development and the pattern opening width can be maintained after thermal curing. Accordingly, the negative photosensitive resin composition according to the present invention is particularly suitable in applications for collectively forming a step shape for a pixel defining layer in an organic EL display. Similarly, the composition is suitable in applications for collectively forming step shapes for an electrode insulation layer, a wiring insulation layer, an interlayer insulation layer, a TFT planarization layer, an electrode planarization layer, a wiring planarization layer, a TFT protective layer, an electrode protective layer, a wiring protective layer, a gate insulation layer, a color filter, a black matrix, or a black column spacer.

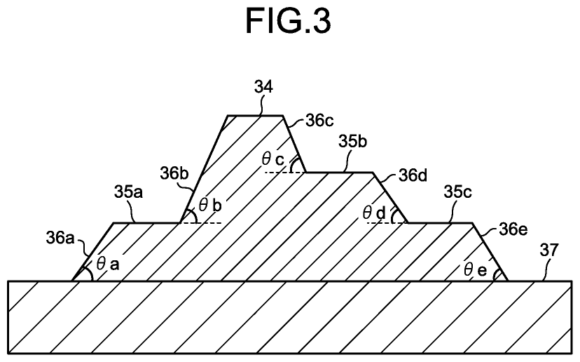

FIG. 3 shows therein a cross section example of a cured pattern which has a step shape, obtained from the negative photosensitive resin composition according to the present invention. A thick film part 34 in the step shape corresponds to a cured part during exposure, and has the maximum film thickness of the cured pattern. Thin film parts 35a, 35b, and 35c in the step shape correspond to halftone exposed parts during exposure, and have film thicknesses smaller than the thickness of the thick film part 34. The respective taper angles θₐ, θ_{b}, θ_{c}, θ_{d}, and θₑ of inclined sides 36a, 36b, 36c, 36d, and 36e in the cross section of the cured pattern with the step shape preferably all have low tapers.

The taper angles θₐ, θ_{b}, θ_{c}, θ_{d}, and θₑ herein refer to, as shown in FIG. 3, angles inside the cross section of the cured pattern with the step shape, which are made by a horizontal side 37 of the underlying substrate with the cured pattern formed, or the horizontal sides of the thin film parts 35a, 35b, and 35c, and the inclined sides 36a, 36b, 36c, 36d, and 36e in the cross section of the cured pattern with the step shape, which intersect the horizontal sides of the thin film parts 35a, 35b, and 35c. In this regard, the forward tapered shape means that the taper angle falls within the range of more than 0° and less than 90°, and the inverse tapered shape means that the taper angle falls within the range of more than 90° and less than 180°. Furthermore, the rectangular shape means that the taper angle is 90°, and the low-taper shape means that the taper angle falls within the range of more than 0° to 60°.

As for the thickness between the planes of the lower surface and upper surface of the cured pattern with a step shape, obtained from the negative photosensitive resin composition according to the present invention, the region with the largest thickness is regarded as a thick film part 34, whereas the region with a thickness smaller than the thickness of the thick film part 34 is regarded as a thin film part 35. In a case where the film thickness of the thick film part 34 is denoted by (T_{FT}) μm, whereas the film thicknesses of the thin film parts 35a, 35b, and 35c disposed on the thick film part 34 with at least one step shape interposed therebetween are denoted by (T_{HT}) μm, the film thickness difference (ΔT_{FT-HT}) μm between (T_{FT}) and (T_{HT}) is preferably 0.5 μm or more, more preferably 1.0 μm or more, still more preferably 1.5 μm or more, even more preferably 2.0 μm or more, particularly preferably 2.5 μm or more, most preferably 3.0 μm or more. When the film thickness difference falls within the range described above, the area of contact with an evaporation mask in the formation of a light-emitting layer can be reduced, thereby allowing the decrease in panel yield due to particle generation to be suppressed, and allowing degradation of the light-emitting element to be suppressed. In addition, the cured pattern with a step shape has a sufficient film thickness difference by itself, thus making it possible to shorten the process time. On the other hand, the film thickness difference (ΔT_{FT-HT}) μm is preferably 10.0 μm or less, more preferably 9.5 μm or less, still more preferably 9.0 μm or less, even more preferably 8.5 μm or less, particularly preferably 8.0 μm or less. When the fil thickness difference falls within the range mentioned above, the exposure energy for the formation of the cured pattern with the step shape can be reduced, thereby allowing the takt time to be shortened.

The film thickness (T_{FT}) μm of the thick film part 34 and the film thicknesses (T_{HT}) μm of the thin film parts 35a, 35b, and 35c preferably satisfy the relations represented by the general formulas (α) to (γ).

2.0≤(T_{FT})≤10.0 (α)

0.20≤(T_{HT})≤7.5 (β)

0.10×(T_{FT})≤(T_{HT})≤0.75×(T_{FT}) (γ)

The film thickness (T_{FT}) μm of the thick film part 34 and the film thicknesses (T_{HT}) μm of the thin film parts 35a, 35b, and 35c preferably further satisfy the relations represented by the general formulas (δ) to (ζ).

2.0≤(T_{FT})≤10.0 (δ)

0.30≤(T_{HT})≤7.0 (ε)

0.15×(T_{FT})≤(T_{HT})≤0.70×(T_{FT}) (ζ)

When the film thickness (T_{FT}) μm of the thick film part 34 and the film thicknesses (T_{HT}) μm of the thin film pars 35a, 35b, and 35c fall within the above-described ranges, degradation of the light-emitting element can be suppressed, and the process time can be shortened.

In the organic EL display according to the present invention, the taper angle of the inclined side in a cross section of the cured pattern with the step shape, obtained from the negative photosensitive resin composition, is preferably 1° or more, more preferably 5° or more, still more preferably 10° or more, even more preferably 12° or more, particularly preferably 15° or more. When the taper angle falls within the range described above, light-emitting elements can be integrated and arranged at high density, and the resolution of the organic EL display can be thus improved. On the other hand, the taper angle of the inclined side in the cross section of the cured pattern is preferably 60° or less, more preferably 55° or less, still more preferably 50° or less, even more preferably 45° or less, particularly preferably 40° or less. When the taper angle falls within the range described above, disconnection can be prevented in forming an electrode such as a transparent electrode or a reflective electrode. Furthermore, the electric field concentration at the edge of the electrode can be suppressed, and degradation of the light emitting elements can be thus suppressed.

### <Manufacturing Process for Organic EL Display>

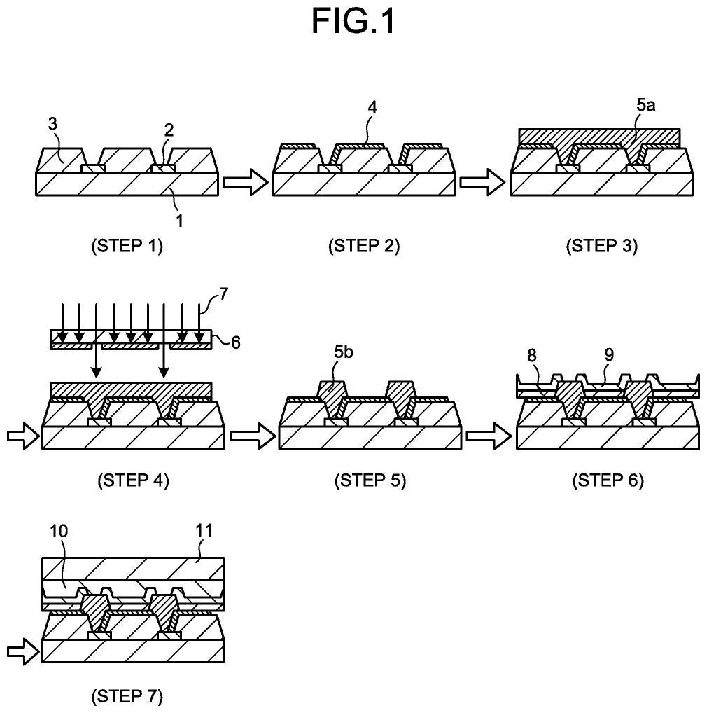

As a process with the negative photosensitive resin composition according to the present invention, a process using the cured film of the composition as a light-shielding pixel defining layer of an organic EL display will be described as an example with the schematic cross-sectional shown in FIG. 1. First, (Step 1) a thin-film-transistor (hereinafter, referred to as a “TFT”) 2 is formed on a glass substrate 1, a photosensitive material for a TFT planarization film is formed, subjected to pattern processing by photolithography, and then thermally cured to a cured film 3 for TFT planarization. Next, (Step 2) a silver-palladium-copper alloy (hereinafter, referred to as “APC”) is deposited by sputtering, and subjected to pattern processing by etching with the use of a photoresist to form an APC layer, and furthermore, as an upper layer on the APC layer, an indium tin oxide (hereinafter, referred to as an “ITO”) is formed by sputtering, and subjected to pattern processing by etching with the use of a photoresist to form the reflective electrode 4 as the first electrode. Thereafter, (Step 3) the negative photosensitive resin composition according to the present invention is applied and prebaked to form a prebaked film 5a. Then, (Step 4) irradiation with active actinic rays 7 is performed through a mask 6 that has a desired pattern. Next, (step 5) after development and pattern processing, bleaching exposure and middle baking are performed, if necessary, and thermal curing is performed, thereby forming, as a light-blocking pixel defining layer, a cured pattern 5b that has a desired pattern. Thereafter, (Step 6) an EL light-emitting material is deposited by vapor deposition through the mask to form an EL light-emitting layer 8, and a magnesium-silver alloy (hereinafter, referred to as “MgAg”) is deposited by vapor deposition, and subjected to pattern processing by etching with the use of a photoresist to form a transparent electrode 9 as the second electrode. Next, (Step 7) a photosensitive material for a planarization film is deposited, subjected to patter processing by photolithography, and then thermally cured to form a cured film 10 for planarization, and thereafter, cover glass 11 is joined, thereby providing an organic EL display including the negative photosensitive resin composition according to the present invention as a light-blocking pixel defining layer.

### <Manufacturing Process for Liquid Crystal Display>

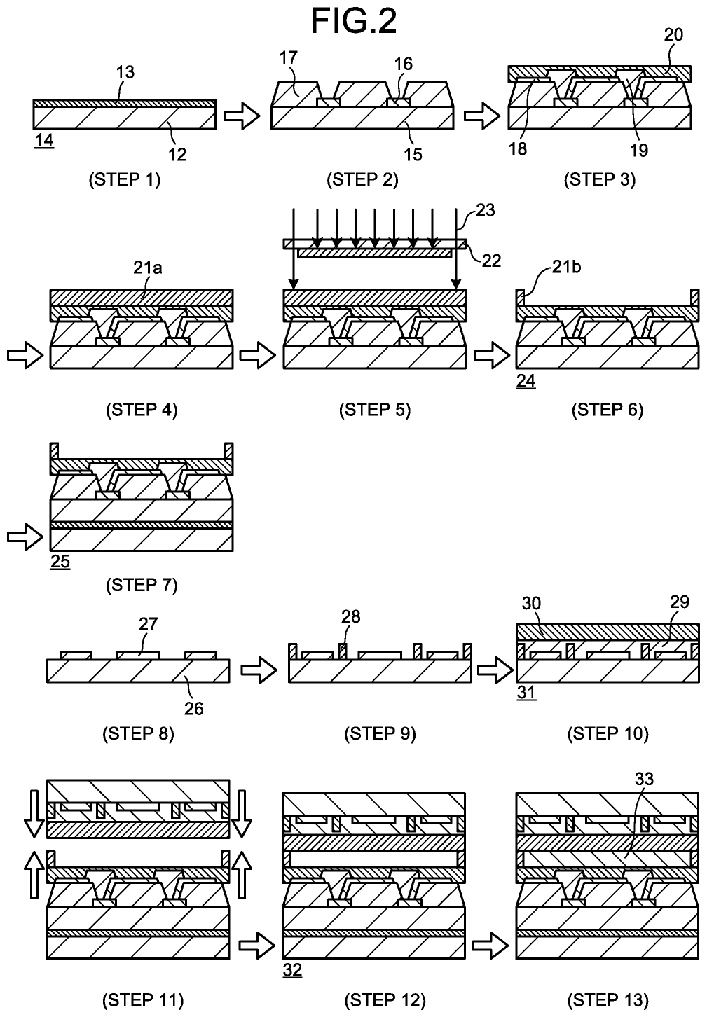

As another process with the negative photosensitive resin composition according to the present invention, a process with the cured film of the composition as a black column spacer (hereinafter, a “BCS”) for a liquid crystal display and a black matrix (hereinafter, a “BM”) for a color filter will be described as an example with the schematic cross-sectional view shown in FIG. 2.

As shown in FIG. 2, first, (Step 1) a backlight unit (hereinafter, referred to as a “BLU”) 13 is formed on a glass substrate 12 to obtain a glass substrate 14 with the BLU.

Furthermore, (Step 2) a TFT 16 is formed on another glass substrate 15, and a photosensitive material for a TFT planarization film is formed, subjected to pattern processing by photolithography, and then thermally cured to form a cured film 17 for TFT planarization. Next, (Step 3) an ITO is deposited by sputtering, and subjected to pattern processing by etching with the use of a photoresist to form a transparent electrode 18, and a planarization film 19 and an alignment layer 20 are formed thereon. Thereafter, (Step 4) the negative photosensitive resin composition according to the present invention is applied and prebaked to form a prebaked film 21a. Then, (step 5) irradiation with active actinic rays 23 is performed through a mask 22 that has a desired pattern. Next, (step 6) after development and pattern processing, bleaching exposure and middle baking are performed, if necessary, and thermal curing is performed, thereby forming, as a light-blocking BCS, a cured pattern 21b that has a desired pattern, and then providing a glass substrate 24 with the BCS. Then, (Step 7) the above-described glass substrate 14 and the glass substrate 24 are joined, thereby providing the glass substrate 25 with the BLU and the BCS.

Furthermore, (Step 8) on another glass substrate 26, color filters 27 of three colors of red, green and blue are formed. Next, (Step 9) a cured pattern 28 that has a desired pattern is formed as a light-blocking BM by the same method as described above from the negative photosensitive resin composition according to the present invention. Thereafter, (Step 10) a photosensitive material for planarization is deposited, subjected to pattern processing by photolithography, and then thermally cured to form a cured film 29 for planarization, and an alignment layer 30 is formed thereon, thereby providing a color filter substrate 31. Next, (Step 11) the above-described glass substrate 25 with the BLU and the BCS and the color filter substrate 31 are joined (Step 12) to obtain a glass substrate 32 with the BLU, the BCS, and the BM. Next, (Step 13) a liquid crystal is injected to form a liquid crystal layer 33, thereby providing a liquid crystal display including the negative photosensitive resin composition according to the present invention as the BCS and the BM.

As described above, the methods for manufacturing an organic EL display and a liquid crystal display with the use of the negative photosensitive resin composition according to the present invention are capable of achieving high heat-resistance and light-blocking cured films containing polyimide and/or polybenzoxazole, subjected to pattern processing, thus leading to improvements in yield, performance, and reliability in the manufacture of organic EL displays and liquid crystal displays.

According to the process with the negative photosensitive resin composition according to the present invention, it is possible for the resin composition to be directly subjected to pattern processing by photolithography, because the composition is photosensitive. Accordingly, the number of steps can be reduced as compared with processes with photoresists, thus making it possible to improv the productivity of organic EL displays and liquid crystal displays, and reduce the process time and the takt time.

<Display Device Including Cured Film Obtained from Negative Photosensitive Resin Composition According to Present Invention>

The cured film obtained from the negative photosensitive resin composition according to the present invention can suitably constitute an organic EL display or a liquid crystal display.

Moreover, the negative photosensitive resin composition according to the present invention is capable of achieving a low-taper pattern shape, thereby making it possible to obtain a cured film which is excellent in high heat resistance. Thus, the composition is suitable for applications which require high heat resistance and low-taper pattern shapes, such as an insulation layer such as a pixel defining layer of an organic EL display, a TFT planarization layer, or a TFT protective layer. In particular, in applications in which problems due to heat resistance and pattern shapes are expected, such as element failures or characteristic degradation due to degassing by thermal decomposition, or electrode wiring disconnection due to high-taper pattern shapes, the use of a cured film of the negative photosensitive resin composition according to the present invention makes it possible to manufacture a highly reliable element where the above-described problems are not caused. Furthermore, the cured film is excellent in light-blocking property, thus allowing electrode wiring to be prevented from becoming visible or allowing external light reflection to be reduced, and the contrast in image display can be thus improved. Accordingly, the use of the cured film obtained from the negative photosensitive resin composition according to the present invention as a pixel defining layer of an organic EL display device, a TFT planarization layer, or a TFT protective layer can improve the contrast, without forming any polarizing plate and a quarter wavelength plate on the light extraction side of the light-emitting element.

Furthermore, the organic EL display according to the present invention preferably has a curved display unit. The curvature radius of the curved surface is preferably 0.1 mm or more, more preferably 0.3 mm or more, from the viewpoint of suppressing the defective display caused by disconnection or the like in the curved display unit. In addition, the curvature radius of the curved surface is preferably 10 mm or less, more preferably 7 mm or less, still more preferably 5 mm or less, from the viewpoint of reduction in size and increase in resolution for the organic EL display.

The method for manufacturing a display device with the use of the negative photosensitive resin composition according to the present invention includes the following steps (1) to (4):

(1) a step of forming, on a substrate, a coating film of the negative photosensitive resin composition according to the present invention;

(2) a step of irradiating the coating film of the negative photosensitive resin composition with an active actinic ray through a photomask;

(3) a step of performing development with the use of an alkaline solution to form a pattern of the negative photosensitive resin composition; and

(4) a step of heating the pattern to obtain a cured pattern of the negative photosensitive resin composition.

### <Step of Forming Coating Film>

The method for manufacturing a display device with the use of the negative photosensitive resin composition according to the present invention includes the (1) step of forming, on a substrate, a coating film of the negative photosensitive resin composition. Examples of the method for depositing the negative photosensitive resin composition according to the present invention include a method of applying the above-described resin composition on a substrate, or a method of applying the above-mentioned resin composition in a pattern on a substrate.

As the substrate, for example, a substrate is used which has an oxide including one or more selected from indium, tin, zinc, aluminum, and gallium, a metal (e.g., molybdenum, silver, copper, aluminum, chromium, or titanium), or a CNT (Carbon Nano Tube) formed as an electrode or a wiring on a glass. Examples of the oxide including one or more selected from indium, tin, zinc, aluminum, and gallium include an indium tin oxide (ITO), an indium zinc oxide (IZO), an aluminum zinc oxide (AZO), an indium gallium zinc oxide (IGZO), and a zinc oxide (ZnO).

### <Method for Applying Negative Photosensitive Resin Composition According to Present Invention on Substrate>

Examples of the method for applying the negative photosensitive resin composition according to the present invention on a substrate include microgravure coating, spin coating, dip coating, curtain flow coating, roll coating, spray coating, and slit coating. The coating film thickness varies depending on the coating method, the solid content concentration and viscosity of the resin composition, and the like, and the composition is typically applied such that the film thickness after coating and prebaking is 0.1 to 30 μm.

The negative photosensitive resin composition according to the present invention is preferably applied on a substrate, and then prebaked to form a film. The prebaking can use an oven, a hot plate, infrared rays, a flash annealing device, a laser annealing device, or the like. The prebaking temperature is preferably 50 to 150° C. The prebaking time is preferably 30 seconds to several hours. Prebaking in two or more stages may be performed, such as prebaking at 80° C. for 2 minutes, and then prebaking at 120° C. for 2 minutes.

### <Method for Applying Negative Photosensitive Resin Composition According to Present Invention in Pattern on Substrate>

Examples of the method of applying the negative photosensitive resin composition according to the present invention in a pattern on a substrate include letterpress printing, intaglio printing, stencil printing, planographic printing, screen printing, ink-jet printing, offset printing, and laser printing. The coating film thickness varies depending on the coating method, the solid content concentration and viscosity of the negative photosensitive resin composition according to the present invention, and the like, and the composition is typically applied such that the film thickness after coating and prebaking is 0.1 to 30 μm.

The negative photosensitive resin composition according to the present invention is preferably applied in a pattern on a substrate, and then prebaked to form a film. The prebaking can use an oven, a hot plate, infrared rays, a flash annealing device, a laser annealing device, or the like. The prebaking temperature is preferably 50 to 150° C. The prebaking time is preferably 30 seconds to several hours. Prebaking in two or more stages may be performed, such as prebaking at 80° C. for 2 minutes, and then prebaking at 120° C. for 2 minutes.

### <Method for Pattern Processing of Coating Film Formed on Substrate>

Examples of the method for pattern processing of the coating film of the negative photosensitive resin composition according to the present invention formed on the substrate include a method of direct pattern processing by photolithography and a method of pattern processing by etching. From the viewpoint of improving productivity by reducing the number of steps and reducing the process time, a method of direct pattern processing by photolithography is preferred.

<Step of Irradiation with Active Actinic Ray Through Photomask>

The method for manufacturing a display device with the use of the negative photosensitive resin composition according to the present invention includes the (2) step of irradiating the above-described coating film of the negative photosensitive resin composition with active actinic rays through a photomask. Onto the substrate, the negative photosensitive resin composition according to the present invention is applied and prebaked to form a film, and then exposed with the use of an exposure machine such as a stepper, a mirror projection mask aligner (MPA), or a parallel light mask aligner (PLA). Examples of the active actinic rays in irradiation for the exposure include ultraviolet light, visible light, electron beams, X-rays, KrF (wavelength: 248 nm) lasers, and ArF (wavelength: 193 nm) lasers. It is preferable to use j-lines (wavelength: 313 nm), i-lines (wavelength: 365 nm), h-lines (wavelength: 405 nm), or g-lines (wavelength: 436 nm) from a mercury lamp. In addition, the exposure energy is typically approximately 100 to 40,000 J/m² (10 to 4,000 mJ/cm²) (i-line illuminance meter value), and exposure can be performed through a photomask that has a desired pattern, if necessary.

After the exposure, post-exposure baking may be performed. By performing the post-exposure baking, effects can be expected, such as an improved resolution after development or increased tolerance for development conditions. The post-exposure baking can use an oven, a hot plate, infrared rays, a flash annealing device, a laser annealing device, or the like. The post-exposure baking temperature is preferably 50 to 180° C., more preferably 60 to 150° C. The post-exposure baking time is preferably 10 seconds to several hours. When the post-exposure baking time is 10 seconds to several hours, the reaction may proceed favorably, thereby shortening the development time.

In the method for producing a display device with the use of the negative photosensitive resin composition according to the present invention, it is preferable to use a half-tone photomask as the photomask. The half-tone photomask refers to a photomask that has a pattern including a light-transmitting portion and a light-blocking portion, the photomask having, between the light-transmitting portion and the light-blocking portion, a partial light-transmitting portion that is lower in transmittance than the value of the light-transmitting portion and higher in transmittance than the value of the light-blocking portion. The exposure with the use of the half-tone photomask makes it possible to form a pattern which has a step shape after development and after thermal curing. It is to be noted that the cured part irradiated with active actinic rays through the light-transmitting portion corresponds to the thick film part, whereas the half-tone exposed part irradiated with active actinic rays through the partial light-transmitting portion corresponds to the thin film part.

In the method for manufacturing a display device with the use of the negative photosensitive resin composition according to the present invention, the halftone photomask has a site where the light-transmitting portion and the partial light-transmitting portion are adjacent to each other. Having the site where the light-transmitting portion and the partial light-transmitting portion are adjacent to each other makes it possible to form a pattern including the thick film part corresponding to the light-transmitting portion on the photomask after development and the thin film part corresponding to the partial light-transmitting portion on the photomask. Moreover, the halftone photomask has a site where the light-blocking portion and the partial light-transmitting portion are adjacent to each other. After development, a pattern including an opening corresponding to the light-blocking portion on the photomask and the thin film part corresponding to the partial light-transmitting portion on the photomask can be formed. The halftone photomask has the site mentioned above, thereby making it possible to form a pattern with a step shape, including the thick film part, the thin film part, and the opening, after development.

As the half-tone photomask, in a case where the transmittance of the light-transmitting portion is denoted by (% T_{FT}) %, the transmittance (% T_{HT})% of the partial light-transmitting portion is preferably 10% or more of (% T_{FT}), more preferably 15% or more thereof, further preferably 20% or more thereof, particularly preferably 25% or more thereof. When the transmittance (% T_{HT})% of the partial light-transmitting portion falls within the range mentioned above, the exposure energy at the time of the formation of the cured pattern which has the step shape can be reduced, thus allowing the cycle time to be shortened. On the other hand, the transmittance (% T_{HT})% of the partial light-transmitting portion is preferably 60% or less of (% T_{FT}), more preferably 55% or less thereof, further preferably 50% or less thereof, particularly preferably 45% or less thereof. When the transmittance (% T_{HT})% of the partial light-transmitting portion falls within the range mentioned above, the film thickness difference between the thick film part and the thin film part and the film thickness difference between adjacent thin film parts on both sides of any step can be made sufficiently large, thus allowing degradation of the light-emitting element to be suppressed. In addition, the cured pattern with a step shape has a sufficient film thickness difference by itself, thus making it possible to shorten the process time.

In the cured pattern with a step shape, obtained by irradiation with active actinic rays through the halftone photomask, the film thickness difference (ΔT_{HT30-HT20}) μm between (T_{HT30}) and (T_{HT20}) is preferably 0.3 μm or more, more preferably 0.5 μm or more, still more preferably 0.7 μm or more, particularly preferably 0.8 μm or more, in a case where the film thickness of the thin film part with which the transmittance (% T_{HT})% of the partial light-transmitting portion is 30% of (% T_{FT}) is denoted by (T_{HT30}) μm, whereas the film thickness of the thin film part with which the transmittance (% T_{HT})% of the partial light-transmitting portion is 20% of (% T_{FT}) is denoted by (T_{HT20}) μm. When the film thickness difference falls within the range mentioned above, the film thickness difference between the thick film part and the thin film part and the film thickness difference between adjacent thin film parts on both sides of any step can be made sufficiently large, thus allowing degradation of the light-emitting element to be suppressed. In addition, the cured pattern with a step shape has a sufficient film thickness difference by itself, thus making it possible to shorten the process time. On the other hand, the film thickness difference (ΔT_{HT30-HT20}) μm is preferably 1.5 μm or less, more preferably 1.4 μm or less, still more preferably 1.3 μm or less, particularly preferably 1.2 μm or less. When the film thickness difference falls within the range mentioned above, the variation in film thickness due to a slight fluctuation in exposure energy, caused by the apparatus or the like, can be reduced, thereby improving the film thickness uniformity and the yield in organic EL display manufacture.

<Step of Performing Development with Alkaline Solution to Form Pattern>

The method for manufacturing a display device with the use of the negative photosensitive resin composition according to the present invention includes the (3) step of performing development with the use of an alkaline solution to form a pattern of the negative photosensitive resin composition described above. After the exposure, development is performed with the use of an automatic development device or the like. The negative photosensitive resin composition according to the present invention has negative photosensitivity, and thus, after the development, the unexposed part is removed with a developer, thereby allowing a relief pattern to be obtained.

As the developer, an alkaline developer is typically used. As the alkaline developer, for example, an organic alkaline solution or an aqueous solution of an alkaline compound is preferred, and an aqueous solution of an alkaline compound, that is, an alkaline aqueous solution is more preferred from the viewpoint of the environment aspect.

Examples of the organic alkaline solution or alkaline compound include 2-aminoethanol, 2-(dimethylamino)ethanol, 2-(diethylamino)ethanol, diethanolamine, methylamine, ethylamine, dimethylamine, diethylamine, triethylamine, acetic acid (2-dimethylamino)ethyl, (meth)acrylic acid (2-dimethylamino)ethyl, cyclohexylamine, ethylenediamine, hexamethylenediamine, ammonia, tetramethylammonium hydroxide, tetraethylammonium hydroxide, sodium hydroxide, potassium hydroxide, magnesium hydroxide, calcium hydroxide, barium hydroxide, sodium carbonate, and potassium carbonate, and from the viewpoint of reducing metal impurities in the cured film and suppressing defective display in the display device, tetramethylammonium hydroxide or tetraethylammonium hydroxide is preferred. As the developer, an organic solvent may be used. As the developer, a mixed solution may be used which contains both the organic solvent and a poor solvent with respect to the negative photosensitive resin composition according to the present invention.

Examples of the development method include paddle development, spray development, and dip development. Examples of the paddle development include a method of applying the above-described developer directly to the exposed film, and then leaving the film for an arbitrary period of time, and a method of applying the above-described developer by spraying in the form of a mist to the exposed film for an arbitrary period of time, and then leaving the film for an arbitrary period of time. Examples of the spray development include a method of keeping on spraying the above-described developer in the form of a mist to the exposed film for an arbitrary period of time. Examples of the dip development include a method of immersing the exposed film in the developer described above for an arbitrary period of time, and a method of immersing the exposed film in the developer described above, and then keeping on irradiation with ultrasonic waves for an arbitrary period of time. From the viewpoint of suppressing device contamination during the development and reducing the process cost by reducing the usage of the developer, the paddle development is preferred as the development method. Device contamination during the development is suppressed, thereby allowing substrate contamination during the development to be suppressed, and then allowing defective display in the display device to be suppressed. On the other hand, from the viewpoint of inhibiting the residue generation after development, the spray development is preferred as the development method. In addition, from the viewpoint of reducing the usage of the developer by reuse of the developer and reducing the process cost, the dip development is preferred as the development method.

The development time is preferably 5 seconds or longer, more preferably 10 seconds or longer, still more preferably 30 seconds or longer, particularly preferably 1 minute or longer. When the development time falls within the range described above, the residue generation during the alkali development can be inhibited. On the other hand, from the viewpoint of reducing the takt time, the development time is preferably 30 minutes or shorter, more preferably 15 minutes or shorter, still more preferably 10 minutes or shorter, particularly preferably 5 minutes or shorter.

After the development, the obtained relief pattern is preferably washed with a rinse solution. As the rinse solution, water is preferred in a case where an alkaline aqueous solution is used as the developer. As the rinsing solution, for example, an aqueous solution of an alcohol such as ethanol or isopropyl alcohol, an aqueous solution of an ester such as propylene glycol monomethyl ether acetate, or an aqueous solution of an acidic compound such as carbon dioxide, hydrochloric acid, or acetic acid may be used. As the rinse solution, an organic solvent may be used.

### <Step of Photo-Curing Pattern>

The method for manufacturing a display device with the use of the negative photosensitive resin composition according to the present invention preferably includes the (3) step of performing development with the use of an alkaline solution to form a pattern of the negative photosensitive resin composition, and then further, a step of photo-curing the pattern of the negative photosensitive resin composition.

The step of photo-curing the pattern improves the crosslink density of the pattern, and then reduces the quantity of low-molecular components which cause degassing, thus improving the reliability of the light-emitting element including the pattern of the negative photosensitive resin composition. Furthermore, in a case where the pattern of the negative photosensitive resin composition is a pattern with a step shape, pattern reflow during thermal curing of the pattern can be suppressed, and a pattern that has a step shape with a sufficient film thickness difference between the thick film part and the thin film part can be formed even after thermal curing. In addition, maintaining the reflow property of the film surface during thermal curing improves the flatness, thereby making it possible to suppress the decrease in panel yield. Moreover, in the manufacture of the organic EL display including the pattern of the negative photosensitive resin composition, the area of contact with an evaporation mask in the formation of an organic EL layer can be reduced, thereby allowing the decrease in panel yield due to particle generation to be suppressed, and allowing degradation of the light-emitting element to be suppressed.

As the step of photo-curing the pattern, it is preferable to irradiate the pattern of the negative photosensitive resin composition with active actinic rays. Examples of the method of irradiation with active actinic rays include a method of bleaching exposure with the use of an exposure machine such as a stepper, a scanner, a mirror projection mask aligner (MPA), or a parallel light mask aligner (PLA). Examples of the lamp for use in irradiation with active actinic rays in the step of photo-curing the pattern include an ultra-high pressure mercury lamp, a high-pressure mercury lamp, a low-pressure mercury lamp, a metal halide lamp, a Xe excimer lamp, a KrF excimer lamp, or an ArF excimer lamp.

Examples of the active actinic rays in the step of photo-curing the pattern include ultraviolet light, visible light, electron beams, X-rays, XeF (wavelength: 351 nm) lasers, XeCl (wavelength: 308 nm) lasers, KrF (wavelength: 248 nm) lasers, or ArF (Wavelength: 193 nm) lasers. From the viewpoints of suppressing pattern reflow during thermal curing of the pattern, improving the step film thickness, and suppressing the decrease in panel yield, the j-lines (wavelength: 313 nm), i-lines (wavelength: 365 nm), h-lines (wavelength: 405 nm), or g-lines (wavelength: 436 nm) of a mercury lamp, or a mixed ray of i-line, h-line and g-line is preferred.

The exposure energy of the active actinic rays in the step of photo-curing the pattern is preferably 100 J/m² (10 mJ/cm²) or more in terms of i-line illuminance value. On the other hand, the exposure energy of the active actinic rays is preferably 50,000 J/m² (5,000 mJ/cm) or less in terms of i-line illuminance value. When the exposure energy falls within the range mentioned above, pattern reflow during thermal curing of the pattern of the negative photosensitive resin composition can be suppressed. In addition, the decrease in panel yield can be suppressed.

In a case where the photomask in the step (2) of irradiating the coating film of the negative photosensitive resin composition with the active actinic rays through the photomask is a halftone photomask, the exposure energy ratio (E_{BLEACH})/(E_{EXPO}) is preferably 0.1 or more, more preferably 0.3 or more, still more preferably 0.5 or more, even more preferably 0.7 or more, particularly preferably 1 or more, where the exposure energy of the active actinic rays in the step of photo-curing the pattern is denoted by (E_{BLEACH}) mJ/cm², whereas the exposure energy in the light-transmitting portion of the photomask in the step (2) of irradiation with the active actinic rays through the photomask is denoted by (E_{EXPO}) mJ/cm². When the exposure energy ratio falls within the range mentioned above, pattern reflow during thermal curing of the pattern of the negative photosensitive resin composition can be suppressed. In addition, the decrease in panel yield can be suppressed. Furthermore, from the viewpoint of improving the step film thickness, the exposure energy ratio is preferably 0.5 or more, more preferably 0.7 or more, still more preferably 1 or more. Furthermore, from the viewpoint of improving the yield, the exposure energy ratio is preferably less than 4, more preferably less than 3.5, still more preferably less than 3.

After obtaining the pattern of the negative photosensitive resin composition according to the present invention, middle baking may be performed. Performing middle baking improves the resolution after thermal curing, and allows the pattern shape after thermal curing to be arbitrarily controlled. The middle baking can use an oven, a hot plate, infrared rays, a flash annealing device, a laser annealing device, or the like. The middle baking temperature is preferably 50 to 250° C., more preferably 70 to 220° C. The middle baking time is preferably 10 seconds to several hours. Middle baking in two or more stages may be performed, such as middle baking at 100° C. for 5 minutes, and then middle baking at 150° C. for 5 minutes.

### <Step of Heating Pattern to Obtain Cured Pattern>

The method for manufacturing a display device with the use of the negative photosensitive resin composition according to the present invention includes the (4) step of heating the pattern of the negative photosensitive resin composition described above to obtain a cured pattern of the negative photosensitive resin composition described above. For heating the pattern of the negative photosensitive resin composition according to the present invention, formed on the substrate, an oven, a hot plate, infrared rays, a flash annealing device, a laser annealing device, or the like can be used. The pattern of the negative photosensitive resin composition according to the present invention is cured and then thermally cured, thereby allowing the heat resistance of the cured film to be improved, and allowing a low-taper pattern shape to be obtained.

The temperature for thermosetting is preferably 150° C. or higher, more preferably 200° C. or higher, and further preferably 250° C. or higher. When the thermal curing temperature is 150° C. or higher, the heat resistance of the cured film can be improved, and the pattern shape after the thermal curing can be further reduced in taper. On the other hand, from the viewpoint of shortening the tact time, the thermosetting temperature is preferably 500° C. or lower, more preferably 450° C. or lower, and further preferably 400° C. or lower.

The time for the thermal curing is preferably 1 minute or longer, more preferably 5 minutes or longer, still more preferably 10 minutes or longer, particularly preferably 30 minutes or longer. When the thermal curing time is 1 minute or longer, the pattern shape after the thermal curing can be further reduced in taper. On the other hand, from the viewpoint of reducing the takt time, the time for the thermal curing is preferably 300 minutes or shorter, more preferably 250 minutes or shorter, still more preferably 200 minutes or shorter, particularly preferably 150 minutes or shorter. Thermal curing in two or more stages may be performed, such as thermal curing at 150° C. for 30 minutes, and then thermal curing at 250° C. for 30 minutes.

Further, the negative photosensitive resin composition a to the present invention makes it possible to obtain cured films which are suitably used for applications such as a pixel defining layer, an electrode insulation layer, a wiring insulation layer, an interlayer insulation layer, a TFT planarization layer, an electrode planarization layer, a wiring planarization layer, a TFT protective layer, an electrode protective layer, a wiring protective layer, a gate insulation layer, a color filter, a black matrix, or a black column spacer. Moreover, it becomes possible to obtain an elements and display devices including the cured films. The organic EL display according to the present invention includes the above-mentioned cured film as one or more selected from the group consisting of a pixel defining layer, an electrode insulation layer, a wiring insulation layer, an interlayer insulation layer, a TFT planarization layer, an electrode planarization layer, a wiring planarization layer, a TFT protective layer, an electrode protective layer, a wiring protective layer, a gate insulation layer, a color filter, a black matrix, and a black column spacer. In particular, the negative photosensitive resin composition according to the present invention is excellent in light-blocking property, and thus more preferred as a light-blocking pixel defining layer, electrode insulation layer, wiring insulation layer, interlayer insulation layer, TFT planarization layer, electrode planarization layer, wiring planarization layer, TFT protective layer, electrode protective layer, wiring protective layer, or gate insulating layer of an organic EL display protective layer, and suitable for applications which require contrast increased by suppression of external light reflection, such as a light-blocking pixel defining layer, interlayer insulation layer, TFT planarization layer, or TFT protective layer.

Furthermore, the methods for manufacturing a display device with the use of the negative photosensitive resin composition according to the present invention are capable of achieving high heat-resistance and light-blocking cured films containing polyimide and/or polybenzoxazole, subjected to pattern processing, thus leading to improvements in yield, performance, and reliability in the manufacture of organic EL displays and liquid crystal displays. In addition, since the negative photosensitive resin composition according to the present invention is capable of being directly subjected to pattern processing by photolithography, the number of steps can be reduced as compared with processes with photoresists, thus making it possible to improv the productivity, and reduce the process time and the takt time.

### EXAMPLES

The present invention will be described more specifically with reference to examples and comparative examples, but the present invention is not limited to the scope thereof. It is to be noted that here are names for the abbreviation used for some of the compounds used.

6FDA: 2,2-(3,4-dicarboxyphenyl)hexafluoropropane dianhydride; 4,4′-hexafluoropropane-2,2-diyl-bis(1,2-phthalic anhydride)

A-BPEF: “NK ESTER” (registered trademark) A-BPEF (manufactured by Shin Nakamura Chemical Co., Ltd.; 9,9-bis[4-(2-acryloxyethoxy)phenyl]fluorene)

A-DCP: “NK ESTER” (registered trademark) A-DCP (manufactured by Shin Nakamura Chemical Co., Ltd.; dimethylol-tricyclodecane diacrylate)

A-DPH-6E: “NK ESTER” (registered trademark) A-DPH-6E (manufactured by Shin Nakamura Chemical Co., Ltd.; ethoxylated dipentaerythritol hexaacrylate having 6 oxyethylene structures in the molecule)

APC: Argentum-Palladium-Cupper (silver-palladium-copper alloy)

BAHF: 2,2-bis(3-amino-4-hydroxyphenyl)hexafluoropropane

BFE: 1,2-bis(4-formylphenyl)ethane

BGPF: 9,9-bis(4-glycidoxyphenyl)fluorene

BHPF: 9,9-bis(4-hydroxyphenyl)fluorene

Bis-A-AF: 2,2-bis(4-aminophenyl)hexafluoropropane

Bk-A1103: “CHROMOFINE” (registered trademark) BLACK A1103 (manufactured by Dainichiseika Color & Chemicals Mfg. Co., Ltd.; azo-based black pigment of 50 to 100 nm in primary particle size)

Bk-S0084: “PALIOGEN” (registered trademark) BLACK S0084 (manufactured by BASF; perylene-based black pigment of 50 to 100 nm in primary particle size)

BLACK S0100CF; “IRGAPHOR” (registered trademark) BLACK S0100CF (manufactured by BASF; benzofuranone-based black pigment of 40 to 80 nm in primary particle size)

cyEpoTMS: 2-(3,4-epoxycyclohexyl)ethyltrimethoxysilane

D.BYK-167: “DISPERBYK” (registered trademark) -167 (manufactured by BYK-Chemie Japan; polyurethane-based dispersant having a tertiary amino group with an amine value of 13 mgKOH/g (solid content concentration: 52% by mass))

DFA: N, N-dimethylformamide dimethyl acetal

DPCA-60; “KAYARAD” (registered trademark) DPCA-60 (manufactured by Nippon Kayaku Co., Ltd.; ε-caprolactone-modified dipentaerythritol hexaacrylate having 6 oxypentylene carbonyl structures in the molecule)

DPHA: “KAYARAD” (registered trademark) DPHA (manufactured by Nippon Kayaku Co., Ltd.; dipentaerythritol hexaacrylate)

EGME: Ethylene glycol bis(2-mercaptoethyl) ether

GMA: Glycidyl methacrylate

HABI-102: 2,2′,5-tris(2-chlorophenyl)-4-(3,4-dimethoxyphenyl)-4′,5′-diphenyl-1,2′-biimidazole (manufactured by Tronly; biimidazole-based photo initiator)

HA: N,N′-bis[5,5′-hexafluoropropane-2,2-diyl-bis(2-hydroxyphenyl)]bis(3-aminobenzoic acid amide)

IC-379EG: “IRGACURE” (registered trademark) 379EG (manufactured by BASF; 2-dimethylamino-2-(4-methylbenzyl)-1-(4-morpholinophenyl)-butan-1-one; α-aminoketone-based photo initiator)

IC-819: “IRGACURE” (registered trademark) 819 (manufactured by BASF; bis(2,4,6-trimethylbenzoyl)-phenylphosphine oxide; acylphosphine oxide photo initiator)

IDN-1: 1,1-bis[4-(2-acryloxyethoxy)phenyl]indane

IGZO: Indium gallium zinc oxide

ITO: Indium tin oxide

MAA: Methacrylic acid

MAP: 3-aminophenol; meta amino phenol

MBA: 3-methoxy-n-butyl acetate

MeTMS: Methyltrimethoxysilane

MgAg: Magnesium-Argentum (magnesium-silver alloy)

NA: 5-norbornene-2,3-dicarboxylic anhydride; nadic anhydride

NC-7300L: Epoxy resin having a structural unit including a naphthalene skeleton, a benzene skeleton, and two epoxy groups (manufactured by Nippon Kayaku Co., Ltd.)

NMP: N-methyl-2-pyrrolidone

ODPA: bis(3,4-dicarboxyphenyl)ether dianhydride; oxydiphthalic dianhydride

OXE-02: “IRGACURE” (registered trademark) OXE-02 (manufactured by BASF; 1-[9-ethyl-6-(2-methylbenzoyl)-9H-carbazol-3-yl]ethanone-1-(O-acetyl)oxime; oxime ester-based photo initiator)

P. B. 15: 6; C. I. Pigment Blue 15: 6

P.B.60: C.I. pigment Blue 60

P.O.43: C.I. pigment Orange 43

P.R.179; C.I. pigment Red 179

P. R. 254: C. I. Pigment Red 254

P. V. 23: C. I. Pigment Violet 23

P.V.37: C.I. pigment Violet 37

P. Y. 139: C. I. Pigment Yellow 139

P.Y.192: C.I. pigment Yellow 192

PGMEA: Propylene glycol monomethyl ether acetate

PHA: phthalic anhydride

PhTMS: Phenyltrimethoxysilane

S-20000: “SOLSPERSE” (registered trademark) 20000 (manufactured by Lubrizol; polyoxyalkylene ether-based dispersant having a tertiary amino group with an amine value of 32 mg KOH/g (solid content concentration: 100% by mass))

SiDA: 1,3-bis(3-aminopropyl)tetramethyldisiloxane

STR: Styrene

TCDM: tricyclo[5.2.1.0^{2,6}]decan-8-yl methacrylate; dimethylol-tricyclodecane dimethacrylate

THPHA: 1,2,3,6-tetrahydrophthalic anhydride

TMAH: Tetramethylammonium hydroxide

TMOS: Tetramethoxysilane

TMMP: Trimethylolpropane tris(3-mercaptopropionate)

TPK-1227; carbon black (manufactured by CABOT) surface-treated for introducing a sulfonic acid group.

WR-301: “ADEKA ARKLS” (registered trademark) WR-301 (polycyclic side chain-containing resin obtained by reacting a carboxylic anhydride with the resin obtained by the ring-opening addition reaction of an aromatic compound having an epoxy group and an unsaturated carboxylic acid, acid equivalent: 560, double bond equivalent: 450)

### Synthesis Example (A)

In a three-neck flask, 18.31 g (0.05 mol) of BAHF and 17.42 g (0.3 mol) of propylene oxide were dissolved in 100 mL of acetone weighed. Into this solution, a solution of 20.41 g (0.11 mol) of 3-nitrobenzoyl chloride dissolved in 10 mL of acetone was delivered by drops. After the completion of dropping, the solution was allowed to undergo a reaction for 4 hours at −15° C., and then, the temperature thereof was returned to room temperature. The precipitated white solid matter was filtered off, and subjected to vacuum dry at 50° C. In a 300 mL stainless-steel autoclave, 30 g of the obtained solid was placed, dispersed in 250 mL of 2-methoxyethanol, and 2 g of 5% palladium-carbon was added to the dispersion. Into the dispersion, hydrogen was introduced with a balloon, thereby allowing for a reaction at room temperature for 2 hours. After 2 hours, it was confirmed that the balloon is not squeezed any more. After completion of the reaction, the palladium compound as a catalyst was removed by filtration, and the filtrate was concentrated by distillation under reduced pressure to obtain a hydroxy group-containing diamine compound (HA) having the following structure.

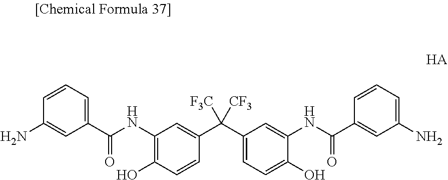 [SMILES: Nc1cccc(C(=O)Nc2cc(C(c3ccc(O)c(NC(=O)c4cccc(N)c4)c3)(C(F)(F)F)C(F)(F)F)ccc2O)c1]

### Synthesis Example 1—Synthesis of Polyimide (PI-1)

Under a dry nitrogen stream, 31.13 g (0.085 mol; 77.3 mol % based on the structural units derived from all of amines and derivatives thereof) of BAHF and 1.24 g (0.0050 mol; 4.5 mol % based on the structural units derived from all of amines and derivatives thereof) of SiDA, 2.18 g (0.020 mol; 18.2 mol % based on the structural units derived from all of amines and derivatives thereof) of MAP as an end-capping agent, and 150.00 g of NMP were weight, and then dissolved in a three-neck flask. To this solution, a solution of 31.02 g (0.10 mol; 100 mol % based on the structural units derived from all of carboxylic acids and derivatives thereof) of ODPA dissolved in 50.00 g of NMP was added, stirred at 20° C. for 1 hour, and then 50° C. for 4 hours. Thereafter, 15 g of xylene was added thereto, and the solution was stirred for 5 hours at 150° C. while azeotroping the water with the xylene. After completion of the reaction, the reaction solution was poured into 3 L of water, and the deposited solid precipitate was obtained by filtration. The obtained solid was washed with water three times, and then dried for 24 hours with a vacuum dryer at 80° C. to obtain a polyimide (PI-1). The Mw of the obtained polyimide was 27,000, and the acid equivalent thereof was 350.

### Synthesis Examples 2-5—Synthesis of Polyimide (PI-2) to Polyimide (PI-5)

With the monomer types and ratios thereof shown in Table 1-1, polymerization was performed in the same manner as in Synthesis Example 1 to obtain a polyimide (PI-2) to a polyimide (PI-5).

### Synthesis Example 6—Synthesis of Polyimide Precursor (PIP-1)

Under a nitrogen stream, 44.42 g (0.10 mol; 100 mol % based on the structural units derived from all of carboxylic acids and derivatives thereof) of 6FDA and 150 g of NMP were weighed, and then dissolved in a three-necked flask. To this solution, a solution of: 14.65 g (0.040 mol; 32.0 mol % based on the structural units derived from all of amines and derivatives thereof) of BAHF; 18.14 g (0.030 mol; 24.0 mol % based on the structural units derived from all of the amines and derivatives thereof) of HA; and 1.24 g (0.0050 mol; 4.0 mol % based on the structural units derived from all of the amines and derivatives thereof) of SiDA dissolved in 50 g of NMP was added, and the solution was stirred at 20° C. for 1 hour, and then 50° C. for 2 hours. Next, a solution of 5.46 g (0.050 mol; 40.0 mol % based on the structural units derived from all of amines and derivatives thereof) of MAP dissolved in 15 g of NMP was added as an end-capping agent, and the solution was stirred at 50° C. for 2 hours. Thereafter, a solution of 23.83 g (0.20 mol) of DFA dissolved in 15 g of NMP was delivered by drops over 10 minutes. After completion of dropping, the solution was stirred at 50° C. for 3 hours. After completion of the reaction, the reaction solution was cooled to room temperature, then, the reaction solution was poured into 3 L of water, and the deposited solid precipitate was obtained by filtration. The obtained solid was washed with water three times, and then dried for 24 hours with a vacuum dryer at 80° C. to obtain a polyimide precursor (PIP-1). The Mw of the obtained polyimide precursor was 20,000, and the acid equivalent thereof was 450.

### Synthesis Example 7—Synthesis of Polyimide Precursor (PIP-2)

With the monomer types and ratios thereof listed in Table 1-1, polymerization was performed in the same manner as in Synthesis Example 6 to obtain a polyimide precursor (PIP-2).

### Synthesis Example 8—Synthesis of Polybenzoxazole (PBO-1)

In a 500 mL round-bottom flask equipped with a Dean-Stark water separator filled with toluene and a cooling tube, 34.79 g (0.095 mol; 95.0 mol % based on the structural units derived from all of amines and derivatives thereof) of BAHF, and 1.24 g (0.0050 mol; 5.0 mol % based on the structural units derived from all of the amines and derivatives thereof) of SiDA, and 75.00 g of NMP were weighed, and then dissolved. To this solution, a solution of 19.06 g (0.080 mol; 66.7 mol % based on the structural units derived from all of carboxylic acids and derivatives thereof) of BFE and 6.57 g (0.040 mol; 33.3 mol % based on the structural units derived from all of the carboxylic acids and derivatives thereof) of NA as an end-capping agent) dissolved in 25.00 g of NMP was added, and the solution was stirred at 20° C. for 1 hour, and then stirred at 50° C. for 1 hour. Thereafter, under a nitrogen atmosphere, the solution was heated and stirred at 200° C. or higher for 10 hours to develop a dehydration reaction. After completion of the reaction, the reaction solution was poured into 3 L of water, and the deposited solid precipitate was obtained by filtration. The obtained solid was washed with water three times, and then dried for 24 hours with a vacuum dryer at 80° C. to obtain a polybenzoxazole (PBO-1). The Mw of the obtained polybenzoxazole was 25,000, and the acid equivalent thereof was 330.

### Synthesis Example 9—Synthesis of Polybenzoxazole Precursor (PBOP-1)

In a 500 mL round-bottom flask equipped with a Dean-Stark water separator filled with toluene and a cooling tube, 34.79 g (0.095 mol; 95.0 mol % based on the structural units derived from all of amines and derivatives thereof) of BAHF, and 1.24 g (0.0050 mol; 5.0 mol % based on the structural units derived from all of the amines and derivatives thereof) of SiDA, and 70.00 g of NMP were weighed, and then dissolved. To this solution, a solution of 19.06 g (0.080 mol; 66.7 mol % based on the structural units derived from all of carboxylic acids and derivatives thereof) of BFE dissolved in 20.00 g of NMP was added, and the solution was stirred at 20° C. for 1 hour, and then stirred at 50° C. for 2 hours. Next, a solution of 6.57 g (0.040 mol; 33.3 mol % based on the structural units derived from all of carboxylic acids and derivatives thereof) of NA dissolved in 10 g of NMP was added as an end-capping agent, and the solution was stirred at 50° C. for 2 hours. Thereafter, the solution was stirred at 100° C. for 2 hours under a nitrogen atmosphere. After completion of the reaction, the reaction solution was poured into 3 L of water, and the deposited solid precipitate was obtained by filtration. The obtained solid was washed with water three times, and then dried for 24 hours with a vacuum dryer at 80° C. to obtain a polybenzoxazole precursor (PBOP-1). The Mw of the obtained polybenzoxazole precursor was 20,000, and the acid equivalent thereof was 330.

### Synthesis Example 10—Synthesis of Polysiloxane Solution (PS-1)

In a three-neck flask, 20.43 g (30 mol %) of MeTMS, 49.57 g (50 mol %) of PhTMS, 12.32 g (10 mol %) of cyEpoTMS, 7.61 g (10 mol %) of TMOS, and 83.39 g of PGMEA were put. Air was allowed to flow through the flask at 0.05 L/min, and the mixed solution was heated to 40° C. in an oil bath while stirring. While further stirring the mixed solution, a phosphoric acid aqueous solution of 0.270 g of phosphoric acid dissolved in 28.83 g of water was delivered by drops over 10 minutes. After completion of the delivery by drops, the solution was stirred at 40° C. for 30 minutes to hydrolyze the silane compound. After completion of the hydrolysis, after stirring for 1 hour at the bath temperature adjusted to 70° C., the bath temperature was then raised to 115° C. After the start of the temperature raise, the internal temperature of the solution reached 100° C. after about 1 hour, and the solution was then heated and stirred for 2 hours (internal temperature from 100 to 110° C.). The resin solution obtained by heating and stirring for 2 hours was cooled in an ice bath to obtain a polysiloxane solution (PS-1). The Mw of the obtained polysiloxane was 4,500.

### Synthesis Example 11—Synthesis of Polysiloxane Solution (PS-2)

In a three-neck flask, 27.24 g (40 mol %) of MeTMS, 49.57 g (50 mol %) of PhTMS, 12.32 g (10 mol %) of cyEpoTMS, and 83.91 g of PGMEA were put. Air was allowed to flow through the flask at 0.05 L/min, and the mixed solution was heated to 40° C. in an oil bath while stirring. While further stirring the mixed solution, a phosphoric acid aqueous solution of 0.267 g of phosphoric acid dissolved in 27.93 g of water was added over 10 minutes. After completion of the addition, the solution was stirred at 40° C. for 30 minutes to hydrolyze the silane compound. After completion of the hydrolysis, after stirring for 1 hour at the bath temperature adjusted to 70° C., the bath temperature was then raised to 115° C. After the start of the temperature raise, the internal temperature of the solution reached 100° C. after about 1 hour, and the solution was then heated and stirred for 2 hours (internal temperature from 100 to 110° C.). The resin solution obtained by heating and stirring for 2 hours was cooled in an ice bath to obtain a polysiloxane solution (PS-2). The Mw of the obtained polysiloxane was 4,000.

### Synthesis Example 12 Synthesis of Polycyclic Side Chain-Containing Resin Solution (CR-1)

In a three-neck flask, 35.04 g (0.10 mol) of BHPF was dissolved in 40.31 g of MBA weighed. To this solution, a solution of 27.92 g (0.090 mol) of ODPA and 2.96 g (0.020 mol) of PHA as an end-capping agent dissolved in 30.00 g of MBA was added, and the solution was stirred at 20° C. for 1 hour. Thereafter, the solution was stirred at 150° C. for 5 hours under a nitrogen atmosphere. After completion of the reaction, to the obtained solution, a solution of 14.22 g (0.10 mol) of GMA, 0.135 g (0.0010 mol) of dibenzylamine, and 0.037 g (0.0003 mol) of 4-methoxyphenol dissolved in 10.00 g of MBA was added, and the solution was stirred at 90° C. for 4 hours to obtain a polycyclic side chain-containing resin solution (CR-1). The Mw of the obtained polycyclic side chain-containing resin was 4,000, the carboxylic acid equivalent thereof was 810 g/mol, and the double bond equivalent was 810 g/mol.

### Synthesis Example 13—Synthesis of Polycyclic Side Chain-Containing Resin Solution (CR-2)

In a three-neck flask, 46.25 g (0.10 mol) of BGPF was dissolved in 54.53 g of MBA weighed. To this solution, a solution of 17.22 g (0.20 mol) of MAA, 0.135 g (0.0010 mol) of dibenzylamine, and 0.03 7 g (0.0003 mol) of 4-methoxyphenol dissolved in 10.00 g of MBA was added, and the solution was stirred at 90° C. for 4 hours. Thereafter, a solution of 27.92 g (0.090 mol) of ODPA and 2.96 g (0.020 mol) of PHA as an end-capping agent dissolved in 30.00 g of MBA was added, and the solution was stirred at 20° C. for 1 hour. Thereafter, under a nitrogen atmosphere, the solution was stirred at 150° C. for 5 hours to obtain a polycyclic side chain-containing resin solution (CR-2). The Mw of the obtained polycyclic side chain-containing resin was 4,700, the carboxylic acid equivalent thereof was 470 g/mol, and the double bond equivalent was 470 g/mol.

### Synthesis Example 14—Synthesis of Acid-Modified Epoxy Resin Solution (AE-1)

In a three-neck flask, 42.00 g of NC-7300L (epoxy equivalent: 210 g/mol) was dissolved in 47.91 g of MBA weighed. To this solution, a solution of 17.22 g (0.20 mol) of MAA, 0.270 g (0.0020 mol) of dibenzylamine, and 0.074 g (0.0006 mol) of 4-methoxyphenol dissolved in 10.00 g of MBA was added, and the solution was stirred at 90° C. for 4 hours. Thereafter, a solution of 24.34 g (0.160 mol) of THPHA dissolved in 30.00 g of MBA was added, and the solution was stirred at 20° C. for 1 hour. Thereafter, under a nitrogen atmosphere, the solution was stirred at 150° C. for 5 hours to obtain an acid-modified epoxy resin solution (AE-1). The Mw of the obtained acid-modified epoxy resin was 5,000, the acid equivalent thereof was 510 g/mol, and the double bond equivalent was 410 g/mol.

### Synthesis Example 15—Synthesis of Acrylic Resin Solution (AC-1)

In a three-neck flask, 0.821 g (1 mol %) of 2,2′-azobis(isobutyronitrile) and 29.29 g of PGMEA were put. Next, 21.52 g (50 mol %) of MAA, 22.03 g (20 mol %) of TCDM, 15.62 g (30 mol) of STR were put, the mixture was stirred at room temperature for a while, and after sufficiently purging the inside of the flask with nitrogen by bubbling, stirred at 70° C. for 5 hours. Next, to the obtained solution, a solution of 59.47 g of PGMEA, 14.22 g (20 mol %) of GMA, 0.676 g (1 mol %) of dibenzylamine, and 0.186 g (0.3 mol %) of 4-methoxyphenol dissolved was added, and the solution was stirred at 90° C. for 4 hours to obtain an acrylic resin solution (AC-1). The Mw of the obtained acrylic resin was 15,000, the carboxylic acid equivalent thereof was 490 g/mol, and the double bond equivalent was 740 g/mol.

The compositions of Synthesis Examples 1 to 15 described above are collectively shown in Table 1-1 to Table 1-3.

TABLE 1-1

| | | | | | | | | | | Structural | |
|---|---|---|---|---|---|---|---|---|---|---|---|
| | | | | | | | | | Structural | Unit derived | |
| | | | | | | | | | Unit derived | from Monomer | |
| | | | | | | | | | from Monomer | having | |
| | | | | | | | | Structural | having | Fluorine Atom | |
| | | | | | | | | Unit derived | Fluorine Atom | to Structural | |
| | | | | | | | | from Monomer | to Structural | Units derived | |
| | | | | | | | | having | Units derived | from structural | |
| | | Monomer [mol ratio] | | | | | | Fluorine Atom | from All of | units derived | |
| | | Tetracarboxylic | | | | | | to All of | Carboxylic | from All of | |
| | | Acid and | | Diamine and | | | End- | Structural | Acid | Amine | Acid |
| | | Derivatives | | Derivatives | | | capping | Units | Derivatives | Derivatives | Equivalent |
| | Polymer | thereof | | thereof | | | Agent | [mol %] | [mol %] | [mol %] | [g/mol] |
| Synthesis | Polyimide | ODPA | — | BAKF | — | SiDA | MAP | 40.5 | 0.0 | 77.3 | 350 |
| Example 1 | (PI-1) | (100) | | (85) | | (5) | (20) | | | | |
| Synthesis | Polyimide | ODPA | — | BAKF | Bis-A-AF | SiDA | MAP | 16.7 | 0.0 | 31.8 | 720 |
| Example 2 | (PI-2) | (100) | | (35) | (50) | (5) | (20) | | | | |
| Synthesis | Polyimide | ODPA | 6FDA | BAKF | — | SiDA | MAP | 59.5 | 40.0 | 77.3 | 380 |
| Example 3 | (PI-3) | (60) | (40) | (85) | | (5) | (20) | | | | |
| Synthesis | Polyimide | — | 6FDA | BAKF | — | SiDA | MAP | 88.1 | 100.0 | 77.3 | 420 |
| Example 4 | (PI-4) | | (100) | (85) | | (5) | (20) | | | | |
| Synthesis | Polyimide | ODPA | — | — | BAPF | SiDA | MAP | 0.0 | 0.0 | 0.0 | 360 |
| Example 5 | (PI-5) | (100) | | | (85) | (5) | (20) | | | | |
| Synthesis | Polyimide | — | 6FDA | BAKF | HA | SiDA | MAP | 75.6 | 100.0 | 56.0 | 450 |
| Example 6 | Precursor | | (100) | (40) | (30) | (5) | (50) | | | | |
| | (PIP-1) | | | | | | | | | | |
| Synthesis | Polyimide | ODPA | 6FDA | BAKF | HA | SiDA | MAP | 57.8 | 60.0 | 56.0 | 420 |
| Example 7 | Precursor | (40) | (60) | (40) | (30) | (5) | (50) | | | | |
| | (PIP-2) | | | | | | | | | | |

TABLE 1-2

| | | Monomer [mol ratio] | | | |
|---|---|---|---|---|---|
| | | Dicarboxylic | | | |
| | | Acid and | Bisaminophenol | | |
| | | Derivatives | Compound and | | |
| | | thereof | Derivatives | | |
| | | Diformyl | thereof | | |
| | | Compound and | Dihydroxydiamine and | | End- |
| | | Derivatives | Derivative | | capping |
| | Polymer | thereof | thereof | | Agent |
| Synthesis | Polybenzoxazole | BFE | BAHF | SiDA | NA |
| Example 8 | (PBO-1) | (80) | (95) | (5) | (40) |
| Synthesis | Polybenzoxazole | BFE | BAHF | SiDA | NA |
| Example 9 | (PBOP-1) | (80) | (95) | (5) | (40) |

| | | | Structural | | |
|---|---|---|---|---|---|
| | | Structural | Unit derived | | |
| | | Unit derived | from Monomer | | |
| | | from Monomer | having | | |
| | Structural | having | Fluorine Atom | | |
| | Unit derived | Fluorine Atom | to Structural | | |
| | from Monomer | to Structural | Units derived | | |
| | having | Units derived | from structural | | |
| | Fluorine Atom | from All of | units derived | | |
| | to All of | Carboxylic | from All of | | Double |
| | Structural | Acid | Amine | Acid | Bond |
| | Units | Derivatives | Derivatives | Equivalent | Equivalent |
| | [mol %] | [mol %] | [mol %] | [g/mol] | [g/mol] |
| Synthesis | 43.2 | 0.0 | 95.0 | 330 | — |
| Example 8 | | | | | |
| Synthesis | 43.2 | 0.0 | 95.0 | 330 | — |
| Example 9 | | | | | |

| | | | | | | | | Structural | | |
|---|---|---|---|---|---|---|---|---|---|---|
| | | | | | | | | Unit derived | | |
| | | Monomer [mol %] | | | | | | from Organosilane | | |
| | | | | | | Tetrafunctional | | having | | |
| | | | | | | Organosilane | Bifunctional | Aromatic | | Double |
| | | | | | | Tetrafunctional | Organosilane | Group to | Acid | Bond |
| | | | | | | Organosilane | Monofunctional | Polysiloxane | Equivalent | Equivalent |
| | Polymer | Trifunctional Organosilane | | | | Oligomer | Organosilane | [mol %] | [g/mol] | [g/mol] |
| Synthesis | Polysiloxane | MeTMS | PhTMS | cyEpoTMS | — | TMOS | — | 50.0 | — | — |
| Example 10 | Solution | (30) | (50) | (10) | | (10) | | | | |
| | (PS-1) | | | | | | | | | |
| Synthesis | Polysiloxane | MeTMS | PhTMS | cyEpoTMS | — | — | — | 50.0 | — | — |
| Example 11 | Solution | (40) | (50) | (10) | | | | | | |
| | (PS-2) | | | | | | | | | |

TABLE 1-3

| | | Monomer [mol ratio] | | | |
|---|---|---|---|---|---|
| | | Compound | Compound | | |
| | | having Two | having Two | | |
| | | or More | or More | | |
| | | Aromatic | Aromatic | | |
| | | Groups | Groups | Tetracarboxylic | |
| | | and | and | Dianhydride | End- |
| | | Hydroxy | Epoxy | Tetracarboxylic | capping |
| | Polymer | Group | Group | Acid | Agent |
| Synthesis | Polycyclic Side | SHPF | — | ODPA | PHA |
| Example 12 | Chain-containing | (100) | | (90) | (20) |
| | Resin Solution | | | | |
| | (CR-1) | | | | |
| Synthesis | Polycyclic Side | — | BGPF | ODPA | PHA |
| Example 13 | Chain-containing | | (100) | (90) | (20) |
| | Resin Solution | | | | |
| | (CR-2) | | | | |

| | | | Structural | | |
|---|---|---|---|---|---|
| | | | Unit derived | | |
| | | | from Monomer | | |
| | | | having | | |
| | Monomer [mol ratio] | | Aromatic | | |
| | Unsaturated | | Group to | | |
| | Compound | | Structural | | |
| | having | Unsaturated | Units derived | | |
| | Ethylenically | Carboxylic | from | | |
| | Unsaturated | Acid | structural | | |
| | Double | having | units derived | | |
| | Bond | Ethylenically | from All of | | |
| | Group | Unsaturated | Carboxylic | | Double |
| | and | Double | Acid | Acid | Bond |
| | Epoxy | Bond | Derivatives | Equivalent | Equivalent |
| | Group | Group | [mol %] | [g/mol] | [g/mol] |
| Synthesis | GMA | — | 100.0 | 810 | 810 |
| Example 12 | (100) | | | | |
| Synthesis | — | MAA | 100.0 | 470 | 470 |
| Example 13 | | (200) | | | |

| | | | | | Structural | | |
|---|---|---|---|---|---|---|---|
| | | | | | Unit derived | | |
| | | Monomer [mol ratio] | | | from Monomer | | |
| | | | | Unsaturated | having | | |
| | | | | Carboxylic | Aromatic Group to | | |
| | | Compound | | Acid | Structural Units | | |
| | | having | | having | derived from | | |
| | | Aromatic | | Ethylenically | structural units | | |
| | | Group | Dicarboxylic | Unsaturated | derived from All | | Double |
| | | and | Anhydride | Double | of Carboxylic | Acid | Bond |
| | | Epoxy | Dicarboxylic | Bond | Acid Derivatives | Equivalent | Equivalent |
| | Polymer | Group | Acid | Group | [mol %] | [g/mol] | [g/mol] |
| Synthesis | Acid-modified | NC-7300L | THPBA | MAA | 0.0 | 510 | 410 |
| Example 14 | Epoxy Resin | (Epoxy Equivalent: | 24.34 g | 17.22 g | | | |
| | Solution | 210 g/mol) | (0.16 mol) | (0.20 mol) | | | |
| | (AE-1) | 42.00 g | (mol ratio: 80) | (mol ratio: 100) | | | |
| | | (Epoxy Group Standard: | | | | | |
| | | 0.2 mol) | | | | | |
| | | (Epoxy Group Standard | | | | | |
| | | mol ratio: 100) | | | | | |

| | | | | | | Structural | | |
|---|---|---|---|---|---|---|---|---|
| | | | | | | Unit derived | | |
| | | | | | | from Monomer | | |
| | | | | | | having | | |
| | | Monomer [mol ratio] | | | | Aromatic | | |
| | | | | | Unsaturated | Group to | | |
| | | | | | Compound | Structural | | |
| | | | | | having | Units derived | | |
| | | | | | Ethylenically | from | | |
| | | | | | Unsaturated | structural | | |
| | | Copolymerization | Copolymerization | Copolymerization | Double Bond | units derived | | |
| | | Component | Component | Component | Group | from All of | | Double |
| | | having | having | having | and | Copolymerization | Acid | Bond |
| | | Acidic | Aromatic | Alicyclic | Epoxy | Components | Equivalent | Equivalent |
| | Polymer | Group | Group | Group | Group | [mol %] | [g/mol] | [g/mol] |
| Synthesis | Acrylic Resin | MAA | STR | TCDM | GMA | 30.0 | 490 | 740 |
| Example 15 | Solution | (50) | (30) | (20) | (20) | | | |
| | (AC-1) | | | | | | | |

### Covering Example 1—Synthesis of Surface-Covered Benzofuranone-Based Black Pigment (Bk-CBF1)

As a black pigment, 150 g of benzofuranone-based black pigment Bk-S0100CF (surface-untreated product; pH 4.5 at the pigment surface) was put into a glass container containing 2,850 g of deionized water, and stirred with a dissolver, thereby providing an aqueous pigment suspension. This suspension was sacked up with a tube pump, fed into a horizontal bead mill filled with 0.4 mmφ zirconia beads (“TORAYCERAM” (registered trademark); manufactured by Toray Industries, Inc.) and subjected to a 2-pass dispersion treatment therein, then entirely discharged into the original glass container, and stirred again with a dissolver. With a pH meter such that the electrode tip was immersed at a depth of 3 to 5 cm from the liquid surface of the aqueous pigment suspension being stirred in the glass container, the pH of the obtained aqueous pigment suspension was measured, and then, the pH meter read pH 4.5 (liquid temperature: 25° C.). Thereafter, the temperature of the aqueous pigment suspension was raised to 60° C. while stirring, and the stirring was temporarily stopped after 30 minutes, then after 2 minutes, it was confirmed that there was no sediment deposited on the bottom of the glass container, and stirring was restarted.

To the aqueous pigment suspension, a sodium silicate aqueous solution (Na₂O-nSiO₂-mH₂O; 30% by mass as sodium oxide, 10% by mass as silicon dioxide) diluted 1/100 with deionized water and a 0.001 mol/L sulfuric acid were added in parallel while adjusting the respective addition rates so as to maintain the pH in the range of 2 or more and less than 7, such that the covering amount of silica was 10.0 parts by mass in terms of SiO₂ with respect to 100 parts by mass of the black pigment, thereby depositing silica on the particle surface of the black pigment to cover the surface. Then, to the aqueous pigment suspension, a sodium aluminate aqueous solution (Na₂O-nAl₂O₃-mH₂O; 40% by mass as sodium oxide, 50% by mass as alumina) diluted 1/100 with deionized water and a 0.001 mol/L sulfuric acid were added in parallel while adjusting the respective addition rates so as to maintain the pH in the range of 2 or more and less than 7, such that the covering amount of alumina was 2.0 parts by mass in terms of Al₂O₃ with respect to 100 parts by mass of the black pigment, thereby depositing alumina on the surface of the silica covering layer to cover the surface. Subsequently, filtration and water washing operations were repeated three times to remove some of water-soluble impurities in the aqueous pigment suspension, and the suspension was fed into a horizontal bead mill filled with 0.4 mmφ zirconia beads, and subjected to a 1-pass dispersion treatment therein. Furthermore, in order to remove ionic impurities, 10 g of a cation exchange resin and 10 g of an anion exchange resin (Amberlite; manufactured by ORGANO CORPORATION) were put in the aqueous pigment suspension, then stirred for 12 hours, and filtered to obtain a black filter cake. This filter cake was dried in a drying oven at 90° C. for 6 hours and in a drying oven at 200° C. for 30 minutes, and then subjected to granulation by dry grinding with the use of a jet mill, thereby providing a surface-covered benzofuranone-based black pigment (Bk-CBF1).

As a result of time-of-flight secondary ion mass spectrometry and X-ray diffraction analysis, the silica and alumina covering amounts of the surface-covered benzofuranone-based black pigment (Bk-CBF1) obtained were respectively 10.0 parts by mass in terms of SiO₂ and 2.0 parts by mass in terms of Al₂θ₃ with respect to 100 parts by mass of the black pigment, and the average coverage of the covering layer with respect to the pigment was 97.5%.

### Preparation Example 1—Preparation of Pigment Dispersion (Bk-1)

After 34.5 g of S-20000 as a dispersant, 782.0 g of MBA as a solvent were weighed, and mixed, and diffused by stirring for 10 minutes, 103.5 g of Bk-S0100CF as a colorant was weighed, and then mixed with the dispersant and the solvent, and stirred for 30 minutes, and subjected to a wet media dispersion treatment with the use of a horizontal bead mill filled with 0.40 mmφ zirconia beads such that the number average particle size was 100 nm, thereby providing a pigment dispersion (Bk-1) with a solid content concentration 15% by mass, and colorant/dispersant=75/25 (mass ratio). The number average particle size of the pigment in the obtained pigment dispersion was 100 nm.

### Preparation Example 2—Preparation of Pigment Dispersion (Bk-2)

After 92.0 g of a 30% by mass MBA solution of the polyimide (PI-1) obtained in accordance with Synthesis Example 1 as a resin, 27.6 g of S-20000 as a dispersant, 717.6 g of MBA as a solvent were weighed, and mixed, and diffused by stirring for 10 minutes, 82.8 g of Bk-S0100CF as a colorant was weighed, and then mixed with the dispersant and the solvent, and stirred for 30 minutes, and subjected to a wet media dispersion treatment with the use of a horizontal bead mill filled with 0.40 mmφ zirconia beads such that the number average particle size was 100 nm, thereby providing a pigment dispersion (Bk-2) with a solid content concentration 15% by mass, and colorant/resin/dispersant=60/20/20 (mass ratio). The number average particle size of the pigment in the obtained pigment dispersion was 100 run.

### Preparation Examples 3 to 18—Preparation of Pigment Dispersion (Bk-3) to Pigment Dispersion (Bk-18)

With the types and ratios of the colorant, (A1) first resin, and (E) dispersant, listed in Table 2-1, pigments were dispersed in the same manner as in Preparation Example 2, thereby providing a pigment dispersion (Bk-3) to a pigment dispersion (Bk-18).

The compositions according to Preparation Examples 1 to 18 are collectively shown in Table 2-1.

TABLE 2-1

| | | | | | | | Number |
|---|---|---|---|---|---|---|---|
| | | | | | | | Average |
| | | | | | | | Particle |
| | | | | | | | Size of |
| | | | | | | | Pigment in |
| | | Composition (% by mass) | | | | | Pigment |
| | | | | | (A1) | (E) | Dispersion |
| | Dispersion | Colorant | | | First Resin | Dispersant | [nm] |
| Preparation | Pigment | Bk-S0100CF | — | — | — | S-20000 | 100 |
| Example 1 | Dispersion | (75) | | | | (25) | |
| | (Bk-1) | | | | | | |
| Preparation | Pigment | Bk-S0100CF | — | — | Polyimide | S-20000 | 100 |
| Example 2 | Dispersion | (60) | | | (PI-1) | (20) | |
| | (Bk-2) | | | | (20) | | |
| Preparation | Pigment | Bk-S0100CF | — | — | Polyimide | S-20000 | 120 |
| Example 3 | Dispersion | (65) | | | (PI-1) | (10) | |
| | (Bk-3) | | | | (25) | | |
| Preparation | Pigment | Bk-S0084 | — | — | Polyimide | D.BYK-167 | 120 |
| Example 4 | Dispersion | (60) | | | (PI-1) | (20) | |
| | (Bk-4) | | | | (20) | | |
| Preparation | Pigment | Bk-A1103 | — | — | Polyimide | D.BYK-167 | 120 |
| Example 5 | Dispersion | (60) | | | (PI-1) | (20) | |
| | (Bk-5) | | | | (20) | | |
| Preparation | Pigment | TPK-1227 | — | — | Polyimide | D.BYK-167 | 120 |
| Example 6 | Dispersion | (60) | | | (PI-1) | (20) | |
| | (Bk-6) | | | | (20) | | |
| Preparation | Pigment | P.R.254 | P.Y.139 | P.B.15:6 | Polyimide | D.BYK-167 | 110 |
| Example 7 | Dispersion | (21) | (9) | (30) | (PI-1) | (20) | |
| | (Bk-7) | | | | (20) | | |
| Preparation | Pigment | P.V.23 | P.Y.139 | — | Polyimide | D.BYK-167 | 110 |
| Example 8 | Dispersion | (36) | (24) | | (PI-1) | (20) | |
| | (Bk-8) | | | | (20) | | |
| Preparation | Pigment | P.R.179 | P.Y.192 | P.B.60 | Polyimide | D.BYK-167 | 110 |
| Example 9 | Dispersion | (18) | (18) | (24) | (PI-1) | (20) | |
| | (Bk-9) | | | | (20) | | |
| Preparation | Pigment | P.V.37 | P.Y.192 | — | Polyimide | D.BYK-167 | 110 |
| Example 10 | Dispersion | (39) | (21) | | (PI-1) | (20) | |
| | (Bk-10) | | | | (20) | | |
| Preparation | Pigment | P.R.179 | P.O.43 | P.B.60 | Polyimide | D.BYK-167 | 110 |
| Example 11 | Dispersion | (18) | (18) | (24) | (PI-1) | (20) | |
| | (Bk-11) | | | | (20) | | |
| Preparation | Pigment | P.V.37 | P.O.43 | P.B.60 | Polyimide | D.BYK-167 | 110 |
| Example 12 | Dispersion | (33) | (18) | (9) | (PI-1) | (20) | |
| | (Bk-12) | | | | (20) | | |
| Preparation | Pigment | Bk-CBF | — | — | Polyimide | S-20000 | 100 |
| Example 13 | Dispersion | (60) | | | (PI-1) | (20) | |
| | (Bk-13) | | | | (20) | | |
| Preparation | Pigment | P.B.60 | — | — | Polyimide | S-20000 | 100 |
| Example 14 | Dispersion | (60) | | | (PI-1) | (20) | |
| | (Bk-14) | | | | (20) | | |
| Preparation | Pigment | P.R.179 | — | — | Polyimide | S-20000 | 100 |
| Example 15 | Dispersion | (60) | | | (PI-1) | (20) | |
| | (Bk-15) | | | | (20) | | |
| Preparation | Pigment | P.V.37 | — | — | Polyimide | S-20000 | 100 |
| Example 16 | Dispersion | (60) | | | (PI-1) | (20) | |
| | (Bk-16) | | | | (20) | | |
| Preparation | Pigment | P.Y.192 | — | — | Polyimide | S-20000 | 100 |
| Example 17 | Dispersion | (60) | | | (PI-1) | (20) | |
| | (Bk-17) | | | | (20) | | |
| Preparation | Pigment | P.O.43 | — | — | Polyimide | S-20000 | 100 |
| Example 18 | Dispersion | (60) | | | (PI-1) | (20) | |
| | (Bk-18) | | | | (20) | | |

It is to be noted that the pigment the respective maximum transmission wavelengths are shown below for the colorant Bk-S0100CF included in the pigment dispersions (Bk-1) to (Bk-3), the Bk-S0084 included in the pigment dispersion (Bk-4), and the colorants (a mixture of P.R.179, P.Y.192, and P.B.60) included in the pigment dispersion (Bk-9) as the (Da) black colorant.

Bk-S0100CF: 340 nm

Bk-S0084: 350 nm

Mixture of P.R.179, P.Y.192, and P.B.60: 390 nm

Table 2-2 and Table 2-3 collectively show a list of: the (C1-1) specific oxime ester-based photo initiators used in the respective examples and comparative examples; and the oxime ester-based photo initiators (OXL-A and OXE-02) used in the comparative examples, and the physical property values thereof.

TABLE 2-2

| | | Physical Property Value of Oxime Ester-based Photo Initiator | | | | | | |
|---|---|---|---|---|---|---|---|---|
| | | Groups | | | | | | |
| | | represented | | | | | | |
| | | by General | | | | | | |
| | | Formulas | | | | | | |
| | | (12) to (13) | | | | | | |
| | | Mother | | | | | | |
| | | Skeleton | | | | | | |
| | | to which | | Groups | | | | |
| | | Group | | represented | | | | |
| | Oxime | having | | by General | | | | |
| | Ester- | Oxime | Substitution | Formulas | | | | Absorbance |
| | based | Ester | Position of | (15) to (18) | Halogen- | | Maximum | (360 nm) |
| | Photo | Structure | Oxime Ester | or Nitro | substituted | Alkenyl | Absorption | 0.01 g/L |
| | Initiator | is Bonded | Structure | Group | Group | Group | Wavelength | in PGMEA |
| 1 | OXL-1 | General Formula (12) | α-oxime | General Formula (15) | — | — | 347 nm | 0.40 |
| | | Fluorene Structure | | Napthalenecarbonyl | | | | |
| | | | | Structure | | | | |
| 2 | OXL-43 | General Formula (12) | α-oxime | Nitro Group | — | — | 347 nm | 0.40 |
| | | Fluorene Structure | | | | | | |
| 3 | OXL-44 | General Formula (12) | α-oxime | Nitro Group | Tetrafluoroproxy | — | 349 nm | 0.41 |
| | | Fluorene Structure | | | Group | | | |
| 4 | OXL-2 | General Formula (12) | α-oxime | General Formula (15) | — | — | 340 nm | 0.26 |
| | | Carbazole Structure | | Napthalenecarbonyl | | | | |
| | | | | Structure | | | | |
| 5 | OXL-58 | General Formula (12) | α-oxime | Nitro Group | — | — | 340 nm | 0.26 |
| | | Carbazole Structure | | | | | | |
| 6 | OXL-63 | General Formula (12) | α-oxime | Nitro Group | Tetrafluoroproxy | — | 342 nm | 0.27 |
| | | Carbazole Structure | | | Group | | | |
| 7 | OXL-7 | General Formula (13) | α-oxime | General Formula (15) | — | — | 356 nm | 0.27 |
| | | Carbazole Napthalene | | Napthalencarbonyl | | | | |
| | | Structure | | Structure | | | | |
| 8 | OXL-54 | General Formula (13) | α-oxime | Nitro Group | — | — | 356 nm | 0.27 |
| | | Carbazole Napthalene | | | | | | |
| | | Structure | | | | | | |
| 9 | OXL-55 | General Formula (13) | α-oxime | Nitro Group | Tetrafluoroproxy | — | 358 nm | 0.28 |
| | | Carbazole Napthalene | | | Group | | | |
| | | Structure | | | | | | |
| 10 | OXL-16 | General Formula (13) | α-oxime | General Formula (16) | — | — | 358 nm | 0.27 |
| | | Carbazole Napthalene | | Trimethylbenzoyl | | | | |
| | | Structure | | Structure | | | | |
| 11 | OXL-19 | General Formula (13) | α-oxime | General Formula (16) | — | — | 358 nm | 0.28 |
| | | Carbazole Napthalene | | Trimethylbenzoyl | | | | |
| | | Structure | | Structure | | | | |
| 12 | OXL-21 | General Formula (13) | α-oxime | General Formula (16) | Tetrafluoroproxy | — | 358 nm | 0.28 |
| | | Carbazole Napthalene | | Trimethylbenzoyl | Group | | | |
| | | Structure | | Structure | | | | |
| 13 | OXL-9 | General Formula (14) | α-oxime | General Formula (15) | — | — | 332 nm | 0.20 |
| | | Diphenyl Sulfide Structure | | Napthalenecarbonyl | | | | |
| | | | | Structure | | | | |
| 14 | OXL-49 | General Formula (14) | α-oxime | Nitro Group | — | — | 332 nm | 0.20 |
| | | Diphenyl Sulfide Structure | | | | | | |
| 15 | OXL-31 | General Formula (13) | α-oxime | General Formula (17) | — | — | 356 nm | 0.27 |
| | | Carbazole Naphthalene | | Thiophenecarbonyl | | | | |
| | | Structure | | Structure | | | | |

TABLE 2-3

| | | Physical Property Value of Oxime Ester-based Photo Initiator | | | | | | |
|---|---|---|---|---|---|---|---|---|
| | | Groups | | | | | | |
| | | represented | | | | | | |
| | | by General | | | | | | |
| | | Formulas | | | | | | |
| | | (12) to (13) | | | | | | |
| | | Mother | | | | | | |
| | | Skeleton | | | | | | |
| | | to which | | Groups | | | | |
| | | Group | | represented | | | | |
| | Oxime | having | | by General | | | | |
| | Ester- | Oxime | Substitution | Formulas | | | | Absorbance |
| | based | Ester | Position of | (15) to (18) | Halogen- | | Maximum | (360 nm) |
| | Photo | Structure | Oxime Ester | or Nitro | substituted | Alkenyl | Absorption | 0.01 g/L |
| | Initiator | is Bonded | Structure | Group | Group | Group | Wavelength | in PGMEA |
| 16 | OXL-37 | General Formula (13) | α-oxime | General Formula (18) | — | — | 356 nm | 0.27 |
| | | Carbazole Naphthalene | | Furancarbonyl | | | | |
| | | Structure | | Structure | | | | |
| 17 | OXL-71 | General Formula (14) | α-oxime | General Formula (18) | — | — | 330 nm | 0.20 |
| | | Diphenyl Sulfide | | Furancarbonyl | | | | |
| | | Structure | | Structure | | | | |
| 18 | OXL-79 | General Formula (12) | β-oxime | Nitro Group | — | — | 327 nm | 0.17 |
| | | Fluorene Structure | | | | | | |
| 19 | OXL-81 | General Formula (14) | β-oxime | Nitro Group | — | — | 325 nm | 0.15 |
| | | Diphenyl Sulfide Structure | | | | | | |
| 20 | OXL-82 | General Formula (12) | α-oxime | Nitro Group | — | — | 345 nm | 0.40 |
| | | Fluorene Structure | | | | | | |
| 21 | OXL-83 | General Formula (12) | α-oxime | General Formula (15) | — | — | 345 nm | 0.40 |
| | | Fluorene Structure | | Naphthalenecarbonyl | | | | |
| | | | | Structure | | | | |
| 22 | OXL-87 | General Formula (12) | α-oxime | Nitro Group | — | Allyl Group | 345 nm | 0.50 |
| | | Fluorene Structure | | | | | | |
| 23 | OXL-96 | General Formula (12) | α-oxime | Nitro Group | — | 3-methyl-2- | 345 nm | 0.50 |
| | | Fluorene Structure | | | | butenyl Group | | |
| 24 | OXL-97 | General Formula (13) | α-oxime | Nitro Group | — | Allyl Group | 360 nm | 0.30 |
| | | Carbazole Naphthalene | | | | | | |
| | | Structure | | | | | | |
| 25 | OXL-99 | General Formula (13) | α-oxime | Nitro Group | — | 3-mehtyl-2- | 360 nm | 0.30 |
| | | Carbazole Naphthalene | | | | butenyl Group | | |
| | | Structure | | | | | | |
| 26 | OXL-73 | General Formula (12) | α-oxime | Nitro Group | — | — | 345 nm | 0.48 |
| | | Fluorene Structure | | | | | | |
| 27 | OXL-100 | General Formula (12) | α-oxime | General Formula (15) | — | — | 340 nm | 0.26 |
| | | Carbazole Structure | | Naphthalenecarbonyl | | | | |
| | | | | Structure | | | | |
| 28 | OXL-101 | General Formula (12) | α-oxime | General Formula (17) | — | — | 340 nm | 0.26 |
| | | Carbazole Structure | | Thiophenocarbonyl | | | | |
| | | | | Structure | | | | |
| 29 | OXL-A | General Formula (12) | α-oxime | Nitro Group | — | — | 373 nm | 0.24 |
| | | Carbazole Structure | | | | | | |
| 30 | OXL-02 | General Formula (12) | α-oxime | — | — | — | 337 nm | 0.15 |
| | | Carbazole Structure | | | | | | |

Furthermore, here are the respective structural formulas of: the (C1-1) specific oxime ester-based photo initiators; and the oxime ester-based photo initiators (OXL-A and OXE-02) used in the comparative examples.

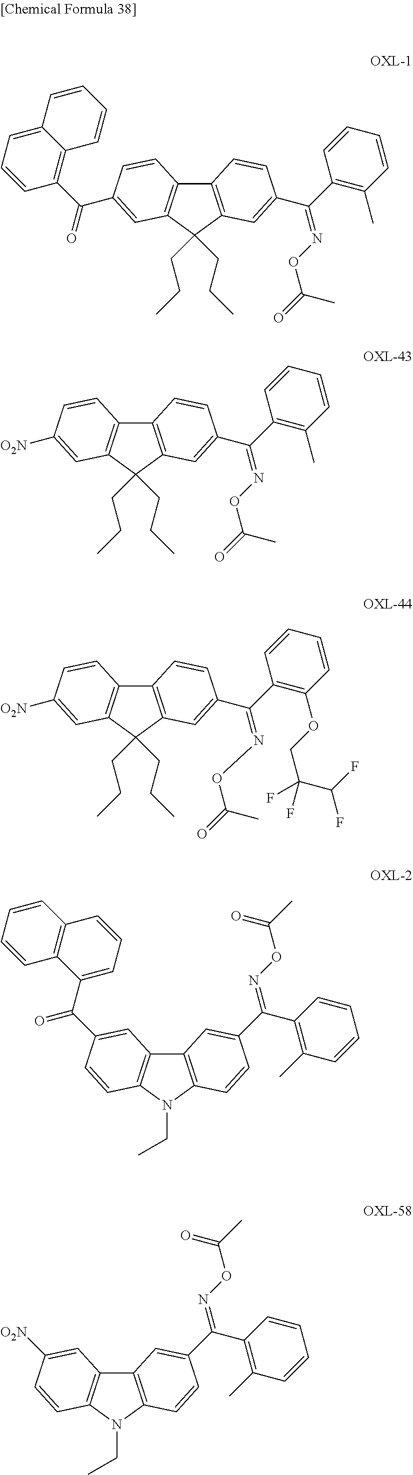 [SMILES: CCCC1(CCC)c2cc(C(=O)c3cccc4ccccc34)ccc2-c2ccc(/C(=N\OC(C)=O)c3ccccc3C)cc21 ; CCCC1(CCC)c2cc(/C(=N\OC(C)=O)c3ccccc3C)ccc2-c2ccc([N+](=O)[O-])cc21 ; CCCC1(CCC)c2cc(/C(=N\OC(C)=O)c3ccccc3OCC(F)(F)C(F)F)ccc2-c2ccc([N+](=O)[O-])cc21 ; CCn1c2ccc(C(=O)c3cccc4ccccc34)cc2c2cc(/C(=N/OC(C)=O)c3ccccc3C)ccc21 ; CCn1c2ccc(/C(=N/OC(C)=O)c3ccccc3C)cc2c2cc([N+](=O)[O-])ccc21]

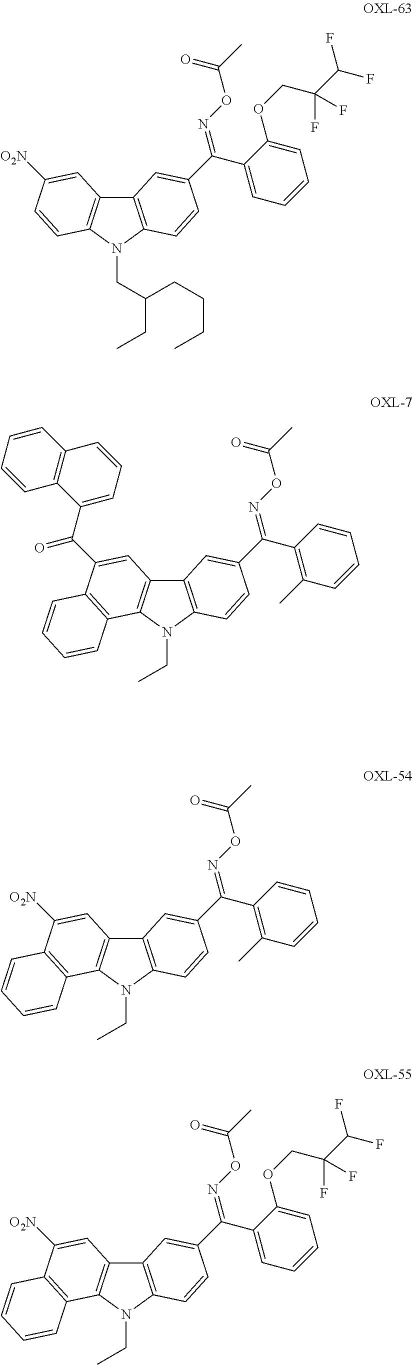 [SMILES: CCCCC(CC)Cn1c2ccc(/C(=N/OC(C)=O)c3ccccc3OCC(F)(F)C(F)F)cc2c2cc([N+](=O)[O-])ccc21 ; CCn1c2ccc(/C(=N/OC(C)=O)c3ccccc3C)cc2c2cc(C(=O)c3cccc4ccccc34)c3ccccc3c21 ; CCn1c2ccc(/C(=N/OC(C)=O)c3ccccc3C)cc2c2cc([N+](=O)[O-])c3ccccc3c21 ; CCn1c2ccc(/C(=N/OC(C)=O)c3ccccc3OCC(F)(F)C(F)F)cc2c2cc([N+](=O)[O-])c3ccccc3c21]

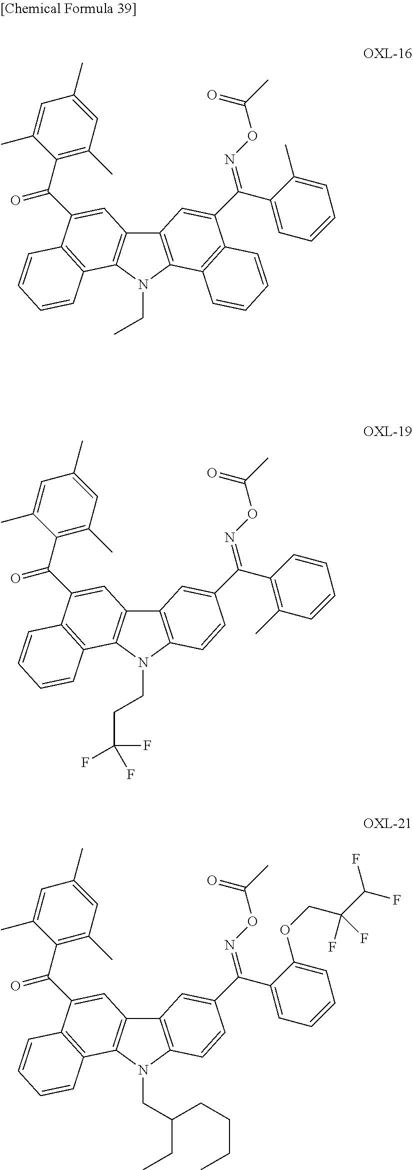 [SMILES: CCn1c2c3ccccc3c(C(=O)c3c(C)cc(C)cc3C)cc2c2cc(/C(=N/OC(C)=O)c3ccccc3C)c3ccccc3c21 ; CC(=O)O/N=C(/c1ccc2c(c1)c1cc(C(=O)c3c(C)cc(C)cc3C)c3ccccc3c1n2CCC(F)(F)F)c1ccccc1C ; CCCCC(CC)Cn1c2ccc(/C(=N/OC(C)=O)c3ccccc3OCC(F)(F)C(F)F)cc2c2cc(C(=O)c3c(C)cc(C)cc3C)c3ccccc3c21]

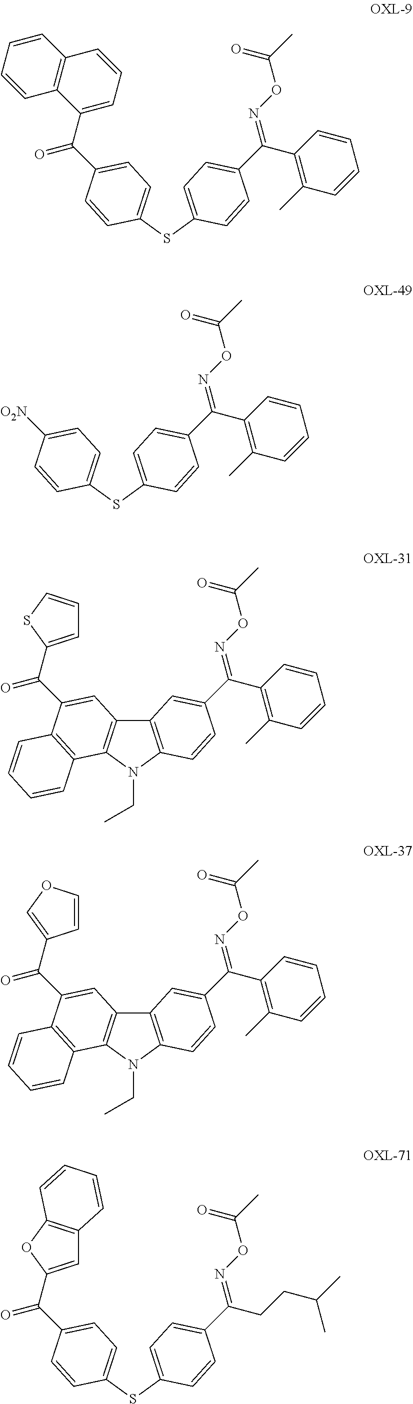 [SMILES: CC(=O)O/N=C(/c1ccc(Sc2ccc(C(=O)c3cccc4ccccc34)cc2)cc1)c1ccccc1C ; CC(=O)O/N=C(/c1ccc(Sc2ccc([N+](=O)[O-])cc2)cc1)c1ccccc1C ; CCn1c2ccc(/C(=N/OC(C)=O)c3ccccc3C)cc2c2cc(C(=O)c3cccs3)c3ccccc3c21 ; CCn1c2ccc(/C(=N/OC(C)=O)c3ccccc3C)cc2c2cc(C(=O)c3ccoc3)c3ccccc3c21 ; CC(=O)O/N=C(\CCC(C)C)c1ccc(Sc2ccc(C(=O)c3cc4ccccc4o3)cc2)cc1]

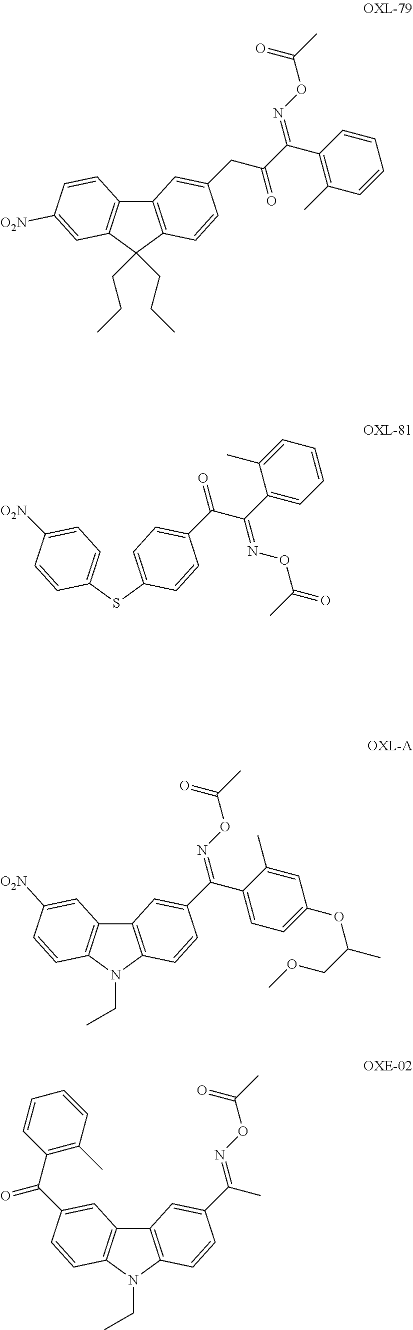 [SMILES: CCCC1(CCC)c2ccc(CC(=O)/C(=N/OC(C)=O)c3ccccc3C)cc2-c2ccc([N+](=O)[O-])cc21 ; CC(=O)O/N=C(/C(=O)c1ccc(Sc2ccc([N+](=O)[O-])cc2)cc1)c1ccccc1C ; CCn1c2ccc(/C(=N/OC(C)=O)c3ccc(OC(C)COC)cc3C)cc2c2cc([N+](=O)[O-])ccc21 ; CCn1c2ccc(C(=O)c3ccccc3C)cc2c2cc(/C(C)=N/OC(C)=O)ccc21]

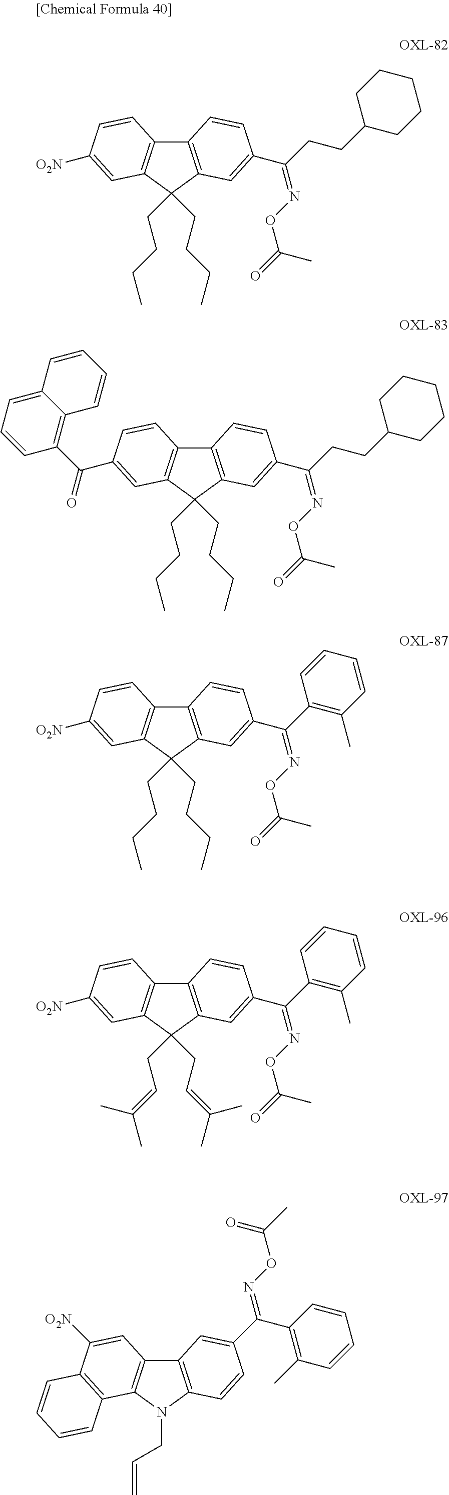 [SMILES: CCCCC1(CCCC)c2cc(/C(CCC3CCCCC3)=N\OC(C)=O)ccc2-c2ccc([N+](=O)[O-])cc21 ; CCCCC1(CCCC)c2cc(C(=O)c3cccc4ccccc34)ccc2-c2ccc(/C(CCC3CCCCC3)=N\OC(C)=O)cc21 ; CCCCC1(CCCC)c2cc(/C(=N\OC(C)=O)c3ccccc3C)ccc2-c2ccc([N+](=O)[O-])cc21 ; CC(=O)O/N=C(\c1ccc2c(c1)C(CC=C(C)C)(CC=C(C)C)c1cc([N+](=O)[O-])ccc1-2)c1ccccc1C ; C=CCn1c2ccc(/C(=N/OC(C)=O)c3ccccc3C)cc2c2cc([N+](=O)[O-])c3ccccc3c21]

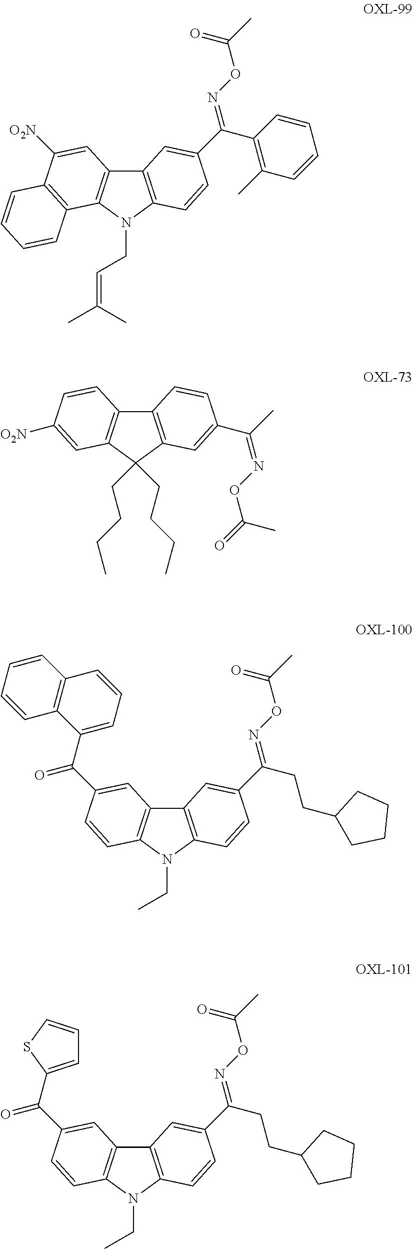 [SMILES: CC(=O)O/N=C(/c1ccc2c(c1)c1cc([N+](=O)[O-])c3ccccc3c1n2CC=C(C)C)c1ccccc1C ; CCCCC1(CCCC)c2cc(/C(C)=N\OC(C)=O)ccc2-c2ccc([N+](=O)[O-])cc21 ; CCn1c2ccc(C(=O)c3cccc4ccccc34)cc2c2cc(/C(CCC3CCCC3)=N/OC(C)=O)ccc21 ; CCn1c2ccc(C(=O)c3cccs3)cc2c2cc(/C(CCC3CCCC3)=N/OC(C)=O)ccc21]

Here is the structural unit of the acid-modified epoxy resin (AE-1) obtained in accordance with Synthesis Example 14. The acid-modified epoxy resin (AE-1) has a structural unit represented by general formula (38a).

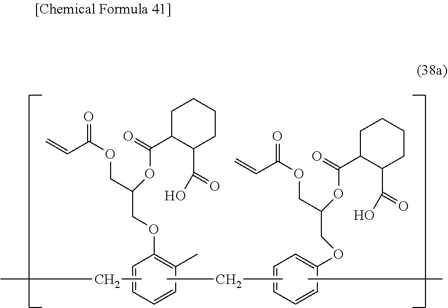

Here are evaluation methods in the respective examples and comparative examples.

### (1) Weight Average Molecular Weight of Resin

With the use of a GPC analyzer (HLC-8220; manufactured by Tosoh Corporation), and with the use of tetrahydrofuran or NMP as a fluidized bed, the weight average molecular weight in terms of polystyrene was measured and then determined by a method at around normal temperature, based on “JIS K7252-3 (2008)”.

### (2) Acid Value, Acid Equivalent

With the use of an automatic potentiometric titrator (AT-510; manufactured by KYOTO ELECTRONICS MANUFACTURING CO., LTD.), and with the use of a 0.1 mol/L sodium hydroxide/ethanol solution as a titration reagent, xylene/N,N-dimethylformamide=1/1 (mass ratio) as a titration solvent, the acid value (unit: mgKOH/g) was measured, and then determined by a potentiometric titration method, based on “JIS K2501 (2003)”. The acid equivalent (unit: g/mol) was calculated from the measured acid value.

### (3) Double Bond Equivalent

With the use of an automatic potentiometric titrator (AT-510; manufactured by KYOTO ELECTRONICS MANUFACTURING CO., LTD.), and with the use of an iodine monochloride solution (mixed solution of iodine trichloride=7.9 g, iodine=8.9 g, acetic acid=1,000 mL) as an iodine source, a 100 g/L potassium iodide aqueous solution as an aqueous solution for trapping unreacted iodine, and a 0.1 mol/L sodium thiosulfate aqueous solution as a titration reagent, the iodine value of the resin was measured by the Wiis method, based on the method described in the “Section 6: Iodine Value” of JIS K0070: 1992 “Method for Testing Acid Value, Saponification Value, Ester Value, Iodine Value, Hydroxyl Value, and Unsaponifiable Matter of Chemical Product”. The double bond equivalent (unit: g/mol) was calculated from the measured iodine value (unit: gl/100 g).

### (4) Content Ratio of Each Organosilane Unit in Polysiloxane

The measurement of ²⁹Si-NMR was performed for calculating the ratio of the integration value of Si derived from a specific organosilane unit to the integration value of the entire Si derived from organosilane, and the content ratios thereof were calculated. The sample (liquid) was injected into an NMR sample tube made of “Teflon” (registered trademark) of 10 mm in diameter, and used for the measurement. Here are the ²⁹Si-NMR measurement conditions.

Apparatus: nuclear magnetic resonance apparatus (JNM-GX270; manufactured by JEOL Ltd.)

Measurement method: gated decoupling method

Measurement nucleus frequency: 53.6693 MHz (²⁹Si nucleus)

Spectrum width: 20000 Hz

Pulse width: 12 μs (45° pulse)

Pulse repetition time: 30.0 seconds

Solvent: acetone-d6

Reference material: Tetramethylsilane

Measurement temperature: 23° C.

Sample rotation speed: 0.0 Hz.

(5) Number Average Particle Size of Pigment With the use of a zeta potential/particle size/molecular weight measuring device (Zetasizer Nano ZS; manufactured by SYSMEX CORPORATION), and with the use of PGMEA as a diluent solvent, the pigment dispersion was diluted to a concentration of 1.0×10⁵ to 40% by volume, the refractive index of the solvent was set to the refractive index of PGMEA, whereas the refractive index of an object to be measured was set to 1.6, and the object was irradiated with laser light with a wavelength of 633 nm to measure the number average particle size of the pigment in the pigment dispersion.

### (6) Pretreatment for Substrate

The glass substrate with an ITO of 100 nm formed by sputtering on glass (GEOMATEC Co., Ltd.; hereinafter, referred to as an “ITO substrate′”) was subjected to a UV—O₃ cleaning treatment for 100 seconds with the use of a tabletop optical surface treatment device (PL16-110; manufactured by SEN LIGHTS Corporation), and then used. The Si wafer (manufactured by ELECTRONICS AND MATERIALS CORPORATION LIMITED) was dehydrated and baked by heating at 130° C. for 2 minutes with the use of a hot plate (HP-1SA; manufactured by AS ONE Corporation), and used.

### (7) Film Thickness Measurement

With the use of a surface texture and contour measuring instrument (SURFCOM 1400D; manufactured by TOKYO SEIMITSU CO., LTD.), at the measurement magnification of 10,000 times, the measurement length of 1.0 mm, the measurement speed of 0.30 mm/s, the film thickness was measured after prebaking, after development, and after curing.

### (8) Sensitivity

In accordance with the method described in Example 1 below, after exposure for patterning with the i-line (wavelength: 365 nm), h-line (wavelength: 405 nm), and g-line (wavelength 436 nm) of an ultra-high pressure mercury lamp through a gray scale mask (MDRM MODEL 4000-5-FS; Opto-Line International, Inc.) for sensitivity measurement with the use of a double-sided alignment single-sided exposure apparatus (Mask Aligner PEM-6M; manufactured by Union Optical Co., Ltd.), development was performed with the use of a small-size development device (AD-2000; TAKIZAWA SANGYO K.K.) for photolithography, thereby preparing a developed film of the negative photosensitive resin composition.

With the use of a FPD/LSI inspection microscope (OPTIPHOT-300; manufactured by NIKON CORPORATION), the resolution pattern of the developed film prepared was observed, and the exposure energy (i-line illuminance meter value) for the formation of the 20 μm line-and-space pattern with a one-to-one width was regarded as the sensitivity. It has been determined as follows that the A+, A, B, and C with the sensitivity of 90 mJ/cm² or less are regarded as pass, the A+, A, and B with the sensitivity of 60 mJ/cm² or less are regarded as favorable sensitivities, and the A+ and A with the sensitivity of 45 mJ/cm² or less are regarded as excellent sensitivities.

A+: The sensitivity is 1 to 30 mJ/cm²

A: The sensitivity is 31 to 45 mJ/cm²

B: The sensitivity is 46 to 60 mJ/cm²

C: The sensitivity is 61 to 90 mJ/cm²

D: The sensitivity is 91 to 150 mJ/cm²

E: The sensitivity is 151 to 500 mJ/cm².

### (9) Development Residue

In accordance with the method described in Example 1 below, after exposure for patterning with the i-line (wavelength: 365 nm), h-line (wavelength: 405 nm), and g-line (wavelength 436 nm) of an ultra-high pressure mercury lamp through a gray scale mask (MDRM MODEL 4000-5-FS; Opto-Line International, Inc.) for sensitivity measurement with the use of a double-sided alignment single-sided exposure apparatus (Mask Aligner PEM-6M; manufactured by Union Optical Co., Ltd.), development was performed with the use of a small-size development device (AD-2000; TAKIZAWA SANGYO K.K.) for photolithography, and a developed film of the negative photosensitive resin composition was then prepared with the use of a high-temperature inert gas oven (INH-9CD-S; manufactured by Koyo Thermo Systems Co., Ltd.).

With the use of a FPD/LSI inspection microscope (OPTIPHOT-300; manufactured by NIKON CORPORATION), the resolution pattern of the cured film prepared was observed to observe the presence or presence of any residue derived from the pigment in the opening of the 20 μm line-and-space pattern. It has been determined as follows that the A+, A, and B where the presence area of the residue in the opening is 10% or lower are regarded as pass, the A+ and A where the presence area of the residue in the opening is 5% or lower are regarded as favorable development residues, and the A+ without the presence area of the residue in the opening is regarded as an excellent development residue.

A+: No residue in the opening

A: The presence area of the residue in the opening is 1 to 5%

B: The presence area of the residue in the opening is 6 to 10%

C: The presence area of the residue in the opening is 11 to 30%

D: The presence area of the residue in the opening is 31 to 50%

E: The presence area of the residue in the opening is 51 to 100%.

(10) Pattern Cross-Section Shape after Development

In accordance with the method described in Example 1 below, after exposure for patterning with the i-line (wavelength: 365 nm), h-line (wavelength: 405 nm), and g-line (wavelength 436 nm) of an ultra-high pressure mercury lamp through a gray scale mask (MDRM MODEL 4000-5-FS; Opto-Line International, Inc.) for sensitivity measurement with the use of a double-sided alignment single-sided exposure apparatus (Mask Aligner PEM-6M; manufactured by Union Optical Co., Ltd.), development was performed with the use of a small-size development device (AD-2000; TAKIZAWA SANGYO K.K.) for photolithography, thereby preparing a developed film of the negative photosensitive resin composition.

With the use of a field-emission scanning electron microscope (S-4800; manufactured by Hitachi High-Technologies Corporation), of the resolution pattern of the developed film prepared, the cross section of the line-and-space pattern with a space width of 20 μm was observed, and the taper angle of the cross section was measured. It has been determined as follows that the A+, A, and B where the taper angle of the cross section is 600 or less are regarded as pass, the A+ and A where the taper angle of the cross section is 45° or less are regarded as favorable pattern shapes, and the A+ where the taper angle of the cross section is 300 or less is regarded as an excellent pattern shape.

A+: The taper angle of the cross section is 1° to 30°.

A: The taper angle of the cross section is 31° to 45°.

B: The taper angle of the cross section is 46° to 60°.

C: The taper angle of the cross section is 61° to 70°.

D: The taper angle of the cross section is 71° to 80°.

E: The taper angle of the cross section is 81° to 179°.

(11) Pattern Cross-Section Shape after Thermal Curing In accordance with the method described in Example 1 below, after exposure for patterning with the i-line (wavelength: 365 nm), h-line (wavelength: 405 nm), and g-line (wavelength 436 nm) of an ultra-high pressure mercury lamp through a gray scale mask (MDRM MODEL 4000-5-FS; Opto-Line International, Inc.) for sensitivity measurement with the use of a double-sided alignment single-sided exposure apparatus (Mask Aligner PEM-6M; manufactured by Union Optical Co., Ltd.), development was performed with the use of a small-size development device (AD-2000; TAKIZAWA SANGYO K.K.) for photolithography, and a cured film of the negative photosensitive resin composition was then prepared with the use of a high-temperature inert gas oven (INH-9CD-S; manufactured by Koyo Thermo Systems Co., Ltd.).

With the use of a field-emission scanning electron microscope (S-4800; manufactured by Hitachi High-Technologies Corporation), of the resolution pattern of the cured film prepared, the cross section of the line-and-space pattern with a space width of 20 μm was observed, and the taper angle of the cross section was measured. It has been determined as follows that the A+, A, and B where the taper angle of the cross section is 60° or less are regarded as pass, the A+ and A where the taper angle of the cross section is 45° or less are regarded as favorable pattern shapes, and the A+ where the taper angle of the cross section is 30° or less is regarded as an excellent pattern shape.

A+: The taper angle of the cross section is 1° to 30°.

A: The taper angle of the cross section is 31° to 45°.

B: The taper angle of the cross section is 46° to 60°.

C: The taper angle of the cross section is 61° to 70°.

D: The taper angle of the cross section is 71° to 80°.

E: The taper angle of the cross section is 81° to 179°.

(12) Change in Pattern Opening Width Between Before and after Thermal Curing

In accordance with the method described in Example 1 below, after exposure for patterning with the i-line (wavelength: 365 nm), h-line (wavelength: 405 nm), and g-line (wavelength 436 nm) of an ultra-high pressure mercury lamp through a gray scale mask (MDRM MODEL 4000-5-FS; Opto-Line International, Inc.) for sensitivity measurement with the use of a double-sided alignment single-sided exposure apparatus (Mask Aligner PEM-6M; manufactured by Union Optical Co., Ltd.), development was performed with the use of a small-size development device (AD-2000; TAKIZAWA SANGYO K.K.) for photolithography, thereby preparing a developed film of the negative photosensitive resin composition.

With the use of a FPD/LSI inspection microscope (OPTIPHOT-300; manufactured by NIKON CORPORATION), the resolution pattern of the developed film prepared was observed, and the opening width of the 20 μm line-and-space pattern was measured, and regarded as a developed pattern opening width (CD_{DEV}).

Thereafter, the developed film described above was thermally cured with the use of a high-temperature inert gas oven (INH-9CD-S; manufactured by Koyo Thermo Systems Co., Ltd.) in accordance with the method described in Example 1 below, thereby preparing a cured film of the negative photosensitive resin composition.

With the use of a FPD/LSI inspection microscope (OPTIPHOT-300; manufactured by NIKON CORPORATION), the resolution pattern of the cured film prepared was observed, and the opening width of the 20 μm line-and-space pattern was measured at the same site as observed after the development, and regarded as a thermally cured pattern opening width (CD_{CURE}).

From the developed pattern opening width and the thermal cured pattern opening width, the change in pattern opening width between before and after thermal curing ((CD_{DEV})−(CD_{CURE})) was calculated. It has been determined as follows that the A+, A, and B where the change in pattern opening width between before and after thermal curing is 0.60 μm or less are regarded as pass, the A+ and A where the change in pattern opening width between before and after thermal curing is 0.40 μm or less are regarded as favorable changes in pattern opening width, and the A+ where the change in pattern opening width between before and after thermal curing is 0.20 μm or less is regarded as an excellent change in pattern opening width.

A+: The change in pattern opening width between before and after thermal curing is 0 to 0.20 μm.

A: The change in pattern opening width between before and after thermal curing is 0.21 to 0.40 μm.

B: The change in pattern opening width between before and after thermal curing is 0.41 to 0.60 μm.

C: The change in pattern opening width between before and after thermal curing is 0.61 to 1.00 μm.

D: The change in pattern opening width between before and after thermal curing is 1.01 to 2.00 μm.

E: The change in pattern opening width between before and after thermal curing is 2.01 μm or more.

### (13) Heat Resistance (Difference in High-Temperature Weight Residual Ratio)

In accordance with the method described in Example 1 below, after exposure for patterning with the i-line (wavelength: 365 nm), h-line (wavelength: 405 nm), and g-line (wavelength 436 nm) of an ultra-high pressure mercury lamp through a gray scale mask (MDRM MODEL 4000-5-FS; Opto-Line International, Inc.) for sensitivity measurement with the use of a double-sided alignment single-sided exposure apparatus (Mask Aligner PEM-6M; manufactured by Union Optical Co., Ltd.), development was performed with the use of a small-size development device (AD-2000; TAKIZAWA SANGYO K.K.) for photolithography, and a cured film of the negative photosensitive resin composition was then prepared with the use of a high-temperature inert gas oven (INH-9CD-S; manufactured by Koyo Thermo Systems Co., Ltd.).

After the thermal curing, the cured film prepared was scraped from the substrate, and about 10 mg of the film was put in an aluminum cell. This aluminum cell was, with the use of a thermogravimetric measurement device (TGA-50; manufactured by Shimadzu Corporation), held at 30° C. for 10 minutes in a nitrogen atmosphere, then heated to 150° C. at a temperature increase rate of 10° C./min, and thereafter, held at 150° C. for 30 minutes, and furthermore, a thermogravimetric analysis was carried out while increasing the temperature up to 500° C. at a temperature increase rate of 10° C./min. With respect to 100% by mass of the weight after heating at 150° C. for 30 minutes, the weight residual ratio at 350° C. in the case of further heating is denoted by (Mₐ)%. by mass, and the weight residual ratio at 400° C. is denoted by (M_{b})% by mass, and the difference ((Mₐ)−(M_{b})) in high-temperature weight residual ratio was calculated as a heat resistance index.

It has been determined as follows that the A+, A, and B where the difference in high-temperature weight residual ratio is 25.0% by mass or lower are regarded as pass, A+ and A where the difference in high-temperature weight residual ratio is 15.0% or lower are regarded as favorable heat resistance, and A+ where the difference in high-temperature weight residual ratio is 5.0% or lower is regarded as excellent heat resistance.

A+: The difference in high-temperature weight residual ratio is 0 to 5.0%.

A: The difference in high-temperature weight residual ratio is 5.1 to 15.0%.

B: The difference in high-temperature weight residual ratio is 15.1 to 25.0%.

C: The difference in high-temperature weight residual ratio is 25.1 to 35.0%.

D: The difference in high-temperature weight residual ratio is 35.1 to 45.04.

E: The difference in high-temperature weight residual ratio is 45.1 to 100%.

### (14) Light-Blocking (Optical Density (Hereinafter, Referred to as an “OD”) Value)

In accordance with the method described in Example 1 below, after exposure for patterning with the i-line (wavelength: 365 nm), h-line (wavelength: 405 nm), and g-line (wavelength 436 nm) of an ultra-high pressure mercury lamp through a gray scale mask (MDRM MODEL 4000-5-FS; Opto-Line International, Inc.) for sensitivity measurement with the use of a double-sided alignment single-sided exposure apparatus (Mask Aligner PEM-6M; manufactured by Union Optical Co., Ltd.), development was performed with the use of a small-size development device (AD-2000; TAKIZAWA SANGYO K.K.) for photolithography, and a cured film of the negative photosensitive resin composition was then prepared with the use of a high-temperature inert gas oven (INH-9CD-S; manufactured by Koyo Thermo Systems Co., Ltd.).

With the use of a transmission densitometer (X-Rite 361T (V); manufactured by X-Rite), the incident light intensity (I₀) and transmitted light intensity (I) of the cured film prepared were measured. The OD value was calculated by the following formula as a light-blocking index.

OD Value=log₁₀(I₀/I).

### (15) Insulation Properties (Surface Resistivity)

In accordance with the method described in Example 1 below, after exposure for patterning with the i-line (wavelength: 365 nm), h-line (wavelength: 405 nm), and g-line (wavelength 436 nm) of an ultra-high pressure mercury lamp through a gray scale mask (MDRM MODEL 4000-5-FS; Opto-Line International, Inc.) for sensitivity measurement with the use of a double-sided alignment single-sided exposure apparatus (Mask Aligner PEM-6M; manufactured by Union Optical Co., Ltd.), development was performed with the use of a small-size development device (AD-2000; TAKIZAWA SANGYO K.K.) for photolithography, and a cured film of the negative photosensitive resin composition was then prepared with the use of a high-temperature inert gas oven (INH-9CD-S; manufactured by Koyo Thermo Systems Co., Ltd.). The surface resistivity (0/0) of the cured film prepared was measured with the use of a high resistivity meter (“HIRESTA” UP; manufactured by Mitsubishi Chemical Corporation).

### (16) Light-Emitting Characteristics of Organic EL Display (Method for Manufacturing Organic EL Display)

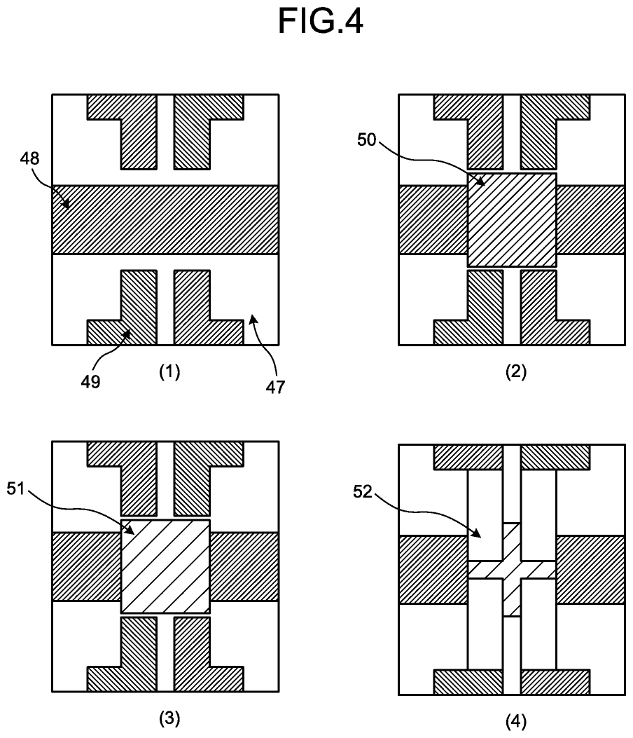

FIG. 4 shows a schematic diagram of the substrate used. First, ITO transparent conductive coatings of 10 nm were formed on the entire surface of a 38×46 mm non-alkali glass substrate 47 by sputtering, and etched as a first electrode 48 to form a transparent electrode. In addition, an auxiliary electrode 49 was formed at the same time to take out a second electrode (FIG. 4 (Step 1)). The obtained substrate was subjected to ultrasonic cleaning for 10 minutes with “Semico Clean” (registered trademark) 56 (manufactured by Furuuchi Chemical Corporation), and washed with ultrapure water. Next, the negative photosensitive resin composition was applied and prebaked on the substrate by the method described in Example 1, subjected to patterning exposure through a photomask with a predetermined pattern, and development and rinsing, and then thermally cured by heating. In accordance with the method mentioned above, openings of 70 μm in width and 260 μm in length were arranged at a pitch of 155 μm in the width direction and a pitch of 465 μm in the length direction, and an insulation layer 50 in a shape for exposing the first electrode through the respective openings was formed only on a substrate effective area in a limited fashion (FIG. 4 (Step 2)). It is to be noted that the openings will finally serve for light-emitting pixels of an organic EL display. Further, the substrate effective area was a square of 16 mm on a side, and the insulation layer 50 was formed to have a thickness of about 1.0 μm.

Next, an organic EL display was manufactured with the use of the substrate with the first electrode 48, auxiliary electrode 49, and insulation layer 50 formed. After the substrate was subjected to a nitrogen plasma treatment as a pretreatment, an organic EL layer 51 including a light-emitting layer was formed by vacuum deposition (FIG. 4 (Step 3)). It is to be noted that the degree of vacuum for the deposition was 1×10⁻³ Pa or less, and during the deposition, the substrate was rotated with respect to a deposition source. First, a compound (HT-1) of 10 nm and a compound (HT-2) of 50 nm were deposited respectively as a hole injection layer and a hole transport layer. Next, for the light-emitting layer, a compound (GH-1) as a host material and a compound (GD-1) as a dopant material were deposited to have a thickness of 40 nm, in such a way that the dope concentration reached 10%. Thereafter, as an electron transport material, a compound (ET-1) and a compound (LiQ) were laminated at a volume ratio of 1:1 to have a thickness of 40 nm. Here are the structures of the compounds used for the organic EL layer.

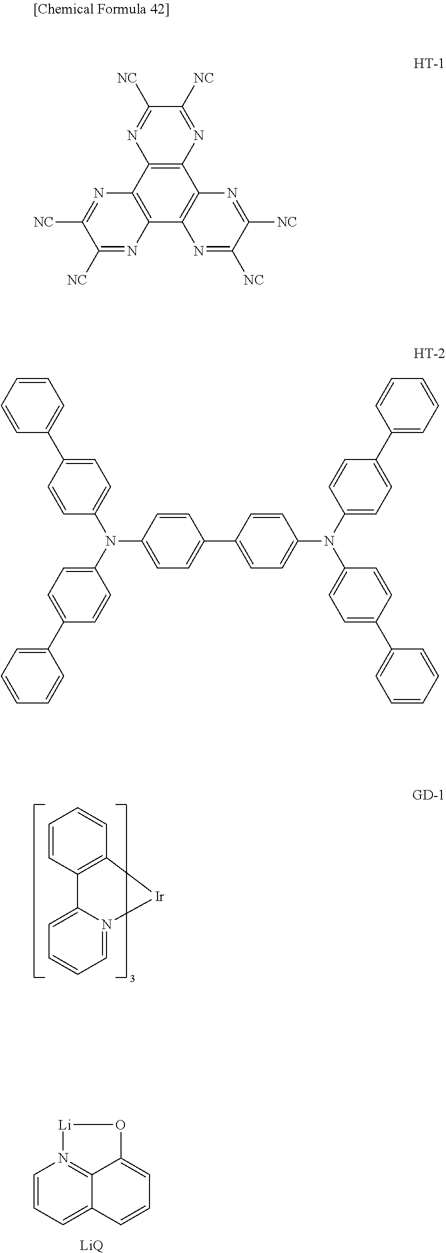

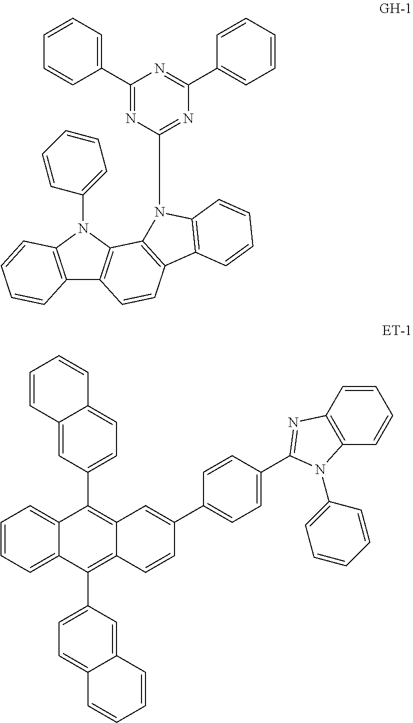 [SMILES: c1ccc(-c2nc(-c3ccccc3)nc(-n3c4ccccc4c4ccc5c6ccccc6n(-c6ccccc6)c5c43)n2)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

Next, after the vapor deposition of the compound (LiQ) of 2 nm, 100 nm MgAg (magnesium/silver=10/1 (volume ratio)) was deposited as a second electrode 52 to form a reflective electrode (FIG. 4 (Step 4)). Thereafter, under a low-humidity nitrogen atmosphere, sealing was performed by bonding a cap-shaped glass plate with the use of an epoxy resin-based adhesive, and four bottom-emission organic EL displays each in a square shape of 5 mm on a side were prepared on one substrate. It is to be noted that the film thickness herein refers to a value displayed on a crystal oscillation-type film thickness monitor.

### (Light-Emitting Characteristic Evaluation)

The organic EL displays prepared by the method described above was driven with a direct current at 10 mA/cm² to emit light, and it was confirmed whether there were defective light emissions such as non-light-emitting areas and uneven luminance. The prepared organic EL displays were held at 80° C. for 500 hours as a durability test. After the durability test, the organic EL displays prepared by the method described above was driven with a direct current at 10 mA/cm² to emit light, and it was confirmed whether there was any change in light-emitting characteristics such as light-emitting areas and uneven luminance. It has been determined as follows that, in a case where the light-emitting area before the durability test is regarded as 100%, the A+, A, and B where the light-emitting area after the durability test is 80% or higher are regarded as pass, A+ and A where the light-emitting area is 90% or higher are regarded as favorable light-emitting characteristics, and A+ where the light-emitting area is 95% or higher is regarded as an excellent light-emitting characteristic.

A+: The light-emitting area after the durability test is 95 to 100%.

A: The light-emitting area after the durability test is 90 to 94%.

B: The light-emitting area after the durability test is 80 to 89-.

C: The light-emitting area after the durability test is 70 to 79%.

D: The light-emitting area after the durability test is 50 to 69%

E: The light emitting area after the durability test is 0 to 49%.

### Example 1

Under a yellow light, 0.152 g of OXL-21 was weighed, 7.274 g of MBA and 5.100 g of PGMEA were added thereto, and dissolved by stirring. Next, 6.566 g of a 30% by mass MBA solution of the polyimide (PI-1) obtained in Synthesis Example 1, 0.606 g of a 50% by mass MBA solution of DPHA, and 1.515 g of a 50% by mass MBA solution of DPCA-60 were added to the solution, and then stirred to obtain a prepared liquid as a homogeneous solution. Next, 7.323 g of the pigment dispersion (Bk-1) obtained in Preparation Example 1 was weighed, and with 17.677 g of the prepared liquid, obtained by the method described above, added thereto, and then stirred to obtain a homogeneous solution. Thereafter, the obtained solution was filtered through a 0.45 μmφ filter to prepare Composition 1.

The prepared composition 1 was applied onto an ITO substrate by spin coating at an arbitrary rotation speed with the use of a spin coater (MS-A100; manufactured by Mikasa Co., Ltd.), and then prebaked at 110° C. for 120 seconds with the use of a buzzer hot plate (HPD-3000BZN; manufactured by AS ONE Corporation) to prepare a prebaked film of about 1.8 μm in film thickness.

The prebaked film prepared was subjected to spray development with a 2.38% by mass TMAH aqueous solution with the use of a small-size development device (AD-2000; manufactured by TAKIZAWA SANGYO K.K.) for photolithography, and the time at which the prebaked film (unexposed part) was completely dissolved (Breaking Point; hereinafter, a “B.P.”) was measured.

in the same manner as described above, a prebaked film was prepared, and the prepared prebaked film was subjected to exposure for patterning with the i-line (wavelength: 365 nm), h-line (wavelength: 405 nm), and g-line (wavelength 436 nm) of an ultra-high pressure mercury lamp through a gray scale mask (MDRM MODEL 4000-5-FS; Opto-Line International, Inc.) for sensitivity measurement with the use of a double-sided alignment single-sided exposure apparatus (Mask Aligner PEM-6M; manufactured by Union Optical Co., Ltd.), development was performed with the use of a small-size development device (AD-2000; TAKIZAWA SANGYO K.K.) for photolithography, thereby preparing a developed film of the photosensitive resin composition. After the exposure, the film was subjected to development with a 2.38% by mass TMAH aqueous solution with the use of a small-size development device (AD-2000; manufactured by TAKIZAWA SANGYO K.K.) for photolithography, and rinsed with water for 30 seconds. The development time was 1.5 times as long as B.P.

After the development, with the use of a high-temperature inert gas oven (INH-9CD-S; manufactured by Koyo Thermo Systems Co., Ltd.), thermal curing was performed at 250° C. to produce a cured film of about 1.2 μm in film thickness. The thermal curing condition was thermal curing at 250° C. for 60 minutes in a nitrogen atmosphere.

### Examples 2 to 83 and Comparative Examples 1 to 8

In the same manner as in Example 1, compositions 2 to 91 were prepared in accordance with the compositions described in Table 3-1 to Table 14-1. With the use of the respective compositions obtained, compositions were deposited on substrates in the same manner as in Example 1, and the photosensitive characteristics and the characteristics of the cured film were evaluated. These evaluation results are collectively shown in Table 3-2 to Table 14-2. It is to be noted that for ease of comparison, the composition and evaluation results according to Example 7 are listed in each of Table 4-1 to Table 5-1, Table 7-1 to Table 13-1, Table 4-2 to Table 5-2, and Table 7-2 to Table 13-2.

TABLE 3-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Radical | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | | Pigment | First | Second | (B3) | (C1-1) | (D) | (E) | [% by | [% by | [% by |
| | Composition | Dispersion | Resin | Resin | compounds | Compound | Colorant | Dispersant | mass] | mass] | mass] |
| Example 1 | 1 | Bk-1 | PI-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 2 | 2 | Bk-1 | PIP-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 3 | 3 | Bk-1 | PBO-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 4 | 4 | Bk-1 | PBOP-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 5 | 5 | Bk-1 | PI-1 | PS-1 | DPHA (10) | OXL-21 | Bk-S0100CF | S-20000 | 100 | 35 | 22.0 |
| | | | (50) | (15) | DPCA-60 (25) | (5) | (32.6) | (10.9) | | | |
| Example 6 | 6 | Bk-2 | PI-1 | — | DPHA (10) | OXL-21 | Bk-S0100CF | S-20000 | 100 | 35 | 22.0 |
| | | | (65) | | DPCA-60 (25) | (2) | (31.8) | (10.6) | | | |
| Example 7 | 7 | Bk-2 | PI-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 8 | 8 | Bk-2 | PI-1 | — | DPHA (10) | OXL-21 | Bk-S0100CF | S-20000 | 100 | 35 | 22.0 |
| | | | (65) | | DPCA-60 (25) | (7) | (33.3) | (11.1) | | | |
| Example 9 | 9 | Bk-2 | PI-1 | — | DPHA (10) | OXL-21 | Bk-S0100CF | S-20000 | 100 | 35 | 22.0 |
| | | | (65) | | DPCA-60 (25) | (13) | (35.1) | (11.7) | | | |
| Example 10 | 10 | Bk-2 | PI-1 | — | DPHA (10) | OXL-21 | Bk-S0100CF | S-20000 | 100 | 35 | 22.0 |
| | | | (65) | | DPCA-60 (25) | (17) | (36.4) | (12.1) | | | |

TABLE 3-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 1 | 1 | 35 | 3 | 37 | 27 | 0.40 | 8.5 |
| | | A | A | A | A+ | A | A |
| Example 2 | 2 | 30 | 3 | 37 | 27 | 0.40 | 18.3 |
| | | A+ | A | A | A+ | A | B |
| Example 3 | 3 | 35 | 3 | 37 | 27 | 0.40 | 8.7 |
| | | A | A | A | A+ | A | A |
| Example 4 | 4 | 30 | 3 | 37 | 27 | 0.40 | 18.5 |
| | | A+ | A | A | A+ | A | B |
| Example 5 | 5 | 25 | 3 | 33 | 23 | 0.40 | 11.6 |
| | | A+ | A | A | A+ | A | B |
| Example 6 | 6 | 50 | 0 | 40 | 25 | 0.65 | 8.6 |
| | | B | A+ | A | A+ | C | A |
| Example 7 | 7 | 35 | 0 | 37 | 27 | 0.40 | 8.3 |
| | | A | A+ | A | A+ | A | A |
| Example 8 | 8 | 30 | 0 | 35 | 30 | 0.35 | 8.1 |
| | | A+ | A+ | A | A+ | A | A |
| Example 9 | 9 | 20 | 0 | 40 | 30 | 0.40 | 8.1 |
| | | A+ | A+ | A | A+ | A | A |
| Example 10 | 10 | 20 | 0 | 45 | 35 | 0.55 | 8.0 |
| | | A+ | A+ | A | A | B | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 1 | 1.0 | >1.0 × | Favorable | 100 |
| | | 10\hat{ }15 | | A+ |
| Example 2 | 1.0 | >1.0 × | Favorable | 100 |
| | | 10\hat{ }15 | | A+ |
| Example 3 | 1.0 | >1.0 × | Favorable | 100 |
| | | 10\hat{ }15 | | A+ |
| Example 4 | 1.0 | >1.0 × | Favorable | 100 |
| | | 10\hat{ }15 | | A+ |
| Example 5 | 1.0 | >1.0 × | Favorable | 90 |
| | | 10\hat{ }15 | | A |
| Example 6 | 1.0 | >1.0 × | Favorable | 100 |
| | | 10\hat{ }15 | | A+ |
| Example 7 | 1.0 | >1.0 × | Favorable | 100 |
| | | 10\hat{ }15 | | A+ |
| Example 8 | 1.0 | >1.0 × | Favorable | 100 |
| | | 10\hat{ }15 | | A+ |
| Example 9 | 1.0 | >1.0 × | Favorable | 100 |
| | | 10\hat{ }15 | | A+ |
| Example 10 | 1.0 | >1.0 × | Favorable | 100 |
| | | 10\hat{ }15 | | A+ |

TABLE 4-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Radical | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | | Pigment | First | Second | (B3) | (C1-1) | (D) | (E) | [% by | [% by | [% by |
| | Composition | Dispersion | Resin | Resin | compounds | Compound | Colorant | Dispersant | mass] | mass] | mass] |
| Example 7 | 7 | Bk-2 | PI-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 11 | 11 | Bk-2 | PI-1 | — | DPHA (10) | OXL-21 | Bk-S0100CF | S-20000 | 100 | 35 | 10.0 |
| | | | (65) | | DPCA-60 (25) | (5) | (12.1) | (4.0) | | | |
| Example 12 | 12 | Bk-2 | PI-1 | — | DPHA (10) | OXL-21 | Bk-S0100CF | S-20000 | 100 | 35 | 32.0 |
| | | | (65) | | DPCA-60 (25) | (5) | (58.5) | (19.5) | | | |
| Example 13 | 13 | Bk-3 | PI-1 | — | DPHA (10) | OXL-21 | Bk-S0100CF | S-20000 | 100 | 35 | 45.0 |
| | | | (65) | | DPCA-60 (25) | (5) | (98.4) | (15.1) | | | |
| Example 14 | 14 | Bk-2 | PI-1 | — | DPHA (10) | OXL-21 | Bk-S0100CF | S-20000 | 100 | 35 | 22.0 |
| | | | (65) | | A-DPH-6E (25) | (5) | (32.6) | (10.9) | | | |
| Example 15 | 15 | Bk-2 | PI-1 | — | DPCA-60 (25) | OXL-21 | Bk-S0100CF | S-20000 | 100 | 35 | 22.0 |
| | | | (65) | | A-BPEF (10) | (5) | (32.6) | (10.9) | | | |
| Example 16 | 16 | Bk-2 | PI-1 | — | DPCA-60 (25) | OXL-21 | Bk-S0100CF | S-20000 | 100 | 35 | 22.0 |
| | | | (65) | | IDN-1 (10) | (5) | (32.6) | (10.9) | | | |
| Example 17 | 17 | Bk-2 | PI-1 | — | DPHA (35) | OXL-21 | Bk-S0100CF | S-20000 | 100 | 35 | 22.0 |
| | | | (65) | | | (5) | (32.6) | (10.9) | | | |

TABLE 4-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 7 | 7 | 35 | 0 | 37 | 27 | 0.40 | 8.3 |
| | | A | A+ | A | A+ | A | A |
| Example 11 | 11 | 25 | 0 | 34 | 25 | 0.40 | 7.4 |
| | | A+ | A+ | A | A+ | A | A |
| Example 12 | 12 | 45 | 2 | 40 | 30 | 0.40 | 10.3 |
| | | A | A | A | A+ | A | A |
| Example 13 | 13 | 65 | 3 | 45 | 35 | 0.45 | 12.5 |
| | | C | A | A | A | B | A |
| Example 14 | 14 | 50 | 5 | 43 | 28 | 0.50 | 9.3 |
| | | B | A | A | A+ | B | A |
| Example 15 | 15 | 25 | 0 | 33 | 25 | 0.15 | 7.0 |
| | | A+ | A+ | A | A+ | A+ | A |
| Example 16 | 16 | 25 | 0 | 35 | 27 | 0.20 | 7.5 |
| | | A+ | A+ | A | A+ | A+ | A |
| Example 17 | 17 | 60 | 7 | 37 | 20 | 0.65 | 8.6 |
| | | B | B | A | A+ | C | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 7 | 1.0 | >1.0 × | Favorable | 100 |
| | | 10\hat{ }15 | | A+ |
| Example 11 | 0.5 | >1.0 × | Favorable | 100 |
| | | 10\hat{ }15 | | A+ |
| Example 12 | 1.5 | >1.0 × | Favorable | 100 |
| | | 10\hat{ }15 | | A+ |
| Example 13 | 2.0 | >1.0 × | Favorable | 100 |
| | | 10\hat{ }15 | | A+ |
| Example 14 | 1.0 | >1.0 × | Favorable | 100 |
| | | 10\hat{ }15 | | A+ |
| Example 15 | 1.0 | >1.0 × | Favorable | 100 |
| | | 10\hat{ }15 | | A+ |
| Example 16 | 1.0 | >1.0 × | Favorable | 100 |
| | | 10\hat{ }15 | | A+ |
| Example 17 | 1.0 | >1.0 × | Favorable | 100 |
| | | 10\hat{ }15 | | A+ |

TABLE 5-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Radical | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | | Pigment | First | Second | (B3) | (C1-1) | (D) | (E) | [% by | [% by | [% by |
| | Composition | Dispersion | Resin | Resin | compounds | Compound | Colorant | Dispersant | mass] | mass] | mass] |
| Example 7 | 7 | Bk-2 | PI-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 18 | 18 | Bk-2 | PI-1 | — | DPHA (10) | OXL-16 | Bk-S0100CF | S-20000 | 100 | 35 | 22.0 |
| | | | (65) | | DPCA-60 (25) | (5) | (32.6) | (10.9) | | | |
| Example 19 | 19 | Bk-2 | PI-1 | — | DPHA (10) | OXL-19 | Bk-S0100CF | S-20000 | 100 | 35 | 22.0 |
| | | | (65) | | DPCA-60 (25) | (5) | (32.6) | (10.9) | | | |
| Example 20 | 20 | Bk-2 | PI-1 | — | DPHA (10) | OXL-1 | Bk-S0100CF | S-20000 | 100 | 35 | 22.0 |
| | | | (65) | | DPCA-60 (25) | (5) | (32.6) | (10.9) | | | |
| Example 21 | 21 | Bk-2 | PI-1 | — | DPHA (10) | OXL-43 | Bk-S0100CF | S-20000 | 100 | 35 | 22.0 |
| | | | (65) | | DPCA-60 (25) | (5) | (32.6) | (10.9) | | | |
| Example 22 | 22 | Bk-2 | PI-1 | — | DPHA (10) | OXL-44 | Bk-S0100CF | S-20000 | 100 | 35 | 22.0 |
| | | | (65) | | DPCA-60 (25) | (5) | (32.6) | (10.9) | | | |
| Example 23 | 23 | Bk-2 | PI-1 | — | DPHA (10) | OXL-2 | Bk-S0100CF | S-20000 | 100 | 35 | 22.0 |
| | | | (65) | | DPCA-60 (25) | (5) | (32.6) | (10.9) | | | |
| Example 24 | 24 | Bk-2 | PI-1 | — | DPHA (10) | OXL-58 | Bk-S0100CF | S-20000 | 100 | 35 | 22.0 |
| | | | (65) | | DPCA-60 (25) | (5) | (32.6) | (10.9) | | | |
| Example 25 | 25 | Bk-2 | PI-1 | — | DPHA (10) | OXL-63 | Bk-S0100CF | S-20000 | 100 | 35 | 22.0 |
| | | | (65) | | DPCA-60 (25) | (5) | (32.6) | (10.9) | | | |

TABLE 5-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 7 | 7 | 35 | 0 | 37 | 27 | 0.40 | 8.3 |
| | | A | A+ | A | A+ | A | A |
| Example 18 | 18 | 40 | 0 | 40 | 28 | 0.45 | 8.4 |
| | | A | A+ | A | A+ | B | A |
| Example 19 | 19 | 35 | 0 | 37 | 27 | 0.40 | 8.4 |
| | | A | A+ | A | A+ | A | A |
| Example 20 | 20 | 50 | 0 | 45 | 30 | 0.55 | 8.5 |
| | | B | A+ | A | A+ | B | A |
| Example 21 | 21 | 50 | 0 | 45 | 30 | 0.55 | 8.5 |
| | | B | A+ | A | A+ | B | A |
| Example 22 | 22 | 45 | 0 | 42 | 28 | 0.50 | 8.5 |
| | | A | A+ | A | A+ | B | A |
| Example 23 | 23 | 50 | 0 | 45 | 30 | 0.55 | 8.5 |
| | | B | A+ | A | A+ | B | A |
| Example 24 | 24 | 50 | 0 | 45 | 30 | 0.55 | 8.5 |
| | | B | A+ | A | A+ | B | A |
| Example 25 | 25 | 45 | 0 | 42 | 28 | 0.50 | 8.5 |
| | | A | A+ | A | A+ | B | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 7 | 1.0 | >1.0 × | Favorable | 100 |
| | | 10\hat{ }15 | | A+ |
| Example 18 | 1.0 | >1.0 × | Favorable | 100 |
| | | 10\hat{ }15 | | A+ |
| Example 19 | 1.0 | >1.0 × | Favorable | 100 |
| | | 10\hat{ }15 | | A+ |
| Example 20 | 1.0 | >1.0 × | Favorable | 100 |
| | | 10\hat{ }15 | | A+ |
| Example 21 | 1.0 | >1.0 × | Favorable | 100 |
| | | 10\hat{ }15 | | A+ |
| Example 22 | 1.0 | >1.0 × | Favorable | 100 |
| | | 10\hat{ }15 | | A+ |
| Example 23 | 1.0 | >1.0 × | Favorable | 100 |
| | | 10\hat{ }15 | | A+ |
| Example 24 | 1.0 | >1.0 × | Favorable | 100 |
| | | 10\hat{ }15 | | A+ |
| Example 25 | 1.0 | >1.0 × | Favorable | 100 |
| | | 10\hat{ }15 | | A+ |

TABLE 6-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Radical | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | | Pigment | First | Second | (B3) | (C1-1) | (D) | (E) | [% by | [% by | [% by |
| | Composition | Dispersion | Resin | Resin | compounds | Compound | Colorant | Dispersant | mass] | mass] | mass] |
| Example 26 | 26 | Bk-2 | PI-1 | — | DPHA (10) | OXL-7 | Bk-S0100CF | S-20000 | 100 | 35 | 22.0 |
| | | | (65) | | DPCA-60 (25) | (5) | (32.6) | (10.9) | | | |
| Example 27 | 27 | Bk-2 | PI-1 | — | DPHA (10) | OXL-54 | Bk-S0100CF | S-20000 | 100 | 35 | 22.0 |
| | | | (65) | | DPCA-60 (25) | (5) | (32.6) | (10.9) | | | |
| Example 28 | 28 | Bk-2 | PI-1 | — | DPHA (10) | OXL-55 | Bk-S0100CF | S-20000 | 100 | 35 | 22.0 |
| | | | (65) | | DPCA-60 (25) | (5) | (32.6) | (10.9) | | | |
| Example 29 | 29 | Bk-2 | PI-1 | — | DPHA (10) | OXL-9 | Bk-S0100CF | S-20000 | 100 | 35 | 22.0 |
| | | | (65) | | DPCA-60 (25) | (5) | (32.6) | (10.9) | | | |
| Example 30 | 30 | Bk-2 | PI-1 | — | DPHA (10) | OXL-49 | Bk-S0100CF | S-20000 | 100 | 35 | 22.0 |
| | | | (65) | | DPCA-60 (25) | (5) | (32.6) | (10.9) | | | |
| Example 31 | 31 | Bk-2 | PI-1 | — | DPHA (10) | OXL-31 | Bk-S0100CF | S-20000 | 100 | 35 | 22.0 |
| | | | (65) | | DPCA-60 (25) | (5) | (32.6) | (10.9) | | | |
| Example 32 | 32 | Bk-2 | PI-1 | — | DPHA (10) | OXL-37 | Bk-S0100CF | S-20000 | 100 | 35 | 22.0 |
| | | | (65) | | DPCA-60 (25) | (5) | (32.6) | (10.9) | | | |
| Example 33 | 33 | Bk-2 | PI-1 | — | DPHA (10) | OXL-71 | Bk-S0100CF | S-20000 | 100 | 35 | 22.0 |
| | | | (65) | | DPCA-60 (25) | (5) | (32.6) | (10.9) | | | |
| Example 34 | 34 | Bk-2 | PI-1 | — | DPHA (10) | OXL-79 | Bk-S0100CF | S-20000 | 100 | 35 | 22.0 |
| | | | (65) | | DPCA-60 (25) | (5) | (32.6) | (10.9) | | | |
| Example 35 | 35 | Bk-2 | PI-1 | — | DPHA (10) | OXL-81 | Bk-S0100CF | S-20000 | 100 | 35 | 22.0 |
| | | | (65) | | DPCA-60 (25) | (5) | (32.6) | (10.9) | | | |

TABLE 6-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 26 | 26 | 40 | 0 | 40 | 28 | 0.45 | 8.4 |
| | | A | A+ | A | A+ | B | A |
| Example 27 | 27 | 40 | 0 | 40 | 20 | 0.45 | 8.5 |
| | | A | A+ | A | A+ | B | A |
| Example 28 | 28 | 35 | 0 | 37 | 27 | 0.40 | 8.4 |
| | | A | A+ | A | A+ | A | A |
| Example 29 | 29 | 50 | 0 | 45 | 30 | 0.55 | 8.7 |
| | | B | A+ | A | A+ | B | A |
| Example 30 | 30 | 50 | 0 | 45 | 30 | 0.55 | 8.7 |
| | | B | A+ | A | A+ | B | A |
| Example 31 | 31 | 40 | 0 | 40 | 28 | 0.45 | 8.5 |
| | | A | A+ | A | A+ | B | A |
| Example 32 | 32 | 40 | 0 | 40 | 28 | 0.45 | 8.5 |
| | | A | A+ | A | A+ | B | A |
| Example 33 | 33 | 50 | 0 | 45 | 30 | 0.55 | 8.7 |
| | | B | A+ | A | A+ | B | A |
| Example 34 | 34 | 60 | 0 | 45 | 27 | 0.60 | 8.7 |
| | | B | A+ | A | A+ | B | A |
| Example 35 | 35 | 60 | 0 | 45 | 27 | 0.60 | 8.7 |
| | | B | A+ | A | A+ | B | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 26 | 1.0 | >1.0 × | Favorable | 100 |
| | | 10\hat{ }15 | | A+ |
| Example 27 | 1.0 | >1.0 × | Favorable | 100 |
| | | 10\hat{ }15 | | A+ |
| Example 28 | 1.0 | >1.0 × | Favorable | 100 |
| | | 10\hat{ }15 | | A+ |
| Example 29 | 1.0 | >1.0 × | Favorable | 100 |
| | | 10\hat{ }15 | | A+ |
| Example 30 | 1.0 | >1.0 × | Favorable | 100 |
| | | 10\hat{ }15 | | A+ |
| Example 31 | 1.0 | >1.0 × | Favorable | 100 |
| | | 10\hat{ }15 | | A+ |
| Example 32 | 1.0 | >1.0 × | Favorable | 100 |
| | | 10\hat{ }15 | | A+ |
| Example 33 | 1.0 | >1.0 × | Favorable | 100 |
| | | 10\hat{ }15 | | A+ |
| Example 34 | 1.0 | >1.0 × | Favorable | 100 |
| | | 10\hat{ }15 | | A+ |
| Example 35 | 1.0 | >1.0 × | Favorable | 100 |
| | | 10\hat{ }15 | | A+ |

TABLE 7-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Radical | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | Compo- | Pigment | First | Second | (B3) | (C1-1) | (D) | (E) | [% by | [% by | [% by |
| | sition | Dispersion | Resin | Resin | compounds | Compound | Colorant | Dispersant | mass] | mass] | mass] |
| Example 7 | 7 | Bk-2 | PI-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 36 | 36 | Bk-2 | PI-2 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 37 | 37 | Bk-2 | PI-3 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 38 | 38 | Bk-2 | PI-4 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 39 | 39 | Bk-2 | PI-1 (50) | — | DPHA (10) | OXL-21 | Bk-S0100CF | S-20000 | 100 | 35 | 22.0 |
| | | | PIP-1 (15) | | DPCA-60 (25) | (5) | (32.6) | (10.9) | | | |
| Example 40 | 40 | Bk-2 | PI-1 (50) | — | DPHA (10) | OXL-21 | Bk-S0100CF | S-20000 | 100 | 35 | 22.0 |
| | | | PIP-2 (15) | | DPCA-60 (25) | (5) | (32.6) | (10.9) | | | |
| Example 41 | 41 | Bk-2 | PI-1 (50) | — | DPHA (10) | OXL-21 | Bk-S0100CF | S-20000 | 100 | 35 | 22.0 |
| | | | PBO-1 (15) | | DPCA-60 (25) | (5) | (32.6) | (10.9) | | | |
| Example 42 | 42 | Bk-2 | PI-1 (50) | — | DPHA (10) | OXL-21 | Bk-S0100CF | S-20000 | 100 | 35 | 22.0 |
| | | | PBOP-1 (15) | | DPCA-60 (25) | (5) | (32.6) | (10.9) | | | |

TABLE 7-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 7 | 7 | 35 | 0 | 37 | 27 | 0.40 | 8.3 |
| | | A | A+ | A | A+ | A | A |
| Example 36 | 36 | 40 | 0 | 40 | 30 | 0.40 | 8.4 |
| | | A | A+ | A | A+ | A | A |
| Example 37 | 37 | 30 | 0 | 35 | 25 | 0.40 | 8.3 |
| | | A+ | A+ | A | A+ | A | A |
| Example 38 | 38 | 25 | 0 | 33 | 23 | 0.40 | 8.4 |
| | | A+ | A+ | A | A+ | A | A |
| Example 39 | 39 | 30 | 0 | 35 | 25 | 0.40 | 9.4 |
| | | A+ | A+ | A | A+ | A | A |
| Example 40 | 40 | 35 | 0 | 37 | 27 | 0.40 | 9.3 |
| | | A | A+ | A | A+ | A | A |
| Example 41 | 41 | 35 | 0 | 37 | 27 | 0.40 | 8.3 |
| | | A | A+ | A | A+ | A | A |
| Example 42 | 42 | 30 | 0 | 35 | 25 | 0.40 | 9.4 |
| | | A+ | A+ | A | A+ | A | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 7 | 1.0 | >1.0 × | Favorable | 100 |
| | | 10\hat{ }15 | | A+ |
| Example 36 | 1.0 | >1.0 × | Favorable | 100 |
| | | 10\hat{ }15 | | A+ |
| Example 37 | 1.0 | >1.0 × | Favorable | 100 |
| | | 10\hat{ }15 | | A+ |
| Example 38 | 1.0 | >1.0 × | Favorable | 100 |
| | | 10\hat{ }15 | | A+ |
| Example 39 | 1.0 | >1.0 × | Favorable | 100 |
| | | 10\hat{ }15 | | A+ |
| Example 40 | 1.0 | >1.0 × | Favorable | 100 |
| | | 10\hat{ }15 | | A+ |
| Example 41 | 1.0 | >1.0 × | Favorable | 100 |
| | | 10\hat{ }15 | | A+ |
| Example 42 | 1.0 | >1.0 × | Favorable | 100 |
| | | 10\hat{ }15 | | A+ |

TABLE 8-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Residual | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | | Pigment | First | Second | (B3) | (C1-1) | (D) | (E) | [% by | [% by | [% by |
| | Composition | Dispersion | Resin | Resin | compounds | Compound | Colorant | Dispersion | mass] | mass] | mass] |
| Example 7 | 7 | Bk-2 | PI-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 43 | 43 | Bk-2 | PI-1 | PS-1 | DPHA (10) | OXL-21 | Bk-S0100CF | S-20000 | 100 | 35 | 22.0 |
| | | | (50) | (15) | DPCA-60 (25) | (5) | (32.6) | (10.9) | | | |
| Example 44 | 44 | Bk-2 | PI-1 | PS-2 | DPHA (10) | OXL-21 | Bk-S0100CF | S-20000 | 100 | 35 | 22.0 |
| | | | (50) | (15) | DPCA-60 (25) | (5) | (32.6) | (10.9) | | | |
| Example 45 | 45 | Bk-2 | PI-1 | CR-1 | DPHA (10) | OXL-21 | Bk-S0100CF | S-20000 | 77 | 35 | 22.0 |
| | | | (50) | (15) | DPCA-60 (25) | (5) | (32.6) | (10.9) | | | |
| Example 46 | 46 | Bk-2 | PI-1 | WR-301 | DPHA (10) | OXL-21 | Bk-S0100CF | S-20000 | 77 | 35 | 22.0 |
| | | | (50) | (15) | DPCA-60 (25) | (5) | (32.6) | (10.9) | | | |
| Example 47 | 47 | Bk-2 | PI-1 | AE-1 | DPHA (10) | OXL-21 | Bk-S0100CF | S-20000 | 77 | 35 | 22.0 |
| | | | (50) | (15) | DPCA-60 (25) | (5) | (32.6) | (10.9) | | | |
| Example 48 | 48 | Bk-2 | PI-1 | AC-1 | DPHA (10) | OXL-21 | Bk-S0100CF | S-20000 | 77 | 35 | 22.0 |
| | | | (50) | (15) | DPCA-60 (25) | (5) | (32.6) | (10.9) | | | |
| Example 49 | 49 | Bk-2 | PI-1 | CR-1 | DPHA (10) | OXL-21 | Bk-S0100CF | S-20000 | 54 | 35 | 22.0 |
| | | | (35) | (30) | DPCA-60 (25) | (5) | (32.6) | (10.9) | | | |
| Example 50 | 50 | Bk-2 | PI-1 | CR-1 | DPHA (10) | OXL-21 | Bk-S0100CF | S-20000 | 92 | 35 | 22.0 |
| | | | (60) | (5) | DPCA-60 (25) | (5) | (32.6) | (10.9) | | | |

TABLE 8-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 7 | 7 | 35 | 0 | 37 | 27 | 0.40 | 8.3 |
| | | A | A+ | A | A+ | A | A |
| Example 43 | 43 | 25 | 0 | 33 | 23 | 0.35 | 11.0 |
| | | A+ | A+ | A | A+ | A | A |
| Example 44 | 44 | 30 | 0 | 35 | 20 | 0.40 | 11.6 |
| | | A+ | A+ | A | A+ | A | A |
| Example 45 | 45 | 20 | 0 | 34 | 24 | 0.40 | 15.4 |
| | | A+ | A+ | A | A+ | A | B |
| Example 46 | 46 | 20 | 0 | 34 | 24 | 0.40 | 15.2 |
| | | A+ | A+ | A | A+ | A | B |
| Example 47 | 47 | 20 | 0 | 34 | 24 | 0.40 | 17.2 |
| | | A+ | A+ | A | A+ | A | B |
| Example 48 | 48 | 20 | 0 | 34 | 24 | 0.40 | 21.2 |
| | | A+ | A+ | A | A+ | A | B |
| Example 49 | 49 | 20 | 0 | 46 | 31 | 0.55 | 19.4 |
| | | A+ | A+ | B | A | B | B |
| Example 50 | 50 | 30 | 0 | 31 | 23 | 0.25 | 11.1 |
| | | A+ | A+ | A | A+ | A | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 7 | 1.0 | >1.0 × | Favorable | 100 |
| | | 10\hat{ }15 | | A+ |
| Example 43 | 1.0 | >1.0 × | Favorable | 100 |
| | | 10\hat{ }15 | | A+ |
| Example 44 | 1.0 | >1.0 × | Favorable | 100 |
| | | 10\hat{ }15 | | A+ |
| Example 45 | 1.0 | >1.0 × | Favorable | 95 |
| | | 10\hat{ }15 | | A |
| Example 46 | 1.0 | >1.0 × | Favorable | 95 |
| | | 10\hat{ }15 | | A |
| Example 47 | 1.0 | >1.0 × | Favorable | 90 |
| | | 10\hat{ }15 | | A |
| Example 48 | 1.0 | >1.0 × | Favorable | 85 |
| | | 10\hat{ }15 | | B |
| Example 49 | 1.0 | >1.0 × | Favorable | 85 |
| | | 10\hat{ }15 | | B |
| Example 50 | 1.0 | >1.0 × | Favorable | 100 |
| | | 10\hat{ }15 | | A+ |

TABLE 9-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Residual | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | | Pigment | First | Second | (B3) | (C1-1) | (D) | (E) | [% by | [% by | [% by |
| | Composition | Dispersion | Resin | Resin | compounds | Compound | Colorant | Dispersion | mass] | mass] | mass] |
| Example 7 | 7 | Bk-2 | PI-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 51 | 51 | Bk-4 | PI-1 | — | DPHA (10) | OXL-21 | Bk-S0084 | D.BYK-167 | 100 | 35 | 22.0 |
| | | | (65) | | DPCA-60 (25) | (5) | (32.6) | (10.9) | | | |
| Example 52 | 52 | Bk-5 | PI-1 | — | DPHA (10) | OXL-21 | Bk-A1103 | D.BYK-167 | 100 | 35 | 22.0 |
| | | | (65) | | DPCA-60 (25) | (5) | (32.6) | (10.9) | | | |
| Example 53 | 53 | Bk-6 | PI-1 | — | DPHA (10) | OXL-21 | TPK-1227 | D.BYK-167 | 100 | 35 | 15.0 |
| | | | (65) | | DPCA-60 (25) | (5) | (19.6) | (6.5) | | | |
| Example 54 | 54 | Bk-7 | PI-1 | — | DPHA (10) | OXL-21 | P.R.254 | D.BYK-167 | 100 | 35 | 32.0 |
| | | | (65) | | DPCA-60 (25) | (5) | (20.5) | (19.5) | | | |
| | | | | | | | P.Y.139 | | | | |
| | | | | | | | (8.8) | | | | |
| | | | | | | | P.B.15:6 | | | | |
| | | | | | | | (29.3) | | | | |
| Example 55 | 55 | Bk-8 | PI-1 | — | DPHA (10) | OXL-21 | P.V.23 | D.BYK-167 | 100 | 35 | 32.0 |
| | | | (65) | | DPCA-60 (25) | (5) | (35.1) | (19.5) | | | |
| | | | | | | | P.Y.139 | | | | |
| | | | | | | | (23.4) | | | | |

TABLE 9-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 7 | 7 | 35 | 0 | 37 | 27 | 0.40 | 8.3 |
| | | A | A+ | A | A+ | A | A |
| Example 51 | 51 | 45 | 0 | 40 | 30 | 0.40 | 9.5 |
| | | A | A+ | A | A+ | A | A |
| Example 52 | 52 | 45 | 0 | 40 | 30 | 0.40 | 9.8 |
| | | A | A+ | A | A+ | A | A |
| Example 53 | 53 | 60 | 0 | 40 | 28 | 0.45 | 10.6 |
| | | B | A+ | A | A+ | B | A |
| Example 54 | 54 | 55 | 0 | 40 | 28 | 0.45 | 10.8 |
| | | B | A+ | A | A+ | B | A |
| Example 55 | 55 | 55 | 0 | 40 | 28 | 0.45 | 10.7 |
| | | B | A+ | A | A+ | B | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 7 | 1.0 | >1.0 × | Favorable | 100 |
| | | 10\hat{ }15 | | A+ |
| Example 51 | 1.0 | >1.0 × | Favorable | 100 |
| | | 10\hat{ }15 | | A+ |
| Example 52 | 1.0 | >1.0 × | Favorable | 100 |
| | | 10\hat{ }15 | | A+ |
| Example 53 | 1.0 | >1.0 × | Favorable | 80 |
| | | 10\hat{ }15 | | B |
| Example 54 | 1.0 | >1.0 × | Favorable | 90 |
| | | 10\hat{ }15 | | A |
| Example 55 | 1.0 | >1.0 × | Favorable | 90 |
| | | 10\hat{ }15 | | A |

TABLE 10-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Residual | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | | Pigment | First | Second | (B3) | (C1-1) | (D) | (E) | [% by | [% by | [% by |
| | Composition | Dispersion | Resin | Resin | compounds | Compound | Colorant | Dispersion | mass] | mass] | mass] |
| Example 7 | 7 | Bk-2 | PI-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 56 | 56 | Bk-9 | PI-1 | — | DPHA (10) | OXL-21 | P.R.179 | D.BYK-167 | 100 | 35 | 32.0 |
| | | | (65) | | DPCA-60 (25) | (5) | (17.6) | (19.5) | | | |
| | | | | | | | P.Y.192 | | | | |
| | | | | | | | (17.6) | | | | |
| | | | | | | | P.B.60 | | | | |
| | | | | | | | (23.4) | | | | |
| Example 57 | 57 | Bk-10 | PI-1 | — | DPHA (10) | OXL-21 | P.V.37 | D.BYK-167 | 100 | 35 | 32.0 |
| | | | (65) | | DPCA-60 (25) | (5) | (38.0) | (19.5) | | | |
| | | | | | | | P.Y.192 | | | | |
| | | | | | | | (20.5) | | | | |
| Example 58 | 58 | Bk-11 | PI-1 | — | DPHA (10) | OXL-21 | P.R.179 | D.BYK-167 | 100 | 35 | 32.0 |
| | | | (65) | | DPCA-60 (25) | (5) | (17.6) | (19.5) | | | |
| | | | | | | | P.O.43 | | | | |
| | | | | | | | (17.6) | | | | |
| | | | | | | | P.B.60 | | | | |
| | | | | | | | (23.4) | | | | |
| Example 59 | 59 | Bk-12 | PI-1 | — | DPHA (10) | OXL-21 | P.V.37 | D.BYK-167 | 100 | 35 | 32.0 |
| | | | (65) | | DPCA-60 (25) | (5) | (32.2) | (19.5) | | | |
| | | | | | | | P.O.43 | | | | |
| | | | | | | | (17.6) | | | | |
| | | | | | | | P.B.60 | | | | |
| | | | | | | | (8.8) | | | | |

TABLE 10-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 7 | 7 | 35 | 0 | 37 | 27 | 0.40 | 8.3 |
| | | A | A+ | A | A+ | A | A |
| Example 56 | 56 | 30 | 0 | 35 | 25 | 0.40 | 9.5 |
| | | A+ | A+ | A | A+ | A | A |
| Example 57 | 57 | 30 | 0 | 35 | 25 | 0.40 | 9.6 |
| | | A+ | A+ | A | A+ | A | A |
| Example 58 | 58 | 30 | 0 | 35 | 25 | 0.40 | 9.5 |
| | | A+ | A+ | A | A+ | A | A |
| Example 59 | 59 | 30 | 0 | 35 | 25 | 0.40 | 9.5 |
| | | A+ | A+ | A | A+ | A | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 7 | 1.0 | >1.0 × | Favorable | 100 |
| | | 10\hat{ }15 | | A+ |
| Example 56 | 1.0 | >1.0 × | Favorable | 100 |
| | | 10\hat{ }15 | | A+ |
| Example 57 | 1.0 | >1.0 × | Favorable | 100 |
| | | 10\hat{ }15 | | A+ |
| Example 58 | 1.0 | >1.0 × | Favorable | 100 |
| | | 10\hat{ }15 | | A+ |
| Example 59 | 1.0 | >1.0 × | Favorable | 100 |
| | | 10\hat{ }15 | | A+ |

TABLE 11-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Residual | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | | Pigment | First | Second | (B3) | (C1-1) | (D) | (E) | [% by | [% by | [% by |
| | Composition | Dispersion | Resin | Resin | compounds | Compound | Colorant | Dispersion | mass] | mass] | mass] |
| Example 7 | 7 | Bk-2 | PI-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 60 | 60 | Bk-2 | PI-1 | — | DPHA (10) | OXL-82 | Bk-S0100CF | S-20000 | 100 | 35 | 22.0 |
| | | | (65) | | DPCA-60 (25) | (5) | (32.6) | (10.9) | | | |
| Example 61 | 61 | Bk-2 | PI-1 | — | DPHA (10) | OXL-83 | Bk-S0100CF | S-20000 | 100 | 35 | 22.0 |
| | | | (65) | | DPCA-60 (25) | (5) | (32.6) | (10.9) | | | |
| Example 62 | 62 | Bk-2 | PI-1 | — | DPHA (10) | OXL-87 | Bk-S0100CF | S-20000 | 100 | 35 | 22.0 |
| | | | (65) | | DPCA-60 (25) | (5) | (32.6) | (10.9) | | | |
| Example 63 | 63 | Bk-2 | PI-1 | — | DPHA (10) | OXL-96 | Bk-S0100CF | S-20000 | 100 | 35 | 22.0 |
| | | | (65) | | DPCA-60 (25) | (5) | (32.6) | (10.9) | | | |
| Example 64 | 64 | Bk-2 | PI-1 | — | DPHA (10) | OXL-97 | Bk-S0100CF | S-20000 | 100 | 35 | 22.0 |
| | | | (65) | | DPCA-60 (25) | (5) | (32.6) | (10.9) | | | |
| Example 65 | 65 | Bk-2 | PI-1 | — | DPHA (10) | OXL-99 | Bk-S0100CF | S-20000 | 100 | 35 | 22.0 |
| | | | (65) | | DPCA-60 (25) | (5) | (32.6) | (10.9) | | | |
| Example 66 | 66 | Bk-2 | PI-1 | — | DPHA (10) | OXL-73 | Bk-S0100CF | S-20000 | 100 | 35 | 22.0 |
| | | | (65) | | DPCA-60 (25) | (5) | (32.6) | (10.9) | | | |
| Example 67 | 67 | Bk-2 | PI-1 | — | DPHA (10) | OXL-100 | Bk-S0100CF | S-20000 | 100 | 35 | 22.0 |
| | | | (65) | | DPCA-60 (25) | (5) | (32.6) | (10.9) | | | |
| Example 68 | 68 | Bk-2 | PI-1 | — | DPHA (10) | OXL-101 | Bk-S0100CF | S-20000 | 100 | 35 | 22.0 |
| | | | (65) | | DPCA-60 (25) | (5) | (32.6) | (10.9) | | | |

TABLE 11-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 7 | 7 | 35 | 0 | 32 | 27 | 0.40 | 8.3 |
| | | A | A+ | A | A+ | A | A |
| Example 60 | 60 | 50 | 0 | 45 | 30 | 0.55 | 8.6 |
| | | B | A+ | A | A+ | B | A |
| Example 61 | 61 | 50 | 0 | 45 | 30 | 0.55 | 8.6 |
| | | B | A+ | A | A+ | B | A |
| Example 62 | 62 | 45 | 0 | 42 | 28 | 0.50 | 8.4 |
| | | A | A+ | A | A+ | B | A |
| Example 63 | 63 | 45 | 0 | 42 | 28 | 0.50 | 8.4 |
| | | A | A+ | A | A+ | B | A |
| Example 64 | 64 | 35 | 0 | 37 | 27 | 0.40 | 8.3 |
| | | A | A+ | A | A+ | A | A |
| Example 65 | 65 | 35 | 0 | 37 | 27 | 0.40 | 8.3 |
| | | A | A+ | A | A+ | A | A |
| Example 66 | 66 | 50 | 0 | 45 | 30 | 0.55 | 8.6 |
| | | B | A+ | A | A+ | B | A |
| Example 67 | 67 | 50 | 0 | 45 | 30 | 0.55 | 8.6 |
| | | B | A+ | A | A+ | B | A |
| Example 68 | 68 | 50 | 0 | 45 | 30 | 0.55 | 8.6 |
| | | B | A+ | A | A+ | B | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 7 | 1.0 | >1.0 × | Favorable | 100 |
| | | 10\hat{ }15 | | A+ |
| Example 60 | 1.0 | >1.0 × | Favorable | 100 |
| | | 10\hat{ }15 | | A+ |
| Example 61 | 1.0 | >1.0 × | Favorable | 100 |
| | | 10\hat{ }15 | | A+ |
| Example 62 | 1.0 | >1.0 × | Favorable | 100 |
| | | 10\hat{ }15 | | A+ |
| Example 63 | 1.0 | >1.0 × | Favorable | 100 |
| | | 10\hat{ }15 | | A+ |
| Example 64 | 1.0 | >1.0 × | Favorable | 100 |
| | | 10\hat{ }15 | | A+ |
| Example 65 | 1.0 | >1.0 × | Favorable | 100 |
| | | 10\hat{ }15 | | A+ |
| Example 66 | 1.0 | >1.0 × | Favorable | 100 |
| | | 10\hat{ }15 | | A+ |
| Example 67 | 1.0 | >1.0 × | Favorable | 100 |
| | | 10\hat{ }15 | | A+ |
| Example 68 | 1.0 | >1.0 × | Favorable | 100 |
| | | 10\hat{ }15 | | A+ |

TABLE 12-1

| | | | | | | | | | | | Content | |
|---|---|---|---|---|---|---|---|---|---|---|---|---|
| | | Composition [parts by mass] | | | | | | | | Content | Ratio of | (D) Content |
| | | | | | (B) Residual | | | | | Ratio of | (B) to | Ratio of |
| | | | | | Polymerizable | | | | (F) | (A1) to | (A1) + | Colorant to |
| | | | | | Compound | (C1) Photo | | | Multi- | (A1) + | (A2) + | Total Solid |
| | | | (A1) | (A2) | (B1) to | Initiator | | (E) | func- | (A2) | (B) | Content |
| | Compo- | Pigment | First | Second | (B3) | (C1-1) | (D) | Disper- | tional | [% by | [% by | [% by |
| | sition | Dispersion | Resin | Resin | compounds | Compound | Colorant | sion | Thiol | mass] | mass] | mass] |
| Example 7 | 7 | Bk-2 | PI-1 | — | DPHA | OXL-21 | Bk-S0100CF | S-20000 | — | 100 | 35 | 22.0 |
| | | | (65) | | (10) | (5) | (32.6) | (10.9) | | | | |
| | | | | | DPCA-60 | | | | | | | |
| | | | | | (25) | | | | | | | |
| Example 69 | 69 | Bk-2 | PI-1 | — | DPHA | OXL-21 | Bk-S0100CF | S-20000 | TMMP | 100 | 35 | 22.0 |
| | | | (65) | | (10) | (5) | (32.6) | (10.9) | (0.3) | | | |
| | | | | | DPCA-60 | | | | | | | |
| | | | | | (25) | | | | | | | |
| Example 70 | 70 | Bk-2 | PI-1 | — | DPHA | OXL-21 | Bk-S0100CF | S-20000 | EGME | 100 | 35 | 22.0 |
| | | | (65) | | (10) | (5) | (32.6) | (10.9) | (0.3) | | | |
| | | | | | DPCA-60 | | | | | | | |
| | | | | | (25) | | | | | | | |
| Example 71 | 71 | Bk-2 | PI-1 | — | DPHA | OXL-21 | Bk-S0100CF | S-20000 | TMMP | 100 | 35 | 22.0 |
| | | | (65) | | (35) | (5) | (32.6) | (10.9) | (0.3) | | | |
| Example 72 | 72 | Bk-2 | PI-1 | — | DPHA | OXL-21 | Bk-S0100CF | S-20000 | EGME | 100 | 35 | 22.0 |
| | | | (65) | | (35) | (5) | (32.6) | (10.9) | (0.3) | | | |
| Example 73 | 73 | Bk-2 | PI-1 | — | DPHA | OXL-21 | Bk-S0100CF | S-20000 | — | 100 | 35 | 22.0 |
| | | | (65) | | (5) | (5) | (32.6) | (10.9) | | | | |
| | | | | | DPCA-60 | | | | | | | |
| | | | | | (25) | | | | | | | |
| | | | | | A-DCP | | | | | | | |
| | | | | | (5) | | | | | | | |
| Example 74 | 74 | Bk-2 | PI-1 | — | DPHA | OXL-21 | Bk-S0100CF | S-20000 | TMMP | 100 | 35 | 22.0 |
| | | | (65) | | (5) | (5) | (32.6) | (10.9) | (0.3) | | | |
| | | | | | DPCA-60 | | | | | | | |
| | | | | | (25) | | | | | | | |
| | | | | | A-DCP | | | | | | | |
| | | | | | (5) | | | | | | | |
| Example 75 | 75 | Bk-2 | PI-1 | — | DPHA | OXL-21 | Bk-S0100CF | S-20000 | — | 100 | 35 | 21.8 |
| | | | (65) | | (10) | (5) | (32.6) | (10.9) | | | | |
| | | | | | DPCA-60 | IC-379EG | | | | | | |
| | | | | | (25) | (1.5) | | | | | | |
| Example 76 | 76 | Bk-2 | PI-1 | — | DPHA | OXL-21 | Bk-S0100CF | S-20000 | — | 100 | 35 | 21.8 |
| | | | (65) | | (10) | (5) | (32.6) | (10.9) | | | | |
| | | | | | DPCA-60 | IC-819 | | | | | | |
| | | | | | (25) | (1.5) | | | | | | |
| Example 77 | 77 | Bk-2 | PI-1 | — | DPHA | OXL-21 | Bk-S0100CF | S-20000 | — | 100 | 35 | 21.8 |
| | | | (65) | | (10) | (5) | (32.6) | (10.9) | | | | |
| | | | | | DPCA-60 | HABI-102 | | | | | | |
| | | | | | (25) | (1.5) | | | | | | |

TABLE 12-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 7 | 7 | 35 | 0 | 37 | 27 | 0.40 | 8.3 |
| | | A | A+ | A | A+ | A | A |
| Example 69 | 69 | 30 | 0 | 34 | 24 | 0.30 | 8.5 |
| | | A+ | A+ | A | A+ | A | A |
| Example 70 | 70 | 30 | 0 | 34 | 24 | 0.30 | 8.5 |
| | | A+ | A+ | A | A+ | A | A |
| Example 71 | 71 | 50 | 3 | 34 | 20 | 0.50 | 8.7 |
| | | B | A | A | A+ | B | A |
| Example 72 | 72 | 50 | 0 | 34 | 20 | 0.50 | 8.7 |
| | | B | A+ | A | A+ | B | A |
| Example 73 | 73 | 35 | 0 | 35 | 25 | 0.35 | 8.0 |
| | | A | A+ | A | A+ | A | A |
| Example 74 | 74 | 30 | 0 | 30 | 20 | 0.25 | 8.0 |
| | | A+ | A+ | A+ | A+ | A | A |
| Example 75 | 75 | 30 | 0 | 37 | 27 | 0.30 | 8.2 |
| | | A | A+ | A | A+ | A | A |
| Example 76 | 76 | 30 | 0 | 37 | 27 | 0.30 | 8.2 |
| | | A | A+ | A | A+ | A | A |
| Example 77 | 77 | 30 | 0 | 37 | 27 | 0.30 | 8.2 |
| | | A | A+ | A | A+ | A | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 7 | 1.0 | >1.0 × | Favorable | 100 |
| | | 10\hat{ }15 | | A+ |
| Example 69 | 1.0 | >1.0 × | Favorable | 100 |
| | | 10\hat{ }15 | | A+ |
| Example 70 | 1.0 | >1.0 × | Favorable | 100 |
| | | 10\hat{ }15 | | A+ |
| Example 71 | 1.0 | >1.0 × | Favorable | 100 |
| | | 10\hat{ }15 | | A+ |
| Example 72 | 1.0 | >1.0 × | Favorable | 100 |
| | | 10\hat{ }15 | | A+ |
| Example 73 | 1.0 | >1.0 × | Favorable | 100 |
| | | 10\hat{ }15 | | A+ |
| Example 74 | 1.0 | >1.0 × | Favorable | 100 |
| | | 10\hat{ }15 | | A+ |
| Example 75 | 1.0 | >1.0 × | Favorable | 100 |
| | | 10\hat{ }15 | | A+ |
| Example 76 | 1.0 | >1.0 × | Favorable | 100 |
| | | 10\hat{ }15 | | A+ |
| Example 77 | 1.0 | >1.0 × | Favorable | 100 |
| | | 10\hat{ }15 | | A+ |

TABLE 13-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Residual | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | | Pigment | First | Second | (B3) | (C1-1) | (D) | (E) | [% by | [% by | [% by |
| | Composition | Dispersion | Resin | Resin | compounds | Compound | Colorant | Dispersion | mass] | mass] | mass] |
| Example 7 | 7 | Bk-2 | PI-1 | — | DPHA (10) | OXL-21 | Bk-S0100CF | S-20000 | 100 | 35 | 22.0 |
| | | | (65) | | DPCA-60 (25) | (5) | (32.6) | (10.9) | | | |
| Example 78 | 78 | Bk-13 | PI-1 | — | DPHA (10) | OXL-21 | Bk-CBF | S-20000 | 100 | 35 | 22.0 |
| | | | (65) | | DPCA-60 (25) | (5) | (32.6) | (10.9) | | | |
| Example 79 | 79 | Bk-2 | PI-1 | — | DPHA (10) | OXL-21 | Bk-S0100CF | S-20000 | 100 | 35 | 22.0 |
| | | Bk-14 | (65) | | DPCA-60 (25) | (5) | (22.8) | (10.9) | | | |
| | | | | | | | P.B.60 | | | | |
| | | | | | | | (9.8) | | | | |
| Example 80 | 80 | Bk-2 | PI-1 | — | DPHA (10) | OXL-21 | Bk-S0100CF | S-20000 | 100 | 35 | 22.0 |
| | | Bk-15 | (65) | | DPCA-60 (25) | (5) | (22.8) | (10.9) | | | |
| | | | | | | | P.R.179 | | | | |
| | | | | | | | (9.8) | | | | |
| Example 81 | 81 | Bk-2 | PI-1 | — | DPHA (10) | OXL-21 | Bk-S0100CF | S-20000 | 100 | 35 | 22.0 |
| | | Bk-16 | (65) | | DPCA-60 (25) | (5) | (22.8) | (10.9) | | | |
| | | | | | | | P.V.37 | | | | |
| | | | | | | | (9.8) | | | | |
| Example 82 | 82 | Bk-2 | PI-1 | — | DPHA (10) | OXL-21 | Bk-S0100CF | S-20000 | 100 | 35 | 22.0 |
| | | Bk-15 | (65) | | DPCA-60 (25) | (5) | (22.8) | (10.9) | | | |
| | | Bk-17 | | | | | P.R.179 | | | | |
| | | | | | | | (4.9) | | | | |
| | | | | | | | P.Y.192 | | | | |
| | | | | | | | (4.9) | | | | |
| Example 83 | 83 | Bk-2 | PI-1 | — | DPHA (10) | OXL-21 | Bk-S0100CF | S-20000 | 100 | 35 | 22.0 |
| | | Bk-15 | (65) | | DPCA-60 (25) | (5) | P.R.179 | (10.9) | | | |
| | | Bk-18 | | | | | (4.9) | | | | |
| | | | | | | | P.O.43 | | | | |
| | | | | | | | (4.9) | | | | |

TABLE 13-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Example 7 | 7 | 35 | 0 | 37 | 27 | 0.40 | 8.3 |
| | | A | A+ | A | A+ | A | A |
| Example 78 | 78 | 35 | 0 | 30 | 22 | 0.30 | 6.0 |
| | | A | A+ | A+ | A+ | A | A |
| Example 79 | 79 | 25 | 0 | 34 | 24 | 0.40 | 8.5 |
| | | A+ | A+ | A | A+ | A | A |
| Example 80 | 80 | 25 | 0 | 34 | 24 | 0.40 | 8.5 |
| | | A+ | A+ | A | A+ | A | A |
| Example 81 | 81 | 30 | 0 | 34 | 24 | 0.40 | 8.5 |
| | | A+ | A+ | A | A+ | A | A |
| Example 82 | 82 | 30 | 0 | 33 | 23 | 0.40 | 8.5 |
| | | A+ | A+ | A | A+ | A | A |
| Example 83 | 83 | 30 | 0 | 33 | 23 | 0.40 | 8.5 |
| | | A+ | A+ | A | A+ | A | A |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Example 7 | 1.0 | >1.0 × | Favorable | 100 |
| | | 10\hat{ }15 | | A+ |
| Example 78 | 1.0 | >1.0 × | Favorable | 100 |
| | | 10\hat{ }15 | | A+ |
| Example 79 | 1.0 | >1.0 × | Favorable | 100 |
| | | 10\hat{ }15 | | A+ |
| Example 80 | 1.0 | >1.0 × | Favorable | 100 |
| | | 10\hat{ }15 | | A+ |
| Example 81 | 1.0 | >1.0 × | Favorable | 100 |
| | | 10\hat{ }15 | | A+ |
| Example 82 | 1.0 | >1.0 × | Favorable | 100 |
| | | 10\hat{ }15 | | A+ |
| Example 83 | 1.0 | >1.0 × | Favorable | 100 |
| | | 10\hat{ }15 | | A+ |

TABLE 14-1

| | | | | | | | | | | Content | |
|---|---|---|---|---|---|---|---|---|---|---|---|
| | | Composition [parts by mass] | | | | | | | Content | Ratio of | (D) Content |
| | | | | | (B) Residual | | | | Ratio of | (B) to | Ratio of |
| | | | | | Polymerizable | | | | (A1) to | (A1) + | Colorant to |
| | | | | | Compound | (C1) Photo | | | (A1) + | (A2) + | Total Solid |
| | | | (A1) | (A2) | (B1) to | Initiator | | | (A2) | (B) | Content |
| | | Pigment | First | Second | (B3) | (C1-1) | (D) | (E) | [% by | [% by | [% by |
| | Composition | Dispersion | Resin | Resin | compounds | Compound | Colorant | Dispersion | mass] | mass] | mass] |
| Comparative | 60 | Bk-2 | — | AC-1 | DPHA (10) | OXL-21 | Bk-S0100CF | S-20000 | 0 | 35 | 22.0 |
| Example 1 | | | | (65) | DPCA-60 (25) | (5) | (32.6) | (10.9) | | | |
| Comparative | 61 | Bk-2 | PI-1 | — | DPHA (10) | OXE-02 | Bk-S0100CF | S-20000 | 100 | 35 | 2.5 |
| Example 2 | | | (65) | | DPCA-60 (25) | (5) | (2.7) | (1) | | | |
| Comparative | 62 | Bk-2 | PI-1 | — | DPHA (10) | OXE-02 | Bk-S0100CF | S-20000 | 100 | 35 | 10.0 |
| Example 3 | | | (65) | | DPCA-60 (25) | (5) | (12.1) | (4.0) | | | |
| Comparative | 63 | Bk-2 | PI-1 | — | DPHA (10) | OXE-02 | Bk-S0100CF | S-20000 | 100 | 35 | 22.0 |
| Example 4 | | | (65) | | DPCA-60 (25) | (5) | (32.6) | (10.9) | | | |
| Comparative | 64 | Bk-2 | PI-1 | — | DPHA (10) | OXL-A | Bk-S0100CF | S-20000 | 100 | 35 | 22.0 |
| Example 5 | | | (65) | | DPCA-60 (25) | (5) | (32.6) | (10.9) | | | |
| Comparative | 65 | Bk-2 | PI-1 | — | DPHA (10) | OXL-A | P.R.254 | D.BYK-167 | 100 | 35 | 32.0 |
| Example 6 | | | (65) | | DPCA-60 (25) | (5) | (20.5) | (19.5) | | | |
| | | | | | | | P.Y.139 | | | | |
| | | | | | | | (8.8) | | | | |
| | | | | | | | P.B.15:6 | | | | |
| | | | | | | | (29.3) | | | | |
| Comparative | 66 | Bk-2 | PI-5 | — | DPHA (35) | OXL-21 | Bk-S0100CF | S-20000 | 100 | 35 | 22.0 |
| Example 7 | | | (65) | | | (5) | (32.6) | (10.9) | | | |
| Comparative | 67 | — | PI-1 | — | DPHA (35) | OXL-21 | — | — | 100 | 35 | 0.0 |
| Example 8 | | | (65) | | | (5) | | | | | |

TABLE 14-2

| | | Photosensitive Characteristics/Cured Film Characteristics | | | | | |
|---|---|---|---|---|---|---|---|
| | | | | | | Change in | |
| | | | | | | Pattern | Heat |
| | | | | | | Opening | Resistance |
| | | | | | | Width | Difference |
| | | | | | Pattern | Dimension | in High- |
| | | | | Pattern | Cross- | between | temperature |
| | | | | Cross- | section | Before and | Weight |
| | | | | section | Shape after | After | Residual |
| | | | Development | Shape after | Thermal | Thermal | Ratio |
| | | Sensitivity | Residue | Development | Curing | Curing | [% by |
| | Composition | [mJ/cm²] | [%] | [°] | [°] | [μm] | mass] |
| Comparative | 60 | 25 | 35 | 50 | 35 | 0.65 | 37.8 |
| Example 1 | | A+ | D | B | A | C | D |
| Comparative | 61 | 60 | 5 | 50 | 20 | 1.00 | 8.5 |
| Example 2 | | B | A | B | A+ | C | A |
| Comparative | 62 | 70 | 10 | 60 | 25 | 1.10 | 9.3 |
| Example 3 | | C | B | B | A+ | D | A |
| Comparative | 63 | 90 | 20 | 65 | 25 | 1.20 | 10.3 |
| Example 4 | | C | C | C | A+ | D | A |
| Comparative | 64 | 50 | B | 60 | 30 | 0.80 | 9.5 |
| Example 5 | | B | 10 | B | A+ | C | A |
| Comparative | 65 | 65 | B | 65 | 25 | 1.30 | 12.5 |
| Example 6 | | C | 10 | C | A+ | D | B |
| Comparative | 66 | 65 | 0 | 50 | 40 | 0.40 | 8.7 |
| Example 7 | | C | A+ | B | A | A | A |
| Comparative | 67 | 20 | 0 | 40 | 30 | 0.45 | 5.0 |
| Example 8 | | A+ | A+ | A | A+ | B | A+ |

| | | | Light-emitting | |
|---|---|---|---|---|
| | | | Characteristics | |
| | Photosensitive Characteristics/Cured Film Characteristics | | of Organic EL | |
| | | Insulation | Display Device | |
| | | Properties | | Characteristics |
| | | Surface | | after |
| | Light- | Resistivity | | Durability |
| | blocking | [Ω/ | Initial | Test |
| | OD Value | □] | Characteristics | [%] |
| Comparative | 1.0 | >1.0 × | Favorable | 30 |
| Example 1 | | 10\hat{ }15 | | E |
| Comparative | 0.15 | >1.0 × | Favorable | 100 |
| Example 2 | | 10\hat{ }15 | | A+ |
| Comparative | 0.5 | >1.0 × | Favorable | 100 |
| Example 3 | | 10\hat{ }15 | | A+ |
| Comparative | 1.0 | >1.0 × | Favorable | 100 |
| Example 4 | | 10\hat{ }15 | | A+ |
| Comparative | 1.0 | >1.0 × | Favorable | 100 |
| Example 5 | | 10\hat{ }15 | | A+ |
| Comparative | 1.0 | >1.0 × | Favorable | 90 |
| Example 6 | | 10\hat{ }15 | | A |
| Comparative | 1.0 | >1.0 × | Favorable | 100 |
| Example 7 | | 10\hat{ }15 | | A+ |
| Comparative | — | >1.0 × | Favorable | 100 |
| Example 8 | | 10\hat{ }15 | | A+ |

### Example 84

(Method for Manufacturing Organic EL Display without Polarizing Layer)

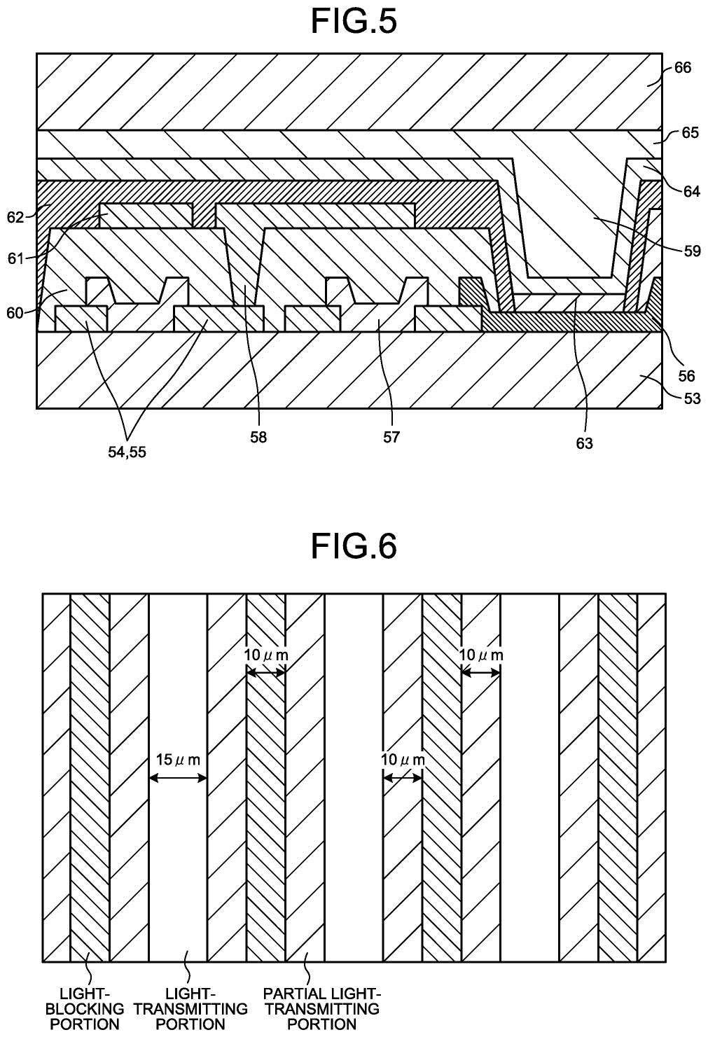

FIG. 5 shows therein an outline of an organic EL display to e prepared. First, a laminated film of chromium and gold was formed by an electron beam evaporation method on 38×46 mm non-alkali glass substrate 53, and a source electrode 54 and drain electrode 55 were formed by etching. Next, APC (silver/palladium/copper=98.07/0.87/1.06 (mass ratio)) of 100 nm was deposited by sputtering, and subjected to pattern processing by etching to form an APC layer, and an ITO of 10 nm was further deposited by sputtering for an upper layer on the APC layer, and etched to form a reflective electrode 56 as a first electrode. After cleaning the electrode surface with oxygen plasma, an amorphous IGZO was deposited by sputtering, and etched to form an oxide semiconductor layer 57 between the source and drain electrodes. Next, a positive photosensitive polysiloxane-based material (SP-P2301; manufactured by Toray Industries, Inc.) was deposited by a spin coating method, and after making a via hole 58 and a pixel region 59 as openings by photolithography, thermally cured to form a gate insulation layer 60. Thereafter, gold was deposited by an electron beam evaporation method, and etched to form a gate electrode 61, thereby providing an oxide TFT array.

In accordance with the above-described method described in Example 1, the composition 7 was applied and prebaked on the oxide TFT array to form a film, and the film was subjected to exposure for patterning through a photomask with a predetermined pattern, and development and rinsing to make a pixel region as an opening, and then thermally cured to form a TFT protective layer/pixel defining layer 62 with light-blocking property. In accordance with the method described above, openings of 70 μm in width and 260 μm in length were arranged at a pitch of 155 μm in the width direction and a pitch of 465 μm in the length direction, and the pixel defining layer in a shape for exposing the reflective electrode through the respective openings was formed only on a substrate effective area in a limited fashion. It is to be noted that the openings will finally serve for light-emitting pixels of an organic EL display. Further, the substrate effective area was a square of 16 mm on a side, and the pixel defining layer was formed to have a thickness of about 1.0 μm.

Next, an organic EL light-emitting layer 63 was formed by the method described in the section (16) described above, with the use of the compound (HT-1) as a hole injection layer, the compound (HT-2) as a hole transport layer, the compound (GH-1) as a host material, the compound (GD-1) as a dopant material, and the compound (ET-1) and the compound (LiQ) as electron transport materials.

Thereafter, MgAg (magnesium/silver=10/1 (volume ratio)) of 10 nm was deposited by a vapor deposition method, and etched to form a transparent electrode 64 as a second electrode. Then, a sealing film 65 was formed with the use of an organic EL sealing material (Structbond (registered trademark) XMF-T; manufactured by Mitsui Chemicals, Inc.) under a low-humidity nitrogen atmosphere. Furthermore, a non-alkali glass substrate 66 was bonded onto the sealing film, thereby preparing, on one substrate, four top-emission organic EL displays each of 5 mm on a side without any polarizing layer. It is to be noted that the film thickness herein refers to a value displayed on a crystal oscillation-type film thickness monitor.

### (Light-Emitting Characteristic Evaluation)

The organic EL display prepared by the method described above was allowed to emit light by direct-current drive at 10 mA/cm², and the luminance (Y′) in the case of irradiating the pixel defining layer part with external light, and the luminance (Y₀) in the case of irradiating the part with no external light were measured. As an index for reduction in external light reflection, the contrast was calculated by the following equation:

Contrast=Y₀/Y′

It has been determined as follows that A+, A, and B where the contrast is 0.80 or more are regarded as pass, A+ and A where the contrast is 0.90 or more are regarded as favorable effects of reduction in external light reflection, and A+ where the contrast is 0.95 or more is regarded as an excellent effect of reduction in external light reflection. It has been confirmed that the organic EL display prepared by the above-described method has contrast of 0.90, and has ability to reduce external light reflection.

A+: The contrast is 0.95 to 1.00.

A: The contrast is 0.90 to 0.94.

B: The contrast is 0.80 to 0.89.

C: The contrast is 0.70 to 0.79.

D: The contrast is 0.50 to 0.69.

E: The contrast is 0.01 to 0.49.

### Example 85

### (Evaluation of Halftone Characteristics)

In accordance with the method described in Example 1 as described above, a prebaked film of the composition 7 was formed to have a film thickness of 5 μm on an ITO substrate, subjected to exposure for patterning with the i-line (wavelength: 365 nm), h-line (wavelength: 405 nm), and g-line (wavelength 436 nm) of an ultra-high pressure mercury lamp through a halftone photomask for halftone characteristic evaluation with the use of a double-sided alignment single-sided exposure apparatus (Mask Aligner PEM-6M; manufactured by Union Optical Co., Ltd.) such that the exposure energy for the light-transmitting portion reached the exposure energy for the sensitivity in the case of the film thickness of 5 μm after prebaking, and developed with the use of a small-size developing device (AD-2000; TAKIZAWA SANGYO K.K.) for photolithography, and a cured film of the composition 7 was then prepared with the use of a high-temperature inert gas oven (INH-9CD-S; manufactured by Koyo Thermo Systems Co., Ltd.).

Photomasks including a light-transmitting portion, a light-blocking portion, and a partial light-transmitting portion between the light-transmitting portion and the light-blocking portion were used as the halftone photomask. The photomasks have sites where the transmittances (% T_{HT})% of the partial light-transmitting portions are respectively 10%, 15%, 20%, 25%, 30%, 35%, 40%, 45%, and 50% of the transmittance (% T_{FT}) of the light-transmitting portion. The light-transmitting portion is adjacent to the partial light-transmitting portion, and the partial light-transmitting portion is adjacent to the light-blocking portion. The pattern shapes of the light-transmitting portion, partial light-transmitting portion, and light-blocking portion each have a site in a line shape. The light-transmitting portion and the light-blocking portion each have a site in a quadrangular shape. The light-transmitting portions each have a site of 2 μm, 5 μm, 10 μm, 15 μm, 20 μm, 30 μm, 40 μm, 50 μm, or 100 μm in pattern width. In addition, the light-blocking portions are 10 μm in pattern width. On the other hand, the partial light-transmitting portions each have a site of 2 μm, 5 μm, 10 μm, 15 μm, 20 μm, 25 μm, 30 μm, 35 μm, 40 μm, 45 μm, 50 μm, and 100 μm in pattern width.

FIG. 6 shows, as an example of the halftone photomask, an example of the arrangement and dimensions of the light-transmitting portion, light-blocking portion, and partial light-transmitting portion.

With the use of a surface texture and contour measuring instrument (SURFCOM 1400D; TOKYO SEIMITSU CO., LTD.), at the measurement magnification of 10,000 times, the measurement length of 1.0 mm, the measurement speed of 0.30 mm/s, the film thickness of the light-transmitting portion after development and the film thickness (T_{FT}) μm thereof after the thermal curing were measured. For the partial light-transmitting portions, the film thicknesses of sites different in transmittance after development and the film thicknesses (T_{HT}) μm thereof after thermal curing were measured, thereby determining the minimum film thickness (T_{HT/min}) μm after thermal curing of the partial light-transmitting portion left as a residual film after development. As an index of halftone characteristics, the maximum step film thickness was calculated by the following formula:

Maximum Step Film Thickness=(T_{FT})−(T_{HT/min}).

It has been determined as follows that A+, A, and B, and C where the maximum step film thickness is 1.0 μm or more are regarded as pass, A+, A, and B where the maximum step film thickness is 1.5 μm or more are regarded as favorable halftone characteristics, and A+ and A where the maximum step film thickness is 2.0 μm or more are regarded as excellent half-tone characteristics. It has been confirmed that the cured film of the composition 7 prepared by the method mentioned above has a light-transmitting portion of 4.0 μm in film thickness (T_{FT}) after thermal curing, and a partial light-transmitting portion of 2.3 μm in minimum film thickness (T_{HT/min}) after thermal curing, and thus have a maximum step film thickness of 1.7 μm, and have excellent halftone characteristics.

A+: The maximum step film thickness is 2.5 μm or more.

A: The maximum step film thickness is 2.0 μm or more and less than 2.5 μm.

B: The maximum step film thickness is 1.5 μm or more and less than 2.0 μm.

C: The maximum step film thickness is 1.0 μm or more and less than 1.5 μm.

D: The maximum step film thickness is 0.5 μm or more and less than 1.0 μm.

E: The maximum step thickness is 0.1 μm or more and less than 0.5 μm.

F: The maximum step film thickness is less than 0.1 μm or not measurable without any residual film after the development.

In accordance with similar manners, the halftone characteristics were evaluated with the use of the compositions 18 to 35, 60 to 68, 1, 5, 43, 45, 47, 48, 51, 53, 54, 56 to 59, and 78 to 81 as Examples 86 to 129, and the compositions 87, 88, 84, 90, and 89 as Comparative Examples 9 to 13. The evaluation results of Examples 85 to 129 and Comparative Examples 9 to 13 are shown in Table 15-1 and Table 15-2.

TABLE 15-1

| | | | Photosensitive Characteristics/ |
|---|---|---|---|
| | | | Cured Film Characteristics |
| | | | Halftone Characteristics |
| | | (C1) Photo Initiator | Maximum Step Film Thickness |
| | Composition | (C1-1) Compound | [μm] |
| Example 85 | 7 | OXL-21 | 1.7 |
| | | (5) | Favorable |
| Example 86 | 18 | OXL-16 | 1.5 |
| | | (5) | Favorable |
| Example 87 | 19 | OXL-19 | 1.7 |
| | | (5) | Favorable |
| Example 88 | 20 | OXL-1 | 1.0 |
| | | (5) | Pass |
| Example 89 | 21 | OXL-43 | 1.0 |
| | | (5) | Pass |
| Example 90 | 22 | OXL-44 | 1.2 |
| | | (5) | Pass |
| Example 91 | 23 | OXL-2 | 1.0 |
| | | (5) | Pass |
| Example 92 | 24 | OXL-58 | 1.0 |
| | | (5) | Pass |
| Example 93 | 25 | OXL-63 | 1.2 |
| | | (5) | Pass |
| Example 94 | 26 | OXL-7 | 1.5 |
| | | (5) | Favorable |
| Example 95 | 27 | OXL-54 | 1.5 |
| | | (5) | Favorable |
| Example 96 | 28 | OXL-55 | 1.7 |
| | | (5) | Favorable |
| Example 97 | 29 | OXL-9 | 1.0 |
| | | (5) | Pass |
| Example 98 | 30 | OXL-9 | 1.0 |
| | | (5) | Pass |
| Example 99 | 31 | OXL-31 | 1.5 |
| | | (5) | Favorable |
| Example 100 | 32 | OXL-37 | 1.5 |
| | | (5) | Favorable |
| Example 101 | 33 | OXL-71 | 1.0 |
| | | (5) | Pass |
| Example 102 | 34 | OXL-79 | 0.6 |
| | | (5) | Defective |
| Example 103 | 35 | OXL-81 | 0.6 |
| | | (5) | Defective |
| Example 104 | 60 | OXL-82 | 1.0 |
| | | (5) | Pass |
| Example 105 | 61 | OXL-83 | 1.0 |
| | | (5) | Pass |
| Example 106 | 62 | OXL-87 | 1.2 |
| | | (5) | Pass |
| Example 107 | 63 | OXL-96 | 1.2 |
| | | (5) | Pass |
| Example 108 | 64 | OXL-97 | 1.8 |
| | | (5) | Favorable |
| Example 109 | 65 | OXL-99 | 1.8 |
| | | (5) | Favorable |
| Example 110 | 66 | OXL-73 | 1.0 |
| | | (5) | Pass |
| Example 111 | 67 | OXL-100 | 1.0 |
| | | (5) | Pass |
| Example 112 | 68 | OXL-101 | 1.0 |
| | | (5) | Pass |
| Comparative | 87 | OXE-02 | 0.2 |
| Example 9 | | (5) | Defective |
| Comparative | 88 | OXL-A | 0.4 |
| Example 10 | | (5) | Defective |

TABLE 15-2

| | | | | | Photosensitive Characteristics/ |
|---|---|---|---|---|---|
| | | (C1) Photo | (A1) First | (A2) Second | Cured Film Characteristics |
| | | Initiator | Resin | Resin | Halftone Characteristics |
| | | (C1-1) | [parts by | [parts by | Maximum Step Film Thickness |
| | Composition | Compound | mass] | mass] | [μm] |
| Example 113 | 1 | OXL-21 | PI-1 | — | 1.6 |
| | | (5) | (65) | | Favorable |
| Example 114 | 5 | OXL-21 | PI-1 | PS-1 | 2.2 |
| | | (5) | (50) | (15) | Excellent |
| Example 85 | 7 | OXL-21 | PI-1 | — | 1.7 |
| | | (5) | (65) | | Favorable |
| Example 115 | 43 | OXL-21 | PI-1 | PS-1 | 2.4 |
| | | (5) | (50) | (15) | Excellent |
| Example 116 | 45 | OXL-21 | PI-1 | CR-1 | 2.3 |
| | | (5) | (50) | (15) | Excellent |
| Example 117 | 47 | OXL-21 | PI-1 | AE-1 | 2.1 |
| | | (5) | (50) | (15) | Excellent |
| Example 118 | 48 | OXL-21 | PI-1 | AC-1 | 1.9 |
| | | (5) | (50) | (15) | Favorable |
| Comparative | 84 | OXL-21 | — | AC-1 | 0.2 |
| Example 11 | | (5) | | (65) | Defective |
| Comparative | 90 | OXL-21 | PI-5 | — | 0.8 |
| Example 12 | | (5) | (65) | | Defective |

| | | | | Photosensitive Characteristics/ |
|---|---|---|---|---|
| | | (C1) Photo | | Cured Film Characteristics |
| | | Initiator | | Halftone Characteristics |
| | | (C1-1) | | Maximum Step Film Thickness |
| | Composition | Compound | (D) Colorant | [μm] |
| Example 85 | 7 | OXL-21 | Bk-S0100CF | 1.7 |
| | | (5) | (32.6) | Favorable |
| Example 119 | 51 | OXL-21 | Bk-S0084 | 1.2 |
| | | (5) | (32.6) | Pass |
| Example 120 | 53 | OXL-21 | TPK-1227 | 0.6 |
| | | (5) | (19.6) | Defective |
| Example 121 | 54 | OXL-21 | P.R.254 (20.5) | 1.0 |
| | | (5) | P.Y.139 (8.8) | Pass |
| | | | P.B.15:6 (29.3) | |
| Example 122 | 56 | OXL-21 | P.R.179 (17.6) | 1.5 |
| | | (5) | P.Y.192 (17.6) | Favorable |
| | | | P.B.60 (23.4) | |
| Example 123 | 57 | OXL-21 | P.V.37 (38.0) | 1.5 |
| | | (5) | P.Y.192 (20.5) | Favorable |
| Example 124 | 58 | OXL-21 | P.R.179 (17.6) | 1.5 |
| | | (5) | P.O.43 (17.6) | Favorable |
| | | | P.B.60 (23.4) | |
| Example 125 | 59 | OXL-21 | P.V.37 (32.2) | 1.5 |
| | | (5) | P.O.43 (17.6) | Favorable |
| | | | P.B.60 (8.8) | |
| Example 126 | 78 | OXL-21 | Bk-CBF | 2.2 |
| | | (5) | (32.6) | Excellent |
| Example 127 | 79 | OXL-21 | Bk-S0100CF (22.8) | 2.0 |
| | | (5) | P.B.60 (9.8) | Excellent |
| Example 128 | 80 | OXL-21 | Bk-S0100CF (22.8) | 2.0 |
| | | (5) | P.R.179 (9.8) | Excellent |
| Example 129 | 81 | OXL-21 | Bk-S0100CF (22.8) | 2.0 |
| | | (5) | P.V.37 (9.8) | Excellent |
| Comparative | 89 | OXL-A | P.R.254 (20.5) | 0.2 |
| Example 13 | | (5) | P.Y.139 (8.8) | Defective |
| | | | P.B.15:6 (29.3) | |

### INDUSTRIAL APPLICABILITY

The negative photosensitive resin composition, the cured film, and the organic EL display and the manufacturing method therefor according to the present invention are suitable for organic EL displays which have display characteristics and reliability improved.

### DESCRIPTION OF REFERENCE SIGNS

1, 12, 15, 26: Glass substrate
2, 16: TFT
3, 17: Cured film for TFT planarization
4: Reflective electrode
5a, 21a: Prebaked film
5b, 21b, 28: Cured pattern
6, 22: Mask
7, 23: Active actinic rays
8: EL light-emitting layer
9, 18, 64: Transparent electrode
10, 29: Cured film for planarization
11: Cover glass
13: BLU
14: Glass substrate with BLU
19: Planarization film
20, 30: Alignment layer
24: Glass substrate with BCS
25: Glass substrate with BLU and BCS
27: Color filter
31: Color filter substrate
32: Glass substrate with BLU, BCS, and BM
33: Liquid crystal layer
34: Thick film part
35a, 35b, 35c: Thin film part
36a, 36b, 36c, 36d, 36e: Inclined side in cross section of cured pattern
37: Horizontal side of underlying substrate
47, 53, 66: Non-alkali glass substrate
48: First electrode
49: Auxiliary electrode
50: Insulation layer
51: Organic EL layer
52: Second electrode
54: Source electrode
55: Drain electrode
56: Reflective electrode
57: Oxide semiconductor layer
58: Via hole
59: Pixel region
60: Gate insulation layer
61: Gate electrode
62: TFT protective layer/pixel defining layer
63: Organic EL light-emitting layer
65: Sealing film

1. A negative photosensitive resin composition comprising an (A) alkali-soluble resin, a (C1) photo initiator, and a (Da) black colorant,
wherein the (A) alkali-soluble resin contains a (A1) first resin including one or more selected from the group consisting of a (A1-1) polyimide, a (A1-2) polyimide precursor, a (A1-3) polybenzoxazole, and a (A1-4) polybenzoxazole precursor,
the one or more selected from the group consisting of the (A1-1) polyimide, the (A1-2) polyimide precursor, the (A1-3) polybenzoxazole, and the (A1-4) polybenzoxazole precursor have a structural unit having a fluorine atom at 10 to 100 mol % of all of structural units,
the (C1) photo initiator contains an (C1-1) oxime ester-based photo initiator, and
the (C1-1) oxime ester-based photo initiator has one or more structures selected from the group consisting of (I), (II), and (III):
(I) one or more structures selected from the group consisting of a naphthalenecarbonyl structure, a trimethylbenzoyl structure, a thiophenecarbonyl structure, and a furancarbonyl structure;
(II) a nitro group, a carbazole structure, and a group represented by the general formula (11);
(III) a nitro group and one or more structures selected from the group consisting of a fluorene structure, a dibenzofuran structure, a dibenzothiophene structure, a naphthalene structure, a diphenylmethane structure, a diphenylamine structure, a diphenyl ether structure, and a diphenyl sulfide structure. 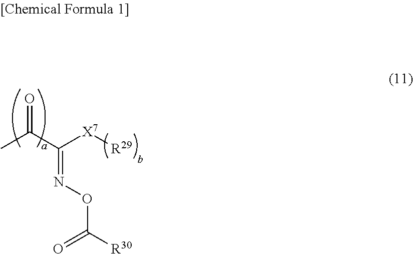
(In the general formula (11), X⁷ represents a direct bond, an alkylene group having 1 to 10 carbon atoms, a cycloalkylene group having 4 to 10 carbon atoms, or an arylene group having 6 to 15 carbon atoms. In a case where X⁷ represents a direct bond, an alkylene group having 1 to 10 carbon atoms, or a cycloalkylene group having 4 to 10 carbon atoms, R²⁹ represents hydrogen, an alkyl group having 1 to 10 carbon atoms, or a cycloalkyl group having 4 to 10 carbon atoms, an alkoxy group having 1 to 10 carbon atoms, a haloalkyl group having 1 to 10 carbon atoms, a haloalkoxy group having 1 to 10 carbon atoms, an acyl group having 2 to 10 carbon atoms, or a nitro group. In a case where X⁷ represents an arylene group having 6 to 15 carbon atoms, R²⁹ represents hydrogen, an alkyl group having 1 to 10 carbon atoms, a cycloalkyl group having 4 to 10 carbon atoms, an aryl group having 6 to 15 carbon atoms, a haloalkyl group having 1 to 10 carbon atoms, a haloalkoxy group having 1 to 10 carbon atoms, a heterocyclic group having 4 to 10 carbon atoms, a heterocyclic oxy group having 4 to 10 carbon atoms, an acyl group having 2 to 10 carbon atoms, or a nitro group. R³⁰ represents hydrogen, an alkyl group having 1 to 10 carbon atoms, a cycloalkyl group having 4 to 10 carbon atoms, or an aryl group having 6 to 15 carbon atoms. a represents 0 or 1, and b represents an integer of 0 to 10.)

2. The negative photosensitive resin composition according to claim 1,
wherein the (Da) black colorant contains a (D1a) black pigment, and
the (D1a) black pigment contains, as a (D1a-1) black organic pigment, one or more selected from the group consisting of a (Da-1a) benzofuranone-based black pigment, a (Da-1b) perylene-based black pigment, and an (D1a-1c) azo-based black pigment.

3. The negative photosensitive resin composition according to claim 2,
wherein the (D1a-1) black organic pigment contains a (D1a-1a) benzofuranone-based black pigment.

4. The negative photosensitive resin composition according to claim 1,
wherein the (Da) black colorant contains a (D1a) black pigment,
the (D1a) black pigment contains, as (D1a-3) coloring pigment mixture of two or more colors, a (D1a-3a) coloring pigment mixture including a blue pigment, a red pigment, and a yellow pigment, a (D1a-3b) coloring pigment mixture including a purple pigment and a yellow pigment, a (D1a-3c) coloring pigment mixture including a blue pigment, a red pigment, and an orange pigment, or a (D1a-3d) coloring pigment mixture including a blue pigment, a purple pigment, and an orange pigment,
the blue pigment contains one or more selected from the group consisting of C.I. pigment blue 15:4, C.I. pigment blue 15:6, and C.I. pigment blue 60,
the red pigment contains one or more selected from the group consisting of C.I. pigment red 123, C.I. pigment red 149, C.I. pigment red 177, C.I. pigment red 179, and C.I. pigment red 190,
the yellow pigment contains one or more selected from the group consisting of C.I. pigment yellow 120, C.I. pigment yellow 151, C.I. pigment yellow 175, C.I. pigment yellow 180, C.I. pigment yellow 181, C.I. pigment yellow 192, and C.I. pigment yellow 194,
the purple pigment contains one or more selected from the group consisting of C.I. pigment violet 19, C.I. pigment violet 29, and C.I. pigment violet 37, and
the orange pigment contains one or more selected from the group consisting of C.I. pigment orange 43, C.I. pigment orange 64, and C.I. pigment orange 72.

5. The negative photosensitive resin composition according to claim 1,
the negative photosensitive resin composition further containing, as a (D1b-1) non-black organic pigment, one or more selected from the group consisting of a blue pigment, a red pigment, a yellow pigment, a purple pigment, an orange pigment, and a green pigment,
wherein the blue pigment contains one or more selected from the group consisting of C.I. pigment blue 15:4, C.I. pigment blue 15:6, and C.I. pigment blue 60,
the red pigment contains one or more selected from the group consisting of C.I. pigment red 123, C.I. pigment red 149, C.I. pigment red 177, C.I. pigment red 179, and C.I. pigment red 190,
the yellow pigment contains one or more selected from the group consisting of C.I. pigment yellow 120, C.I. pigment yellow 151, C.I. pigment yellow 175, C.I. pigment yellow 180, C.I. pigment yellow 181, C.I. pigment yellow 192, and C.I. pigment yellow 194,
the purple pigment contains one or more selected from the group consisting of C.I. pigment violet 19, C.I. pigment violet 29, and C.I. pigment violet 37, and
the orange pigment contains one or more selected from the group consisting of C.I. pigment orange 43, C.I. pigment orange 64, and C.I. pigment orange 72.

6. The negative photosensitive resin composition according to claim 1,
wherein the (C1-1) oxime ester-based photo initiator has a halogen-substituted group.

7. The negative photosensitive resin composition according to claim 1,
wherein the (Da) black colorant has a maximum transmission wavelength of 330 to 410 nm,
the (C1-1) oxime ester-based photo initiator has a maximum absorption wavelength of 330 to 410 nm, and
in a 0.01 g/L propylene glycol monomethyl ether acetate solution of the (C1-1) oxime ester-based photo initiator, an absorbance at a wavelength of 360 nm is 0.20 or more.

8. The negative photosensitive resin composition according to claim 7,
wherein the (C1-1) oxime ester-based photo initiator has a maximum absorption wavelength of 340 to 400 nm, and
in a 0.01 g/L propylene glycol monomethyl ether acetate solution of the (C1-1) oxime ester-based photo initiator, an absorbance at a wavelength of 360 nm is 0.25 or more.

9. The negative photosensitive resin composition according to claim 1,
wherein the (A) alkali-soluble resin further contains a (A2) second resin including one or more selected from the group consisting of a (A2-1) polysiloxane, a (A2-2) polycyclic side chain-containing resin, an (A2-3) acid-modified epoxy resin, and an (A2-4) acrylic resin.

10. The negative photosensitive resin composition according to claim 1,
wherein the (C1-1) oxime ester-based photo initiator contains one or more selected from the group the consisting of a compound represented by the general formula (12), a compound represented by the general formula (13), and a compound represented by the general formula (14). 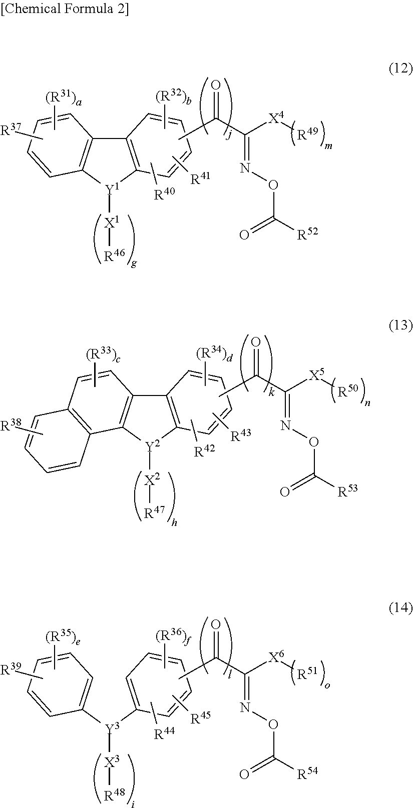
(In the general formulas (12) to (14), X¹ to X⁶ each independently represent a direct bond, an alkylene group having 1 to 10 carbon atoms, a cycloalkylene group having 4 to 10 carbon atoms, or an arylene group having 6 to 15 carbon atoms. Y¹ to Y³ each independently represent carbon, nitrogen, oxygen, or sulfur. R³¹ to R³⁶ each independently represent an alkyl group having 1 to 10 carbon atoms, a cycloalkyl group having 4 to 10 carbon atoms, an aryl group having 6 to 15 carbon atoms, an alkoxy group having 1 to 10 carbon atoms, or a hydroxyalkyl group having 1 to 10 carbon atoms. R³⁷ to R³⁹ each independently represent a group represented by the general formula (15), a group represented by the general formula (16), a group represented by the general formula (17), a group represented by the general formula (18), or a nitro group. R⁴⁰ to R⁴⁵ each independently represent hydrogen, an alkyl group having 1 to 10 carbon atoms, a cycloalkyl group having 4 to 10 carbon atoms, an aryl group having 6 to 15 carbon atoms, or a group that forms a ring having 4 to 10 carbon atoms. R⁴⁶ to R⁴⁸ each independently represent hydrogen, an alkyl group having 1 to 10 carbon atoms, a cycloalkyl group having 4 to 10 carbon atoms, an aryl group having 6 to 15 carbon atoms, an alkenyl group having 1 to 10 carbon atoms, an alkoxy group having 1 to 10 carbon atoms, a haloalkyl group having 1 to 10 carbon atoms, a haloalkoxy group having 1 to 10 carbon atoms, or an acyl group having 2 to 10 carbon atoms. R⁴⁹ to R⁵¹ each independently represent hydrogen, an alkyl group having 1 to 10 carbon atoms, a cycloalkyl group having 4 to 10 carbon atoms, an aryl group having 6 to 15 carbon atoms, an alkoxy group having 1 to 10 carbon atoms, a haloalkyl group having 1 to 10 carbon atoms, a haloalkoxy group having 1 to 10 carbon atoms, a heterocyclic group having 4 to 10 carbon atoms, a heterocyclic oxy group having 4 to 10 carbon atoms, an acyl group having 2 to 10 carbon atoms, or a nitro group. R⁵² to R⁵⁴ each independently represent hydrogen, an alkyl group having 1 to 10 carbon atoms, a cycloalkyl group having 4 to 10 carbon atoms, or an aryl group having 6 to 15 carbon atoms. a represents an integer of 0 to 3, b represents 0 or 1, c represents an integer of 0 to 5, d represents 0 or 1, e represents an integer of 0 to 4, f represents an integer of 0 to 2, g, h, and i each independently represent an integer of 0 to 2, j, k, and l each independently represent 0 or 1, and m, n, and o each independently represent an integer of 0 to 10. In a case where Y¹ represents nitrogen, R³⁷ represents a nitro group, X⁴ represents an arylene group having 6 to 15 carbon atoms, R⁴⁹ represents hydrogen, an alkyl group having 1 to 10 carbon atoms, a cycloalkyl group having 4 to 10 carbon atoms, an aryl group having 6 to 15 carbon atoms, a haloalkyl group having 1 to 10 carbon atoms, a haloalkoxy group having 1 to 10 carbon atoms, a heterocyclic group having 4 to 10 carbon atoms, a heterocyclic oxy group having 4 to 10 carbon atoms, an acyl group having 2 to 10 carbon atoms, or a nitro group.) 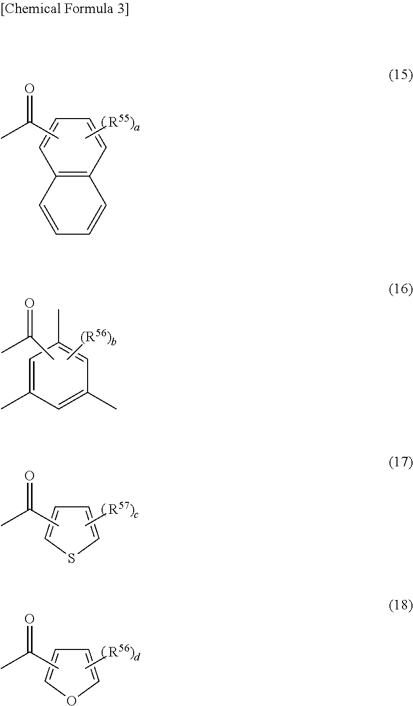
(In the general formula (15) to (18), R⁵⁵ to R⁵⁸ each independently represent an alkyl group having 1 to 10 carbon atoms, acycloalkyl group having 4 to 10 carbon atoms, an aryl group having 6 to 15 carbon atoms, an alkoxy group having 1 to 10 carbon atoms, or a hydroxyalkyl group having 1 to 10 carbon atoms, a represents an integer of 0 to 7, b represents an integer of 0 to 2, and c and d each independently represent an integer of 0 to 3.)

11. The negative photosensitive resin composition according to claim 10,
wherein the (C1-1) oxime ester-based photo initiator contains a compound represented by the general formula (13).

12. The negative photosensitive resin composition according to claim 10,
wherein the (C1-1) oxime ester-based photo initiator contains a compound represented by the general formula (12) and/or a compound represented by the general formula (13), and
in the general formula (12) and the general formula (13), Y¹ and Y² represent carbon or nitrogen, R⁴⁶ and R⁴⁷ include at least an alkenyl group having 1 to 10 carbon atoms, and R⁴⁹ and R⁵⁰ include at least an alkenyl group having 1 to 10 carbon atoms.

13. The negative photosensitive resin composition according to claim 1,
the negative photosensitive resin composition further comprising, as a (F) polyfunctional thiol compound, one or more selected from the group consisting of a compound represented by the general formula (83), a compound represented by the general formula (84), and a compound represented by the general formula (85). 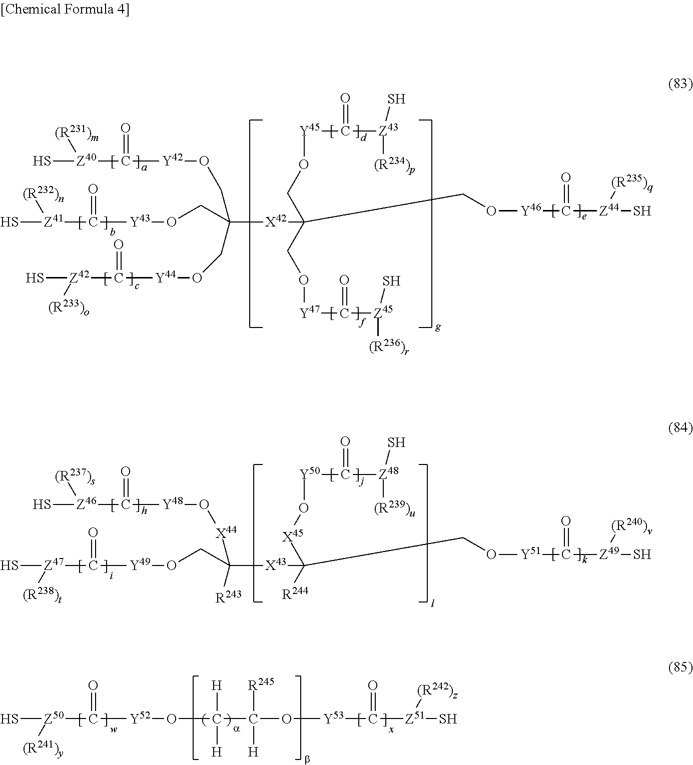
(In the general formulas (83) to (85), X⁴² and X⁴³ represent a divalent organic group. X⁴⁴ and X⁴⁵ each independently represent a direct bond or an alkylene chain having 1 to 10 carbon atoms. Y⁴² to Y⁵³ each independently represent a direct bond, an alkylene chain having 1 to 10 carbon atoms, or a group represented by general formula (86). Z⁴⁰ to Z⁵¹ each independently represent a direct bond or an alkylene chain having 1 to 10 carbon atoms. R²³¹ to R²⁴² each independently represent an alkylene chain having 1 to 10 carbon atoms. R²⁴³ to R²⁴⁵ each independently represent hydrogen or an alkyl group having 1 to 10 carbon atoms. a, b, c, d, e, f, h, i, j, k, w, and x each independently represent 0 or 1. g and l each independently represent an integer of 0 to 10. m, n, o, p, q, r, s, t, u, v, y, and z each independently represent an integer of 0 to 10. α and β each independently represent an integer of 1 to 10.) 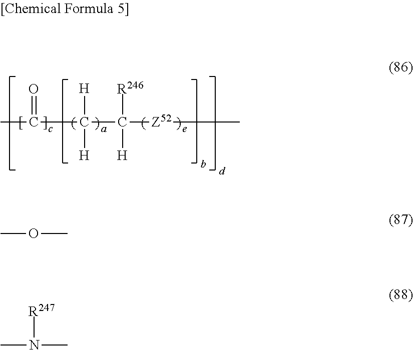
(In the general formula (86), R²⁴⁶ represents hydrogen or an alkyl group having 1 to 10 carbon atoms. Z⁵² represents a group represented by general formula (87) or a group represented by general formula (88). a represents an integer of 1 to 10, b represents an integer of 1 to 4, c represents 0 or 1, d represents an integer of 1 to 4, and e represents 0 or 1. In a case where c is 0, d represents 1 In the general formula (88), R²⁴⁷ represents hydrogen or an alkyl group having 1 to 10 carbon atoms.)

14. The negative photosensitive resin composition according to claim 13,
the negative photosensitive resin composition further comprising, as a (B) radical polymerizable compound, an (B4) alicyclic group-containing radical polymerizable compound,
wherein the (B4) alicyclic group-containing radical polymerizable compound contains a polycyclic condensed alicyclic skeleton.

15. The negative photosensitive resin composition according to claim 1,
wherein the (C1) photo initiator further contains one or more selected from the group consisting of an (C1-2) α-aminoketone-based photo initiator, an (C1-3) acylphosphine oxide-based photo initiator, and a (C1-4) biimidazole-based photo initiator, and
a content ratio of the (C1-) oxime ester-based photo initiator in the (C) photo initiator is 55 to 95% by mass.

16. The negative photosensitive resin composition according to claim 1,
the negative photosensitive resin composition further comprising, as a (B) radical polymerizable compound, one or more selected from the group consisting of a (B1) fluorene skeleton-containing radical polymerizable compound and/or an (B2) indane skeleton-containing radical polymerizable compound.

17. The negative photosensitive resin composition according to claim 1,
the negative photosensitive resin composition further comprising, as a (B) radical polymerizable compound, a (B3) flexible chain-containing aliphatic radical polymerizable compound, and
the (B3) flexible chain-containing aliphatic radical polymerizable compound has at least one lactone-modified chain and/or at least one lactam-modified chain.

18. The negative photosensitive resin composition according to claim 1,
wherein the negative photosensitive resin composition is used for collectively forming a step shape for a pixel defining layer in an organic EL display.

19. A cured film obtained by curing the negative photosensitive resin composition according to claim 1.

20. An organic EL display,
wherein an optical density of the cured film according to claim 19 per film thickness of 1 μm is 0.3 to 5.0, and
the cured film is included as one or more selected from the group consisting of a pixel defining layer, an electrode insulation layer, a wiring insulation layer, an interlayer insulation layer, a TFT planarization layer, an electrode planarization layer, a wiring planarization layer, a TFT protective layer, an electrode protective layer, a wiring protective layer, and a gate insulation layer.

21. The organic EL display according to claim 20,
wherein the cured film has a cured pattern with a step shape.

22. The organic EL display according to claim 21,
wherein in the cured pattern with the step shape, a film thickness difference (ΔT_{FT-HT}) μm between (T_{FT}) and (T_{HT}) is 1.5 to 10.0 μm, where a film thickness of a thick film part is denoted by (T_{FT}) μm, whereas a film thickness of a thin film part is denoted by (T_{HT}) μm.

23. A method for manufacturing an organic EL display, the method comprising:
a step of forming, on a substrate, a coating film of the negative photosensitive resin composition according to claim 1;
a step of irradiating the coating film of the negative photosensitive resin composition with an active actinic ray through a photomask;
a step of developing with an alkaline solution to form a pattern of the negative photosensitive resin composition; and
a step of heating the pattern to obtain a cured pattern of the negative photosensitive resin composition.

24. The method for manufacturing an organic EL display according to claim 23,
wherein the photomask is a photomask that has a pattern including a light-transmitting portion and a light-blocking portion, the photomask being a half-tone photomask including, between the light-transmitting portion and the light-blocking portion, a partial light-transmitting portion that is lower in transmittance than a value of the light-transmitting portion and higher in transmittance than a value of the light-blocking portion.
